(19) United States
(12) Patent Application Publication
(10) Pub. No.: US 2021/0363545 A1
(43) Pub. Date: Nov. 25, 2021

(54) GENETIC SELECTION MARKERS BASED ON ENZYMATIC ACTIVITIES OF THE PYRIMIDINE SALVAGE PATHWAY

(71) Applicant: Medizinische Universität Innsbruck, Innsbruck (AT)

(72) Inventors: Fabio GSALLER, Innsbruck (AT); Hubertus HAAS, Innsbruck (AT)

(73) Assignee: Medizinische Universität Innsbruck, Innsbruck (AT)

(21) Appl. No.: 17/252,164

(22) PCT Filed: Jun. 7, 2019
(86) PCT No.: PCT/EP2019/065020

§ 371 Date: Dec. 14, 2020

(30) Foreign Application Priority Data
Jun. 19, 2018 (GB) 1810053.7

(51) Int. Cl. C12N 15/90; C12N 15/65

(52) U.S. Cl. CPC C12N 15/902; C12Y 305/04001; C12N 15/65

(57) ABSTRACT

The present invention relates to a method of site-directed integration into a genetic locus encoding at least one activity of the pyrimidine salvage pathway in a host cell, wherein said activity of the pyrimidine salvage pathway is purine/cytosine permease (FcyB), cytosine deaminase (FcyA), uracil-phosphoribosyl-transferase (Uprt), concentrative nucleoside transporter (CntA) or uridine kinase (UK), comprising: a) providing a host cell comprising a functional copy of the genetic locus encoding at least one activity of the pyrimidine salvage pathway; (b) introducing a gene or sequence of interest into said host cell via transformation of an integrative nucleic acid construct which comprises 3′ and/or 5′ of the gene or sequence of interest flanks being homologous to said genetic locus or which carries a sequence being homologous to said genetic locus of the pyrimidine salvage pathway and thus allowing for a homologous recombination at said genetic locus, wherein said homologous recombination is capable of causing an inactivation or reduction of the activity encoded by said genetic locus; (c) growing a transformed host cell under selective medium conditions, wherein said medium comprises an efficient amount of 5-flucytosine (5-FC), 5-fluorouracil (5-FU) or 5-fluorouridine (5-FUR); and (d) selecting a host cell which is capable of growing under the medium conditions of step (c). Also envisaged is a host cell, comprising at least one gene or sequence of interest in one or more genetic loci encoding an activity of the pyrimidine salvage pathway wherein said gene or sequence of interest replaces or partially replaces the sequence encoding said at least one activity of the pyrimidine salvage pathway at said locus, the use of such a host cell for the production of several activities, as well as the use of a genetic locus encoding at least one activity of the pyrimidine salvage pathway in a host cell in a process of transforming said host cell or a process of genetically modifying said host cell.

### CROSS-REFERENCE TO RELATED APPLICATIONS

This application is a national stage application, filed under 35 U.S.C. § 371 of International Application No. PCT/EP2019/065020, filed Jun. 7, 2019, which claims the benefit of priority under 35 U.S.C. § 119(e) to Great Britain Application No. 1810053.7, filed Jun. 19, 2018, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 2, 2021, is named 32949US—Aspergillus Knockin-Vers03-US Verfahren_ST25.txt and is 364 KB bytes in size.

### FIELD OF THE INVENTION

The present invention relates to a method of site-directed integration into a genetic locus encoding at least one activity of the pyrimidine salvage pathway in a host cell, wherein said activity of the pyrimidine salvage pathway is purine/cytosine permease (FcyB), cytosine deaminase (FcyA), uracil-phosphoribosyl-transferase (Uprt), concentrative nucleoside transporter (CntA) or uridine kinase (UK), comprising: a) providing a host cell comprising a functional copy of the genetic locus encoding at least one activity of the pyrimidine salvage pathway; (b) introducing a gene or sequence of interest into said host cell via transformation of an integrative nucleic acid construct which comprises 3′ and/or 5′ of the gene or sequence of interest flanks being homologous to said genetic locus or which carries a sequence being homologous to said genetic locus of the pyrimidine salvage pathway and thus allowing for a homologous recombination at said genetic locus, wherein said homologous recombination is capable of causing an inactivation or reduction of the activity encoded by said genetic locus; (c) growing a transformed host cell under selective medium conditions, wherein said medium comprises an efficient amount of 5-flucytosine (5-FC), 5-fluorouracil (5-FU) or 5-fluorouridine (5-FUR); and (d) selecting a host cell which is capable of growing under the medium conditions of step (c). Also envisaged is a host cell, comprising at least one gene or sequence of interest in one or more genetic loci encoding an activity of the pyrimidine salvage pathway wherein said gene or sequence of interest replaces or partially replaces the sequence encoding said at least one activity of the pyrimidine salvage pathway at said locus, the use of such a host cell for the production of several activities, as well as the use of a genetic locus encoding at least one activity of the pyrimidine salvage pathway in a host cell in a process of transforming said host cell or a process of genetically modifying said host cell.

### BACKGROUND OF THE INVENTION

On the brink of a new genomic sequencing approach, the Earth BioGenome project (EBP), which aims at the determination of the genomic sequence of a member of each eukaryotic family, followed by the sequencing of at least one species of the up to 200,000 genera and finally the sequencing of all known eukaryotic sequences (Sciences, doi:10.1126/science.aal0824), the study of gene functions becomes more and more important. In addition, functional manipulation is a perquisite for biotechnological approaches involving, for example, the expression of recombinant proteins or production of medically important compounds including life-saving antibiotics.

However, while the sequencing technology has made tremendous progress in the last decades, in the field of functional genomics in many cases the same tools which were designed 40 years ago are still in use. Typically, genes are simply replaced by antibiotic resistance markers to see what phenotypic effects result. The generation of a strain with multiple deletions, however, cannot be achieved with this method, because the availability of resistance markers is limited. In consequence, additional gene disruption methods based on homologous recombination have been developed. For example, by flanking a resistance cassette with recognition sites for site-specific recombinases, such as Flp/Flp recombination target (FRT) and Cre/loxP, the cassette can precisely be removed and used again for the next deletion step. Yet, remnants of these excisions are left in the chromosome after each deletion step in the form of FRT or loxP sites, respectively. These may cause problems and lead to chromosomal deletions or inversions since the recognition sites in the chromosome can become recombined. Another disadvantage of this approach is the time-consuming selection for positive clones that have lost the resistance cassette.

A solution to this problem is provided by counter-selectable marker systems. Known examples of such systems include the fusaric acid (tetAR), streptomycin (rpsL), and sucrose (sacB) sensitivity systems in bacteria (Reyrat et al., 1998. Infect. Immun. 66: 4011-4017). One of the most popular and widely used markers, in particular in yeast genetics, is URA3, which encodes orotidine 5′-phosphate decarboxylase (ODCase), an enzyme of the de novo pyrimidine biosynthesis pathway. Loss of ODCase activity typically leads to a lack of cell growth unless uracil or uridine is supplemented. The presence of the URA3 gene in yeast restores ODCase activity, facilitating growth on media not supplemented with uracil or uridine, thus allowing for a selection of organisms carrying the gene. In contrast, if the compound 5-FOA (5-Fluoroorotic acid) is added to the media, the active ODCase will convert 5-FOA into the toxic suicide inhibitor 5-fluorouracil causing cell death, which allows for selection against organisms carrying the gene, albeit only in the presence of uracil or uridine (Heslot and Gaillardin, Molecular Biology and Genetic Enegineering of Yeasts, 1992).

Yet, the number and adaptability of suitable counter-selection markers is still low. Furthermore, auxotrophic markers such as URA3, require constant complementation with essential compounds. There is hence a need for the provision of new, versatile counter-selection methods based on markers, which are non-auxotrophic and thus allow for an efficient selection-marker free transformation of suitable organisms.

### OBJECTS AND SUMMARY OF THE INVENTION

The present invention addresses this need and presents a method of site-directed integration into a genetic locus encoding at least one activity of the pyrimidine salvage pathway in a host cell, wherein said activity of the pyrimidine salvage pathway is purine/cytosine permease (FcyB), cytosine deaminase (FcyA), uracil-phosphoribosyl-transferase (Uprt), concentrative nucleoside transporter (CntA) or uridine kinase (UK), comprising: (a) providing a host cell comprising a functional copy of the genetic locus encoding at least one activity of the pyrimidine salvage pathway; (b) introducing a gene or sequence of interest into said host cell via transformation of an integrative nucleic acid construct which comprises 3′ and/or 5′ of the gene or sequence of interest flanks being homologous to said genetic locus or which carries a sequence being homologous to said genetic locus of the pyrimidine salvage pathway and thus allowing for a homologous recombination at said genetic locus, wherein said homologous recombination is capable of causing an inactivation or reduction of the activity encoded by said genetic locus(c) growing a transformed host cell under selective medium conditions, wherein said medium comprises an efficient amount of 5-flucytosine (5-FC), 5-fluorouracil (5-FU) or 5-fluorouridine (5-FUR); and (d) selecting a host cell which is capable of growing under the medium conditions of step (c).

The present inventors surprisingly found that genes of the pyrimidine salvage pathway such as purine/cytosine permease (FcyB), cytosine deaminase (FcyA), uracil-phosphoribosyl-transferase (Uprt), concentrative nucleoside transporter (CntA) or uridine kinase (UK) can advantageously be used as counter-selection markers for several organism groups including bacteria, fungi and plants. In the pyrimidine salvage pathway—in contrast to the pyrimidine de novo synthesis pathway—pyrimidine nucleotides are synthesized from intermediates in the degradation pathway of nucleotides. Accordingly, the pyrimidine salvage pathway is used to recover bases and nucleosides formed during degradation or RNA and DNA. An interruption of the pathway, e.g. by deletion of a gene encoding a pathway enzyme, will however not lead to lethal consequences, because the pyrimidine supply is ensured by the activities of the de novo synthesis pathway. In contrast, if prodrug suicide inhibitor compounds like 5-flucytosine (5-FC), 5-fluorouracil (5-FU) or 5-fluorouridine (5-FUR) are added to growth media, active purine/cytosine permease (FcyB), cytosine deaminase (FcyA), concentrative nucleoside transporter (CntA), uracil-phosphoribosyl-transferase (Uprt), or uridine kinase (UK) will transport and ultimately convert these compounds into the toxic substance 5-fluorouridine monophosphate (5-FUMP) or 5-fluoro deoxyuridine monophosphate (5-FdUMP) which are further converted into 5-FUTP or 5-FdUTP, respectively and eventually interfere with RNA and DNA biosynthesis as well as protein metabolism (see also FIG. 1).

On the basis of these markers, homologous, site directed integration of any gene or sequence of interest into the genetic loci of fcyB, fcyA, cntA, uprt, or uk becomes possible. Employment of 5-FC, 5-FU and/or 5-FUR (depending on the genetic locus used) during a growth phase very effectively selects only those organisms which comprise a functional disruption of the fcyB, fcyA, cntA, uprt, or uk locus. This functional disruption is typically associated with the integration of a gene or sequence of interest at said locus. The employment of additional selection markers such as antibiotics markers etc. is advantageously not necessary, but may—under certain, very specific circumstances—additionally be envisaged. Subsequent to the integration event, the usage of 5-FC, 5-FU and/or 5-FUR is no longer required. Transformants can, thus, without any supplementation or additional selection pressure be grown in variable media—depending on the envisaged use and the gene or sequence of interest inserted. The mentioned markers can further, advantageously, be used as single counter-selection markers, or, due to the employment of different prodrug suicide inhibitors, as multiple selection markers, e.g. in various combinations (see also FIG. 1 and the details therein with respect to the combination of markers).

The described approach is believed to significantly advance and facilitate genetic manipulation of several groups of organisms comprising the mentioned activities in the pyrimidine salvage pathway in order to study specific gene functions and to genetically engineer biotechnological relevant species.

In a preferred embodiment of the method of the present invention, the integrative nucleic acid construct as mentioned above comprises a control element such as a promoter or a terminator sequence which are operably linked to the gene or sequence of interest or the sequence to be expressed.

In a further preferred embodiment of the method of the present invention, said integrative nucleic acid construct does not comprise a nucleic acid sequence encoding a marker gene for selection of a genetically transformed host cell.

In a further preferred embodiment of the method of the present invention said site-directed integration into a genetic locus encoding an activity of the pyrimidine salvage pathway in a host cell comprises the integration into two or more genetic loci encoding an activity of the pyrimidine salvage pathway in a host cell. The present inventors accordingly and quite surprisingly could make use of more than one locus and the associated activity of the pyrimidine salvage pathway such as purine/cytosine permease (FcyB), cytosine deaminase (FcyA), uracil-phosphoribosyl-transferase (Uprt), concentrative nucleoside transporter (CntA) or uridine kinase (UK) as counter-selection markers per host cell for several organism groups including bacteria, fungi and plants. Thus, homologous, site directed integration of any gene or sequence of interest into two or more of the genetic loci of fcyB, fcyA, cntA, uprt, or uk becomes possible while using the same group of prodrug suicide inhibitor compounds like 5-flucytosine (5-FC), 5-fluorouracil (5-FU) or 5-fluorouridine (5-FUR).

In a further preferred embodiment of the present invention said site-directed integration is performed in a sequential order in said two or more genetic loci encoding an activity of the pyrimidine salvage pathway in a host cell. Advantageously, by using an already transformed host cell to introduce a second or further gene of interest into a genetic locus encoding another activity of the pyrimidine salvage pathway it becomes possible to further engineer and modify said host cell. The presence of more than gene or sequence of interest accordingly allows for the provision of complex pathways and/or the implementation of complex biotechnological production pattern etc.

In a particularly preferred embodiment of the present invention said two or more genetic loci encoding an activity of the pyrimidine salvage pathway in a host cell are used for site-directed integration in one of the following orders and/or combinations:
(i) (1) fcyB; (2) fcyA;
(ii) (1) fcyB; (2) uprt;
(iii) (1) fcyB; (2) cntA, or uk;
(iv) (1) fcyA; (2) uprt;
(v) (1) fcyA; (2) cntA, or uk;
(vi) (1) uprt; (2) cntA, or uk;
(vii) (1) fcyB; (2) fcyA; (3) uprt;
(viii) (1) fcyB; (2) fcyA; (3) cntA, or uk;
(ix) (1) fcyB, (2) uprt; (3) cntA, or uk;
(x) (1) fcyA, (2) uprt; (3) cntA, or uk;
(xi) (1) fcyB; (2) fcyA; (3) uprt; (4) cntA, or uk

In yet another preferred embodiment of the present invention, said gene or sequence of interest encodes for one or more enzymatic activities, wherein said enzymatic activity comprises an isomerase, oxidase, reductase, oxidoreductase, hydrolase, ligase, lyase, cellulase, chitinase, amylase, lactase, glucosidase, xylanase, transferase, esterase, lipase, mannosidase, glucanase, protease, phytase, invertase, peroxidase, peptidase, pectinase, chymosin or pepsin.

In a further preferred embodiment, said gene or sequence of interest encodes one or more of: (i) an activity involved in the production of carbohydrates, fatty acids or lipids, (ii) a pharmaceutically active protein or peptide, (iii) an antibiotic or an activity involved in the production of an antibiotic, (iv) an activity involved in the production of biofuels, (v) an activity involved in the production of foodstuff or animal feedstuff, (vi) an activity involved in production of vitamins or dietary supplements, (vii) an activity involved in the production of amino acids, (viii) an activity involved in the production of cosmetic ingredients, (ix) an activity involved in the production of organic raw materials, or (x) a protein used in metabolic engineering or synthetic biology such as in cell factory generation or optimization.

In yet another preferred embodiment it is envisaged that the gene or sequence of interest encodes a homologous activity of the host cell, which is provided in a modified amount, preferably in an increased amount, or in a differently controlled manner.

In another preferred embodiment, said gene or sequence of interest encodes a biomolecular marker protein. It is particularly preferred that the biomolecular marker protein is a fluorescent protein. Envisaged examples are GFP or derivatives thereof.

In a further preferred embodiment, said gene or sequence of interest comprises, essentially consists of or consist of an RNA expression cassette, wherein said RNA expression cassette provides one or more elements required for RNA gene silencing.

It is further preferably envisaged that said gene or sequence of interest has a codon usage or a dicodon usage, which is adapted to the codon usage or dicodon usage of the host cell.

In a specific embodiment of the method according to the present invention, said host cell is a bacterium, preferably of the genus Klebsiella, Clostridium, Bacillus, Arthobacter, Streptomyces, Corynebacterium, Erwinia, Xanthomonas, Lactobacillus, Caldicellulosiruptor, Pseudomonas, Alcanivorax, Brevibacterium, Bifidobacterium, Escherichia, or Staphylococcus; or a fungus, preferably of the genus Aspergillus, Candida, Saccharomyces, Ustilago, Cryptococcus, Fusarium, Rhizopus, Magnaporthe, Komagataella, Trichderma, Penicillium, Acremonium, Mucor, Alternaria, Botrytis, Endothia, Rhizoctonia, Sclerotinia, Klyveromyces, Torulopsis, Sporotrichum, Geotrichum, Verticillium, Botryosphaeria, Trichothecium, Hansenula, Schizosaccharomyces, Brettanomyces, or Neurospora; or a plant; or an alga.

It is particularly preferred that the host cell is an Aspergillus niger, Aspergillus oryzae, Aspergillus fumigatus, Komagataella phaffii, Trichoderma reesei, Penicillium chrysogenum, Acremonium chrysogenum, Candida albicans, Ustilago maydis, Cryptococcus neoformans, Fusarium oxysporum, Rhizopus delemar, or Magnaporthe oryzae cell.

In a further preferred embodiment, the method as mentioned above comprises additionally genetically modifying said host cell.

It is particularly preferred that said additional genetic modification is a blocking of a further activity, an increase or decrease of the expression of a gene, a silencing of a gene, a deletion of one or more genes or loci or gene clusters, or an introduction of one or more additional homologous genes or of one or more heterologous genes.

In a further aspect the present invention relates to a host cell, comprising at least one gene or sequence of interest as mentioned above in one or more genetic loci encoding an activity of the pyrimidine salvage pathway, wherein said gene or sequence of interest replaces or partially replaces the sequence encoding said at least one activity of the pyrimidine salvage pathway at said locus.

In a preferred embodiment of said host cell said one or more genetic loci encoding an activity of the pyrimidine salvage pathway are at least two genetic loci selected from the following group and used in the indicated order:
(i) (1) fcyB; (2) fcyA;
(ii) (1) fcyB; (2) uprt;
(iii) (1) fcyB; (2) cntA, or uk;
(iv) (1) fcyA; (2) uprt;
(v) (1) fcyA; (2) cntA, or uk;
(vi) (1) uprt; (2) cntA, or uk;
(vii) (1) fcyB; (2) fcyA; (3) uprt;
(viii) (1) fcyB; (2) fcyA; (3) cntA, or uk;
(ix) (1) fcyB, (2) uprt; (3) cntA, or uk;
(x)(1) fcyA, (2) uprt; (3) cntA, or uk; and
(xi) (1) fcyB; (2) fcyA; (3) uprt; (4) cntA, or uk.

In a further aspect the present invention relates to the use of a host cell as defined above for the production of an enzymatic activity, an activity involved in the production of carbohydrates, fatty acids or lipids, a pharmaceutically active protein or peptide, an antibiotic or an activity involved in the production of an antibiotic, an activity involved in the production of biofuels, an activity involved in the production of foodstuff or animal feedstuff, an activity involved in productions of vitamins or dietary supplements, an activity involved in the production of amino acids, an activity involved in the cosmetic ingredients, an activity involved in the production of organic raw material, or of proteins used in metabolic engineering or synthetic biology.

Finally, in a further aspect the present invention relates to the use of a genetic locus encoding at least one activity of the pyrimidine salvage pathway in a host cell, wherein said activity of the pyrimidine salvage pathway is purine/cytosine permease (FcyB), uracil-phosphoribosyl-transferase (Uprt), concentrative nucleoside transporter (CntA) or uridine kinase (UK) as selection marker in a process of transforming said host cell or a process of genetically modifying said host cell.

### BRIEF DESCRIPTION OF THE DRAWINGS

FIG. 1 shows the metabolic conversion of 5-FC, 5-FU and 5-FUR into cell toxic metabolites by enzymes of the pyrimidine salvage pathway. Protein activities used for 5-FC, 5-FU or 5-FUR based selection are displayed as grey arrows. The order for a potential sequential use of respective loci for multiple knock-in is indicated. A detailed description of the selective conditions for each locus is provided below and in the Examples.

FIG. 2 shows a representation of the generation of knock-in constructs for genetic loci according to the present invention.

FIG. 3 depicts the deletion of fcyB/uprt/cntA/uk through homologous recombination with simultaneous integration of a DNA cassette of interest.

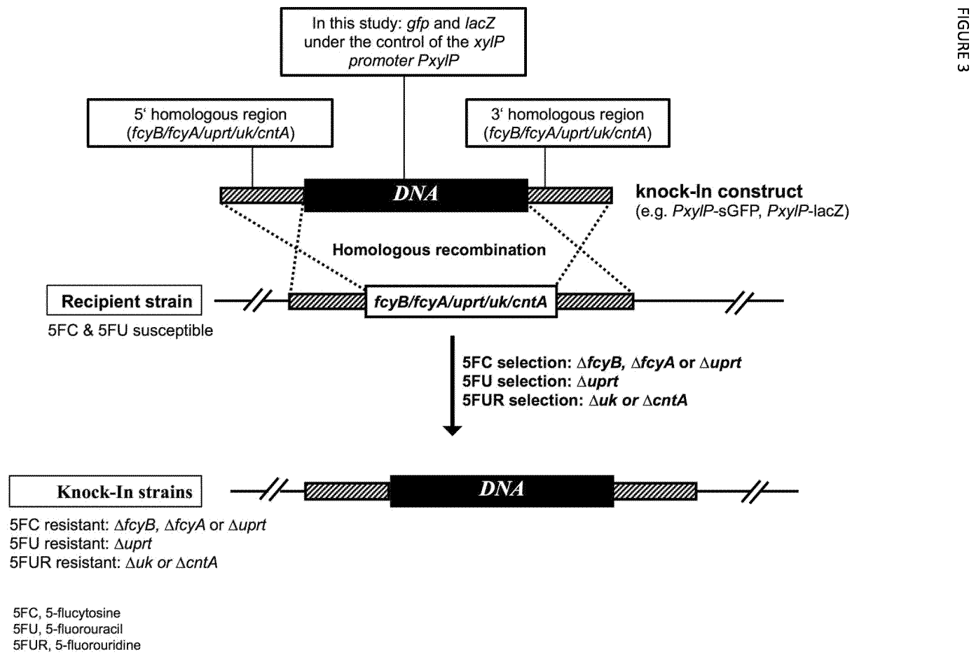

FIG. 4 shows resistance of the generated GFP and lacZ reporter strains, which have been tested on solid Aspergillus minimal medium. Disruptions of fcyB (fcyBsGFP & fcyBlacZ) results in resistance to 5-FC. Replacement of uprt (uprtsGFP & uprtlacZ) by a knock-in construct results in 5-FC but also 5-FU resistance. For detection of sGFP expression as well as lacZ activity a control plate 5-FC (0 mg/ml) was used. A GFP signal (see panel on the right side of the Figure) could be detected in all strains expressing sGFP. lacZ expression was detected by adding an X-Gal containing layer of Agar on the top of colonies. X-Gal was successfully converted to its blue product by lacZ expressing strains.

FIG. 5 shows Southern analyses of strains that have been transformed with either the fcyB (GFP and lacZ) or the uprt (GFP and lacZ) knock-in cassettes.

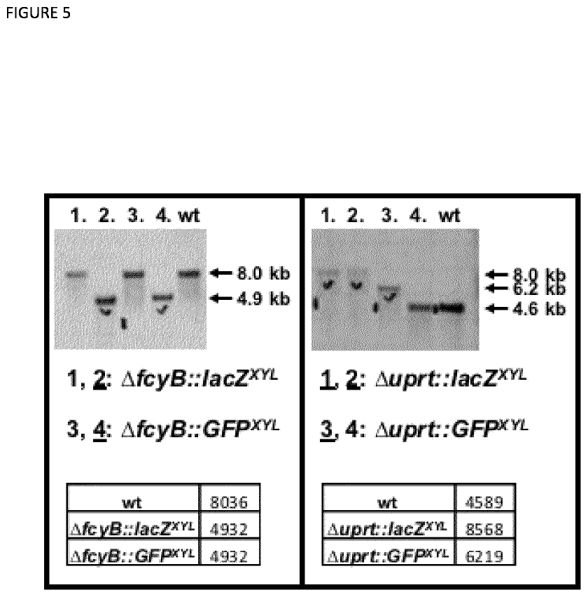

FIG. 6 illustrates the restriction pattern detected in the Southern analyses shown in FIG. 5.

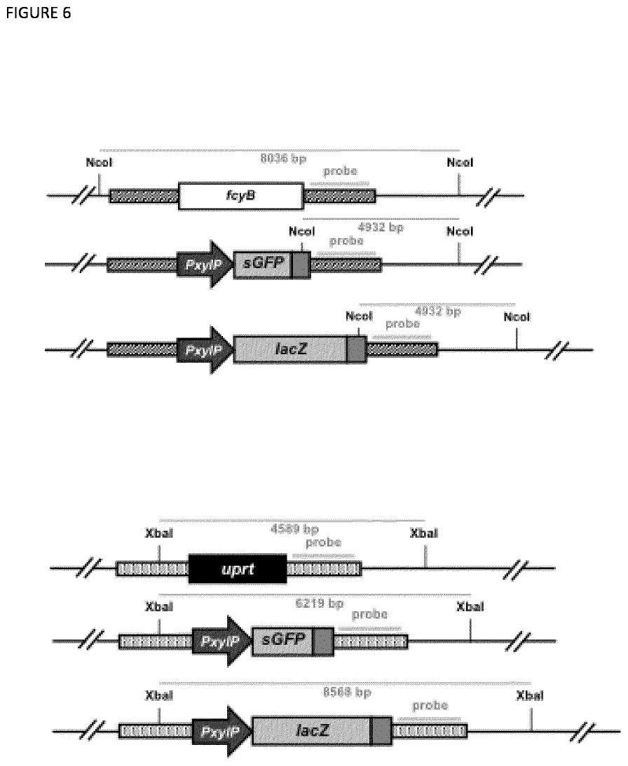

FIG. 7 shows wild-type (wt) and single mutants ΔfcyB, ΔfcyA and Δuprt, as well as their 5-FC and 5-FU resistance phenotypes.

FIG. 8 Illustrates visual confirmation of functional fluorescent proteins in the strain RFP^{PER}GFP^{MIT}BFP^{CYT} (left panel). The encoding genes have been introduced sequentially into wt following the use of loci in the order fcyB, fcyA and uprt. RFP (mKate2) localizes to the peroxisome, GFP (sGFP) to the mitochondrium and BFP (mTagBFP2) to the cytoplasm. Phenotypic comparison of wt and RFP^{PER}GFP^{MIT}BFP^{CYT} at pH5 and pH7 is shown in the right panel. PTS1=peroxisomal targeting sequence; MTS=mitochondrial targeting sequence.

FIG. 9 shows in (a) resistance phenotypes of ΔcntA and Δuk, in (b) visualization of luciferase activity in ΔcntA and Δuk knock-ins and in (c) Southern analyses as well as the corresponding restriction length pattern of strains that have been transformed with either the cntA or the uk knock-in cassettes. Each construct was transformed in wt (mutants 1 and 2) and the triple deletion background ΔfcyBΔfcyAΔuprt (mutants 5 and 6).

FIG. 10 provides schematic drawings of the genomic situation after transformation depicted in FIG. 9.

FIG. 11 depicts the replacement of self-encoded selectable markers fcyB, fcyA and uprt by DOI. (a) Scheme of the generation of knock-in constructs. 5′ and 3′ NTR (PCR1) of the respective loci as well as the DOI (PCR2; GFP or lacZ reporter cassette) are amplified from genomic DNA (gDNA) and plasmid DNA, respectively. Both, NTRs and DOI contain overlapping DNA (grey line) for subsequent, connection via fusion PCR, yielding the knock-in constructs. (b) Double crossover homologous recombination based replacement of fcyB, fcyA or uprt by DOI. Transformation selection was carried out using 5FC (fcyB and fcyA locus) or 5FU (uprt locus). (c) Visualization of GFP as well as LacZ expression in the corresponding knock-in strain.

FIG. 12 depicts the genomic insertion of the PcCluster A. fumigatus and expression analysis of penicillin G biosynthetic genes. (a) To facilitate genomic integration of the PcCluster at the fcyB locus, the plasmid pfcyB-PcCluster comprising the respective DNA (^{˜}17 kb) as well as fcyB 5′ and 3′ NTRs was generated. Linearization of this plasmid with PmeI allows homologous recombination based replacement of fcyB coding sequence with DNA containing the PcCluster. (b) Expression of functional pcbAB, pcbC and penDE was monitored using Northern analysis (gpdA was used as reference).

FIG. 13 shows P. chrysogenum and F. oxysporum strains with genomically replaced selectable markers CD and UPRT and the resulting resistance phenotype. For the visualization of GFP expression 10⁴ spores of each strain were point-inoculated on solid AMM (P. chrysogenum) and PDA (F. oxysporum) followed by 72 h incubation at 25° C. 5FC/5FU resistance phenotypes of the respective mutants are illustrated.

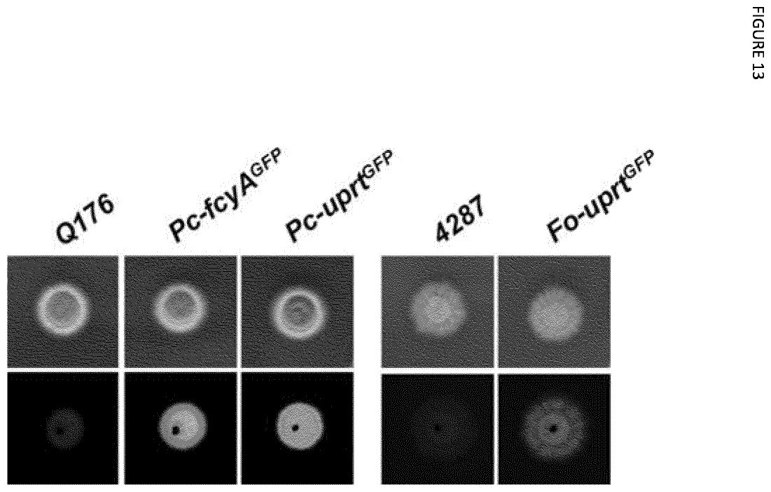

FIG. 14 depicts Southern analyses of strains described in Examples 6 to 10.

FIG. 15 shows 5-FC and 5-FU susceptibility in test with knock-in strains of A. fumigatus using GFP and LacZ. For each strain 10⁴ spores were point inoculated on solid medium. A. fumigatus strains were grown on solid AMM. A. fumigatus strains were incubated for 48 h at 37° C. Resistance phenotypes of all mutants analyzed were in accordance with the absence of individual salvage activities.

FIG. 16 shows 5-FC and 5-FU susceptibility in test with knock-in strains of A. fumigatus using RFP^{PER}GFP^{MIT}BFP^{CYT}. For each strain 10⁴ spores were point inoculated on solid medium. A. fumigatus strains were grown on solid AMM. A. fumigatus strains were incubated for 48 h at 37° C. Resistance phenotypes of all mutants analyzed were in accordance with the absence of individual salvage activities.

FIG. 17 shows 5-FC and 5-FU susceptibility in test with knock-in strains of P. chrysogenum and F. oxysporum using GFP. For each strain 10⁴ spores were point inoculated on solid medium. P. chrysogenum strains were grown on solid AMM, whereas PDA was used for F. oxysporum. P. chrysogenum and F. oxysporum for 72 h at 25° C. Resistance phenotypes of all mutants analyzed were in accordance with the absence of individual salvage activities.

FIG. 18 depicts beta-galactosidase staining to screen for LacZ-positive transformants. After determining LacZ activities of each transformant (a), for each mutant locus 10 transformants showing LacZ-positive phenotypes underwent Southern analysis (b). Strains were grown for 48 h at 37° C. on solid AMM before pouring an additional layer (5 ml) of X-Gal containing agar on the top of colonies.

FIG. 19 depicts the generation of a pfcyB-PcCluster. After amplification of fcyB 5′ and 3′ NTRs from A. fumigatus genomic DNA (Af-gDNA) (A), the purified products were assembled (NEBuilder®) into pUC19L (B). The yielding plasmid pfcyB was then linearized (C) using primers BB-pfcyB-FW/RV. Simultaneously, two overlapping fragments comprising the penicillin G biosynthetic cluster were amplified from P. chrysogenum genomic DNA (Pc-gDNA) employing primer pairs PcFrag1-FW/RV and PcFrag2-FW/RV (D). In the last step PcFrag1, PcFrag2 and linear pfcyB were assembled (E) giving rise to pfcyB-PcCluster.

FIG. 20 shows plasmid templates used for the generation of DOIs used in this work. For the amplification of the reporter cassettes comprising sGFP, lacZ, mKate2PER, sGFPMIT the primer pair P1/P2 was used. As templates pX-sGFP, pX-mKate2PER, pX-sGFPMIT, pX-lacZ were. An mTagBFP2 containing cassette was amplified from pAN-mTagBFP2 using primers hph-FW/hph-RV. For F. oxysporum, the GFP reporter cassette was amplified from pgpdA-GFP using primers FoGFP-Fw/Rv. MTS=mitochondrial targeting sequence; PTS=peroxisomal targeting sequence.

### DETAILED DESCRIPTION OF THE EMBODIMENTS

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method, procedure or use there is no time or time interval coherence between the steps, i.e. the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect a method of site-directed integration into a genetic locus encoding at least one activity of the pyrimidine salvage pathway in a host cell, wherein said activity of the pyrimidine salvage pathway is purine/cytosine permease (FcyB), cytosine deaminase (FcyA), uracil-phosphoribosyl-transferase (Uprt), concentrative nucleoside transporter (CntA) or uridine kinase (UK), comprising: a) providing a host cell comprising a functional copy of the genetic locus encoding at least one activity of the pyrimidine salvage pathway; (b) introducing a gene or sequence of interest into said host cell via transformation of an integrative nucleic acid construct which comprises 3′ and/or 5′ of the gene or sequence of interest flanks being homologous to said genetic locus or which carries a sequence being homologous to said genetic locus of the pyrimidine salvage pathway and thus allowing for a homologous recombination at said genetic locus, wherein said homologous recombination is capable of causing an inactivation or reduction of the activity encoded by said genetic locus; (c) growing a transformed host cell under selective medium conditions, wherein said medium comprises an efficient amount of 5-flucytosine (5-FC), 5-fluorouracil (5-FU) or 5-fluorouridine (5-FUR); and (d) selecting a host cell which is capable of growing under the medium conditions of step (c).

The term “pyrimidine salvage pathway” as used herein refers to a pathway in bacteria or eukaryotes which leads to the synthesis of pyrimidine nucleotides from intermediates occurring during the degradation of nucleotides. The pyrimidine salvage pathway may, for example, typically comprise several of the following enzymatic activities: CTP synthase (EC 6.3.4.2), nucleoside triphosphate phosphatase (EC 3.6.1.15), nucleotide diphosphate kinase (EC 2.7.4.6), apyrase (EC 3.6.1.5), nucleoside diphosphate phosphatase (EC 3.6.1.6), uridylate/cytidylate kinase (EC 2.7.4.14), pyrimidine specific 5′ nucleotidase (EC 3.1.3.5), uridine/cytidine kinase (EC 2.7.1.48), cytosine deaminase (EC 3.5.4.1), cytidine deaminase (EC 3.5.4.5), uridine nucleosidase (EC 3.2.2.3), uridine phosphorylase (EC 2.4.2.3) and uracil phosphoribosyl-transferase (EC 2.4.2.9). In specific embodiments, the present invention also envisages the employment of a combination of the above mentioned enzymatic activities of the pyrimidine salvage pathway and their corresponding genetic loci. For example, envisaged are combinations or 2, 3 or more of the following enzymatic activities or their corresponding genetic loci: CTP synthase (EC 6.3.4.2), nucleoside triphosphate phosphatase (EC 3.6.1.15), nucleotide diphosphate kinase (EC 2.7.4.6), apyrase (EC 3.6.1.5), nucleoside diphosphate phosphatase (EC 3.6.1.6), uridylate/cytidylate kinase (EC 2.7.4.14), pyrimidine specific 5′ nucleotidase (EC 3.1.3.5), uridine/cytidine kinase (EC 2.7.1.48), cytosine deaminase (EC 3.5.4.1), cytidine deaminase (EC 3.5.4.5), uridine nucleosidase (EC 3.2.2.3), uridine phosphorylase (EC 2.4.2.3) and uracil phosphoribosyl-transferase (EC 2.4.2.9).

In addition to these activities, the pathway usually comprises accessory transporters or permeases such as purine/cytosine permease, uridine permease or uracil permease and concentrative nucleoside transporter. In further specific embodiments, the present invention also envisages the employment of a combination of the above mentioned accessory transporters or permeases and the above mentioned enzymatic activities. For example, envisaged are combinations or 2, 3 or more of the following enzymatic activities or their corresponding genetic loci: CTP synthase (EC 6.3.4.2), nucleoside triphosphate phosphatase (EC 3.6.1.15), nucleotide diphosphate kinase (EC 2.7.4.6), apyrase (EC 3.6.1.5), nucleoside diphosphate phosphatase (EC 3.6.1.6), uridylate/cytidylate kinase (EC 2.7.4.14), pyrimidine specific 5′ nucleotidase (EC 3.1.3.5), uridine/cytidine kinase (EC 2.7.1.48), cytosine deaminase (EC 3.5.4.1), cytidine deaminase (EC 3.5.4.5), uridine nucleosidase (EC 3.2.2.3), uridine phosphorylase (EC 2.4.2.3) and uracil phosphoribosyl-transferase (EC 2.4.2.9), a purine/cytosine permease, an uridine permease, an uracil permease and a concentrative nucleoside transporter.

According to the present invention, at least the following enzymatic activities of the pyrimidine salvage pathway and their corresponding genetic loci may be used: purine/cytosine permease (e.g. FcyB or functional homologues, or functional orthologues), cytosine deaminase (FcyA or functional homologues, or functional orthologues), uracil permease or uridine permease, concentrative nucleoside transporter (CntA or functional homologues, or functional orthologues), uracil-phosphoribosyl-transferase (e.g. Uprt1 or functional homologues, or functional orthologues) or uridine kinase (UK or functional homologues or functional orthologues).

It is further envisaged that one or more further specific enzymes (and their corresponding genetic loci) of the pyrimidine salvage pathway, e.g. as mentioned above, be used in a method according to the present invention if these enzymes contribute to the toxicity of 5-FC, 5-FU or 5-FUR, for instance by transporting said compounds into a cell or by converting them into a toxic substance. In certain, specific embodiments, the specific enzyme (and its corresponding genetic locus) is not cytosine deaminase (EC 3.5.4.5) or FcyA or a functional homologue, or functional orthologue thereof. In further, specific embodiments, the specific enzyme is not a cytosine deaminase, e.g. FcyA, of Aspergillus niger.

The “purine/cytosine permease” as used herein relates to a polypeptide comprising, essentially consisting of or consisting of the amino acid sequence of SEQ ID NO: 1 or functional parts or fragments thereof, or is encoded by a nucleic acid comprising, essentially consisting of or consisting of the nucleotide sequence of SEQ ID NO: 2 or functional parts or fragments thereof, or is provided by a polypeptide comprising, essentially consisting of or consisting of an amino acid having at least about 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 1 or functional parts or fragments thereof, or is encoded by a nucleic acid comprising, essentially consisting of or consisting of a nucleotide sequence having at least about 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 2 or functional parts or fragments thereof, or by a polynucleotide encoding a polypeptide domain of SEQ ID NO: 1, or polynucleotide encoding a variant of SEQ ID NO: 1, or a polynucleotide encoding an allelic variant of SEQ ID NO: 1, a polynucleotide encoding a species homologue of SEQ ID NO: 1, a polynucleotide encoding a species orthologue of SEQ ID NO: 1 or encoded by a polynucleotide which is a variant of SEQ ID NO: 2, or by a polynucleotide which is an allelic variant of SEQ ID NO: 2, or by a polynucleotide which is a species homologue or orthologue of SEQ ID NO: 2, or by a polynucleotide which is capable of hybridizing under stringent conditions to any polynucleotide as defined in the above passage. In a preferred embodiment any homologous or orthologous sequence or variant as mentioned above has or comprises the same or a very similar function as a polypeptide comprising, essentially consisting of or consisting of the amino acid sequence of SEQ ID NO: 1, i.e. purine/cytosine permease activity. Examples of preferred orthologous sequences are provided in Table A, infra.

In preferred embodiments the purine/cytosine permease is a fungal polypeptide. In more preferred embodiments, the purine/cytosine permease is the Aspergillus fumigatus polypeptide AfFcyB. A genetic locus comprising the nucleotide sequence of SEQ ID NO: 2 may comprise additional 1 kb, 2 kb, 3 kb, 4 kb, 5 kb or more at the 5′ or 3′ termini of the mentioned sequence, or its homologue or orthologue. It is particularly preferred that said locus comprises all elements which are necessary for the function or expression of the polypeptide. This includes, besides the coding sequence of the polypeptide, also any regulatory sequence either 3′ or 5′ of the coding sequence. The genetic locus of the coding sequence for Aspergillus fumigatus polypeptide AfFcyB including its 5′ and 3′ neighboring region may further be derived from genomic database GenBank assembly by referring to the position information supercontig: ASM15014v1:DS499595:2495865:2497570:1. Based on the indicated location as starting point, 5′ and 3′ sequences (e.g. 30 bp, 50 bp, 100 bp, 200 bp, 300 bp, 400 bp, 500 bp, 600 bp, 750 bp, 1 kb, 2 kb, 3 kb, 4 kb, 5 kb or more or any value in between the mentioned values) may be obtained from assembly ASM15014v1. Genomic location information on an envisaged orthologous sequence may be derived from Table A, infra, in particular from the column labelled “Genomic location”.

The “uracil-phosphoribosyl-transferase” as used herein relates to a polypeptide comprising, essentially consisting of or consisting of the amino acid sequence of SEQ ID NO: 3 or functional parts or fragments thereof, or is encoded by a nucleic acid comprising, essentially consisting of or consisting of the nucleotide sequence of SEQ ID NO: 4 or functional parts or fragments thereof, or is provided by a polypeptide comprising, essentially consisting of or consisting of an amino acid having at least about 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 3 or functional parts or fragments thereof, or is encoded by a nucleic acid comprising, essentially consisting of or consisting of a nucleotide sequence having at least about 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 4 or functional parts or fragments thereof, or by a polynucleotide encoding a polypeptide domain of SEQ ID NO: 3, or polynucleotide encoding a variant of SEQ ID NO: 3, or a polynucleotide encoding an allelic variant of SEQ ID NO: 3, a polynucleotide encoding a species homologue of SEQ ID NO: 3, a polynucleotide encoding a species orthologue of SEQ ID NO: 3 or encoded by a polynucleotide which is a variant of SEQ ID NO: 4, or by a polynucleotide which is an allelic variant of SEQ ID NO: 4, or by a polynucleotide which is a species homologue or orthologue of SEQ ID NO: 4, or by a polynucleotide which is capable of hybridizing under stringent conditions to any polynucleotide as defined in the above passage. In a preferred embodiment any homologous or orthologous sequence or variant as mentioned above has or comprises the same or a very similar function as a polypeptide comprising, essentially consisting of or consisting of the amino acid sequence of SEQ ID NO: 3, i.e. uracil-phosphoribosyl-transferase activity. Examples of preferred orthologous sequences are provided in Table B, infra.

In preferred embodiments the uracil-phosphoribosyl-transferase is a fungal polypeptide. In more preferred embodiments, the uracil-phosphoribosyl-transferase is the Aspergillus fumigatus polypeptide AfUprt. A genetic locus comprising the nucleotide sequence of SEQ ID NO: 4 may comprise additional 1 kb, 2 kb, 3 kb, 4 kb, 5 kb or more at the 5′ or 3′ termini of the mentioned sequence, or its homologue or orthologue. It is particularly preferred that said locus comprises all elements which are necessary for the function or expression of the polypeptide. This includes, besides the coding sequence of the polypeptide, also any regulatory sequence either 3′ or 5′ of the coding sequence. The genetic locus of the coding sequence for Aspergillus fumigatus polypeptide AfUprt including its 5′ and 3′ neighboring region may further be derived from genomic database GenBank assembly by referring to the position information supercontig: ASM15014v1:DS499597:1174905:1175833:-1. Based on the indicated location as starting point, 5′ and 3′ sequences (e.g. 30 bp, 50 bp, 100 bp, 200 bp, 300 bp, 400 bp, 500 bp, 600 bp, 750 bp, 1 kb, 2 kb, 3 kb, 4 kb, 5 kb or more or any value in between the mentioned values) may be obtained from assembly ASM15014v1. Genomic location information on an envisaged orthologous sequence may be derived from Table B, infra, in particular from the column labelled “Genomic location”.

The “concentrative nucleoside transporter” as used herein relates to a polypeptide comprising, essentially consisting of or consisting of the amino acid sequence of SEQ ID NO: 5 or functional parts or fragments thereof, or is encoded by a nucleic acid comprising, essentially consisting of or consisting of the nucleotide sequence of SEQ ID NO: 6 or functional parts or fragments thereof, or is provided by a polypeptide comprising, essentially consisting of or consisting of an amino acid having at least about 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 5 or functional parts or fragments thereof, or is encoded by a nucleic acid comprising, essentially consisting of or consisting of a nucleotide sequence having at least about 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 6 or functional parts or fragments thereof, or by a polynucleotide encoding a polypeptide domain of SEQ ID NO: 5, or polynucleotide encoding a variant of SEQ ID NO: 5, or a polynucleotide encoding an allelic variant of SEQ ID NO: 5, a polynucleotide encoding a species homologue of SEQ ID NO: 5, a polynucleotide encoding a species orthologue of SEQ ID NO: 5 or encoded by a polynucleotide which is a variant of SEQ ID NO: 6, or by a polynucleotide which is an allelic variant of SEQ ID NO: 6, or by a polynucleotide which a species homologue or orthologue of SEQ ID NO: 6, or by a polynucleotide which is capable of hybridizing under stringent conditions to any polynucleotide as defined in the above passage. In a preferred embodiment any homologous or orthologous sequence or variant as mentioned above has or comprises the same or a very similar function as a polypeptide comprising, essentially consisting of or consisting of the amino acid sequence of SEQ ID NO: 5, i.e. concentrative nucleoside transporter activity. Examples of preferred orthologous sequences are provided in Table C, infra.

In preferred embodiments the concentrative nucleoside transporter is a fungal polypeptide. In more preferred embodiments, the concentrative nucleoside transporter is the Aspergillus fumigatus polypeptide AfCntA. A genetic locus comprising the nucleotide sequence of SEQ ID NO: 6 may comprise additional 1 kb, 2 kb, 3 kb, 4 kb, 5 kb or more at the 5′ or 3′ termini of the mentioned sequence, or its homologue or orthologue. It is particularly preferred that said locus comprises all elements which are necessary for the function or expression of the polypeptide. This includes, besides the coding sequence of the polypeptide, also any regulatory sequence either 3′ or 5′ of the coding sequence. The genetic locus of the coding sequence for Aspergillus fumigatus polypeptide AfCntA including its 5′ and 3′ neighboring region may further be derived from genomic database GenBank assembly by referring to the position information supercontig: ASM15014v1:DS499594:432155:434174:-1. Based on the indicated location as starting point, 5′ and 3′ sequences (e.g. 30 bp, 50 bp, 100 bp, 200 bp, 300 bp, 400 bp, 500 bp, 600 bp, 750 bp, 1 kb, 2 kb, 3 kb, 4 kb, 5 kb or more or any value in between the mentioned values) may be obtained from assembly ASM15014v1. Genomic location information on an envisaged orthologous sequence may be derived from Table C, infra, in particular from the column labelled “Genomic location”.

The “uridine kinase” as used herein relates to a polypeptide comprising, essentially consisting of or consisting of the amino acid sequence of SEQ ID NO: 7 or functional parts or fragments thereof, or is encoded by a nucleic acid comprising, essentially consisting of or consisting of the nucleotide sequence of SEQ ID NO: 8 or functional parts or fragments thereof, or is provided by a polypeptide comprising, essentially consisting of or consisting of an amino acid having at least about 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 7 or functional parts or fragments thereof, or is encoded by a nucleic acid comprising, essentially consisting of or consisting of a nucleotide sequence having at least about 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 8 or functional parts or fragments thereof, or by a polynucleotide encoding a polypeptide domain of SEQ ID NO: 7, or polynucleotide encoding a variant of SEQ ID NO: 7, or a polynucleotide encoding an allelic variant of SEQ ID NO: 7, a polynucleotide encoding a species homologue of SEQ ID NO: 7, a polynucleotide encoding a species orthologue of SEQ ID NO: 7 or encoded by a polynucleotide which is a variant of SEQ ID NO: 8, or by a polynucleotide which is an allelic variant of SEQ ID NO: 8, or by a polynucleotide which is a species homologue or orthologue of SEQ ID NO: 8, or by a polynucleotide which is capable of hybridizing under stringent conditions to any polynucleotide as defined in the above passage. In a preferred embodiment any homologous or orthologous sequence or variant as mentioned above has or comprises the same or a very similar function as a polypeptide comprising, essentially consisting of or consisting of the amino acid sequence of SEQ ID NO: 7, i.e. uridine kinase activity. Examples of preferred orthologous sequences are provided in Table D, infra.

In preferred embodiments the uridine kinase is a fungal polypeptide. In more preferred embodiments, the uridine kinase is the Aspergillus fumigatus polypeptide AfUK. A genetic locus comprising the nucleotide sequence of SEQ ID NO: 8 may comprise additional 1 kb, 2 kb, 3 kb, 4 kb, 5 kb or more at the 5′ or 3′ termini of the mentioned sequence, or its homologue or orthologue. It is particularly preferred that said locus comprises all elements which are necessary for the function or expression of the polypeptide. This includes, besides the coding sequence of the polypeptide, also any regulatory sequence either 3′ or 5′ of the coding sequence. The genetic locus of the coding sequence for Aspergillus fumigatus polypeptide AfUK including its 5′ and 3′ neighboring region may further be derived from genomic database GenBank assembly by referring to the position information supercontig: ASM15014v1:DS499595:1507188:1509002:-1. Based on the indicated location as starting point, 5′ and 3′ sequences (e.g. 30 bp, 50 bp, 100 bp, 200 bp, 300 bp, 400 bp, 500 bp, 600 bp, 750 bp, 1 kb, 2 kb, 3 kb, 4 kb, 5 kb or more or any value in between the mentioned values) may be obtained from assembly ASM15014v1. Genomic location information on an envisaged orthologous sequence may be derived from Table D, infra, in particular from the column labelled “Genomic location”.

The “cytosine deaminase” as used herein relates to a polypeptide comprising, essentially consisting of or consisting of the amino acid sequence of SEQ ID NO: 135 or functional parts or fragments thereof, or is encoded by a nucleic acid comprising, essentially consisting of or consisting of the nucleotide sequence of SEQ ID NO: 136 or functional parts or fragments thereof, or is provided by a polypeptide comprising, essentially consisting of or consisting of an amino acid having at least about 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 135 or functional parts or fragments thereof, or is encoded by a nucleic acid comprising, essentially consisting of or consisting of a nucleotide sequence having at least about 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 136 or functional parts or fragments thereof, or by a polynucleotide encoding a polypeptide domain of SEQ ID NO: 135, or polynucleotide encoding a variant of SEQ ID NO: 135, or a polynucleotide encoding an allelic variant of SEQ ID NO: 135, a polynucleotide encoding a species homologue of SEQ ID NO: 135, a polynucleotide encoding a species orthologue of SEQ ID NO: 135 or encoded by a polynucleotide which is a variant of SEQ ID NO: 136, or by a polynucleotide which is an allelic variant of SEQ ID NO: 136, or by a polynucleotide which is a species homologue or orthologue of SEQ ID NO: 136, or by a polynucleotide which is capable of hybridizing under stringent conditions to any polynucleotide as defined in the above passage. In a preferred embodiment any homologous or orthologous sequence or variant as mentioned above has or comprises the same or a very similar function as a polypeptide comprising, essentially consisting of or consisting of the amino acid sequence of SEQ ID NO: 135, i.e. cytosine deaminase activity. Examples of preferred orthologous sequences are provided in Table E, infra.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence or any fragment as described herein. Whether any particular nucleic acid molecule is at least 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% etc. identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., 1990, Comp. App. Biosci. 6: 237-245. In a nucleotide sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=l, Gap Penalty=5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage may then be subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence. Whether any particular polypeptide is at least at least 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% etc. identical to, for instance, an amino acid sequence of the present invention can be determined conventionally by using known computer programs. A preferred method for determining the best overall match between a query sequence (a sequence of the present invention) and a subject sequence, also referred to as a global sequence alignment, can be determined using the FASTDB computer program based on the algorithm of Brutlag et al., 1990, Comp. App. Biosci. 6: 237-245. In an amino acid sequences alignment the query and subject sequences are both amino acid sequences. The result of said global sequence alignment is given in percent identity. Preferred parameters used in a FASTDB amino acid alignment are: Matrix=PAM 0, k-tuple=2, Mismatch Penalty=l, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned may be determined by the results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

The term “polypeptide” as used herein refers to a continuous and unbranched peptide chain of a certain length. A polypeptide may, for example, have a length of more than 20 to 50 amino acids. The term “protein” as used herein relates to an arrangement of one or more polypeptides. Accordingly, a protein may comprise or consist of one polypeptide and thus by synonymous to polypeptide. In other embodiments, a protein may comprise 2 or more polypeptides which may be organized in units or subunits of a higher order structure in the form of a protein.

The term “homologous sequence” as used herein generally means that the sequence has a certain (high) degree of similarity with another sequence. This similarity can either be derived from nucleic acid or amino acid sequence information. Such a high similarity is typically a strong evidence that two sequences are related by evolutionary changes from a common ancestral sequence. The two sequences compared may be derived from the same organism, or from different organisms, e.g. different species. A “functional homologue” as used herein implies that not only the sequence of the homologue is similar to another sequence, but also that the function of the encoded polypeptide, e.g. an enzymatic activity or a transporter activity, is similar or identical to the function of a polypeptide encoded by said other sequence.

An “orthologue” as used herein generally refers to a homologous sequence which is inferred to be descended from the same ancestral sequence separated by a speciation event. Accordingly, orthologous genes are genes in different species that originated by vertical descent from a single gene of the last common ancestor. A “functional orthologue” as used herein accordingly implies that not only the sequence (e.g. nucleic acid or amino acid sequence) of the orthologue is similar to a sequence in a different species, but also that the function of the encoded polypeptide, e.g. an enzymatic activity or a transporter activity, is similar or identical to the function of a polypeptide encoded by said sequence in a different species.

The present invention specifically envisages the use of the following orthologues of the purine/cytosine permease as defined herein, e.g. having the amino acid sequence of SEQ ID NO: 1, the uracil-phosphoribosyl-transferase as defined herein, e.g. having the amino acid sequence of SEQ ID NO: 3, the concentrative nucleoside transporter as defined herein, e.g. having amino acid sequence of SEQ ID NO: 5, the uridine kinase as defined herein, e.g. having amino acid sequence of SEQ ID NO: 7, or the cytosine deaminase as defined herein, e.g. having the amino acid sequence of SEQ ID NO: 135. Also envisaged is the use of further orthologous sequences as shown in Tables A to E, e.g. a sequence having the amino acid sequence of SEQ ID NO: 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 137, 139, 141, 143, 145, 147 or 149, or a sequence having the nucleotide sequence of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 138, 140, 142, 144, 146, 148, or 150.

TABLE A
Orthologous sequences of the purine/cytosine permease of SEQ ID NO: 1

| | | Protein | | | | |
|---|---|---|---|---|---|---|
| | | Accession | Gene ID | | Amino acid | Nucleotide |
| Species | Identity | (NCBI) | (Ensembl Fungi) | Genomic Location | SEQ ID NO: | SEQ ID NO: |
| Aspergillus fumigatus | 100% | EDP54513.1* | AFUB_025700 | DS499595: 2495865- | 1 | 2 |
| A1163 | | | | 2497570 | | |
| Aspergillus | 80% | XP_001826247.1 | AO090011000649 | 7: 1685838-1687535 | 9 | 10 |
| oryzae RIB40 | | | | | | |
| Aspergillus niger | 77% | EHA22089.1 | ASPNIDRAFT_200921 | ACJE01000013: 1503743- | 11 | 12 |
| ATCC 1015 | | | | 1505426 | | |
| Penicillium | 75% | KZN90676.1 | EN45_007980 | I: 2260282-2262177 | 13 | 14 |
| chrysogenum | | | | | | |
| Saccharomyces | 42% | EWH18811.1 | P283_E21041 | V: 257657-259258 | 15 | 16 |
| cerevisiae | | | | | | |
| P283 | | | | | | |
| Candida albicans | 46% | XP_714531.2 | CAALFM_C209950WA | 2: 2034730-2036274 | 17 | 18 |
| SC5314 | | | | | | |
| Saccharomyces | 43% | EWH18815.1 | P283_E21141 | V: 267717-269309 | 19 | 20 |
| cerevisiae | | | | | | |
| P283 | | | | | | |
| Komagataella | 42% | XP_002493949.1 | PAS_chr4_0514 | 4: 1006621-1008180 | 21 | 22 |
| phaffli GS115 | | | | | | |
| Candida albicans | 37% | KHC78891.1 | W5Q_02853 | supercont4.12: 238343- | 23 | 24 |
| SC5314 | | | | 239923 | | |
| Candida albicans | 37% | XP_720028.2 | CAALFM_C303360WA | 3: 704145-705725 | 25 | 26 |
| SC5314 | | | | | | |
| Candida albicans | 37% | KHC87114.1 | I503_02846 | supercont3.29: 2101- | 27 | 28 |
| SC5314 | | | | 3681 | | |
| Cryptococcus | 40% | XP_012052683.1 | CNAG_01681 | 11: 591464-594607 | 29 | 30 |
| neoformans | | | | | | |
| var. grubii | | | | | | |
| H99 | | | | | | |

*The respective A. fumigatus protein sequence has been used for sequence comparisons (BLAST analysis)

TABLE B
Orthologous sequences of the uracil-phosphoribosyl-transferase of SEQ ID NO: 3

| | | Protein | | | | |
|---|---|---|---|---|---|---|
| | | Accession | Gene ID | | Amino acid | Nucleotide |
| Species | Identity | (NCBI) | (Ensembl Fungi) | Genomic Location | SEQ ID NO: | SEQ ID NO: |
| Aspergillus fumigatus | 100% | EDP51298.1* | AFUB_053020 | DS499597: 1174905- | 3 | 4 |
| A1163 | | | | 1175833 | | |
| Aspergillus niger | 94% | EHA22482.1 | ASPNIDRAFT_54952 | ACJE01000012: 922852- | 31 | 32 |
| ATCC 1015 | | | | 923959 | | |
| Penicillium | 89% | KZN87537.1 | EN45_060980 | II: 3551944-3553262 | 33 | 34 |
| chrysogenum | | | | | | |
| Aspergillus | 90% | XP_023088768.1 | AO090009000714 | 1: 1906721-1907646 | 35 | 36 |
| oryzae RIB40 | | | | | | |
| Trichoderma | 78% | XP_006967593.1 | TRIRE- | GL985073: 642771- | 37 | 38 |
| reesei QM6a | | | DRAFT_22945 | 643947 | | |
| Acremonium | 76% | KFH46319.1 | ACRE_028730 | scaffold21: 52093- | 39 | 40 |
| chrysogenum | | | | 53065 | | |
| ATCC 11550 | | | | | | |
| Fusarium | 79% | EWY99635.1 | FOYG_03618 | super- | 41 | 42 |
| oxysporum | | | | cont1.2: 4214353- | | |
| FOSC 3-a | | | | 4216334 | | |
| Ustilago maydis | 70% | XP_011390366.1 | UMAG_03873 | 11: 7304-7999 | 43 | 44 |
| 521 | | | | | | |
| Komagataella | 67% | XP_002489914.1 | PAS_chr1- | 1: 1059188-1059838 | 45 | 46 |
| phaffii GS115 | | | 1_0262 | | | |
| Cryptococcus | 69% | XP_012050086.1 | CNAG_02337 | 6: 574416-576232 | 47 | 48 |
| neoformans var. | | | | | | |
| grubii H99 | | | | | | |
| Candida albicans | 66% | XP_712023.1 | CAALFM_C503390CA | 5: 766244-766900 | 49 | 50 |
| SC5314 | | | | | | |
| Saccharomyces | 66% | EWH18153.1 | P283_H11296 | VIII: 343077-343727 | 51 | 52 |
| cerevisiae P283 | | | | | | |
| Rhizopus | 63% | EIE83761.1 | RO3G_08466 | CH476737: 1216779- | 53 | 54 |
| delemar RA 99-880 | | | | 1217859 | | |

*The respective A. fumigatus protein sequence has been used for sequence comparisons (BLAST analysis)

TABLE C
Orthologous sequences of the concentrative nucleoside transporter of SEQ ID NO: 5

| | | Protein | | | | |
|---|---|---|---|---|---|---|
| | | Accession | Gene ID | | Amino acid | Nucleotide |
| Species | Identity | (NCBI) | (Ensembl Fungi) | Genomic Location | SEQ ID NO: | SEQ ID NO: |
| Aspergillus | 100% | EDP55462.1* | AFUB_001570 | DS499594: 432155- | 5 | 6 |
| fumigatus A1163 | | | | 434174 | | |
| Aspergillus | 76% | XP_001819624.1 | AO090003000443 | 2: 3270188-3272161 | 55 | 56 |
| oryzae RIB40 | | | | | | |
| Penicillium chrysogenum | 72% | KZN86056.1 | EN45_102530 | III: 4855297-4857465 | 57 | 58 |
| Aspergillus niger | 73% | EHA18479.1 | ASPNIDRAFT_176590 | ACJE01000021: 2465325- | 59 | 60 |
| ATCC 1015 | | | | 2467199 | | |
| Trichoderma | 57% | XP_006967067.1 | TRIRE- | GL985070: 154283- | 61 | 62 |
| reesei QM6a | | | DRAFT_49970 | 156793 | | |
| Acremonium | 55% | KFH45688.1 | ACRE_034270 | scaffold28: 6018-8020 | 63 | 64 |
| chrysogenum | | | | | | |
| ATCC 11550 | | | | | | |
| Fusarium | 57% | EWY90018.1 | FOYG_07655 | supercont1.5: 636146- | 65 | 66 |
| oxysporum FOSC 3-a | | | | 639938 | | |
| Candida albicans | 48% | KHC81642.1 | W5Q_02029 | super- | 67 | 68 |
| SC5314 | | | | cont4.6: 1263887- | | |
| | | | | 1265713 | | |
| Candida albicans | 48% | XP_714288.1 | CAALFM_C206020WA | 2: 1232702-1234528 | 69 | 70 |
| SC5314 | | | | | | |
| Candida albicans | 48% | KHC87973.1 | I503_02043 | super- | 71 | 72 |
| SC5314 | | | | cont3.23: 578397- | | |
| | | | | 580223 | | |
| Rhizopus | 46% | EIE91231.1 | RO3G_15942 | CH476749: 191259- | 73 | 74 |
| delemar RA 99-880 | | | | 193093 | | |
| Rhizopus | 42% | EIE78985.1 | RO3G_03690 | CH476733: 3722926- | 75 | 76 |
| delemar RA 99-880 | | | | 3724762 | | |

*The respective A. fumigatus protein sequence has been used for sequence comparisons (BLAST analysis)

TABLE D
Orthologous sequences of the uridine kinase of SEQ ID NO: 7

| | | Protein | | | | |
|---|---|---|---|---|---|---|
| | | Accession | Gene ID | | Amino acid | Nucleotide |
| Species | Identity | (NCBI) | (Ensembl Fungi) | Genomic Location | SEQ ID NO: | SEQ ID NO: |
| Aspergillus | 100% | EDP54194.1* | AFUB_022460 | DS499595: 1507188- | 7 | 8 |
| fumigatus A1163 | | | | 1509002 | | |
| Aspergillus niger | 83% | EHA19972.1 | ASPNIDRAFT_53035 | ACJE01000019: 22706 | 77 | 78 |
| ATCC 1015 | | | | 25-2272432 | | |
| Penicillium chrysogenum | 80% | KZN85485.1 | EN45096700 | III: 3225466-3227388 | 79 | 80 |
| Acremonium | 62% | KFH42094.1 | ACRE_071950 | scaffold105: 45307- | 81 | 82 |
| chrysogenum | | | | 47161 | | |
| ATCC 11550 | | | | | | |
| Trichoderma | 62% | XP_006962453.1 | TRIRE- | GL985057: 1868863- | 83 | 84 |
| reesei QM6a | | | DRAFT_75056 | 1870827 | | |
| Fusarium oxysporum | 61% | EWY91549.1 | FOYG_08619 | super- | 85 | 86 |
| FOSC 3-a | | | | cont1.5: 3321407- | | |
| | | | | 3324115 | | |
| Aspergillus oryzae | 79% | XP_023089753.1 | AO090001000654 | 2: 1724956-1725793 | 87 | 88 |
| RIB40 | | | | | | |
| Candida albicans | 41% | KHC87190.1 | I503_02926 | super- | 89 | 90 |
| SC5314 | | | | cont3.29: 150827- | | |
| | | | | 152464 | | |
| Candida albicans | 41% | KHC78966.1 | W5Q_02932 | super- | 91 | 92 |
| SC5314 | | | | cont4.12: 388435- | | |
| | | | | 390072 | | |
| Candida albicans | 41% | XP_723080.1 | CAALFM_C304220CA | 3: 875584-877221 | 93 | 94 |
| SC5314 | | | | | | |
| Komagataella | 42% | XP_002491704.1 | PAS_chr2- | 2: 1467184-1468638 | 95 | 96 |
| phaffii GS115 | | | 1_0770 | | | |
| Saccharomyces | 42% | EWH16251.1 | P283_N20816 | XIV: 684310-685815 | 97 | 98 |
| cerevisiae P283 | | | | | | |
| Cryptococcus | 39% | XP_012051210.1 | CNAG_03367 | 8: 763309-765904 | 99 | 100 |
| neoformans var. | | | | | | |
| grubii H99 | | | | | | |
| Rhizopus delemar | 42% | EIE82575.1 | RO3G_07280 | CH476736: 1377537- | 101 | 102 |
| RA 99-880 | | | | 1379356 | | |

*The respective A. fumigatus protein sequence has been used for sequence comparisons (BLAST analysis)

TABLE E
Orthologous sequences of the cytosine deaminase of SEQ ID NO: 135

| | | Protein | | | | |
|---|---|---|---|---|---|---|
| | | Accession | Gene ID | | Amino acid | Nucleotide |
| Species | Identity | (NCBI) | (Ensembl Fungi) | Genomic Location | SEQ ID NO: | SEQ ID NO: |
| Aspergillus fumigatus | 100% | EDP55842.1* | AFUB_005410 | DS499594: 1527020- | 135 | 136 |
| A1163 | | | | 1527671 | | |
| Aspergillus niger | 91% | EHA26383.1 | ASPNIDRAFT_206127 | ACJE01000004: 11381 | 137 | 138 |
| ATCC1015 | | | | 50-1138910 | | |
| Penicillium chrysogenum | 91% | KZN93743.1 | EN45_039280 | I: 11,041,067- | 139 | 140 |
| | | | | 11,041,939 | | |
| Aspergillus oryzae | 93% | XP_001819938.3 | AO090003000802 | 2: 4,237,640- | 141 | 142 |
| RIB40 | | | | 4,238,143 | | |
| Komagataella | 63% | XP_002490927.1 | PAS_chr2- | 2: 78,280-78,732 | 143 | 144 |
| phaffii GS115 | | | 1_0047 | | | |
| Candida albicans | 61% | KHC73214.1 | W5Q_04651 | supercont4.28: | 145 | 146 |
| SC5314 | | | | 113,519-114,041 | | |
| Saccharomyces | 61% | EWH15533.1 | P283_P21541 | XVI: 824,996-825,472 | 147 | 148 |
| cerevisiae P283 | | | | | | |
| Cryptococcus | 49% | XP_012046842.1 | CNAG_00613 | 1: 1,575,877- | 149 | 150 |
| neoformans var. | | | | 1,578,119 | | |
| grubii H99 | | | | | | |

*The respective A. fumigatus protein sequence has been used for sequence comparisons (BLAST analysis)

The present invention further relates to and envisages the use of orthologous sequences of the pyrimidine salvage pathway which are derived from bacteria or plants.

Examples of such sequences are provided in the following Table F.

TABLE F
Orthologous sequences derived from bacteria and plants

| | | Protein | | | Amino acid | Nucleotide |
|---|---|---|---|---|---|---|
| | Similarity | Accession | Gene | | SEQ ID | SEQ ID |
| Species | to/function | (NCBI) | Name | Genomic Location | NO: | NO: |
| E. coli | purine/ | AKD59926.1 | codB | ASM97440v1: | 129 | 130 |
| K-12 | cytosine | | | Chromosome: 1116673: | | |
| | permease | | | 1117932: 1 | | |
| E. coli | cytosine | AKK16692.1 | codA | ASM80076v1: | 151 | 152 |
| K-12 | deaminase | | | Chromosome: | | |
| | | | | 358069: 359352: 1 | | |
| E. coli | uracil | AKD62005.1 | upp | ASM97440v1: | 131 | 132 |
| K-12 | phos- | | | Chromosome: | | |
| | phoribosyltrans- | | | 3369083: | | |
| | ferase | | | 3369709: −1 | | |
| A. thaliana | uracil | Q9FKS0.1 | ukl1 | TAIR10: 5: | 133 | 134 |
| | Phos- | | | 16374799: 1 | | |
| | phoribosyltrans- | | | 6378652: 1 | | |
| | ferase | | | | | |

The genetic locus of the coding sequence for the polypeptides mentioned in Tables A to F, including its 5′ and 3′ neighboring regions, may specifically be derived from genomic databases indicated in column “Genomic location” of Tables A to F. In said column the genomic sequence assembly reference is indicated, as well as the information on the start and end position of the coding sequence for the polypeptide. By locating said sequence and by correspondingly deriving neighboring sequences (e.g. 30 bp, 50 bp, 100 bp, 200 bp, 300 bp, 400 bp, 500 bp, 600 bp, 750 bp, 1 kb, 2 kb, 3 kb, 4 kb, 5 kb or more or any value in between the mentioned values) in 5′ and/or 3′ direction elements required for homologous integration, e.g. flanking sequences, can be derived.

The term “site directed integration” as used herein relates to a type of genetic recombination in which DNA strand exchange takes place between segments possessing a high degree of sequence homology. Such recombination events may typically make use of enzymatic machinery already present in a host cell. The integration is typically based on events of homologous recombination between two similar or identical molecules of DNA. The homologous recombination may, in eukaryotes, involve activities of the DSBR pathway or the SDSA pathway. Also envisaged is machinery of the SSA pathway. In bacteria host cell activities of the RecBCD or RecF or the RecB, RecC and SbcB pathway may be employed. Further information can be derived from suitable literature sources such as Bird et al., Mol Gen Genet. 1997; 255(2):219-25 or Winans et al., Journal of Bacteriology, 1985; 161(3):1219-21.

In the method according to the present invention the site-directed integration makes use of an integrative nucleic acid construct which comprises one or two homologous flanks to a genetic locus of the pyrimidine salvage pathway as defined herein. For example, the homologous flank may be a 3′ flank or a 5′ flank. It is preferred that two flanks, a 3′ and a 5′ flank are present. The size of the flanks can vary, e.g. dependent on the host cell, the size of the integrative construct, the identity of the targeted genetic locus etc. In specific embodiments, the homologous flank may have a size of about 50 bp to about 10,000 bp. It is preferred that the homologous flank has a size of about 100 bp to about 400 bp. It is more preferred that the homologous flank has a size of about 200 bp to about 400 bp. Also envisaged are all size values in between the mentioned values. In case of two flanks, a 3′ and a 5′ flank, the size of the flanks may either be identical or similar (symmetric flanks), e.g. a 3′ flank with 300 bp and a 5′ flank with about 300 bp or about 320 bp or vice versa etc. Alternatively, the flanks may not be similar in size (asymmetric flanks). For example, the 3′ flank may have a size of about 100 bp and the 5′ flank may have a size of about 400 bp or vice versa etc.

The term “integrative nucleic acid construct” as used herein refers to any nucleic acid molecule, which has the capacity to be inserted at a predefined location in the genome of a host cell by homologous recombination. The construct typically comprises one or more homologous flanks or sections as defined herein. The construct may further comprise one or more gene or sequence of interest, which is intended to be introduced into a genomic site as described herein. The construct may be composed of DNA. In certain embodiments, also the provision of RNA constructs is envisaged. The DNA construct may be provided as single stranded or double stranded construct. It is preferred that a double stranded construct be used. The construct may either be provided as linearized or as circular molecule. The circular molecule may be used as such or may be accompanied by the presence of a restriction enzyme, which leads to linearization upon transformation of a host cell.

The term “homologous flank” as used herein relates to sequences which show a high degree of sequence identity with the sequence portion where the recombination is planned to take place, e.g. the genetic loci as defined herein. A high degree of identity may, for example, be a sequence identity of 80%, 85%, 95%, 96%, 97%, 98%, 99%, or more between the homologous flank and sequence at the genomic locus where recombination is planned to take place.

The exact position of the homologous flanks within the genetic locus of the pyrimidine salvage pathway member is variable. Any suitable position, which leads, upon homologous recombination, to an inactivation or reduction of the activity encoded by said genetic locus is encompassed by the present invention. In certain embodiments, the integrative nucleic acid construct may, for example, simply carry a sequence being homologous to said genetic locus of the pyrimidine salvage pathway as defined above. The locus which, as defined above, may comprise, besides a coding sequence, also regulatory sequences, e.g. a sequence which is required for the correct expression of the polypeptide or the coding mRNA may, in further specific embodiments, be targeted by the provision of homologous flanks residing in, or in the vicinity of, said regulatory sequences. By deleting or modifying said regulatory sequences a de facto non expression may result, which is functionally equivalent to the removal of a coding sequence or a part of the coding sequence. Similarly, the homologous flanks may also be provided within the coding sequence, thus resulting in a truncated version of the polypeptide or a fusion with a different coding sequence provided by the integration construct.

The wording “integration into two or more genetic loci encoding an activity of the pyrimidine salvage pathway in a host cell” as used herein means that within the same host cell, two or more loci of the pyrimidine salvage pathway can be used for transformation and thus inclusion of genes or sequences of interest in to said genetic loci. It is, in particular, preferred that the two of more genetic loci relate to those coding for purine/cytosine permease (FcyB), cytosine deaminase (FcyA), uracil-phosphoribosyl-transferase (Uprt), concentrative nucleoside transporter (CntA) or uridine kinase (UK). Without wishing to be bound by theory, it is believed that two or more of the mentioned loci may be used in the context of a counter-selection approach in a host cell on the basis of the same group of prodrug suicide inhibitor compounds: 5-flucytosine (5-FC), 5-fluorouracil (5-FU) and 5-fluorouridine (5-FUR). It is preferred that said integration is performed sequentially, e.g. firstly one locus, e.g. one of fcyB, fcyA, cntA, uprt, or uk, is used and subsequently a different locus is used. A preferred order (firstly (1), secondly (2) etc.) and preferred combinations of integration events are depicted in the following list (i) to (xi):
(i) (1) fcyB; (2) fcyA;
(ii) (1) fcyB; (2) uprt;
(iii) (1) fcyB; (2) cntA, or uk;
(iv) (1) fcyA; (2) uprt;
(v) (1) fcyA; (2) cntA, or uk;
(vi) (1) uprt; (2) cntA, or uk;
(vii) (1) fcyB; (2) fcyA; (3) uprt;
(viii) (1) fcyB; (2) fcyA; (3) cntA, or uk;
(ix) (1) fcyB, (2) uprt; (3) cntA, or uk;
(x) (1) fcyA, (2) uprt; (3) cntA, or uk;
(xi) (1) fcyB; (2) fcyA; (3) uprt; (4) cntA, or uk

The term “coding sequence” refers to a DNA sequence which codes for a specific amino acid sequence. The term “regulatory sequence” refer to a nucleotide sequence located upstream (5′ non-coding sequences), within, or downstream (3′ non-coding sequences) of a coding sequence, and which influences the transcription, RNA processing or stability, or translation of the associated coding sequence. Regulatory sequences may include promoters, enhancers, translation leader sequences, introns, polyadenylation recognition sequences, RNA processing sites, effector binding sites and stem-loop structures.

The term “promoter” refers to a DNA sequence capable of controlling the expression of a coding sequence or functional RNA. Typically, a coding sequence is located 3′ to a promoter sequence. Promoters may be derived in their entirety from a native gene, or be composed of different elements derived from different promoters found in nature, or even comprise synthetic DNA segments. It is understood by a person skilled in the art that different promoters may direct the expression of a gene at different stages of development, or in response to different environmental or physiological conditions. Promoters that cause a gene to be expressed in most cell types at most times are commonly referred to as constitutive promoters. Typically, since the exact boundaries of regulatory sequences have not been completely defined, DNA fragments of different lengths may have identical promoter activity. A promoter may be operably linked with a coding sequence. In a preferred embodiment, the term “promoter” refers to DNA sequence capable of controlling the expression of a coding sequence, which is active in a host cell according to the present invention.

The term “3′ non-coding sequences” refers to DNA sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, i.e. the presence of RNA transcripts, the RNA processing or stability, or translation of the associated coding sequence. The term “RNA transcript” refers to the product resulting from RNA polymerase catalyzed transcription of a DNA sequence. When the RNA transcript is a perfect complementary copy of the DNA sequence, it is referred to as the primary transcript or it may be an RNA sequence derived from post-transcriptional processing of the primary transcript and is referred to as the mature RNA. The term “mRNA” refers to messenger RNA, i.e. RNA that is without introns and that can be translated into protein by the cell.

The term “operably linked” refers to the association of nucleic acid sequences on a single nucleic acid fragment so that the function of one is affected by the other. In the context of a promoter the term means that a coding sequence is rendered capable of affecting the expression of that coding sequence, i.e., the coding sequence is under the transcriptional control of the promoter.

A “host cell” as used herein refers to any cell which comprises at least one functional member of the pyrimidine salvage pathway, preferably at least two functional members of the pyrimidine salvage pathway, more preferably one of purine/cytosine permease (FcyB), cytosine deaminase (FcyA), uracil-phosphoribosyl-transferase (Uprt), concentrative nucleoside transporter (CntA) or uridine kinase (UK), which is amenable to gene introduction and which allows for counterselection of a (functional) absence of a functional member of the pyrimidine salvage pathway via the use of 5-FC, 5-FU and/or 5-FUR. In further preferred embodiments, the host cell comprises at least two or more of purine/cytosine permease (FcyB), cytosine deaminase (FcyA), uracil-phosphoribosyl-transferase (Uprt), concentrative nucleoside transporter (CntA) or uridine kinase (UK), which are amenable to gene introduction and which allow for counterselection of a (functional) absence of a functional member of the pyrimidine salvage pathway via the use of 5-FC, 5-FU and/or 5-FUR. The host cell may be a bacterium or a eukaryotic organism, e.g. a fungus or plant or an alga.

In particularly preferred embodiments the host cell is a bacterium of the genus Klebsiella, Clostridium, Bacillus, Arthobacter, Streptomyces, Corynebacterium, Erwinia, Xanthomonas, Lactobacillus, Caldicellulosiruptor, Pseudomonas, Alcanivorax, Brevibacterium, Bifidobacterium, Escherichia, or Staphylococcus.

In a further preferred embodiment the host cell is a fungus of the genus Aspergillus, Candida, Saccharomyces, Ustilago, Cryptococcus, Fusarium, Rhizopus, Magnaporthe, Komagataella, Trichderma, Penicillium, Acremonium, Mucor, Alternaria, Botrytis, Endothia, Rhizoctonia, Sclerotinia, Klyveromyces, Torulopsis, Sporotrichum, Geotrichum, Verticillium, Botryosphaeria, Trichothecium, Hansenula, Schizosaccharomyces, Brettanomyces, or Neurospora.

In a further preferred embodiment, the host cell may be a plant, e.g. a plant of the genus Arabidopsis, more preferably Arabidopsis thaliana.

In yet another preferred embodiment, the host cell may be an alga.

In particularly preferred embodiments the host cell is an Aspergillus niger, Aspergillus oryzae, Aspergillus fumigatus, Komagataella phaffii, Trichoderma reesei, Penicillium chrysogenum, Acremonium chrysogenum, Candida albicans, Ustilago maydis, Cryptococcus neoformans, Fusarium oxysporum, Rhizopus delemar, or Magnaporthe oryzae cell. In the most preferred embodiment, the host cell is an Aspergillus fumigatus cell.

The language “introducing a gene or sequence of interest into a host cell” as used herein relates to a transformation of the host cell, i.e. the transfer of a genetic element, typically of a nucleic acid molecule, e.g. an integrative nucleic acid construct into said host cell, wherein said transfer results in a genetically stable inheritance. Conditions for a transformation of a host cell, e.g. of bacterial or fungal cells and corresponding techniques are known to the person skilled in the art. These techniques include chemical transformation, protoplast fusion, ballistic impact transformation, electroporation, microinjection, or any other method that introduces the construct into the host cell.

In a specific embodiment, the transformation of fungal cells, e.g. of Aspergillus cells, may be performed by carrying out the following procedure: A: The following media and solution are used: 15 ml Sabouraud liquid medium (SAB) may be used as growth medium for a recipient strain. The transformation may be carried out in Trafo solution 1 comprising 0.6 M KCl+50 mM CaCl₂+5 mM Tris-HCl (pH 7.5) solution (KCl/CaCl₂). Additional Trafo solution 2 comprises Trafo solution 1 including additionally 40% polyethylene glycol (PEG6000 oder PEG4000). A digestion solution may comprise 5% Vinotaste in Trafo solution 1, which is sterile filtered through 0.2 μm filter just before digestion. It may be used with 10 ml/strain. For regeneration solid media, e.g. solid Aspergillus minimal medium, containing 1M Sucrose and 0.7% Agar are used. 20 ml medium is typically poured in a petri dish containing selective conditions. For example, for fcyB locus deletion: the solid medium, e.g. AMM, pH 5, is supplemented with 10 μg/ml 5-FC, or for uprt locus deletion: the solid medium, e.g. AMM, supplemented with 100 μg/ml 5-FC or 5-F may be used. B: The preparation of suitable protoplasts may comprise the following steps: inoculation of 15 ml of SAB with the recipient strain 1×10⁶/ml spores and transfer to a Petri dish (e.g. 9 cm diameter, static cultures). Incubation for e.g. 18 h at e.g. 37° C. Filtering through miracloth and transfer of the mycelium to e.g. 10 ml of filter sterile Trafo Solution 1+5% Vinotase. Incubation for 2 h at 30° C. with mixing (round shaker—speed 70 rpm). Filtering of protoplasts through miracloth. Centrifugation at e.g. 3000 rpm (1600×g—Eppendorf centrifuge 5804 R) for 10 min at 4° C. Resuspension of pellet in 10 ml Trafo Solution 1 by pipetting, and repeating of centrifugation step as described above. Resuspension of pellet in 0.5 ml of Trafo Solution 1 solution (depending on pellet size) and transfer on ice. Subsequently, the number protoplasts may be counted. The protoplasts are adjusted to 0.5×-1×10⁷/ml with Trafo Solution 1. C: The transformation may comprise the following steps: a suitable volume, e.g. 105 μl protoplasts prepared as described above are mixed with 20 μl of a linear DNA fragment. 25 μl of Trafo solution 2 are added. The mixture is pipetted gently 3-4 to homogenize and subsequently incubated on ice for 25 min. Then, 200-300 μl Trafo solution 2 are added and the solution is mixed and incubated for 1-5 min at RT. Subsequently, the solution is transferred into a 15 ml tube, 5-6 ml transformation medium as defined above are added and the solution is poured on the transformation medium as defined above. It is left there for 1-2 h at RT, then transferred to an environment having 37° C. It is incubated there for 2-4 days until colonies start to grow and sporulate. Possible controls may comprise the preparation of 2 tubes, each containing 100 μl of protoplasts, 20 μl A.d. (no DNA) and 25 μl of Trafo Solution 2. One tube may be transferred on media containing antibiotic (negative control) and the other on the media without antibiotic (recovery plate=positive control).

The term “growing a transformed host cell” as used herein refers to the use of any suitable means and methods known to the person skilled in the art, which allows the growth of a host cell as defined herein and which is suitable for host cell under selective medium conditions. The culture medium may, for example, be adapted to the growth pattern of the host cell, e.g. comprise a carbon source or, in case of autotrophic organisms lack a carbon source.

In specific embodiments for bacterial host cells of the Escherichia group, e.g. E. coli and related organisms, media such as Terrific Broth (TB), Luria-Bertani Medium (LB), or M9 minimal medium may be used. The skilled person would further be aware of other media which are suitable for bacteria, also envisaged herein, as well as their preparation, e.g. from suitable literature sources or databases. Typically, the TB medium may comprise in a 1 liter unit 12 g Bacto tryptone, 24 g Bacto yeast extract, 4 mL Glycerol, add distilled water ad 900 ml, which is autoclaved and subsequently completed with the addition of 100 mL sterile 0.17M KH₂PO₄ and 0.72M K₂HPO₄. Typically, the LB medium may comprise in a 1 liter unit. Typically, the LB medium may comprise in a 1 liter unit 10 g Bacto-tryptone, 5 g yeast extract, 10 g NaCl, distilled water ad 1000 ml, which is subsequently autoclaved. Typically, a M9 minimal medium in a 1 liter unit may comprise 880 ml sterile water, 100 ml M9 salts stock solution, 1 ml autoclaved 1 M MgSO₄, 0.1 ml autoclaved 1 M CaCl₂ and 20 ml 20% glucose (sterile), wherein the M9 salts stock solution (10×) comprises 60 g Na₂HPO₄ x 7 H₂O, 30 g KH₂PO₄, 5 g NaCl, 10 g NH₄Cl to which water ad 1000 ml is added and which is subsequently autoclaved. The medium may be provided as liquid medium, or alternatively as solid medium, e.g. by adding a suitable amount of agar.

In specific embodiments for streptomycetal host cells and related organisms media such as TSB and R2YE Medium may be used. The skilled person would further be aware of other media which are suitable for streptomycetes, also envisaged herein, as well as their preparation, e.g. from suitable literature sources or databases. The media may further be modified, e.g. in view of the specific strain to be used. Corresponding information would be known to the skilled person or can be derived from suitable literature sources. Typically, the TSB medium may comprise in a 1 liter unit 17 g Tryptone, 3 g Phytone, 5 g NaCl, 2.5 g K₂HPO₄, 2.5 g glucose, and distilled water ad 1 L, wherein the ingredients are dissolved under gentle heat and then autoclaved for 15 minutes at 121° C. Typically, the R2YE medium may comprise as (i) medium A in a 1 liter unit 103 g Sucrose, 0.25 g K₂SO₄, 10.12 g MgCl₂.6H₂O, 10 g Glucose, 0.1 g Difco casamino acids, 800 mL Distilled water and 5 g Difco yeast extract; and as (ii) medium B 2 mL Trace element solution, 100 mL TES buffer (5.73%, w/v), 10 mL KH₂PO₄ (0.5%, w/v), 80 mL CaCl₂x2H₂O (3.68%, w/v), 15 mL L-proline (20%, w/v), 5 mL 1 M NaOH, wherein said Trace element solution comprises in a 1 liter unit 40 mg ZnCl₂, 200 mg FeCl₃x 6H₂O, 10 mg CuCl₂x 2H₂O, 10 mg MnCl₂ x 4H₂O, 10 mg Na₂B₄O₇x 10H₂O, and 10 mg (NH₄)₆Mo₇O₂₄x 4H₂O, wherein a bottle containing medium A is autoclaved, subsequently cooled to at least 50° C. and added to medium B, preferably in a biological safety cabinet. The medium may be provided as liquid medium, or alternatively as solid medium, e.g. by adding a suitable amount of agar. Further information or alternative media definitions would be known to the skilled person or can be derive from suitable literature sources such as Kawai et al., Bioeng Bugs. 2010; 1(6):395-403 for Saccharomyces cerevisiae, or Weigel and Glazebrook, CSH Protoc. 2006; 2006(7) for Arabidopsis.

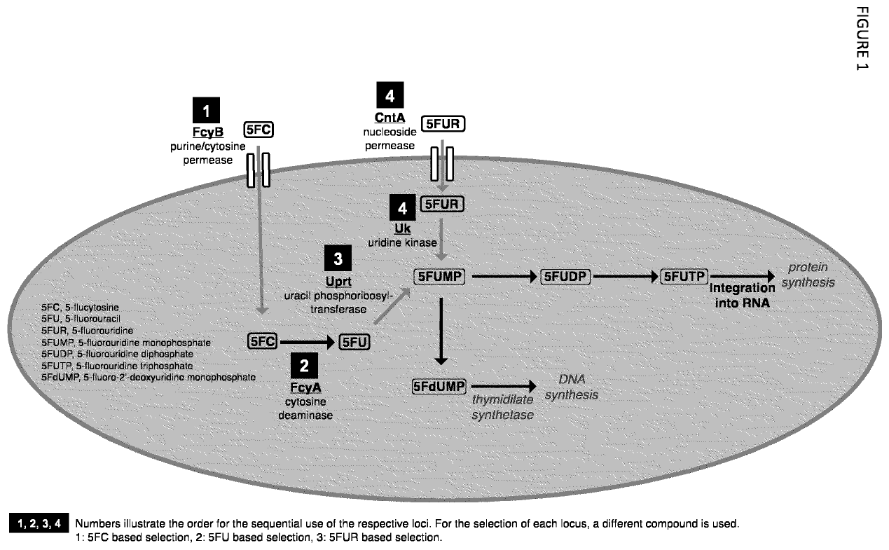

The present invention specifically envisages that the growth takes place in a selective medium. The term “selective medium” as used herein relates to a medium which comprises an efficient amount of 5-flucytosine (5-FC), 5-fluorouracil (5-FU) and/or 5-fluorouridine (5-FUR). 5-FC, 5-FU and 5-FUR are prodrug suicide inhibitors which are transported into a host cell and which are converted into the toxic substance 5-fluorouridine monophosphate (5-FUMP) or 5-fluoro deoxyuridine monophosphate (5-FdUMP) which are further converted into 5-FUTP or 5-FdUTP, respectively and eventually interfere with RNA and DNA biosynthesis as well as protein metabolism and thereby exert their cell toxic properties. The selective characteristics of 5-FC, 5-FU and 5-FUR within the methods of the present invention depend on the member of the pyrimidine salvage pathway targeted or employed for the introduction of an integrative nucleic acid construct. For example, as is also shown in FIG. 1, 5-FC may be used as selective compound in a selective medium according to the present invention in case purine/cytosine permease is targeted. In a further embodiment, 5-FU may be used as selective compound in a selective medium according to the present invention in case uracil-phosphoribosyl-transferase is targeted. In a further embodiment, 5-FC may be used as selective compound in a selective medium according to the present invention in case uracil-phosphoribosyl-transferase is targeted. In this embodiment, 5-FC is converted to 5-FU by a different enzymatic activity (FcyA). In another embodiment, 5-FUR may be used as selective compound in a selective medium according to the present invention in case concentrative nucleoside transporter is targeted. In another embodiment, 5-FUR may be used as selective compound in a selective medium according to the present invention in case uridine kinase is targeted.

In specific embodiments, genetic loci of the pyrimidine salvage pathway may be used for second, third or fourth site-directed integration events. For example, if purine/cytosine permease has already been targeted and its activity is no longer present in a host cell, a secondary site-directed integration may be performed by targeting uracil-phosphoribosyl-transferase. In this embodiment, 5-FU may be used as selective compound in a selective medium according to the present invention. Alternatively, in case purine/cytosine permease has already been targeted and its activity is no longer present in a host cell, a secondary site-directed integration may be performed by targeting concentrative nucleoside transporter or uridine kinase. In this embodiment, 5-FUR may be used as selective compound in a selective medium according to the present invention. Similarly, in case purine/cytosine permease and/or uracil-phosphoribosyl-transferase has already been targeted and the activity is no longer present in a host cell, a secondary or tertiary site-directed integration may be performed by targeting concentrative nucleoside transporter or uridine kinase. In this embodiment, 5-FUR may be used as selective compound. An example of the order of multiple sequential site-directed integration events to comprehensively exploit the potential of the pyrimidine salvage pathway knock-in strategy can be derived from FIG. 1. For example, a site-directed integration may start with event 1, i.e. a targeting of purine/cytosine permease (based on the use of 5-FC). In a second event 2, e.g. in a strain in which event 1 has already occurred, uracil-phosphoribosyl-transferase may be targeted (based on the use of 5-FU). In a third event 3, e.g. in a strain in which event 1 and/or event 2 have already occurred, either nucleon) side permease (based on the use of 5-FUR) or uridine kinase (based on the use of 5-FUR) may be targeted.

In further alternative embodiments, two or more of the mentioned genetic loci of the pyrimidine salvage pathway may be used for simultaneous site-directed integration events.

The amount of 5-FC, 5-FU or 5-FUR to be used in the selective medium according to the present invention varies and may typically be adapted to the host cell used, the medium used, the growth conditions selected etc. In specific embodiments, the concentration of 5-FC to be used in the selective medium is between about 1 μg/ml to 200 μg/ml, preferably 10 μg/ml 5-FC, e.g. for a transformation of A. fumigatus, on minimal media such as AMM pH 5 (with a preferred AMM Composition of: 55.5 mM D-glucose, 20.0 mM ammonium tartrate, 7 mM KCl, 2.1 mM MgSO₄x 7H₂O, 11.2 mM KH₂PO₄, 0.09 LIM Na₂B₄O₇ x 10H₂O, 1 μM CuSO₄ x 5H₂O, 10 μM FeSO₄ x 7H₂O, 4.5 μM MnSO₄ x 4H₂O, 3.1 μM Na₂MoO₄ x 10H₂O, 10 μm ZnSO₄ x 7H₂O, 0.7% Agar; adjusted to pH 6.5 using NaOH before autoclaving). In further specific embodiments, the concentration of 5-FU to be used in the selective medium is between about 10 μg/ml to 500 μg/ml, preferably 100 μg/ml 5-FU for transformation e.g. for a transformation of A. fumigatus on minimal media such as AMM as defined above. In other specific embodiments, the concentration of 5-FUR to be used in the selective medium is between about 10 μg/ml to 200 μg/ml, preferably 100 μg/ml 5-FUR for transformation e.g. for a transformation of A. fumigatus on minimal media such as AMM as defined above.

It is particularly preferred to use 5-FC and/or 5-FU in certain concentration ranges for loci of the pyrimidine salvage pathway. For example, for a knock-in in the fcyB locus a range of 10 and 50 μg/ml 5-FC may be used. It is particularly preferred to use a concentration of 10 μg/ml 5-FC. In a further example, for a knock-in in the fcyA locus a range of 10 and at least 100 μg/ml 5-FC may be used. It is particularly preferred to use a concentration of 100 μg/ml 5-FC. In yet another embodiment, for a knock-in in the uprt locus a range of 10 and at least 100 μg/ml 5-FC may be used. Furthermore, it is preferred to additionally use at least at least 100 μg/ml 5-FU.

In further embodiments, it is particularly preferred to use 5-FUR in certain concentration ranges for loci of the pyrimidine salvage pathway. For example, for a knock-in in the cntA locus a range of 10 and 100 μg/ml 5-FUR may be used. It is particularly preferred to use a concentration of 10 μg/ml 5-FUR. In yet another embodiment, for a knock-in in the uk locus a range of about 10 and at least 100 μg/ml 5-FUR may be used. It is particularly preferred to use a concentration of 10 μg/ml 5-FUR.

In further embodiments, the selection conditions may be varied via the pH of the medium and/or inhibitor. It is preferred to use the inhibitor 5-FC at a pH of about 5. In further embodiments, 5-FU may be used at a pH of about between 5 or 7. Similarly, 5-FUR may be used at a pH of about between 5 and 7.

The growth of a transformed hot cell may be performed according to any suitable method. For example, the growth may be a batch or continuous fermentation process, which would be well known to the person skilled in the art and is described in the literature, e.g. in Li et al., Microb Cell Fact, 2015, 14 (83). The culturing may be carried out under specific temperature conditions, e.g. between 15° C. and 37° C., preferably between 20° C. and 30° C. or 15° C. and 30° C., more preferably between 20° C. and 30° C. and most preferably at about 24° C. In another embodiment, the culturing may be carried out at a broad pH range, e.g., between pH 6 and pH 9, preferably between pH 6.5 and 8.5, more preferably between 6.7 and 7.5 and most preferably between 6.8 and 7, e.g. at about 7. Further details may be derived from suitable literature sources such as Li et al., Microb Cell Fact, 2015, 14 (83). The growth period may vary in dependence on the dimension of the fermentation approach, the medium used, the host cell used, the selective compound used etc.

In certain embodiments of the present invention, a growth period of about 2 to 4 days may be used, e.g. 48 to 72 h, e.g. 50, 55, 60, 65, 70, 75, 80, 85, 90 or 96 h. Also envisaged are growth periods of about 10 to 24 h such as 12, 14, 16, 18 or 20 h or any value in between the mentioned values.

In further specific embodiments, the culture medium may comprise additional substances. An example of such an additional substance is an antibiotic, e.g. tetracyclin, ampicillin, kanamycin. Such antibiotics may be used as selection instruments for extrachromosomal elements comprising a corresponding resistance cassette, or as inducers for corresponding regulated promoters, e.g. as defined herein below in specific embodiments. They may be used in any suitable concentration, e.g. in a suitable concentration range of 50 to 400 μg/ml in the case of ampicillin such as 50, 100, 150 μg/ml, or in a suitable range of 25 to 50 μg/ml in the case of kanamycin, such as 25 or 50 μg/ml. Further details would be known to the skilled person, or can be derived from suitable literature sources. Antibiotics may, in particular, be used in embodiments, in which the currently described method of site-directed integration is combined with a traditional marker-based integration approach, e.g. employing antibiotics resistance cassettes for site-directed integration at different locations in the genome of a host cell, as described further below.

The final step of the method according to the present invention is the selection of a host cells which is capable of growing under the medium conditions as mentioned above, i.e. which is capable of growing in a medium comprising 5-FC, 5-FU or 5-FUR. The selection may, for example be the identification and subsequent isolation of a cell which is capable of growing on a solid medium plate, e.g. as a colony, or which is growing in a liquid medium, e.g. showing an increased growth rate. The selection may, in certain embodiments, be accompanied with the usage of suitable control experiments, e.g. the use of non-transformed or WT host cells to have comparison standards.

In preferred embodiments, the integrative nucleic acid construct comprises a control element linked to a gene of interest or a sequence of interest or a sequence to be expressed. The control element may, for example, be a promoter as defined herein or a terminator sequence as defined herein. These sequences may be operably linked to the gene of interest or a sequence of interest or a sequence to be expressed. Also envisaged is the presence of a regulatory sequence as defined herein. For example, an enhancers, a translation leader sequence, a polyadenylation recognition sequences, an RNA processing site, an effector binding sites and/or a stem-loop structure may be present in the integrative nucleic acid construct.

It is particularly preferred that the integrative nucleic acid construct does not comprise a nucleic acid sequence encoding a marker gene for selection of a genetically transformed host cell. Such marker gene may, in a typical example, be an antibiotics resistance cassette.

In preferred embodiments, the integrative nucleic acid construct comprises a gene of interest or a sequence of interest. The term “gene of interest” as used herein refers to any gene or genetic element which provides a function or activity considered to be of interest for skilled person and which is planned to be integrated into the genome of a host cell. The term “genetic element” as used herein means any molecular unit which is able to transport genetic information. It accordingly relates to a gene and the term also refers to a homologous or native gene, a chimeric gene, a heterologous or foreign gene, a transgene or a codon-optimized gene. The term “gene” refers to a nucleic acid molecule or fragment that expresses a specific protein, preferably it refers to nucleic acid molecules including regulatory sequences, e.g. as defined above, preceding (5′ non-coding sequences) and following (3′ non-coding sequences) the coding sequence. A “native gene” or “homologous gene” as used herein means a gene which is derived from the same organism, or the same species or species variant. It shows hence no sequence difference with respect to the gene present in the genome. However, the homologous gene may be provided, in certain embodiments, in a different genomic context or be provided in different numbers than given in the WT situation. The term “chimeric gene” refers to any gene that is in its present form not a native gene, comprising regulatory and coding sequences that are not found together in nature, e.g. comprising a native regulatory sequence and a foreign coding sequence or vice versa. Accordingly, a chimeric gene may comprise regulatory sequences and coding sequences that are derived from different sources, or regulatory sequences and coding sequences derived from the same source, but arranged in a manner different than that found in nature. According to the present invention a “foreign gene” or “heterologous gene” refers to a gene not normally found in the organism but that is introduced into said organism, or has been modified in the organism to correspond to said foreign gene. Foreign genes can comprise native genes inserted into a non-native organism, or chimeric genes. The term “transgene” refers to a gene that has been introduced into the genome by a transformation procedure. A “sequence of interest” as used herein relates to any nucleic acid sequence, which provides one or more functions or activities considered to be of interest for a skilled person and which is/are planned to be integrated into the genome of a host cell. Accordingly, a sequence of interest may comprise a gene or interest as defined herein. In further embodiments, it may comprise more than one gene or more than one coding sequence. Examples of such sequences are gene clusters comprising several genes or elements comprising all or many genes of a pathway, or chromosomal regions comprising several genes etc. The size of these genes or interest or sequences of interest is variable. For example the gene of interest may have a size of about 100 bp to about 15 kb. Preferred size ranges are form about 100 bp to about 500 bp, from about 100 bp to about 1000 bp, form about 100 bp to about 1500 bp, from about 100 bp to about 2000 bp, from about 100 bp to about 2500 bp, from about 1000 bp to about 3000 bp, from about 1000 bp to about 3500 bp, from about 1000 bp to about 4000 bp, from about 1000 bp to about 4500 bp, from about 1000 bp to about 5000 bp. Also envisaged are any values in between the mentioned values. A sequence of interest may have any suitable size of between about 20 bp to about 500 kbp. For example, the sequence of interest may have a size of about 5 kbp to about 15 kbp, from about 5 kbp to about 20 kbp, from about 5 kbp to about 30 kbps, from about 5 kbp to about 40 kbp, from about 5 kbp to about 50 kbp, from about 5 kbp to about 60 kbp, from about 5 kbp to about 75 kbp, from about 5 kbp to about 100 kpb, from about 100 kbp to about 500 kbpt, from about 100 kbp to about 250 kbp or from about 250 kbp to about 500 kbp. Also envisaged are any values in between the mentioned values. The present invention also contemplates small sequences which have a size of about 20 bp to about 100 bp, e.g. about 20 bp to about 50 bp, or about 30 bp to about 70 bp, or about 40 bp to about 100 bp.

In specific embodiment of the present invention the gene interest encodes an enzymatic activity, or the sequence of interest may encode more than one enzymatic activity. The term “enzymatic activity” relates to any suitable enzymatic activity known to the skilled person. The term comprises extracellular and intracellular enzymes. In case a secretion of the enzyme is necessary for its proper function, also transporter or secretion machinery components may be comprised in the sequence of interest. Also envisaged is the provision of such elements on two or more different sequences of interest which may be inserted at different positions of the genome, e.g. on the basis of two or more pyrimidine salvage pathway members as described herein.

Envisaged examples of such activities, which are however not limiting, are an isomerase, oxidase, reductase, oxidoreductase, hydrolase, ligase, lyase, cellulase, chitinase, amylase, lactase, glucosidase, xylanase, transferase, esterase, lipase, mannosidase, glucanase, protease, phytase, invertase, peroxidase, peptidase, pectinase, chymosin and pepsin. Further examples of suitable enzymatic activities may be known to the skilled person or can be derived from internet resources such as Brenda (http://www.brenda-enzymes.org/) or ExplorEnz (http://www.enzyme-database.org/).

The enzymatic activity may be provided as transgene or foreign gene, or it may be provided as native or homologous gene. It may preferably be operably linked to a regulatory sequence, preferably a promoter sequence as defined herein above. Also the presence of further regulatory sequences such as a terminator sequence or an enhancer is envisaged. In a preferred embodiment, the gene or interest or sequence of interest encodes a homologous activity of the host cell. This activity may be provided in a way, that amount of enzyme or protein or the enzymatic activity is modified. Typically, the amount of enzyme or protein or the enzymatic activity is increased. The homologous gene may, for example, be provided in a multicopy fashion, it may be inserted at a different genomic location than in the WT situation, it may be provided with different regulatory sequences leading to a differently controlled gene expression, e.g. via a constitutive promoter or a regulable or tunable promoter.

The integration of the gene of interest or the sequence of interest may advantageously lead to the expression of the mentioned enzymatic activity or activities. The term “expression” or “expressed” as used herein refers to the transcription and accumulation of sense strand (mRNA) derived from nucleic acid molecules or genes as mentioned herein, e.g. of genes or genetic elements. Preferably, the term also refers to the translation of mRNA into a polypeptide or protein and the corresponding provision of such polypeptides or proteins within the cell and/or the provision an enzymatic or functional activity conveyed by said polypeptides or proteins.

In a further preferred embodiment, said expression as mentioned herein above is an overexpression. The term “overexpression” relates to the accumulation of more transcripts and in particular of more polypeptides and activities than upon the expression of a native copy of the genetic element which gives rise to said polypeptide or activity in the context of the organism of origin. In further, alternative embodiments, the term may also refer to the accumulation of more transcripts and in particular of more polypeptides or activities than upon the expression of typical, moderately expressed housekeeping genes such as cysG, hcaT or rssA, e.g. in E. coli, or scoF2, kasOP, ermE, rpsi or sucA in Streptomycetes. In preferred embodiments, the overexpression as mentioned above may lead to an increase in the transcription rate of a gene of about 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% or any value in between these values in comparison to the corresponding WT or native transcription (without modification or over-expression) in the context of the organism of origin.

In a further preferred embodiment, the gene of interest or the sequence of interest encodes one or more activities involved in the production of carbohydrates, fatty acids or lipids. Examples of such activities, which are however not limiting, are acyl-CoA synthetase, or enzymes involved in beta oxidation such as acyl CoA dehydrogenase, enoyl-CoA hydratase, 3-hydroxyacyl-CoA dehydrogenase, and 3-ketoacyl-CoA thiolase.

In a further preferred embodiment, the gene of interest or the sequence of interest encodes one or more activities involved in the production of a pharmaceutically active protein or peptide, or a pharmaceutically active protein or peptide. Examples of envisaged pharmaceutically active proteins or peptides, which are however not limiting, are hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, cytokines and immune system proteins (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, T cell receptors, immunoglobulins, soluble major histocompatibility complex antigens, immunologically active antigens such as bacterial, parasitic, or viral antigens or allergens), autoantigens, antibodies), enzymes (tissue plasminogen activator, streptokinase, cholesterol biosynthetic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (sterpod binding proteins, growth hormone or growth factor binding proteins and the like), transcription and translation factors, oncoprotiens or proto-oncoprotiens (e.g., cell cycle proteins), muscle proteins (myosin or tropomyosin and the like), myeloproteins, neuroactive proteins, tumor growth suppressing proteins (angiostatin or endostatin, both which inhibit angiogenesis), anti-sepsis proteins (bactericidal permeability-increasing protein), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, Protein C, von Wilebrand factor, antithrombin III, glucocerebrosidase, erythropoietin granulocyte colony stimulating factor (GCSF) or modified Factor VIII, anticoagulants such as huridin) etc.

In a further preferred embodiment, the gene of interest or the sequence of interest encodes an antibiotic or an activity involved in the production of an antibiotic. Examples of envisaged antibiotics, which are however not limiting, are bacitracin, colistin or polymyxin B. It is further envisaged that the gene of interest or sequence of interest encodes an activity or group of activities capable of producing and/or modifying antibiotics such as aminoglycosides, ansamycins, carbapenems, cephalosporins, glycopeptides, lincosamides, lipopeptides, macrolides, monobactams, nitrofurans, oxazolidinones, penicillines, quinolones, sulfonamides or tetracyclines.

In a further preferred embodiment, the gene of interest or the sequence of interest encodes an activity or activities involved in the production of biofuels. Envisaged examples of such activities, which are however not limiting, include lipases and phospholipases.

In a further preferred embodiment, the gene of interest or the sequence of interest encodes an activity or activities involved in the production of foodstuff or animal feedstuff. Envisaged examples of such activities, which are however not limiting, include amyloglucosidases, carbhydrases, cellulases, catalases, esterase-lipases, galactosideases, milkclotting enzymes, amylases, bromelain, peptide hydrolases, lactases, lipases, chymosin, aminopeptidase, and invertases.

In a further preferred embodiment, the gene of interest or the sequence of interest encodes an activity or activities involved in the production of vitamins or dietary supplements. Envisaged examples of such activities, which are however not limiting, include FMN adenyltransferase, flavokinase, 2,5-diketo-D-gluconic acid reducatase, lactonohydralase, nitril hydratase, nitriliase, NAD kinase, formic acid dehydrogenase, glucose dehydrogenase, FAD synthase, S-adenosylmethionine synthetase, S-adenosylhomocysteine hydrolase, beta-oxidation-line enzymes, aldehyde reductase, pyridoxamine oxidase, CDP-choline pyrophosphorylase, NDP-glucose pyrophosphorylase. Further envisaged is the employment of multiple enzyme systems, e.g. based on gene clusters or on biochemical pathway member encoding sequences. Examples include a multiple enzyme system from Geotrichum candidum for the production of vitamin E and K1 side chains, a multiple enzyme system from Flavobacterium sp. For the production of vitamin K2 or a multiple enzyme system from Mortiella alpina for the production of eicosapentaenoic acid.

In a further preferred embodiment, the gene of interest or the sequence of interest encodes an activity or activities involved in the production of amino acids. Envisaged, non-limiting, examples of such activities include aspartase, L-aspartate beta-decarboxylase, L-AAC-hydrolase, AAC racemase, phenylalanine ammonia lyase and transaminase.

In a further preferred embodiment, the gene of interest or the sequence of interest encodes an activity or activities involved in the production of cosmetic ingredients. Envisaged examples of such activities, which are however not limiting, include enzymes, e.g. lipases, involved in the production or modification of cosmetic esters such as glyceryl stearate, isopropyl palmitate, 2-ethylhexyl palmitate, isopropyl myeristate, myristyl myristate, glyceryl oleate, isononyl isononanoate, isostearyl linoleate, hexal laureate, cetyl ricinoleate, cetyl palmitate or isopropyl isostearate.

In a further preferred embodiment, the gene of interest or the sequence of interest encodes an activity or activities involved in the production of organic raw materials. Envisaged examples of such activities, which are however not limiting, include laccase, ligninase, hemicellulase, cellulase, pectinase, amylase, beta-glucanase, inulinase, invertase, lactase, mannanase, xylanase, beta-xylosidase, beta-fructofuranosidase, phytase, polygalacturonidase.

In a further preferred embodiment, the gene of interest or the sequence of interest encodes a protein used in metabolic engineering or synthetic biology such as in cell factory generation or optimization. The term “metabolic engineering” as used herein refers to the modification of the endogenous metabolic network of an organism, e.g. in order to harness it for a useful biotechnological task, for example, production of a value-added compound etc. This may, for example, include the creation of synthetic metabolic networks that are able to outcompete naturally evolved pathways or redirect flux toward non-natural products. Further information can be derived from suitable literature sources such as Erb et al., 2017, 37, 56-62. Envisaged examples of such metabolic engineering components include enzymatic activities involved in the production of scylloinositol, e.g. as described in detail in Tanaka et al, Microbial Cell Factories, 2017, 16, 67.

The present invention further contemplates biomolecular marker protein encoding sequences or genes as genes of interest or sequences of interest. Examples of such biomolecular markers include, but are not limited to, fluorescent or color emitting proteins or peptides, e.g. green fluorescent protein (GFP), luciferin, luciferase, mCherry, mOrange, TagBFP, Cerulean, Citrine, mTurquoise, red fluorescene protein (RFP), yellow fluorescence protein (YFP) and derivatives thereof such as EGFP, ECFP, BFP, EBFP, EBFP2 or BFP.

Also envisaged is the provision of genes or sequences of interest comprising, essentially consisting of or consisting of an RNA expression cassette. The RNA expression cassette may, for example, be designed to express an antagonist of an expression product such as an antisense RNA molecule, a miRNA, a siRNA molecule or a catalytic RNA molecule, which can, for example, be used for gene silencing. Accordingly, the RNA expression cassette may comprise or provide one or more elements required for RNA gene silencing.

The “antisense RNA” of the invention typically comprises a sequence complementary to at least a portion of an RNA transcript of a gene to be silenced. However, absolute complementarity, although preferred, is not required. A sequence “complementary to at least a portion of an RNA transcript” as referred to herein, means a sequence having sufficient complementarity to be able to hybridize with the RNA, forming a stable duplex triplex formation in the case of double stranded antisense nucleic acids. The ability to hybridize will depend on both the degree of complementarity and the length of the antisense nucleic acid. Generally, the larger the hybridizing nucleic acid, the more base mismatches with a RNA sequence of the invention it may contain and still form a stable duplex or triplex. A person skilled in the art can ascertain a tolerable degree of mismatch by use of standard procedures to determine the melting point of the hybridized complex. Preferably antisense molecules complementary to the 5′ end of the transcript, e.g., the 5′ untranslated sequence up to and including the AUG initiation codon may be used for the inhibition of translation. In a further preferred embodiment, sequences complementary to the 3′ untranslated sequences of mRNAs may also be used.

The term “siRNA” refers to a particular type of antisense-molecules, namely small inhibitory RNA duplexes that induce the RNA interference (RNAi) pathway. These molecules can vary in length and may be between about 18-28 nucleotides in length, e.g. have a length of 18, 19, 20, 21, 22, 23, 24, 25, 26, 27 or 28 nucleotides. Preferably, the molecule has a length of 21, 22 or 23 nucleotides. The siRNA molecule according to the present invention may contain varying degrees of complementarity to their target mRNA, preferably in the antisense strand. siRNAs may have unpaired overhanging bases on the 5′ or 3′ end of the sense strand and/or the antisense strand. The term “siRNA” includes duplexes of two separate strands, as well as single strands that can form hairpin structures comprising a duplex region. Preferably the siRNA may be double-stranded wherein the double-stranded siRNA molecule comprises a first and a second strand, each strand of the siRNA molecule is about 18 to about 23 nucleotides in length, the first strand of the siRNA molecule comprises nucleotide sequence having sufficient complementarity to the target RNA via RNA interference, and the second strand of said siRNA molecule comprises nucleotide sequence that is complementary to the first strand. Methods for designing suitable siRNAs directed to a given target nucleic acid are known to person skilled in the art, e.g. from Elbashir et al., 2001, Genes Dev. 15, 188-200.

The term “miRNA” refers to a short single-stranded RNA molecule of typically 18-27 nucleotides in length, which regulate gene expression. miRNAs are encoded by genes from whose DNA they are transcribed but are not translated into a protein. In a natural context miRNAs are first transcribed as primary transcripts or pri-miRNA with a cap and poly-A tail and processed to short, 70-nucleotide stem-loop structures known as pre-miRNA in the cell nucleus. This processing is typically performed by a protein complex known as the Microprocessor complex, consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). This complex is responsible for the gene silencing observed due to miRNA expression and RNA interference. Either the sense strand or antisense strand of DNA can function as templates to give rise to miRNA. Typically, efficient processing of pri-miRNA by Drosha requires the presence of extended single-stranded RNA on both 3′- and 5′-ends of hairpin molecule. These ssRNA motifs could be of different composition while their length is of high importance if processing is to take place at all. Generally, the Drosha complex cleaves the RNA molecule ^{˜}22 nucleotides away from the terminal loop. Pre-miRNAs may not have a perfect double-stranded RNA (dsRNA) structure topped by a terminal loop. When Dicer cleaves the pre-miRNA stem-loop, typically two complementary short RNA molecules are formed, but only one is integrated into the RISC complex. This strand is known as the guide strand and is typically selected by the argonaute protein, the catalytically active RNase in the RISC complex, on the basis of the stability of the 5′ end. The remaining strand, known as the anti-guide or passenger strand, is typically degraded as a RISC complex substrate. After integration into an active RISC complex, miRNAs may base pair with their complementary mRNA molecules and inhibit translation or may induce mRNA degradation by the catalytically active members of the RISC complex, e.g. argonaute proteins. Mature miRNA molecules are typically at least partially complementary to mRNA molecules corresponding to the expression product of the present invention, and fully or partially down-regulate gene expression. Preferably, miRNAs according to the present invention, for instance as identifiable and obtainable according to assays and methods described in Hüttenhofer and Vogel, 2006, NAR, 34(2): 635-646, may be 100% complementary to their target sequences. Alternatively, they may have 1, 2 or 3 mismatches, e.g. at the terminal residues or in the central portion of the molecule. miRNA molecules according to the present invention may have a length of between about 18 to 27 nucleotides, e.g. 18, 19, 20, 21, 22, 23, 24, 25, 26 or 27 nucleotides. Preferred are 21 to 23 mers. miRNAs having 100% complementarity may preferably be used for the degradation of nucleic acids according to the present invention, whereas miRNAs showing less than 100% complementarity may preferably be used for the blocking of translational processes.

The term “catalytic RNA” or “ribozyme” refers to a non-coding RNA molecule, which is capable of specifically binding to a target mRNA and of cutting or degrading said target mRNA, e.g. a transcript comprising the nucleotide sequence of SEQ ID NO: 1, 4, 7, 8 or 9. Typically, ribozymes cleave mRNA at site specific recognition sequences and may be used to destroy mRNAs corresponding to the polynucleotides of the invention. A preferred example of ribozymes are hammerhead ribozymes. Hammerhead ribozymes cleave mRNAs at locations dictated by flanking regions that form complementary base pairs with the target mRNA. The construction and production of hammerhead ribozymes is known in the art and is described in Haseloff and Gerlach, 1988, Nature, 334: 585-591. Preferably, the ribozyme may be engineered so that the cleavage recognition site is located near the 5′ end of the mRNA to be destroyed.

In a specific embodiment the gene of interest or sequence of interest is modified with respect to the codon usage of the coding sequence. This modification is typically an adaptation of the codon usage of a gene or genetic element as defined herein above to the codon usage of the genes which are transcribed or expressed most often in the target organism, i.e. a host cell as defined herein, or which are most highly expressed (in comparison to a housekeeping gene, e.g. as defined herein above). The term “adapted” as used herein means that on the basis of the degeneration of the genetic code and the fact that most amino acids are encoded by more than one codon triplet, the preferred codons of the host cell may be determined or derived from suitable literature sources. The gene of interest or sequence of interest may accordingly be modified without change of the amino acid sequence by replacing rarely used codons with more frequently used codons of the host cell. Examples of such codon-usage of highly expressed genes may, for example, comprise the codon-usage of a group of the 5, 10, 15, 20, 25 or 30 or more most highly expressed genes of the organism is which the expression takes place.

Also envisaged is the adaptation of the dicodon usage, i.e. of the frequency of all two consecutive codons within a coding sequence. By adapting the dicodon usage in the nucleotide sequences of a gene of interest or sequence of interest to the situation in the host cell, potential translational problems as well as potentially problematic recognition regions or sites in the mRNA transcript (typically being in the size of about 4 to 6 nucleotides) may be avoided. Correspondingly redesigned sequences may be synthesized de novo and subsequently introduced into the host cell by site directed integration into the genetic loci of the pyrimidine salvage pathway as described herein.

In a further specific embodiment, the approach and methods of the present invention include the additional genetic modification of a host cell. Such an additional genetic modification may, for example, comprise the integration of genes or sequences, e.g. of one or more additional homologous genes or of one or more heterologous genes or sequences, the provision of a further activity, e.g. enzymatic activity, an increase or decrease of the expression of a gene, a silencing of a gene, a deletion of one or more genes or loci or gene clusters. This modification preferably involves genomic locations which are not associated with the pyrimidine salvage pathway.

Corresponding modifications may, for example, be based on the usage of typically antibiotics resistance marker cassettes, e.g. providing resistance to kanamycin, hygromycin, pyrithiamine, phleomycine (e.g. zeocin, bleomycin, etc.) and derivatives thereof, the amino glycoside G418, or nourseothricin (also termed NTC or ClonNAT). Furthermore, selection for auxotrophic markers e.g. based on the ability to grow on media lacking uracil, leucine, histidine, methionine, lysine or tryptophane may be employed. When using a selection marker as mentioned above or any other suitable marker, sequences of the Cre-lox system may be used in addition to the marker. This system allows upon expression of the Cre recombinase after the insertion of the genetic element, e.g. the deletion cassette, an elimination and subsequent reuse of the selection marker. The term “Cre-lox system” as used herein relates to the combination of Cre recombinase and its respective recognition sites (lox sites). Alternatively, the system may be composed of FLP recombinase and its respective recognition sites (FRT sites). By providing the recognition sites in a direct repeated manner a deletion of sequences between the repeats can be achieved. Similarly, by providing other orientations or more than two recognition sites further rearrangement pattern may become possible, e.g. an inversion of the sequences. Further details may be derived from Ryder et al., 2004, Genetics, 167,797-813 or Ito et al., 1997, Development, 771,761-771. Also envisaged is the use of other, similar recombinase systems, which would be known the skilled person.

In further specific embodiments, the employment of genomic editing systems, which may be used to provide genomic modifications without the necessity of inserting antibiotics resistance cassettes or any additional selection marker, is envisaged. Such genomic editing approaches may, for example, be the CRISPR/Cas system, a TALEN-based system, or a zinc finger nuclease (ZFN)-based system.

Particularly preferred is the use of the CRISPR (Clustered Regularly Interspaced Short Palindromic Repeats)/Cas system. CRISPR/Cas can be utilized to reduce expression of specific genes (or groups or similar genes) or to edit genomic sequences. This is typically achieved through the expression of single stranded RNA in addition to a CRISPR gene or nuclease. The technique typically relies on the expression of a CRISPR gene such as Cas9, or other similar genes in addition to an RNA guide sequences (see, for example, Cong et al. 2013, Science, 339 (6121), 819-823). Double stranded cleavage may accordingly be targeted to specific sequences using the expression of appropriate flanking RNA guide sequences, which may be provide as one component of the multicomponent system, e.g. together with Cas9 or a similar functionality. In a preferred embodiment RNA guide sequences and CRISPR gene expression (e.g. Cas9) may be included as part of an expression construct.

The term “TALEN-based system” relates to the use of TALEN, i.e. the Transcription Activator-Like Effector Nuclease, which is an artificial restriction enzyme, generated by fusing the TAL effector DNA binding domain to a DNA cleavage domain. TAL effectors are proteins which are typically secreted by Xanthomonas bacteria or related species, or which are derived therefrom and have been modified. The DNA binding domain of the TAL effector may comprise a highly conserved sequence, e.g. of about 33-34 amino acid sequence with the exception of the 12th and 13th amino acids which are highly variable (Repeat Variable Diresidue or RVD) and typically show a strong correlation with specific nucleotide recognition. The TALEN DNA cleavage domain may be derived from suitable nucleases. For example, the DNA cleavage domain from the Fokl endonuclease or from Fokl endonuclease variants may be used to construct hybrid nucleases. TALENs may preferably be provided as separate entities due to the peculiarities of the Fokl domain, which functions as a dimer. TALENs or TALEN components may preferably be engineered or modified in order to target any desired DNA sequence. Such engineering may be carried out according to suitable methodologies, e.g. Zhang et al., Nature Biotechnology, 1-6 (2011), or Reyon et al., Nature Biotechnology, 30, 460-465 (2012).

The term “zinc finger nuclease (ZFN)-based system” as used herein refers to a system of artificial restriction enzymes, which are typically generated by fusing a zinc finger DNA-binding domain to a DNA-cleavage domain. Zinc finger domains may preferably be engineered or modified in order to target any desired DNA sequence. Such engineering methods would be known to the skilled person or can be derived from suitable literature sources such as Bae et al., 2003, Nat Biotechnol, 21, 275-80; Wright et al., 2006, Nature Protocols, 1, 1637-1652.) Typically, the non-specific cleavage domain from type IIs restriction endonucleases, e.g. from Fokl, may be used as the cleavage domain in ZFNs. Since this cleavage domain dimerizes in order to cleave DNA a pair of ZFNs is typically required to target non-palindromic DNA sites. ZFNs envisaged by the present invention may further comprise a fusion of the non-specific cleavage to the C-terminus of each zinc finger domain. For instance, in order to allow two cleavage domains to dimerize and cleave DNA, two individual ZFNs are typically required to bind opposite strands of DNA with C-termini provided in a specific distance. It is to be understood that linker sequences between the zinc finger domain and the cleavage domain may requires the 5′ terminus of each binding site to be separated by about 5 to 7 bp. The present invention envisages any suitable ZNF form or variant, e.g. classical Fokl fusions, or optimized version of the Fokl, as well as enzymes with modified dimerization interfaces, improved binding functionality or variants, which are able to provide heterodimeric species.

In certain embodiments, the additional modification of a host cell as described above includes the employment of a host cell for a method of the present invention, i.e. a site directed integration into a genetic locus of the pyrimidine salvage pathway, wherein said host cell comprises such an additional modification already when said site directed integration into a genetic locus of the pyrimidine salvage pathway according to the present invention is performed. In other embodiments, the additional modifications are performed after the site directed integration into a genetic locus of the pyrimidine salvage pathway of the present invention have been performed. Also envisaged is a parallel or simultaneous performance of the site directed integration into a genetic locus of the pyrimidine salvage pathway and an additional modification of the host cells as described above.

In a further specific embodiment, the present invention also envisages the integration into the genomic loci of the pyrimidine salvage pathway as described above of genes of interest or sequences of interest, which comprise or encode components of genomic editing systems as described above. It is particularly preferred that components of the CRISPR/Cas system be provided in a sequence of interest and thus be genomically integrated into a host cell. In a further specific embodiment, the CRISPR/Cas system may alternatively be used to cleave mRNA, thereby reducing expression or silencing a gene.

In another aspect the present invention relates to a host cell, comprising at least one gene or sequence of interest in one or more genetic loci encoding an activity of the pyrimidine salvage pathway, wherein said gene or sequence of interest replaces or partially replaces the sequence encoding said at least one activity of the pyrimidine salvage pathway at said locus. The host cell may accordingly be a result or product of the method of the present invention. The gene or sequence of interest may be or comprise any of the above mentioned activities. The host cell may be any of the above mentioned host cells. It is particularly preferred that the host cell is an Aspergillus niger, Aspergillus oryzae, Aspergillus fumigatus, Komagataella phaffii, Trichoderma reesei, Penicillium chrysogenum, Acremonium chrysogenum, Candida albicans, Ustilago maydis, Cryptococcus neoformans, Fusarium oxysporum, Rhizopus delemar, or Magnaporthe oryzae cell. In the most preferred embodiment, the host cell is an Aspergillus fumigatus cell. Preferably, the host cell comprises the gene or sequence of interest within the genomic locus of the purine/cytosine permease and/or the uracil-phosphoribosyl-transferase and/or the concentrative nucleoside transporter and/or the uridine kinase.

The host cell may, in certain embodiments, additionally comprise further genetic modifications as described herein.

In a further aspect the present invention relates to the use of a host cell comprising at least one gene or sequence of interest as defined above, or a host cell produced, obtained or obtainable according to a method of the present invention for the production of an enzymatic activity as defined above; for the production of an activity involved in the generation of carbohydrates, fatty acids or lipids as defined above; for the production of carbohydrates, fatty acids or lipids; for the production of a pharmaceutically active protein or peptide as defined above; for the production of an antibiotic or of an activity or protein involved in the production of an antibiotic as defined above; for the production of an activity or protein involved in the synthesis of biofuels, as defined above, for the generation of biofuels; for the production of an activity involved in foodstuff or animal feedstuff generation, as defined above; for the production of foodstuff or animal foodstuff; for the production of an activity involved in the synthesis of vitamins or dietary supplements, as defined above; for the production of vitamins or dietary supplements; for the production of an activity involved in the synthesis of amino acids as defined above; for the production of amino acids; for the production of an activity involved in the generation of cosmetic ingredients, as defined above; for the production of cosmetic ingredients; for the production of an activity involved in the generation of organic raw material as defined above; for the generation of organic raw material; for the production of proteins used in metabolic engineering or synthetic biology as defined above; or for the provision of a host cell which has been metabolically engineered or which has been designed according to synthetic biological approaches. The present invention envisages any further suitable use of the host cell, e.g. as starting organism for further genetic modifications, as research tool etc.

In a final aspect the present invention relates to the use of a genetic locus encoding at least one activity of the pyrimidine salvage pathway in a host cell, wherein said activity of the pyrimidine salvage pathway is purine/cytosine permease (FcyB), uracil-phosphoribosyl-transferase (Uprt), concentrative nucleoside transporter (CntA) or uridine kinase (UK) as selection marker in a process of transforming said host cell or a process of genetically modifying said host cell. The host cell may be any host cell as mentioned herein above. It is preferred that the host cell is an Aspergillus niger, Aspergillus oryzae, Aspergillus fumigatus, Komagataella phaffii, Trichoderma reesei, Penicillium chrysogenum, Acremonium chrysogenum, Candida albicans, Ustilago maydis, Cryptococcus neoformans, Fusarium oxysporum, Rhizopus delemar, or Magnaporthe oryzae cell. In the most preferred embodiment, the host cell is an Aspergillus fumigatus cell. The genetic locus may accordingly be used for any site directed integration of a sequence, e.g. of a gene or sequence of interest as described herein. The use involves the employment of substances such as 5-FC, 5-FU and/or 5-FUR as selection medium against the presence of a functional copy of a member of the pyrimidine salvage pathway in a host cell, in particular purine/cytosine permease (FcyB), cytosine deaminase (FcyA), uracil-phosphoribosyl-transferase (Uprt), concentrative nucleoside transporter (CntA) or uridine kinase (UK). The process of transformation or genetic modification may be performed as defined herein above. For certain species the transformation procedure may be adapted, e.g. in accordance with corresponding information known to the skilled person, or derivable from suitable literature sources such as Laboratory Protocols in Fungal Biology, 2013, ed. Gupta et al., Springer-Verlag New York, or Genetic Transformation Systems in Fungi, 2015, Vol. 1 and 2, ed. Van den Berg and Maruthachalam, Springer International Publishing.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

### Use of the fcyB Locus

pH 5 transcriptionally activates fcyB mediated uptake of 5FC. Therefore the drug is much more active at pH5. Inactivation of fcyB leads to resistance at 10 μg/ml 5FC at pH 5. Inactivation of fcyB leads to resistance on AMM (AMM Composition: 55.5 mM D-glucose, 20.0 mM ammonium tartrate, 7 mM KCl, 2.1 mM MgSO₄ x 7H₂O, 11.2 mM KH₂PO₄, 0.09 μM Na₂B₄O₇ x 10H₂O, 1 μM CuSO₄ x 5H₂O, 10 μM FeSO₄ x 7H₂O, 4.5 μM MnSO₄ x 4H₂O, 3.1 μM Na₂MoO₄ x 10H₂O, 10 Linn ZnSO₄ x 7H₂O, 0.7% Agar; finally adjusted to pH 6.5 using NaOH before autoclaving) supplemented with 10 μg/ml 5FC and 100 mM citrate buffer pH 5. The WT isolate in contrast, is highly susceptible to the drug at this medium composition at pH 5 in the presence of 10 μg/ml 5FC.

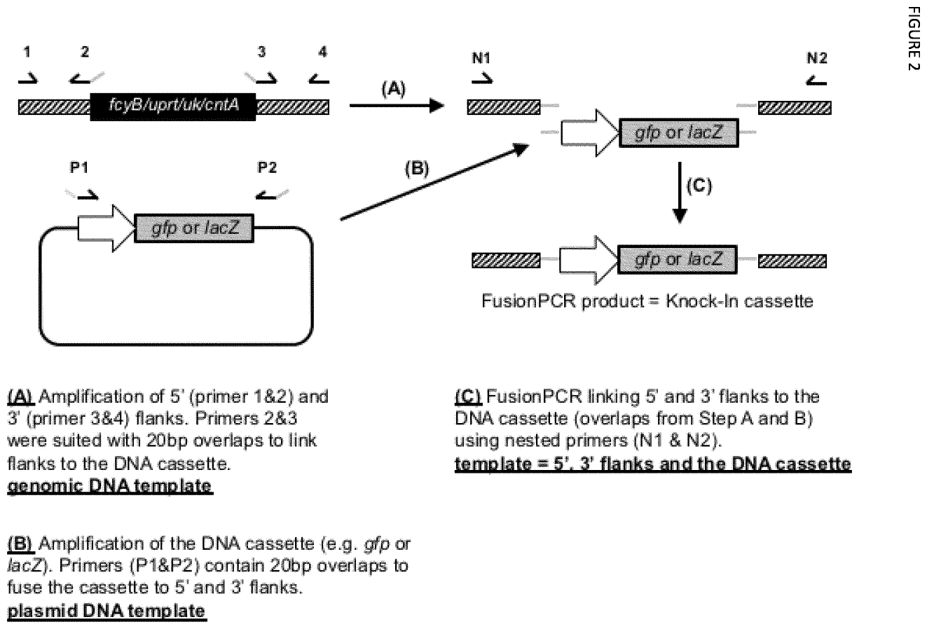

For efficient homologous recombination with simultaneous gene deletion in A. fumigatus, around 1 kb 5′ and 3′ flank of the respective gene were used (see Szewczyk et al.; Fusion PCR and gene targeting in Aspergillus nidulans; Nat Protoc 2006; 1:3111-20). 5′ and 3′ flank of fcyB were amplified and fused to the xylose inducible gfp as well as lacZ gene cassette using FusionPCR (see also FIG. 2) as described recently in Szewczyk et al., 2006. For deletion of the fcyB locus with simultaneous knock-in, transformation was carried out on the medium as listed in Table 1.

For the generation of fusion PCR based gene deletion constructs (fcyB), with simultaneous introduction of a knock-in cassette, around 1 kb of 5′ and 3′ UTR of fcyB gene flanking region were amplified using primer pairs fcyB-1/fcyB-2 (5′) and fcyB-3/fcyB-4 (3′). For the amplification of the respective reporter genes (gfp and lacZ) under control of PxylP followed by the terminator sequence of AtTrpC, primers P1/P2 were used (see scheme provided in FIG. 2). Subsequently, cassettes were PCR purified and linked to 5′ and 3′ gene flanking region of fcyB employing fusion PCR as described previously (Szewczyk et al., 2006). The amplified deletion cassettes were transformed into the recipient strain A1160P+ (Szewczyk et al., 2006) leading to 5FC resistance.

TABLE 1
Medium and final drug concentration of 5-FC
used for selection of fcyB deletion strains

| | Drug for | Concentration | Medium incl. |
|---|---|---|---|
| Genetic locus | selection | (μg/ml) | 1M Sucrose |
| fcyB | 5-FC | 10 | AMM + 100 mM Citrate |
| | | | Buffer pH 5 |

For the experiment the oligonucleotides shown in the following Table 2 were used:

TABLE 2
Oligonucleotides for the generation
of the fcyB knock-in construct

| | SEQ | |
|---|---|---|
| Oligo Name | ID NO: | Sequence 5′ to 3′ |
| fcyB-1 | 103 | CGCTATCCCAGCAATAGAGC |
| fcyB-2 | 104 | TAGTTCTGTTACCGAGCCGG |
| | | ACTGAGTCAATCCCCACCAC |
| fcyB-3 | 105 | GCTCTGAACGATATGCTCCC |
| | | TGCGGTTTTTGGGTTTTATC |
| fcyB-4 | 106 | CACACTGGGTCTGAAGACGA |
| fcyB-N1 | 107 | CAGAGAATTGCCAAGCTGGT |
| fcyB-N2 | 108 | GCGGTATGAAACAACGGTCT |
| P1 | 109 | CCGGCTCGGTAACAGAACTA |
| (reporter | | CTGATGCGAGCAACAGTATG |
| cassette) | | C |
| P2 | 110 | GGGAGCATATCGTTCAGAGC |
| (reporter | | tgagggttgagtacgagatt |
| cassette) | | gg |

### Example 2

### Use of the Uprt Locus

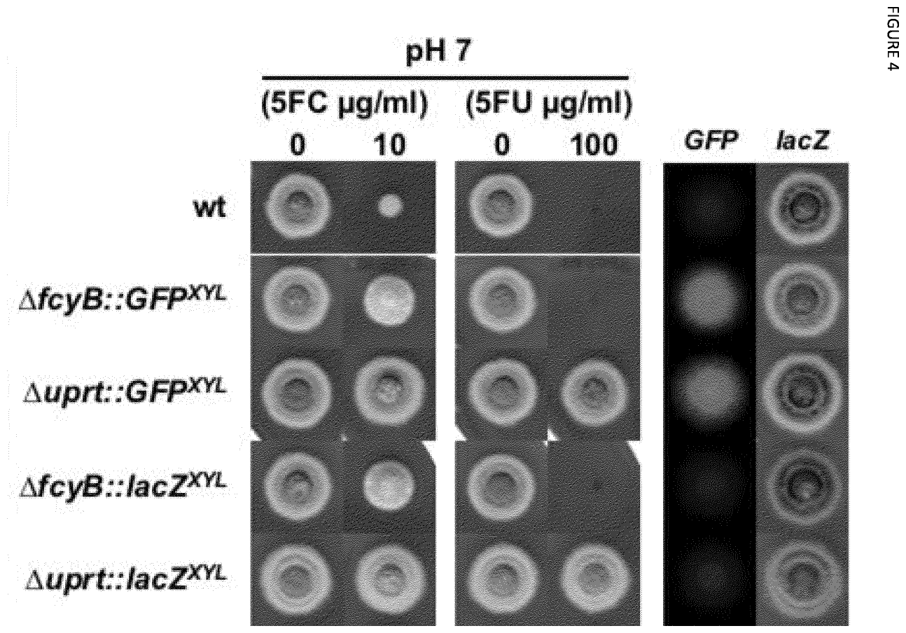

5-FU acts pH independent. Concentrations >100 μg/ml 5FU typically fully inhibit A. fumigatus growth. Furthermore, 5-FU supplementation inhibits ΔfcyB (see FIG. 4). Inactivation of uprt allows growth of A. fumigatus on AMM supplemented with 100 μg/ml 5FC or 5FU at pH7 (see FIG. 4). At concentrations 10-500 μg/ml 5FC or 5FU, independent of the pH (200 μg/ml 5FC as well as 5FU were also tested and the Δuprt strains grow).

For efficient homologous recombination with simultaneous gene deletion in A. fumigatus, around 1 kb 5′ and 3′ flank of uprt are used (Szewczyk et al., 2006). 5′ and 3′ flank of uprt were amplified and fused to the xylose inducible gfp as well as lacZ gene cassette using Fusion PCR as described recently (Szewczyk et al., 2006). For deletion of the uprt locus with simultaneous knock-in, transformation was carried out on specific medium listed in Table 3.

TABLE 3
Medium and final drug concentration of 5-FC
used for selection of uprt deletion strains

| | Drug for | Concentration | Medium incl. |
|---|---|---|---|
| Genetic locus | selection | (μg/ml) | 1M Sucrose |
| uprt | 5-FC or 5-FU | 100 | AMM (pH 6.5) |

For the experiment the oligonucleotides shown in the following Table 4 were used:

TABLE 4
Oligonucleotides for the generation
of the uprt knock-in construct

| | SEQ | |
|---|---|---|
| Oligo Name | ID NO: | Sequence 5′ to 3′ |
| uprt-1 | 111 | GGAAGGACAGGTACGCCATA |
| uprt-2 | 112 | TAGTTCTGTTACCGAGCCGG |
| | | CGGAGCACTCTGAAAATTGG |
| uprt-3 | 113 | GCTCTGAACGATATGCTCCC |
| | | TCCCATCGTGTAGCGACATA |
| uprt-4 | 114 | TACTACCTTCGCCCTCTGGA |
| uprt-N1 | 115 | TTTGAGCGATTAAGGTGCAA |
| uprt-N2 | 116 | GCCCCACTACTTGTTTCCAG |
| P1 | 109 | CCGGCTCGGTAACAGAACTA |
| (reporter | | CTGATGCGAGCAACAGTATG |
| cassette) | | C |
| P2 | 110 | GGGAGCATATCGTTCAGAGC |
| (reporter | | tgagggttgagtacgagatt |
| cassette) | | gg |

### Example 3

### Use of the cntA Locus

5-FUR acts pH independent. Concentrations >100 μg/ml 5-FUR significantly inhibit A. fumigatus growth. Inactivation of cntA significantly increases resistance of A. fumigatus on AMM supplemented with 100 μg/ml 5-FUR.

For efficient homologous recombination with simultaneous gene deletion in A. fumigatus, around 1 kb 5′ and 3′ flank of cntA are used (Szewczyk et al., 2006). 5′ and 3′ flank of cntA are amplified and fused to the xylose inducible gfp as well as lacZ gene cassette using FusionPCR as described recently (Szewczyk et al., 2006). For deletion of the cntA locus with simultaneous knock-in, transformation is carried out on specific medium listed in Table 5.

TABLE 5
Medium and final drug concentration of 5-
FUR for selection of cntA deletion strains

| | Drug for | Concentration | Medium ind. |
|---|---|---|---|
| Genetic locus | selection | (μg/ml) | 1M Sucrose |
| cntA | 5-FUR | 100 | AMM |

For the experiment the oligonucleotides shown in the following Table 6 are used:

TABLE 6
Oligonucleotides for the generation
of the cntA knock-in construct

| | SEQ | |
|---|---|---|
| Oligo Name | ID NO: | Sequence 5′ to 3′ |
| cntA-1 | 117 | ACTGGGGCTTTTTCTGGACT |
| cntA-2 | 118 | TAGTTCTGTTACCGAGCCGG |
| | | TTAAGAACGCGACGACCTTT |
| cntA-3 | 119 | GCTCTGAACGATATGCTCCC |
| | | TGCCTGCAAATCACAAGAAC |
| cntA-4 | 120 | ATACATCGTCCACGGAGAGC |
| cntA-N1 | 121 | TTTAACGCGACGACAGAATG |
| cntA-N2 | 122 | CAAGGTGGGTGGATTTGTCT |
| P1 | 109 | CCGGCTCGGTAACAGAACTA |
| (reporter | | CTGATGCGAGCAACAGTATG |
| cassette) | | C |
| P2 | 110 | GGGAGCATATCGTTCAGAGC |
| (reporter | | tgagggttgagtacgagatt |
| cassette) | | gg |

### Example 4

### Use of the Uk Locus

5-FUR acts pH independent. Inactivation of uk increases resistance of A. fumigatus on AMM supplemented with 100 μg/ml 5-FUR.

For efficient homologous recombination with simultaneous gene deletion in A. fumigatus, around 1 kb 5′ and 3′ flank of uk are used (Szewczyk et al., 2006). 5′ and 3′ flank of uk are amplified and fused to the xylose inducible gfp as well as lacZ gene cassette using FusionPCR as described recently (Szewczyk et al., 2006). For deletion of the uk locus with simultaneous knock-in, transformation is carried out on specific medium listed in Table 7.

TABLE 7
Medium and final drug concentration of 5-
FUR for selection of uk deletion strains

| | Drug for | Concentration | Medium incl. |
|---|---|---|---|
| Genetic locus | selection | (μg/ml) | 1M Sucrose |
| uk | 5-FUR | 100 | AMM |

For the experiment the oligonucleotides shown in the following Table 8 are used:

TABLE 8
Oligonucleotides for the generation
of the uk knock-in construct

| | SEQ | |
|---|---|---|
| Oligo Name | ID NO: | Sequence 5′ to 3′ |
| uk-1 | 123 | ATAGGTGGTAGGGCAGGAGG |
| uk-2 | 124 | TAGTTCTGTTACCGAGCCGG |
| | | ATTAGAATGCGGCGCAACAG |
| uk-3 | 125 | GCTCTGAACGATATGCTCCC |
| | | GGTCTATAGTGTCAGGCGGC |
| uk-4 | 126 | GCCAAACTCACTCGGGTACA |
| uk-N1 | 127 | GCCAGAATGAATCGCAGTGC |
| uk-N2 | 128 | TGCGATTCGTGACTTCTCCC |
| P1 | 109 | CCGGCTCGGTAACAGAACTA |
| (reporter | | CTGATGCGAGCAACAGTATG |
| cassette) | | C |
| P2 | 110 | GGGAGCATATCGTTCAGAGC |
| (reporter | | tgagggttgagtacgagatt |
| cassette) | | gg |

### Example 5

### Experimental Conditions

For the experiments described in Examples 6 to 10 the following conditions were used:

### Growth Conditions and Fungal Transformation

Plate growth assay based susceptibility testing of A. fumigatus and P. chrysogenum was carried out using solid AMM, for F. oxysporum solid PDA was employed. Low pH medium contained 100 mM citrate buffer (pH5), neutral pH medium 100 mM MOPS buffer (pH7). For strains carrying PxylP tunable reporter genes (sGFP, mKate2PER, sGFPMIT, lacZ), 0.5% xylose was supplemented to the medium to induce gene expression.

For fungal manipulations, 2 μg DNA of each construct was transformed into protoplasts of the respective recipient. For the regeneration of transformants, solid AMM (A. fumigatus and P. chrysogenum) or PDA (F. chrysogenum) supplemented with 342 g/l or 200 g/l sucrose, respectively, were used. Selection procedures using conventional selectable marker genes (hph, ble) were carried out as described previously for A. fumigatus (Gsaller et al., Antimicrob Agents Chemother 62 (2018)).

Deletion of A. fumigatus fcyA and Uprt

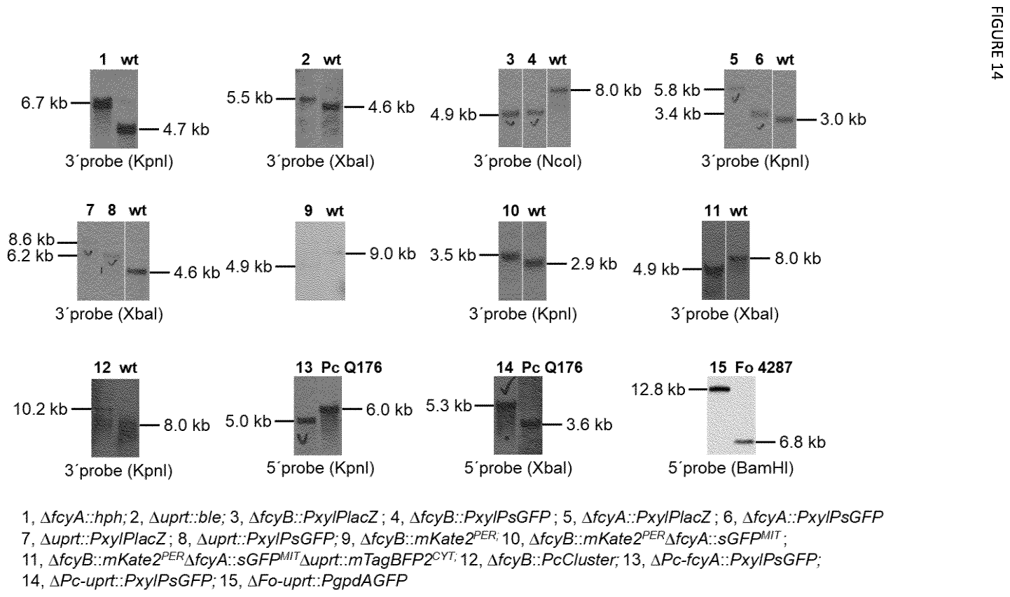

Strains and primers used in this study are listed in Table S3 and S4. Coding sequences of fcyA and uprt were disrupted in wt (A1160P+) using hygromycin B and zeocin resistance cassettes, respectively. Therefore, deletion constructs comprising approximately 1 kb of 5′ and 3′ NTR linked to the central antibiotic resistance cassette were generated using fusion PCR as previously described (Fraczek, et al., The Journal of antimicrobial chemotherapy 68, 1486-1496 (2013)). Correct integration of constructs was confirmed by Southern analysis (see FIG. 14).

Generation of A. fumigatus Knock-in Strains

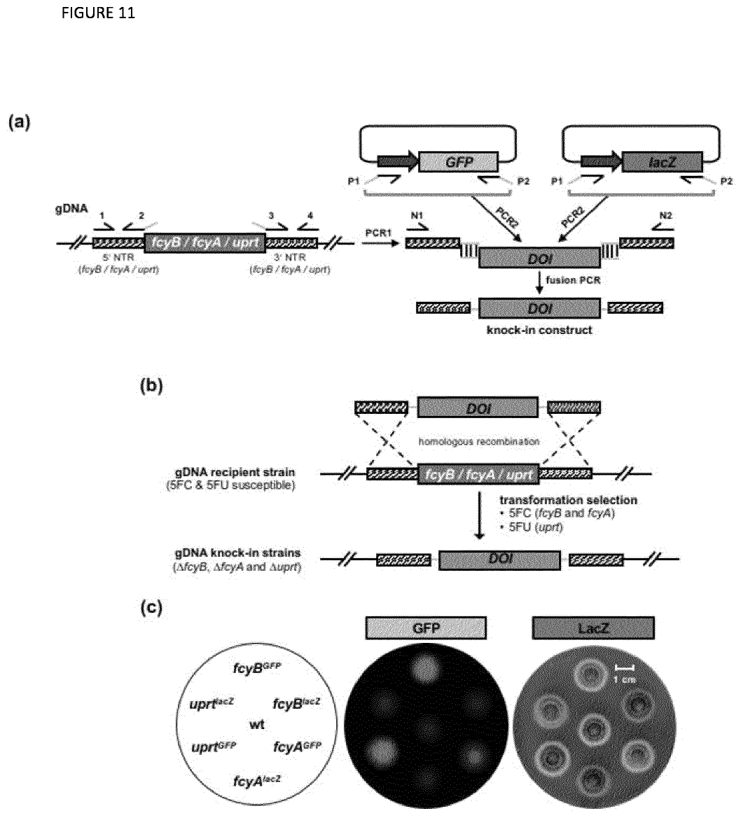

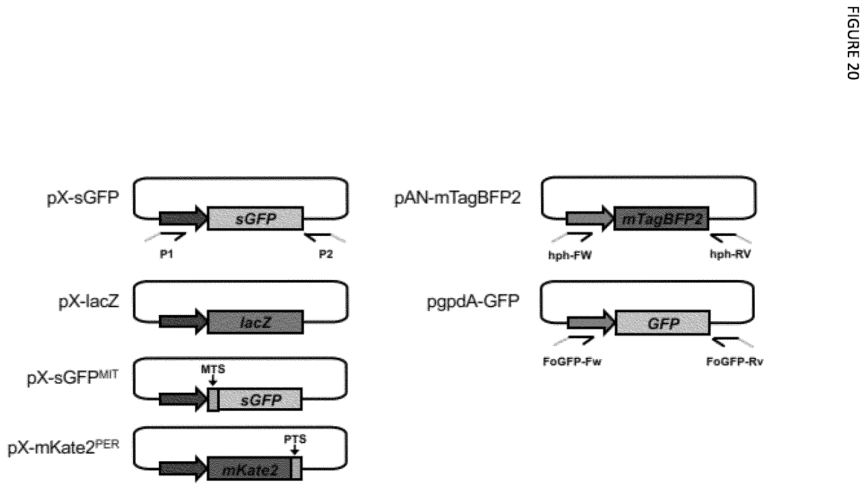

Knock-in constructs for A. fumigatus loci fcyB, fcyA and uprt, P. chrysogenum loci Pc-fcyA and Pc-uprt as well as F. oxysporum Fo-uprt were generated similarly to the gene deletion fragments described above using fusion PCR. Here, instead of the antibiotic resistance cassettes, DOIs (reporter cassettes, see also FIG. 20) were connected to approximately 1 kb 5′ and 3′ NTR of the respective locus (see FIG. 11 (a)).

LacZ based colorimetric assay and fluorescence imaging

For the detection of LacZ activity (conversion of X-Gal into the blue compound 5,5′-Dibrom-4,4′-dichlor-indigo) (Horwitz, et al., J Med Chem 7, 574-575 (1964)), a 5 ml layer of a 1 mM X-Gal/1% agar/1% N-lauroylsarcosin solution was poured over fungal colonies. GFP expression of fungal colonies was visualized using the laser scanner Typhoon FLA9500 (Ex 473 nm; Em≥510 nm).

Expression and subcellular localization of mKate2PER, sGFP^{MIT} and mTagBFP^{CYT} in RFP^{PER}GFP^{MIT}BFP^{CYT} were monitored using confocal laser scanning microscopy (LEICA TCS SP8). Acquired images were processed using ImageJ (2D images), Huygens (deconvolution) and Imaris (3D images).

### Detection of Penicillin G in Culture Supernatants

To detect the potential production of penicillin, strains are grown in AMM for 48 h at 25° C. 2 ml culture supernatants are shock-frozen, freeze-dried and resuspended in 400 μl water. Penicillin G is extracted from the aqueous phase using 1 volume butyl acetate. 500 μl of concentrated supernatant were mixed vigorously. Subsequent to centrifugation (12.000 rpm, 5 min) 400 μl of the organic phase is collected in a new reaction tube and dried. Subsequently, the detection of penicillin G is carried out by HPLC-MS.

For the experiment the oligonucleotides shown in the following Table 9 are used:

TABLE 9
Oligonucleotides used in Examples 5 to 10

| | SEQ | |
|---|---|---|
| Oligo Name | ID NO: | Sequence 5′ to 3′ |
| P1 forward | 153 | CCGGCTCGGTAACAGAACTACTGATGCGA |
| | | GCAACAGTATGC |
| P2 reverse | 154 | GGGAGCATATCGTTCAGAGCTGAGGGTTG |
| | | AGTACGAGATTGG |
| hph-FW | 155 | CCGGCTCGGTAACAGAACTAACGGCGTAA |
| | | CCAAAAGTCAC |
| hph-RV | 156 | GGGAGCATATCGTTCAGAGCTCTTGACGA |
| | | CCGTTGATCTG |
| FoGFP-FW | 157 | GTTGTAGGGGCTGTATTAGGTCTCGGCTG |
| | | TTGTTAGTGTTCGAGG |
| FoGFP-RV | 158 | GAGTCGTTTACCCAGAATGCACAGGGAAG |
| | | GAATCAGCGCAAAG |
| 5′ fcyB-FW | 159 | TGTGGCGGCCGCGTTTAAACCGCTATCCC |
| | | AGCAATAGAGC |
| 5′ fcyB-RV | 160 | TTACGCCAAGCTTGCATGCCACTGAGTCA |
| | | ATCCCCACCAC |
| 3′ fcyB-FW | 161 | AGTGAATTCGAGCTCGGTACTGCGGTTTT |
| | | TGGGTTTTATC |
| 3′ fcyB RV | 162 | AGCGGTTTAAACGCGGCCGCCACACTGGG |
| | | TCTGAAGACGA |
| BB-pfcyB-FW | 163 | TGTGAAATTGTTATCCGCTCACAA |
| BB-pfcyB RV | 164 | AAACAGCTATGACCATGATTACGC |
| PcFrag1-FW | 165 | AATCATGGTCATAGCTGTTTAAAGGGGAG |
| | | AGAGCGAAAAG |
| PcFrag1-RV | 166 | GCATGGGGACAATCTCACTT |
| PcFrag2-FW | 167 | AAGTGAGATTGTCCCCATGCAG |
| PcFrag2-RV | 168 | GAGCGGATAACAATTTCACACGCGTGATA |
| | | TCCTGTCTTCA |
| Pc-fcyA-1 | 169 | TGACCTTGATGGCATCTGAA |
| Pc-fcyA-2 | 170 | TAGTTCTGTTACCGAGCCGGTCAGTGCGG |
| | | GCTACAGAGTA |
| Pc-fcyA-3 | 171 | GCTCTGAACGATATGCTCCCGGCCTGCAC |
| | | ATATCATAGCC |
| Pc-fcyA-4 | 172 | AGCCGTAAAATTCGCATCAC |
| Pc-fcyA-N1 | 173 | GTCGAGGTGCTCAATGTGAA |
| Pc-fcyA N2 | 174 | TTGTTTTGACTTCCCCTTCG |
| Pc-uprt-1 | 175 | GGACAGTTTGGACAATGCAG |
| Pc-uprt-2 | 176 | TAGTTCTGTTACCGAGCCGGTTTGAAGGG |
| | | CAAGAGTCCAG |
| Pc-uprt-3 | 177 | GCTCTGAACGATATGCTCCCACCACGTTG |
| | | AAAGGAGCATC |
| Pc-uprt-4 | 178 | AGACCGTGGAAGTTGGTCAG |
| Pc-uprt-N1 | 179 | TTTTGCAAGGGTCGAGAAAG |
| Pc-uprt N2 | 180 | CAGTTCTTGCCCTGGATCTC |
| Fo-uprt-1 | 181 | CATACGTCACCACCTTGC |
| Fo-uprt-2 | 182 | TAGTTCTGTTACCGAGCCGGGCTGTTGTT |
| | | AGTGTTCGAGG |
| Fo-uprt-3 | 183 | GCTCTGAACGATATGCTCCCGAAGGAATC |
| | | AGCGCAAAG |
| Fo-uprt-4 | 184 | CACGTATAGAATCACGGAGG |
| Fo-uprt-N1 | 185 | GACGCCATAGTGTGCTC |
| Fo-uprt N2 | 186 | GCTTGATGCATGCACTAG |

### Example 6

### Cytosine Deaminase FcyA and Uracil Phosphoribosyltransferase Uprt are Crucial for the Metabolic Activation of 5FC in Aspergillus fumigatus

While 5FC found its use in the treatment of fungal infections (Vermes et al., The Journal of antimicrobial chemotherapy 46, 171-179 (2000); Chandra et al., Infect Dis, 313-326 (2009)), 5FU, an intermediate product of the 5FC metabolic pathway, plays an important role as anti-cancer therapeutic (Longley et al., Nature reviews. Cancer 3, 330-338 (2003)). Metabolization of 5FC has been well-studied in the model yeast Saccharomyces cerevisiae: 5FC is converted by the CD Fcy1p to 5FU (Whelan, Critical reviews in microbiology 15, 45-56 (1987) and Polak et al., Chemotherapy 22, 137-153 (1976)) and subsequently phosphoribosylated to 5FUMP by the UPRT Fur1p (Kern et al., Gene 88, 149-157 (1990)). Inactivation of each of these steps resulted in 5FC resistance, whereby inactivation of Fur1p also conferred 5FU resistance (Kern et al., Gene 88, 149-157 (1990)). Regarding its uptake, orthologous proteins from S. cerevisiae (Fcy2p), A. nidulans (FcyB) and A. fumigatus (FcyB), respectively, have been identified as major 5FC cellular importers (Gsaller et al., Antimicrob Agents Chemother 62 (2018); Paluszynski et al., Yeast 23, 707-715 (2006); Vlanti & Diallinas, Molecular microbiology 68, 959-977 (2008)).

Among other fungal species, A. fumigatus is susceptible to 5FC (Te Dorsthorst et al., Antimicrob Agents Chemother 48, 3147-3150 (2004); Te Dorsthorst et al., Antimicrobial agents and chemotherapy 49, 3341-3346 (2005)) and is therefore anticipated to harbor genes encoding CD and UPRT in addition to 5FC uptake. BLASTP based in silico predictions revealed A. fumigatus FcyA (AFUB_005410) and Uprt (AFUB_053020) as putative orthologs of yeast Fcy1p and Fur1p, respectively. To analyze their role in 5FC as well as 5FU activity, fcyA and uprt was inactivated in the A. fumigatus strain A1160P+ (Fraczek, et al., The Journal of antimicrobial chemotherapy 68, 1486-1496 (2013)), termed wt here, using hygromycin and phleomycine resistance based deletion cassettes. Due to the interdependency of 5FC activity and environmental pH (Gsaller et al., Antimicrob Agents Chemother 62 (2018); Te Dorsthorst et al., Antimicrob Agents Chemother 48, 3147-3150 (2004)) the contribution of both enzymes as well as FcyB to 5FC and 5FU activity at both pH5 and pH7 was investigated.

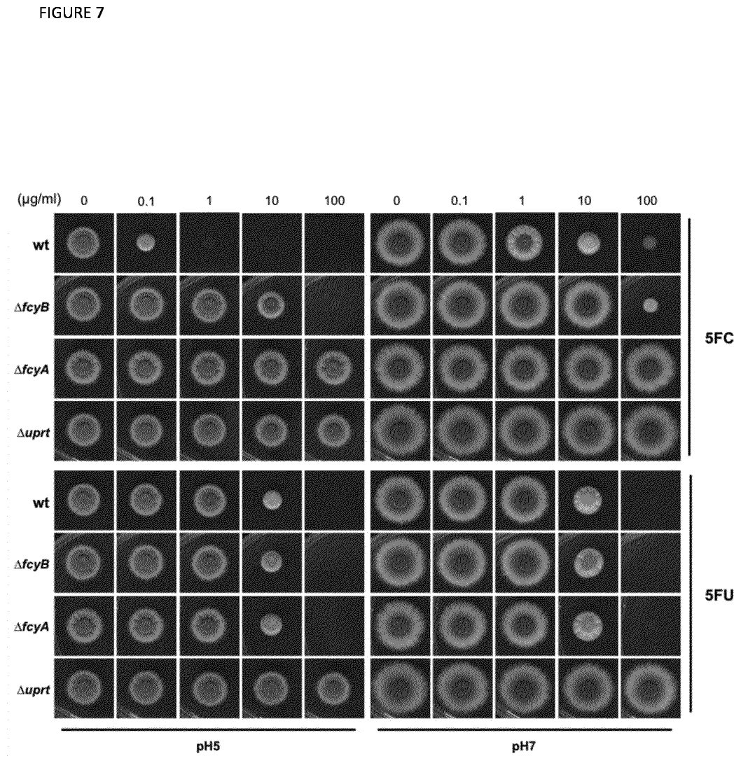

Plate growth based susceptibility testing revealed that 5FC levels ≥1 μg/ml blocked wt growth at pH5, while 100 μg/ml 5FC were required at pH7 (see FIG. 7). Although FcyB illustrates the major 5FC uptake protein, at 100 μg/ml 5FC ΔfcyB was not able to grow at pH5 and showed severe growth inhibition at pH7. In contrast to ΔfcyB, ΔfcyA and Δuprt displayed full resistance to 5FC up to 100 μg/ml, regardless of the pH. 100 μg/ml 5FU blocked growth of wt, ΔfcyA and ΔfcyB at pH5 as well as pH7, while Δuprt displayed high resistance at this concentration level.

These data confirm the role of FcyB as major 5FC cellular importer and indicate the presence of additional uptake mechanisms. Similar to the orthologous proteins in S. cerevisiae, the findings reveal the essential role of FcyA and Uprt for 5FC activity and demonstrate the crucial role of Uprt for metabolic activation of 5FU.

### Example 7

### Self-Encoded Loci fcyB, fcyA and Uprt can be Used for 5FC/5FU Based Transformation Selection

Genes coding for CD and UPRT activities have been described for the use as negative selectable markers (Mullen et al., Proceedings of the National Academy of Sciences of the United States of America 89, 33-37 (1992), Orr et al., Malaria J 11 (2012), Fox et al., Mol Biochem Parasit 98, 93-103 (1999), Shi, T. et al., PloS one 8, e81370 (2013), van der Geize et al., Nucleic acids research 36 (2008)). The A. fumigatus genome encodes activities for both CD (FcyA) and UPRT (Uprt). Based on the fact that lack of FcyB, FcyA or Uprt confers resistance to 5FC (ΔfcyB, ΔfcyA and Δuprt) or 5FU (Δuprt)(see FIG. 7), it was tested if these loci can be employed for integration of DOI based on 5FC/5FU selection for loss of the respective salvage pathway activity. Moreover, the approach took advantage of the different degree in 5FC resistance observed for ΔfcyB and ΔfcyA, which suggested that 5FC can be used for selection of loss of FcyB at low 5FC concentrations (10 μg/ml) and loss of FcyA at high 5FC levels (100 μg/ml) (see FIG. 7). Selection for loss of Uprt was carried out at 100 μg/ml 5FU.

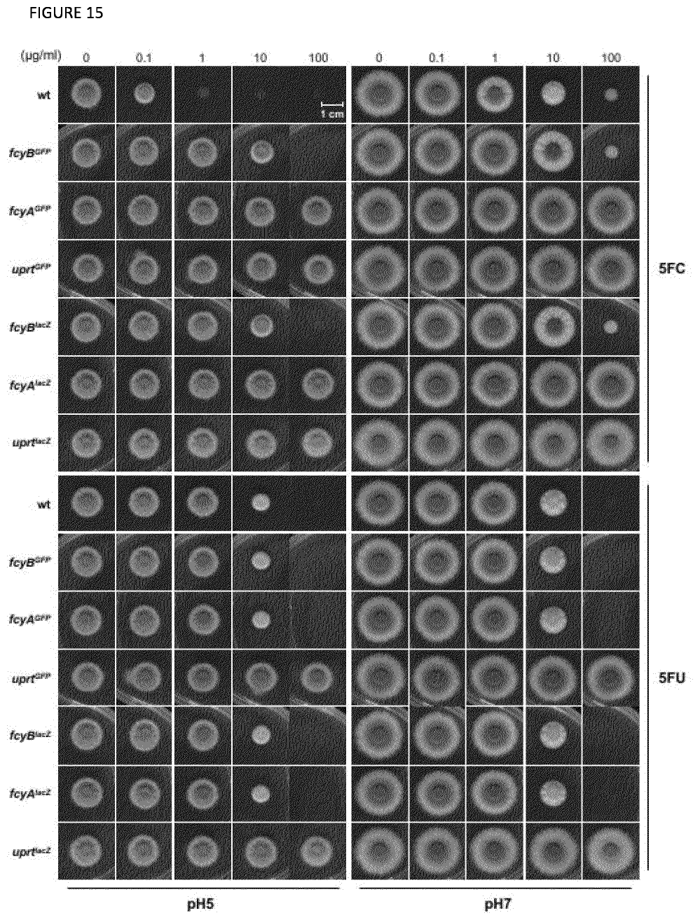

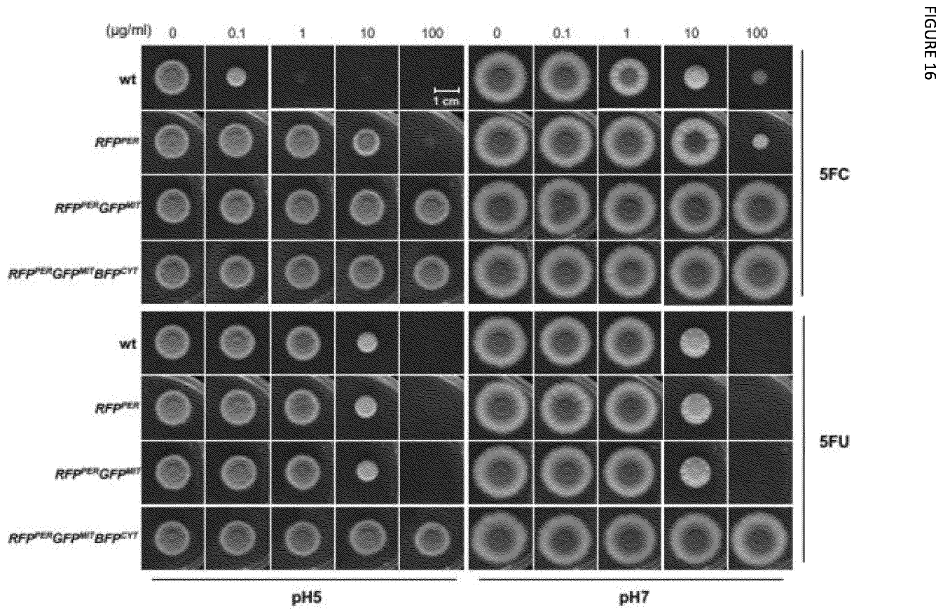

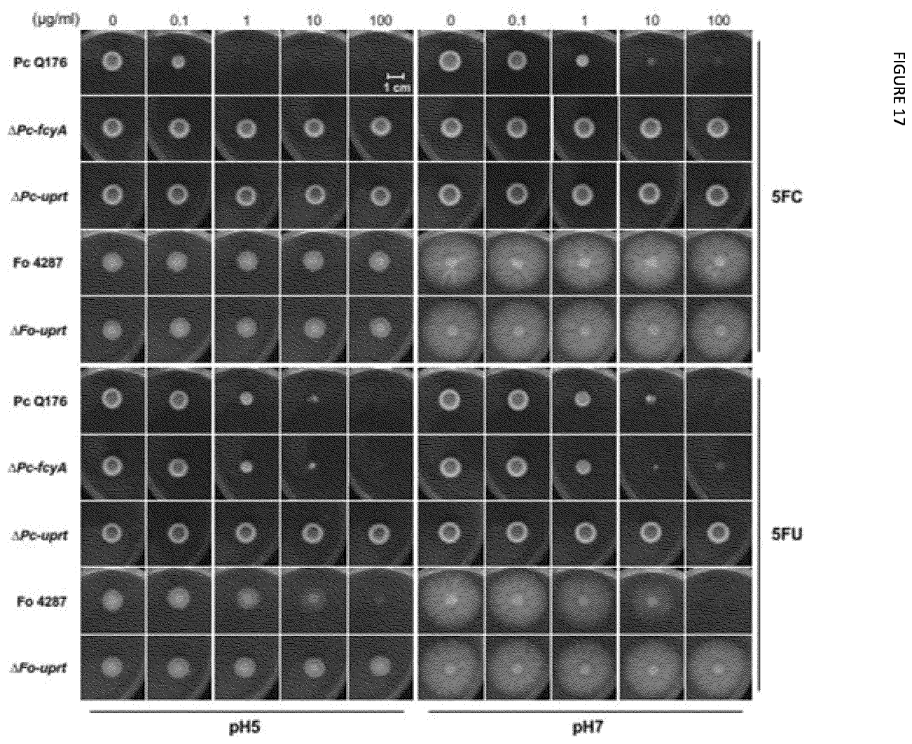

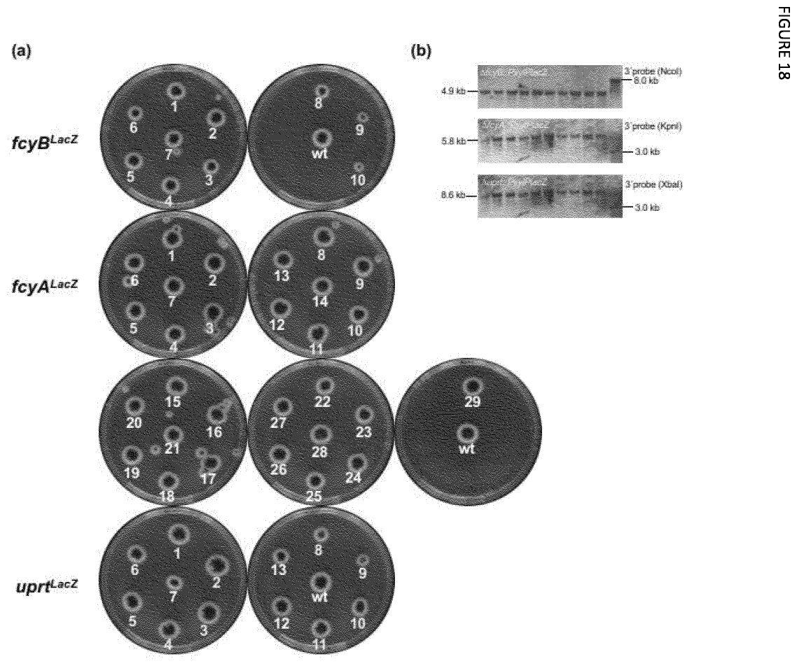

For proof-of-principle, both green fluorescent protein (GFP) and R-galactosidase (LacZ) expression cassettes were used to replace fcyB, fcyA as well as uprt. To achieve homologous recombination-mediated replacement of these loci with the reporter cassettes, approximately 1 kb 5′ and 3′ non-translated regions (NTRs) of the respective gene were linked to each cassette via fusion PCR (see FIG. 11 (a)). The yielding knock-in constructs were transformed into protoplasts of the recipient (wt) which underwent selection for resistance to 5FC and FU (see above and FIG. 11 (b)). Southern blot analyses confirmed site-specific integration of the DOIs in each of the three loci (see FIG. 14). In agreement, all knock-in strains displayed resistance phenotypes according to their absent pyrimidine salvage activity (see FIGS. 15 to 17). Exemplary fluorescence imaging and R-galactosidase staining confirmed functionality of the knock-in cassettes (see FIG. 11 (c)). To determine the transformation efficiency using individual selectable marker genes the corresponding LacZ knock-in constructs was employed for each locus. In addition to monitoring the LacZ activity of all transformants (fcyB^{lacZ}: 10; fcyA^{lacZ}: 27; uprt^{lacZ}: 13; see FIG. 18), Southern analysis was carried out for 10 LacZ positive transformants confirming correct integrations (see FIG. 14).

5FC and 5FU mediated selection allowed replacement of each of the three salvage pathway loci by either GFP- or lacZ-expression cassettes, which demonstrates the suitability of fcyB, fcyA and uprt as selectable markers for integrative transformation in A. fumigatus.

### Example 8

### fcyB, fcyA, Uprt and cntA or Uk can be Consecutively Used for Genomic Knock-Ins

Due the fact that inactivation of fcyB, fcyA, uprt and uk lead to different levels of resistance to 5-FC, 5-FU and 5-FUR it was investigated if these marker genes can be sequentially employed for transformation selection in a wildtype A. fumigatus strain. As an exemplary application, a strain expressing three fluorescent proteins for multicolor imaging was generated. The fluorescent proteins GFP (sGFP), red fluorescent protein (RFP, mKate2) and blue fluorescent protein (BFP, mTagBFP2) were used. The RFP expression cassette was introduced into the fcyB locus, the GFP cassette into the fcyA locus and the BFP cassette into the uprt locus. Moreover, a luciferase expression cassette was introduced into the cntA locus in a ΔfcyBΔfcyAΔuprt triple mutant. Alternatively a luciferase expression cassette was introduced into the uk locus in a ΔfcyBΔfcyAΔuprt triple mutant.

The pursued strategy for the first approach, generating the triple knock-in using fcyB, fcyA and uprt loci, was based on the considerations that: (i) in contrast to wt, ΔfcyB can grow in the presence of 10 μg/ml 5-FC at pH5; (ii) in contrast to ΔfcyB, ΔfcyA can grow at 100 μg/ml 5FC, which allows discrimination of ΔfcyA (or ΔfcyBΔfcyA) from ΔfcyB, and (iii) ΔfcyB and ΔfcyA are still able to import and metabolize 5-FU, which is expected to allow discrimination of ΔfcyBΔfcyA and ΔfcyBΔfcyAΔuprt in the presence of 100 μg/ml 5FU. Accordingly, the loci were targeted in the following order and selection: fcyB with 10 μg/ml FC selection, fcyA with 100 μg/ml 5FC selection and uprt with 100 μg/ml FU selection.

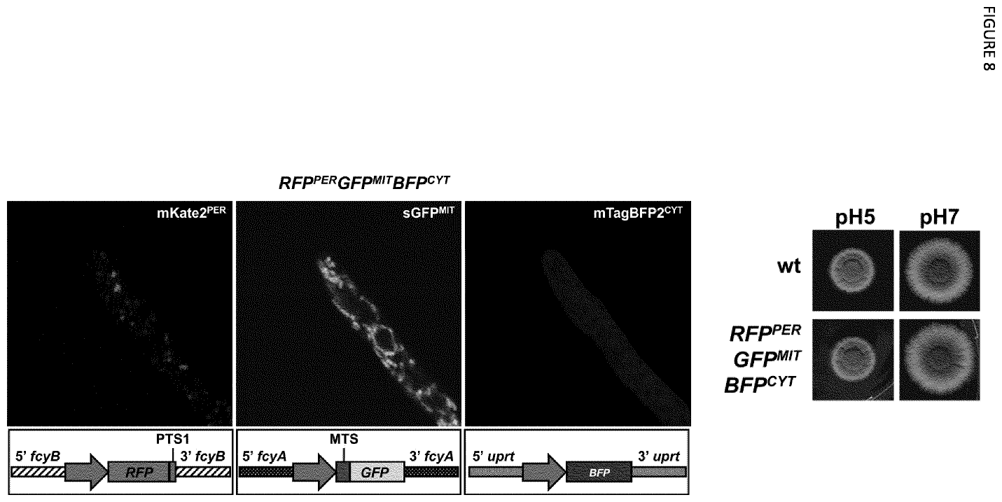

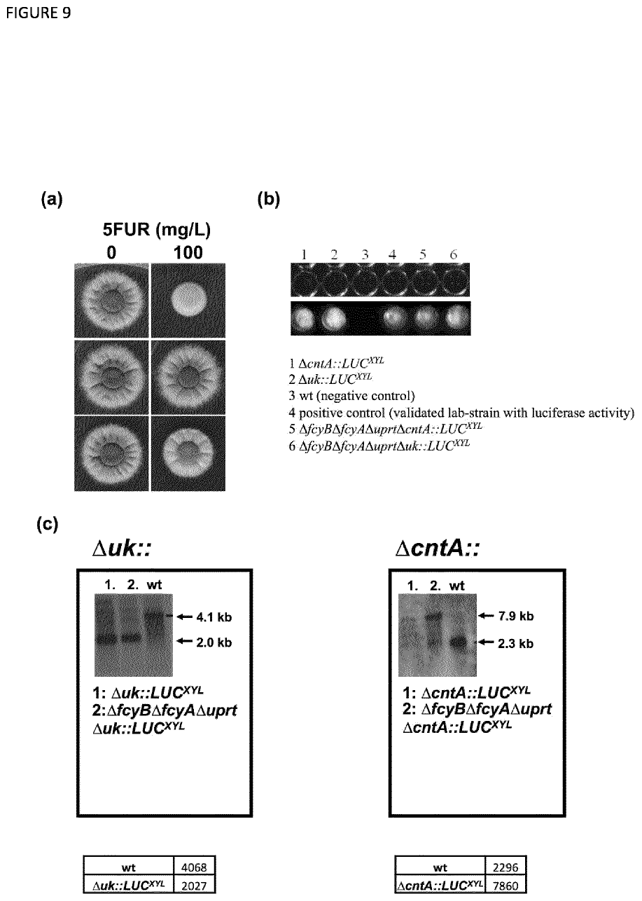

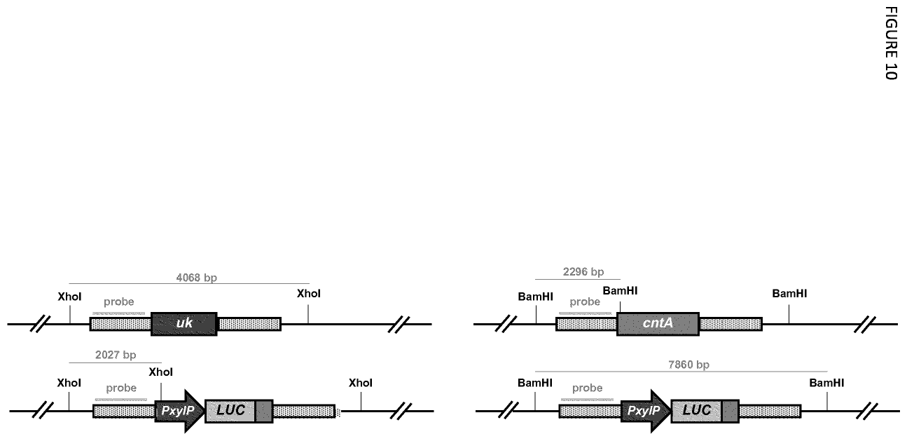

To this end an expression cassette encoding mKate2 carrying the C-terminal peroxisomal targeting sequence (PTS1, tripeptide SKL) (Olivier and Krisans, Biochim Biophys Acta 1529:89-102 (2000)) was integrated into the fcyB locus, yielding strain RFP^{PER} (ΔfcyB::mKate2^{PER}). In this strain, an expression cassette encoding sGFP containing an N-terminal mitochondrial targeting sequence from citrate synthase (Min et al., J Microbiol 48:188-98 (2010)) was targeted to the fcyA locus, yielding strain RFP^{PER}GFP^{MIT} (ΔfcyB::mKate2^{PER}ΔfcyA::sGFP^{MIT}). In a last step, an expression cassette encoding mTagBFP2 with expected cytoplasmic localization was targeted to the uprt locus in RFPPERGFPMIT, yielding strain RFP^{PER}GFP^{MIT}BFP^{CYT} (ΔfcyB::mKate2^{PER}ΔfcyA::sGFP^{MIT} Δuprt::mTagBFP2^{CYT}). Multicolor laser scanning microscopy visualized all three fluorescent proteins in RFP^{PER}GFP^{MIT}BFP^{CYT} in the expected cellular compartments (see FIG. 8, left panel). Noteworthy, the lack of FcyB, FcyA and Uprt (strain RFP^{PER}GFP^{MIT}BFP^{CYT}) did not affected growth (see FIG. 8, right panel). A fourth knock-in, carrying a firefly luciferase encoding gene, was generated using either cntA or uk as target locus (see FIGS. 9 and 10). Therefore, a fcyBΔfcyAΔuprt was used as recipient alongside wt.

### Example 9

### Loci can be Used for the Integration of Biotechnological Relevant, Large DNA Fragments

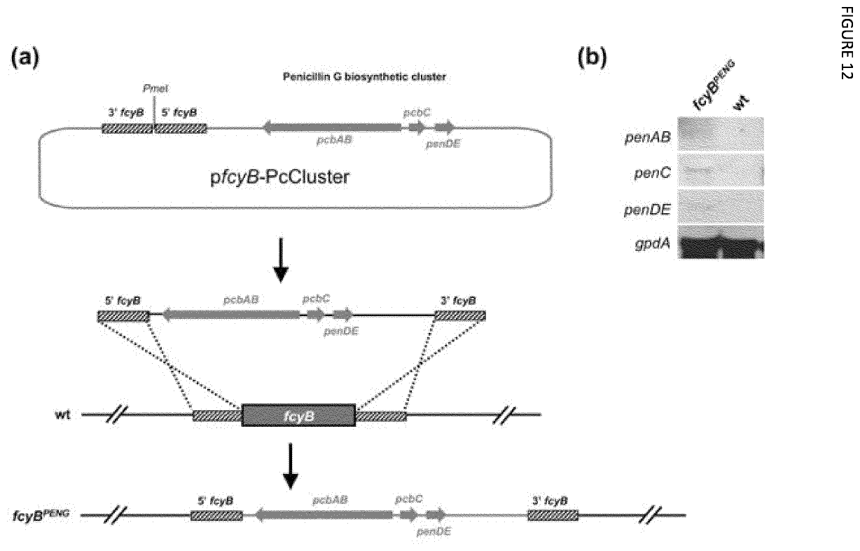

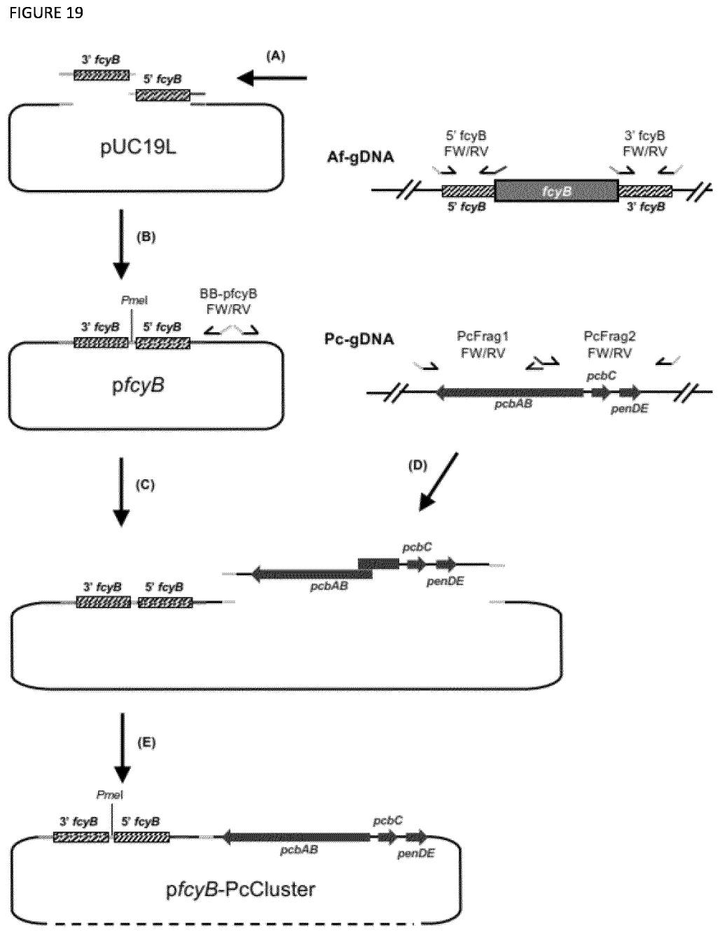

Fungi play important roles as cell factories for the production of a variety of products in food industry as well as medicine. It was therefore tested if the whole penicillin biosynthetic cluster (PcCluster) of P. chrysogenum (^{˜}17 kb) can be integrated into the genome of A. fumigatus transforming this mold into a penicillin producer. The PcCluster contains genes coding for PcbAB (N-5-amino-5-carboxypentanoyl-L-cysteinyl-D-valine synthase), PcbC (Isopenicillin N synthase) and PenDE (acyl-coenzyme A:isopenicillin N acyltransferase). Accordingly, an fcyB knock-in plasmid was developed (pfcyB-PcCluster; for experimental details see FIG. 19) comprising the PcCluster as well as 5′ and 3′ fcyB flanking region (see FIG. 12 (a)). After linearization (PmeI digest opening the plasmid between 3′ and 5′ fcyB flanking region), the fragment resembles a knock-in construct for homologous recombination mediated replacement of the fcyB locus with the PcCluster.

Subsequent to transformation of this construct in wt (selection: 10 μg/ml 5FC, pH5), its site-specific integration at the fcyB locus was confirmed using Southern analysis (see FIG. 14). The resulting strain was termed fcyBPENG. Next, the expression of pcbAB, pcbC and penDE was confirmed by Northern analysis (see FIG. 12 (b)). This was followed by analyzing the penicillin activity according to a bioassay based on the growth inhibitory effects of penicillin on the Gram-positive bacterium Micrococcus luteus.

HPLC-MS is used to confirm the production of penicillin in these strains.

### Example 10

### Implementation of the 5FC/5FU Transformation Selection in Penicillium chrysogenum and Fusarium oxysporum

To identify encoded A. fumigatus FcyB, FcyA, Uprt, Uk and CntA activities in further fungal species, it was searched for A. fumigatus orthologs in biotechnology-relevant species (Aspergillus niger, Aspergillus oryzae, P. chrysogenum, Komagataella phaffii alias Pichia pastoris, S. cerevisiae, Trichoderma reesei) and in virulence-relevant species (Candida albicans, Cryptococcus neoformans, F. oxysporum). Orthologs to A. fumigatus proteins with an overall identity 40% were considered as putative orthologs if activities could be confirmed by susceptibility testing following a broth microdilution based method according to EUCAST (see Table 10).

The applicability of target loci encoding CD and UPRT for the integration of DNA was tested by applying the described selection strategy in P. chrysogenum and F. oxysporum. In line with the genomic data and 5FC/5FU susceptibility (see Table 10), P. chrysogenum expresses both CD (Pc-FcyA, EN45_039280) and UPRT (Pc-Uprt, EN45_060980), while F. oxysporum lacks CD but expresses UPRT (Fo-Uprt, FOYG_03618). Employing the same protocol as used for A. fumigatus enabled the integration of GFP expression cassettes flanked by 5′ and 3′ NTR of the respective P. chrysogenum genes in both the Pc-fcyA and the Pc-uprt loci In F. oxysporum, the same strategy enabled to target a GFP expression cassette to the Fo-uprt locus. The presence and functionality of the GFP reporter cassettes was visualized as described above. These data demonstrate the suitability of loci encoding pyrimidine salvage enzymes as markers for transformation selection also in P. chrysogenum and F. oxysporum.

TABLE 10
susceptibility of different fungal strains to 5-FC, 5-FU and 5-FUR

| | MIC (μg/ml) | | | | | |
|---|---|---|---|---|---|---|
| | 5FC | | 5FU | | 5FUR | |
| | pH 5 | pH 7 | pH 5 | pH 7 | pH 5 | pH 7 |
| C. neoformans | 0.39 | 25 | 6.25 | 12.5 | 3.125 | 12.5 |
| C. albicans | 0.39 | 0.39 | 50 | 0.39 | 0.39 | 0.39 |
| A. niger | 0.39 | 6.25 | 50 | 0.39 | 0.8 | 0.39 |
| A. fumigatus | 0.39 | 400 | 50 | >400 | 6.25 | >400 |
| A. oryzae | 0.39 | 400 | 200 | 12.5 | 6.25 | 12.5 |
| T. reesei | >400 | >400 | 100 | 0.39 | 0.39 | 0.39 |
| S. cersvisiae | 0.39 | 0.39 | 0.8 | 100 | 6.25 | 25 |
| P. chrysogenum | 0.39 | 3.12 | 3.12 | 3.12 | 3.125 | 3.125 |
| P. pastoris | 0.39 | 0.39 | 0.39 | >400 | 0.39 | >400 |
| F. oxysporum | >400 | >400 | 400 | >400 | >400 | >400 |

1. A method of site-directed integration into a genetic locus encoding at least one activity of the pyrimidine salvage pathway in a host cell, wherein said activity of the pyrimidine salvage pathway is purine/cytosine permease (FcyB), cytosine deaminase (FcyA), uracil-phosphoribosyl-transferase (Uprt), concentrative nucleoside transporter (CntA) or uridine kinase (UK), comprising:
(a) providing a host cell comprising a functional copy of the genetic locus encoding at least one activity of the pyrimidine salvage pathway;
(b) introducing a gene or sequence of interest into said host cell via transformation of an integrative nucleic acid construct which comprises 3′ and/or 5′ of the gene or sequence of interest flanks being homologous to said genetic locus or which carries a sequence being homologous to said genetic locus of the pyrimidine salvage pathway and thus allowing for a homologous recombination at said genetic locus, wherein said homologous recombination is capable of causing an inactivation or reduction of the activity encoded by said genetic locus;
(c) growing a transformed host cell under selective medium conditions, wherein said medium comprises an efficient amount of 5-flucytosine (5-FC), 5-fluorouracil (5-FU) or 5-fluorouridine (5-FUR); and
(d) selecting a host cell which is capable of growing under the medium conditions of step (c).

2. The method of claim 1, wherein said integrative nucleic acid construct comprises a control element such as a promoter or a terminator sequence which are operably linked to the gene or sequence of interest or the sequence to be expressed.

3. The method of claim 1, wherein said integrative nucleic acid construct does not comprise a nucleic acid sequence encoding a marker gene for se-lection of a genetically transformed host cell.

4. The method of claim 1, wherein said site-directed integration into a genetic locus encoding an activity of the pyrimidine salvage pathway in a host cell comprises the integration into two or more genetic loci encoding an activity of the pyrimidine salvage pathway in a host cell.

5. The method of claim 4, wherein said site-directed integration is performed in a sequential order in said two or more genetic loci encoding an activity of the pyrimidine salvage pathway in a host cell.

6. The method of claim 4, wherein said two or more genetic loci encoding an activity of the pyrimidine salvage pathway in a host cell are used for site-directed integration in one of the following orders and/or combinations:
(i) (1) fcyB; (2) fcyA;
(ii) (1) fcyB; (2) uprt;
(iii) (1) fcyB; (2) cntA, or uk;
(iv) (1) fcyA; (2) uprt;
(v) (1) fcyA; (2) cntA, or uk;
(vi) (1) uprt; (2) cntA, or uk;
(vii) (1) fcyB; (2) fcyA; (3) uprt;
(viii) (1) fcyB; (2) fcyA; (3) cntA, or uk;
(ix) (1) fcyB, (2) uprt; (3) cntA, or uk;
(x) (1) fcyA, (2) uprt; (3) cntA, or uk;
(xi) (1) fcyB; (2) fcyA; (3) uprt; (4) cntA, or uk.

7. The method of claim 1, wherein said gene or sequence of interest encodes for one or more enzymatic activities, wherein said enzymatic activity comprises an isomerase, oxidase, reductase, oxidoreductase, hydrolase, ligase, lyase, cellulase, chitinase, amylase, lactase, glucosidase, xylanase, transferase, esterase, lipase, mannosidase, glucanase, protease, phytase, invertase, peroxidase, peptidase, pectinase, chymosin or pepsin.

8. The method of claim 1, wherein said gene or sequence of interest encodes one or more of: (i) an activity involved in the production of carbohydrates, fatty acids or lipids, (ii) a pharmaceutically active protein or peptide, (iii) an antibiotic or an activity involved in the production of an anti-biotic, (iv) an activity involved in the production of biofuels, (v) an activity involved in the production of foodstuff or animal feedstuff, (vi) an activity involved in production of vitamins or dietary supplements, (vii) an activity involved in the production of amino acids, (viii) an activity involved in the production of cosmetic ingredients, (ix) an activity involved in the production of organic raw materials, or (x) a protein used in metabolic engineering or synthetic biology such as in cell factory generation or optimization.

9. The method of claim 1, wherein said gene or sequence of interest encodes a homologous activity of the host cell, which is provided in a modified amount, preferably in an increased amount, or in a differently controlled manner.

10. The method of claim 1, wherein said gene or sequence of interest encodes a biomolecular marker protein, preferably a fluorescent protein such as GFP or derivatives thereof.

11. The method of claim 1, wherein said gene or sequence of interest comprises, essentially consists of or consist of an RNA expression cassette, wherein said RNA expression cassette provides one or more elements required for RNA gene silencing.

12. The method of claim 1, wherein said gene or sequence of interest has a codon usage or a dicodon usage, which is adapted to the co-don usage or dicodon usage of the host cell.

13. The method claim 1, wherein said host cell is a bacterium, preferably of the genus Klebsiella, Clostridium, Bacillus, Arthobacter, Streptomyces, Corynebacterium, Erwinia, Xanthomonas, Lactobacillus, Caldicellulosiruptor, Pseudomonas, Alcanivorax, Brevibacterium, Bifidobacterium, Escherichia, or Staphylococcus; or a fungus, preferably of the genus Aspergillus, Candida, Saccharomyces, Ustilago, Cryptococcus, Fusarium, Rhizopus, Magnaporthe, Komagataella, Trichderma, Penicillium, Acremonium, Mucor, Alternaria, Botrytis, Endothia, Rhizoctonia, Sclerotinia, Klyveromyces, Torulopsis, Sporotrichum, Geotrichum, Verticillium, Botryosphaeria, Trichothecium, Hansenula, Schizosaccharomyces, Brettanomyces, or Neurospora; or a plant; or an alga.

14. The method of claim 13, wherein said host cell is an Aspergillus niger, Aspergillus oryzae, Aspergillus fumigatus, Komagataella phaffii, Trichoderma reesei, Penicillium chrysogenum, Acremonium chrysogenum, Candida albicans, Ustilago maydis, Cryptococcus neoformans, Fusarium oxysporum, Rhizopus delemar, or Magnaporthe oryzae cell.

15. The method of claim 1, wherein said method comprises additionally genetically modifying said host cell.

16. The method of claim 15, wherein said additional genetic modification is a blocking of a further activity, an increase or decrease of the expression of a gene, a silencing of a gene, a deletion of one or more genes or loci or gene clusters, or an introduction of one or more additional homologous genes or of one or more heterologous genes.

17. A host cell, comprising at least one gene or sequence of interest as defined in claim 7 in one or more genetic loci encoding an activity of the pyrimidine salvage pathway, wherein said gene or sequence of interest replaces or partially replaces the sequence encoding said at least one activity of the pyrimidine salvage pathway at said locus.

18. The host cell of claim 17, wherein said one or more genetic loci encoding an activity of the pyrimidine salvage pathway are at least two genetic loci selected from the following group and used in the indicated order:
(i) (1) fcyB; (2) fcyA;
(ii) (1) fcyB; (2) uprt;
(iii) (1) fcyB; (2) cntA, or uk;
(iv) (1) fcyA; (2) uprt;
(v) (1) fcyA; (2) cntA, or uk;
(vi) (1) uprt; (2) cntA, or uk;
(vii) (1) fcyB; (2) fcyA; (3) uprt;
(viii) (1) fcyB; (2) fcyA; (3) cntA, or uk;
(ix) (1) fcyB, (2) uprt; (3) cntA, or uk;
(x)(1) fcyA, (2) uprt; (3) cntA, or uk; and
(xi) (1) fcyB; (2) fcyA; (3) uprt; (4) cntA, or uk.

19. Use of the host cell of claim 17 for the production of an enzymatic activity, an activity involved in the production of carbohydrates, fatty acids or lipids, a pharmaceutically active protein or peptide, an antibiotic or an activity involved in the production of an antibiotic, an activity involved in the production of biofuels, an activity involved in the production of foodstuff or animal feedstuff, an activity involved in productions of vitamins or dietary supplements, an activity involved in the production of amino acids, an activity involved in the cosmetic ingredients, an activity involved in the production of organic raw material, or of proteins used in metabolic engineering or synthetic biology.

20. Use of a genetic locus encoding at least one activity of the pyrimidine salvage pathway in a host cell, wherein said activity of the pyrimidine salvage pathway is purine/cytosine permease (FcyB), uracil-phosphoribosyl-transferase (Uprt), concentrative nucleoside transporter (CntA) or uridine kinase (UK) as selection marker in a process of transforming said host cell or a process of genetically modifying said host cell.

21. The use of claim 20, wherein said one or more genetic loci encoding an activity of the pyrimidine salvage pathway are at least two genetic loci select-ed from the following group and used in the indicated order:
(i) (1) fcyB; (2) fcyA;
(ii) (1) fcyB; (2) uprt;
(iii) (1) fcyB; (2) cntA, or uk;
(iv) (1) fcyA; (2) uprt;
(v) (1) fcyA; (2) cntA, or uk;
(vi) (1) uprt; (2) cntA, or uk;
(vii) (1) fcyB; (2) fcyA; (3) uprt;
(viii) (1) fcyB; (2) fcyA; (3) cntA, or uk;
(ix) (1) fcyB, (2) uprt; (3) cntA, or uk;
(x) (1) fcyA, (2) uprt; (3) cntA, or uk; and
(xi) (1) fcyB; (2) fcyA; (3) uprt; (4) cntA, or uk.


SEQUENCE LISTING (186 sequences)

SEQ ID NO: 1 - protein, 523 aa, Aspergillus fumigatus
MFGGVEHDLEKAPQVEKRPFDSSSDGAVPGETFVYGDSWYARLQRLAGKLNIEQRGIERVPADERTDTSYFNIGSMWLAANMVVSSFAIGVLGKSLFDLGFVDAILTCLFFNLLGVLTVCFFSCFGAAFGLRQMVLSRFWFGWGPTKFSADILVPSVAILNVLACVGWSAANAIVGAQLINAVNSNVPGFAAILIIAICTFVITFAGYKVVHAYEYWSWIPTFIVFMIVLGTFAHSGDFRNLPMEVGTSEMGGVLSFGSTVYGFATGWTSYAADYTVYQPANRSRRKIFLSAWIGLIIPLLFCQMLGIAVMTATGIDDGNNKYQMGYDASGNGGLLNAVLEPLGGFGKFCLVILALSIIANNCPNIYSVALTLQVLSRYSQRVPRFVWVFLGSCASVAIAIPGYSHFETVLENFMNFIAYWLSIYSGIALTDHFLFKRGFGGYRPEIYDKRDKLPLGIAASIAFGFGVAGMITGMSQSWWVGPIALHAGQAPFGGDVGFELGFAFSAVMYAVLRPIEIKIFGR

SEQ ID NO: 2 - DNA, 1706 bp, Aspergillus fumigatus
atgttcggtggcgttgagcacgaccttgagaaagccccccaggtagagaagaggcctttcgacagtagcagcgatggtgctgtgcctggggaaacctttgtatatggcgattcgtggtacgccaggctacaacggctggccggaaagctcaacattgagcagcgaggtatagaacgtgtaccagccgatgaacggacggacacgtcatattttaacattggcagcatggtgtgttcctgaatgcaattgatcaaggctttgccagctcagggctggtaccgctgggccaatgtccaaaactgactcaacttagtggctggcggccaacatggtggtgagctccttcgccattggcgtcctcggaaagtcgctctttgatctcggcttcgtcgatgcaattctcacctgcctctttttcaatctcttgggtgtcttgaccgtttgcttcttctcatgcttcggtgctgccttcgggttacggcagatggtgctttcgaggttctggttcggctgggggcccaccaagttcagtatgtatttttttgcataatctcacttattgtcttttcaaagtagcaggtgctgacatcctcgttccttcagttgctattctgaatgtgctcgcttgtgtgggctggtccgctgccaacgccatcgtcggtgctcagctcatcaatgcagtgaatagcaacgtccccggattcgctgccattcttataattgctatttgcacgtttgtcatcacattcgctgggtacaaggttgtgcatgcctatgaatactggagttggatccctaccttcatcgtgtttatgattgtccttggtaccttcgcccactcaggagacttcaggaacctcccaatggaagttggcacatcagaaatgggcggtgtcttgtcctttggttcgactgtgtacggtttcgctactggatggactagctacgccgccgattatactgtgtaccagcccgctaatcggagccgtcgcaagatatttctctccgcctggattggcctcatcattccgcttttgttctgtcagatgctgggaatcgcggtcatgaccgctacaggaatcgacgatggcaataacaagtatcaaatgggctacgacgcctccggaaacggaggcttgttgaatgctgtgctagagcctctcggcggattcggtaaattctgcctcgtcatcctagcactctccattatcgccaacaactgtccgaacatctattcggtggccttaactcttcaggttctgagccgctactcccagcgtgtaccccgattcgtctgggttttcctcggctcctgtgcctctgtggctattgctattcccggatactctcacttcgagactgtgttggaaaatttcatgaacttcattgcgtactggctgtccatttattccggcattgcacttaccgatcatttcctcttcaagcgtggatttggtggatatcggcctgagatttacgacaagcgcgacaagcttcctcttggaatcgctgcctccattgcctttggattcggcgtggctggtatgatcactggtatgagccaatcatggtgggtcggacccattgctctccatgccggtcaagctccattcggtggcgacgttggttttgagctgggattcgctttctcggctgtgatgtatgccgtcctaaggcctattgagataaagattttcggtcggtaa

SEQ ID NO: 3 - protein, 242 aa, Aspergillus oryzae
MNAAPSPAQGVGPIYRPEGEKPTATVSKDISYENVHVLPQTPQLIALLTMIRDKNTSRADFIFYSNRIIRLLVEEGLNHLPVVERSVTTPVGREYLGVRFEGKICGVSIMRAGEAMEQGLRDCCRSVRIGKILIQRDEETCKPKLFYEKLPLDIANRWVLLLDPMFATGGSATLAVETLKAKGVPEDRILFLNLIASPSGVADFAERFPNLRVVTAFIDQGLNEKKYIIPGLGDFGDRYYTM

SEQ ID NO: 4 - DNA, 929 bp, Aspergillus oryzae
atgaatgctgcgccttcgcccgcacaaggtgttggcccaatttatagacctgaaggcgagaaacccacagcaacagtgtccaaagacatttcttatgagaatgtccatgtgctgccacaaactccgcaactaattgcgcttctgacgtaagaggaatactcagacgctctaccctaaactgatattgatgagcattaaaatcatatggagaagcatatcctctgaccaattgcagtatgataagggataaaaatacaagccgtgccgattttattttttactccaacagaattatccgactcttggtggaagagggattaaatcatctaccagttgtcgagcgatccgttacaactcctgtgggtcgtgaatacctcggggtcagatttgaaggaaagatatgtggtgtctcaataatgagggccggagaagcaatggaacagggtcttagggattgttgtcggtcggttcgaattgggaaaattttgattcagagagacgaagaaacctgcaagccgaaactcttctacgaaaagctcccccttgatatcgccaaccgctgggtgctactccttgatccgatgtttgcaaccggtgcgtcaggtcctgtattttcgatatgattgaaagctacccatgttgacgactcgtaggaggctctgcaacgcttgctgttgaaaccctgaaggcgaagggtgtgcccgaagatcgaatactatttctcaacctcattgcaagtccctctggggtcgcagatttcgcagaacgctttccaaaccttagagtagtaacagcgttcattgaccaaggcttgaacgaaaagaagttggttgaaatctctcccagtggatagaacactaacttatcgatccaacagatacatcatccctggtttaggtgattttggtgatcggtattataccatgtag

SEQ ID NO: 5 - protein, 620 aa, Aspergillus fumigatus
MADDKAMEVNEASPHETTRHADPALDPANQHNHGHPHHSGLAQKGPDDDVIYAKDVKGLVHDPAVEDKHAHSQSSKDLEEAGESYPSRPFYRRYSRYIKHVVYAVVWLLFTGWWIAGLILHRYDLGWLIPFLLYLCITLRLLTLYVPVSIVTRPAYWVWDHTARPFVRLIPEKLRIPGAALLTIAVILIGSFASEETADNTRANRAVSLFGLIVFVFALWLSSRNRKKIVWHTVIVGMLVQFIVALFVLRTKAGYDIFNFVSTLARELLGFADDGVDFLTETGFASKHSWFLVTVIPAIIFFVSLVQLLYYSGVLQWAIGKFAVFFFWSMRVSGAEAVVAAASPFIGQGESAMLIKPFVAHLTMAEIHQVMCSGFATIAGSVLVAYIGMGVNPQALVSSCVMSIPASLAASKLRWPEEEETLTAGRVVVPEDEEHKAENALHAFANGAWLGIKIAGTIGATLLCIISFVALINGLLTWWGRYLNINDPPLTLDLILGYICYPIAFLLGVSRDGDLLKVGKLIGVKLVMNEFVAYSDLQTKPEYQELSVRSRIIVTYALCGFANIGSLGNQIGVLNQLAPQRAGDVSRVAVSAMITGALATFTSAAIAGLLITEEGKYLSQ

SEQ ID NO: 6 - DNA, 2020 bp, Aspergillus fumigatus
atggcggacgacaaggctatggaggtgaacgaggcatctccccacgagacaacgcgacatgcagatcctgcgctggatcccgcaaatcaacataaccacggtcatccccaccattcgggactggcccagaagggtccagatgatgatgtgatctatgccaaagatgtcaaaggcctggttcacgatccggctgtggaggacaagcacgctcatagccagagttcgaaggacctcgaggaagcaggtgaaagctatccgtcgcggcccttctatcgccgctatagcaggtacatcaagcatgtcgtgtacgctgtcgtgtggctgttgttcaccgggtgagttgtcttccctttcaactgcaccgcatcttctggcagtcctgtttgatcaaaatccaaactcatccgttgtcgtccctcttagatggtggattgctggtttgatcctccatcggtatgacttgggctggctcatcccgttcctgctgtatctgtgcatcaccttgcggcttcttaccctctacgtgcccgtgtccatcgtgaccagaccagcctattgggtctgggaccataccgctcgaccattcgttcgcctgatccccgagaagttgaggattcccggcgcggcgttattgacgattgcggtcattctgatcggctcatttgcgtcggaagagaccgccgataacaccagagccaacagagctgtcagtctcttcggacttatcgtcttcgtcttcgccttgtggctttcgtcaagaaaccgcaagaagatcgtctggcacaccgttattgtaggaatgctggtgcaattcatcgttgctctctttgtgcttcgtaccaaggcgggttacgacatcttcaacttcgtctcgacgctggccagagagcttctgggctttgccgacgacggcgttgatttcttgacggagactggcttcgcaagcaaacactcgtggttcttggtgaccgtcatcccggctatcatcttcttcgtatctcttgtgcagcttctgtactactctggcgtcctccagtgggctattggtaaattcgccgttttcttcttctggagtatgcgggtctccggcgccgaggccgtcgtggctgctgcctctcctttcatcggacaaggtgaatcggccatgcttatcaagccgtttgtggcgcaccttaccatggccgaaatccaccaggtgatgtgctccggtttcgctaccattgcaggctccgtactggtcgcctacattggaatgggcgttaaccctcaagcgctggtttcctcctgtgtgatgagtatccctgcttctctggccgcatccaagctgcggtggcctgaggaggaagaaactctgacggctggccgtgtcgtcgtgcccgaggacgaggaacacaaggctgaaaatgcgcttcacgctttcgccaacggtgcctggctcggcatcaagatcgcgggtaccattggggcaaccctcctctgtatcatctccttcgttgccttgatcaacggactgttgacctggtggggacgttacctcaacatcaacgacccccccttgaccctcgacctgattcttggatacatctgttatcccatcgccttccttctcggcgtctcccgcgacggcgatctgctcaaagtgggcaaactgattggggtgaagcttgtcatggtaagattctatccattcacgctccgctcaccttcagggaggaccagacactaatccaattattttcagaatgaattcgtcgcctatagcgaccttcaaaccaagcccgagtatcaggaactctccgtccgctcccggatcatcgtaacgtatgctctctgcggtttcgccaacatcggctccctgggtaaccaaatcggtgtgcttaatcaacttgccccccaacgtgcgggcgatgtgtcccgtgttgcagttagtgccatgattacgggcgccctggctacttttaccagtgcggctatcgcgggtctgctgattacggaggagggcaagtaccttagtcagtga

SEQ ID NO: 7 - protein, 453 aa, Aspergillus fumigatus
MESLSPLYSSATEKRYYSPPWADLSIIGIAGSSGSGKTSVAMEIVKSLNLPWVVILDMDSFYKSLTPEQHAKAHANEFDFDCPDAIDFDALVQTLRDLKQGKKANIPVYSFAEHQRQPQTTTLYSPHVLILEGILALHDPRIMEMLDVKIFVEADMDVCLGRRILRDVRERGRDIDGIIKQWFQFVKPSYKQYVEPQRSVSDIIIPRGIENRTAIDMVVKHIQRKLQEKSDKHSAELQKLEMIASEEQLSANVFLMPQTPQFISMNTILQDPATEQVDFVFYFDRLACLLIEKALDCTRYQPVKVETPQGMNYNGLHPEGLVSAVAILRGGSCLETALKRTIPDCITGRLLIQTNERNEEPELHYLKLPPGIEEHATVMLLDPQMASGGAALMAVRVLVDHGVAEDRIVFVTCAAGKVGLKRLSTVYPEVRVIVGRIEEEREPRWLEKRYFGC

SEQ ID NO: 8 - DNA, 1815 bp, Aspergillus fumigatus
atggagtctctctctcctctttactcctcggccacggagaagagatactactctccaccatgggcggatttgagtatcattggaattgcaggcagctcgggctccggcaagacgtctgtggccatggagattgttaagtccttgaatctgccatgggttgtgattcttgatatggtactaaggacccgttcgaacatcatttctatagatataagccattttcgtgcatcctcgtgacttctaaccccttctggtacaggactcgttctataagagtttgactccagagcaacatgccaaagcgcatgcaaatgaattcgatttcgactgtccggatgcaatcgacttcgatgcattagtacagacgttaagggatctcaagcaagggtaattcctctccttgctcctcgtaatccaccaagctctccggaaatttgcacgcagctaacagcaaatgcgtcaaccaaataaagaaagaaagcaaacatacctgtctactcttttgctgaacatcagcgccaaccgcagacgaccactctttactcgccgcatgtcttgatcttagagggaattctggcgctccatgatcctcggatcatggagatgttggacgtcaaggtaattctactttcacccacttactacctcaagatacttatcagacccttttcagatttttgtcgaggcagatatggatgtttgccttggacgcagaagtgagtggaaaatcatacccagtatgattaactgtcctaaccaaggcggcgccagtattgcgtgatgtccgggagagaggcagagacattgatggtatcattaaacaatggttccaatttgtcaagccatcgtataagcaatacgtcgagcctcagcggtcggtttcgggttagctagcacaaaattccctcccgctctgcttgtctgacggaagtagatattatcatcccccgcggaattgagaacagaacggctatcggtaagcgaagatggtgaacttcagtttgcacgcctttgctgaatagtcccagatatggttgtgaaacatatccagcgcaaacttcaggagaaatccgacaaacacagcgcagagttacagaagctggagatgatcgcttcagaagaacaattgtcggcaaatgtgtttctcatgcctcagaccccgcaattcatcagcatgaacacaatattgcaggaccctgccacggaacaggttgattttgtcttttattttgaccgcctcgcttgtctactaatagagaagtgcgttgacccttatgtgccagcgcctgaaaagttcaagtttgggactgaccataacatcggattaatagggccttggactgtactcggtatcaaccggtgaaggttgagacccctcaagggatgaattataacgggttacatccggaaggtctagtgtctgcagtagcaattttgcgaggtggttcttgtcttgaaacagctctcaagcgaactattcctgactgtattactggtcgactgctcattcaaacgaacgagcggaacgaggagccagagctgcattacttgaaactgccgccaggcattgaagagcacgcaacagtcatgctccttgatcctcaaatggctagcggaggagctgccctgatggcggttcgggtcttagttgatcacggagtcgcggaagacagaattgtctttgtgacctgtgccgctggaaaggttggacttaagcggcttagcaccgtttaccctgaagtcagggtcattgtagggaggatcgaagaggagcgagagcctcgatggttggagaagagatatttcggatgttag

SEQ ID NO: 9 - protein, 515 aa, Aspergillus oryzae
MFGRKDQDLEKGPQVAKTALDTSSDGAVPGETFVYGDSLYAKLQRLAGKINIEQRGIERVPPDEQTDTSYFNIGSMWLAANMVVSSFAIGVLGKSLFALGFVDAILVNLFFNLLGIMTVGFFSCFGPPFGLRQMVLSRFWFGYWGTKFIACLNVLACIGWSAANAIVGAQLLHAVNTDVPGFAGVLIIAFCTFIITFAGYKVVHMYEYWSWVPTFIVFMIVFGMFAHSGDFVNIPMGVGKSELGSCLSFGSTVYGFATGWTSYAADYTVYQPRDRSRRKIFFSAWAGLIVPLIFTQFLGIAIMTATSLNDGDNKYQAGYTASGNGGLLAAVLDPLGGFGKFCLVILALSIVANNCPNIYSVSLTLQVLSRYTQRVPRFIWVFLGSCASVAIAIPGYSHFETILENFMNIIAYWLAIYSGISLTDHFVFKRGFGGYRVEIYDKPNKLPPGIAAAVAFCFGIAGMVTGMSQSWWIGPIAKHAGALPSGGDVGFELAFAFASVSYIPLRMAELKVFGR

SEQ ID NO: 10 - DNA, 1698 bp, Aspergillus oryzae
atgttcggaaggaaagatcaagaccttgaaaaaggtcctcaggtggcgaagacggcattggacaccagcagcgatggcgcggtacccggagagacctttgtatacggcgattcgctgtatgctaagcttcaacggctcgcaggaaagatcaatatcgagcagcgtggaattgaacgggtaccaccagacgagcagacggatacgtcgtacttcaatattggcagcatggtgtgtttatgattccgttgcgttaattttgtctgtgccagctattgtctggcatttcaagaccgcctgtccaactgacccaacacgcagtggttggcggccaacatggtagtcagttcctttgccattggtgtcctcggaaaatccctcttcgctcttggctttgtggatgccatcctggtaaatctcttcttcaacttgttgggtatcatgactgtgggattcttttcgtgctttggtcctcctttcggtctgcgacaaatggtactctccagattctggttcggatattgggggacaaagttcagtgagtatatccaatttgtggtgtcttattatagttaaacagccattaatacttgtctagttgcctgcctcaacgttttggcctgtattggttggtctgcggccaatgcaattgttggtgctcagctcctccatgcggtcaataccgacgtcccaggatttgcaggtgtgctaattatcgccttctgcacgtttattattacttttgccggatataaggtcgtccacatgtacgagtactggagttgggtgccgacatttatcgtcttcatgatcgtctttggcatgttcgcccactccggagattttgtgaacatccccatgggcgtcgggaaatccgaactcggcagctgcctatccttcggttcaactgtgtacggatttgccactggttggaccagctacgcggctgattacacggtgtaccagccccgggaccgcagtcgccgcaagatcttcttctccgcatgggccggtcttatcgttcctctgatcttcacacaattcctcggaatcgccattatgaccgccaccagtctcaacgatggtgacaacaagtaccaagctggctatacagcttctggaaacggaggtttgctcgctgctgttctcgatcctcttggtggattcggcaagttctgcctcgtgatcctggccctttccattgtcgccaacaactgccccaacatctactccgtctccctgacccttcaagttctgagccgttatacccagagagttcctcgtttcatctgggtgttcttgggttcctgcgcctctgtggccattgccatccccggatattcccatttcgagaccatcctggagaacttcatgaacatcattgcttattggctcgccatttactctgggatctcactcactgaccacttcgtcttcaaacgtggattcggcggataccgtgttgagatctacgataagcccaacaagcttcccccgggtattgcggccgcggttgctttctgcttcggtatcgccggtatggttactggaatgagccagtcgtggtggatcggacccattgcaaagcatgcgggtgcgctacccagtggtggtgacgtcgggttcgagttagcgtttgcttttgcttccgtgtcgtatatccccttgagaatggctgaattgaaggtgtttggtcggtaa

SEQ ID NO: 11 - protein, 514 aa, Aspergillus niger
MFGGVESDLEKAPQVGKTPMDDSSDGAVPGESFMYGNSLYAKLQRLAGKLNIEQRGVERVPDNERTDTSYFNISSMWLAANMVSSSFAIGVLGYSTFALGFVDAILVCFFFNLLGVLTVCWFSCFGPAFGLRQMVLSRFWFGYWPTKISKFLFALLNVLSCLGWSAANSIVGAQLLNAVNSDVPGFAGILIIAICTLFVTFAGYKVVHLYEYYSWIPTFIVFMIVFGLFAHSGDFRNLPMGTGTSELGSCLSYGATVYGFATGWTSYAADYTVYQPANRSRRKIFFAAWGGLIPPLLFTQFLGIAVMTATSINGGDNKYARGYSESGNGGLLGAVLDPVGGFGKFCLVILSLSIIANNCPNIYSVSLTLQVLSRYSQRVPRFIWVLLASGVSIAVAIPGYSHFEEVLENLMDFIAYWLAIYSGVALADHIVRRGFSGYRPETYDKPNKLPYGIAACISFAFGVAGMVTGMSETWYTGPIAKHAGNGDVGFELALLFSFVVHSVLRPIELKMLGR

SEQ ID NO: 12 - DNA, 1684 bp, Aspergillus niger
atgttcggaggagtcgagtccgatctcgaaaaagccccccaggtgggcaaaacgcccatggatgacagcagcgatggcgctgtccctggggagagttttatgtacggcaattcgctgtacgctaagctgcaacggttggcggggaagctcaatattgagcagcgaggtgtcgaacgtgtcccggacaatgaacggactgacacgtcgtacttcaatatcagcagcatggtgtgttcctaactcttgtttgccttgtgctttcgtctccgtcggctctctcgtcggctgctccaagaccgccctcccaaactgacccgtcgcagtggctggcggccaacatggtgagcagttctttcgctatcggcgtgcttggttactccaccttcgctctcggcttcgtcgatgccatcctcgtctgcttcttcttcaacctgctgggtgtcttgactgtctgctggttctcctgctttggtcccgccttcggtctgcgccagatggtgctttcccggttctggtttggttactggcctaccaagatcagtaagtttctctgtgacaatattgatatcataaccagatgcttatatcctctgctacagttgctcttctcaacgttctctcctgtttgggctggtccgccgccaactccatcgtcggcgcccagcttctcaatgctgtgaactccgacgttcctggatttgcaggcatcttgatcatcgccatctgcaccctcttcgtcaccttcgccggatacaaggtcgtccacttgtacgaatactacagctggatccccaccttcattgtcttcatgatcgtgttcggattgttcgcccactccggtgacttcagaaacctccccatgggaactggaacctctgagctcggctcttgcctgtcttacggtgcgactgtctatggtttcgccactggctggaccagttacgccgccgattacaccgtctaccagcccgcgaaccgcagccgccgcaagatcttcttcgccgcctggggaggtctgatcccccctcttctcttcacccagttcctgggtattgccgtcatgaccgccacttccatcaatggcggagacaacaagtacgcccgtggttatagcgagtctggaaacggaggtcttctgggcgctgtccttgaccccgttggcggattcggcaagttctgtctggtgattctgtctctgtccatcatcgccaacaactgccccaacatctactccgtctccttgaccctccaggtcctgagtcgctactcccagcgcgttccccgtttcatctgggtcctccttgcctctggtgtctccatcgccgtcgccatccccggttattcccacttcgaagaggttctggaaaacctaatggacttcatcgcctattggctggccatctactccggtgtcgctcttgccgaccacatcgttcgccgtggtttctccggatatcgccccgagacatatgacaagcccaacaagctgccctatggtatcgccgcttgcatctcctttgctttcggtgtcgcaggtatggtcacgggcatgagtgagacgtggtacactggtcccatcgccaagcatgctggtaatggtgatgtcggtttcgagttggctcttctcttttctttcgtcgtgcattccgttctgagacccattgagctgaaaatgctgggtcgg

SEQ ID NO: 13 - protein, 531 aa, Penicillium chrysogenum
MLSKFAKPFRSSNEDLEKAPQVNNSSDLNSNEVFDNSSDGAVPGESFEYGDSMYAKIQRVAGKFNIEQRGVERVPDYERTDTSYLNIGSMWLAANMVVSSFAIGVLGKSAFNLGFVDAILVCLFFNLLGVMTVCFFSCFGPPFGLRQMVLSRFWFGWWAVKFIAILNIIACVGWSAANAIVGAQLLNAVNDEVPGYAGILIITFCTLFVTFCGYKFVHMYEYWSWIPTCIVFIIVLGVFAHSGDFINIPMGAGVSEMGACLSFGSTVYGFATGWTSYAADYTVYQPKTQSRRLVFFSAWLGLIIPLLFTQFLGIAIMSATEMGDGVNNKYAEGYRASVNGGLIAAVLEPLGGFGKFCLVILALSIIANNCPNIYSVGLTLQVLSRATQRVPRFIWTFLASCVSLAIGIPGYDHFETVLENFMSLIAYWLAIYSGIALVDHFVFRRGFDGYRPEDYDNPAKLPMGIAASIAFVFGIVGVVVGMSQSWWTGPIAKHAGDPASGGGDIGFELGFAFSSAVYCVLRPIELRMIGR

SEQ ID NO: 14 - DNA, 1896 bp, Penicillium chrysogenum
atgctttcaaagttcgccaaaccgttccgcagctcaaatgaggacctggaaaaagcaccccaggtgaacaattcctccgatctaaatagcaatgaggtattcgacaactccagcgatggcgccgtgcccggtgagagcttcgaatacggcgattcgatgtatgccaagatccagcgcgtagctggcaagttcaacatcgagcagcgaggtgttgagcgggttccggattatgagcggaccgatacgtcctatctcaatattggtagcatggtgtgttcctgcaacaattaccgggcgtgatcgccttggcctggaggccttttccactgaccaaacctagtggttggccgccaacatggtcgtcagctccttcgccattggcgtccttggaaaatctgcattcaatttgggcttcgttgatgccattttggtctgtctcttcttcaacctcctcggtgtcatgaccgtgtgcttcttctcgtgcttcggtcctccctttgggctgcgccaaatggtgctctcacgattctggtttggatggtgggctgtgaaattcagtaagtcatctcgggatgttcattcagtgtgaaatggtcatgttaactagtagctttctaagttgccatccttaacattatcgcgtgtgtcggttggtctgccgccaacgccatcgtcggtgcacagctcctcaacgctgtgaacgacgaagtccccggatacgccggtatcctcatcatcacattctgcacactatttgtcaccttttgtgggtacaaattcgttcacatgtatgagtactggagctggattcccacctgtatcgtgtttatcatcgtgttgggcgtattcgcccactcgggcgatttcatcaacatcccaatgggagcaggcgtttctgaaatgggagcctgtctatcctttggatctactgtctatggtttcgcaacaggctggaccagctacgctgccgactacaccgtctaccagccaaagacccagagccgtcgtctggtcttcttttctgcatggcttggtctgattatccccctgctcttcactcaattcctcggcatcgccatcatgtccgcgacggagatgggcgacggagttaacaacaagtacgccgaaggctaccgagcctccgtcaacggcggcctgatcgcagccgtgcttgagcctctcggcggtttcggcaagttctgcctcgtcatcctggccctgtccattatcgccaacaactgccccaacatctactctgtcggcttgacgttgcaggtgttgagccgggctacgcaacgcgtgccgcggttcatctggactttcctcgcctcgtgcgtctcccttgccatcggtatccccggttacgaccactttgagaccgtcctcgagaatttcatgagccttatcgcctactggctggctatttactccggcattgcgctcgttgaccactttgttttccgacgtggattcgatgggtatcgtccggaggactacgataaccccgccaaactccccatgggaatcgccgcttctattgcgtttgtctttggcattgtcggtgttgttgtcggcatgagccagtcgtggtggacgggtcctatcgccaagcatgctggagacccggcgtctggtggtggtgacatcggatttgagttgggctttgccttctcttccgcagtgtattgcgtcttgcgacctatcgagcttcgtatgattggtcggtaggatcgactcttatagtatttgcgttttgcgatgtacatattgggttggttcgggatgggttcttggatttgtgcataggttcggttgcattagtgtattatacatttgcatagttgcatacagcatattgacgttcaaatctatatttaactgtaagtttagcacacc

SEQ ID NO: 15 - protein, 533 aa, Saccharomyces cerevisiae
MLEEGNNVYEIQDLEKRSPVIGSSLENEKKVAASDTFTATSEDDQQYIVESSEATKLSWFHKFFASLNAETKGVEPVTEDEKTDDSILNAASMWFSANMVIASYALGALGPMVYGLNFGQSVLVIIFFNIMGLIFVAFFSVFGAELGLRQMILSRYLVGNVTARIFSLINVIACVGWGIVNTSVSAQLLNMVNEGSGHVCPIWAGCLIIIGGTVLVTFFGYSVIHAYEKWSWVPNFAVFLVIIAQLSRSGKFKGGEWVGGATTAGGVLSFGSSIFGFAAGWTTYAADYTVYMPKSTNKYKIFFSLVAGLAFPLFFTMILGAASAMAALNDPTWKAYYDKNAMGGVIYAILVPNSLNGFGQFCCVLLALSTIANNVPNMYTVALSAQALWAPLAKIPRVVWTMAGNAATLGISIPATYYFDGFMENFMDSIGYYLAIYIAISCSEHFFYRRSFSAYNIDDWDNWEHLPIGIAGTAALIVGAFGVALGMCQTYWVGEIGRLIGKYGGDIGFELGASWAFIIYNILRPLELKYFGR

SEQ ID NO: 16 - DNA, 1602 bp, Saccharomyces cerevisiae
atgttggaagagggaaataatgtttacgaaatccaagacttggagaagagatctcctgtaataggctcaagcttggaaaacgaaaagaaggtagccgcttctgatactttcacagcaacttccgaagatgaccaacagtatatcgttgaatcatcagaggccacaaaattatcgtggttccataagttctttgccagtttgaatgcagaaacaaagggtgttgaaccagttacagaggatgaaaaaacggacgattccatactgaatgctgcgtccatgtggttttcagctaatatggtcattgcatcatatgctttgggtgccttaggacccatggtgtacggtctaaattttggtcaaagtgttttagttatcatatttttcaatataatggggttgatatttgttgcgttcttttctgtctttggtgcagaactgggtttaagacagatgattttatcaagatatctggttggtaatgtaacggcaagaattttctctcttattaacgttattgcttgtgtcggttggggtattgtgaatacctcagtatccgcacaactgctgaatatggtgaatgaaggctccggccacgtctgtcctatttgggccggttgtctgatcattattggtggtaccgtgcttgtgactttttttggttacagtgtcattcatgcatacgagaaatggtcatgggtacccaattttgccgtctttttggttattattgcccaactatcgagatcaggaaagtttaaaggtggtgaatgggtaggaggtgcaactactgcaggtggtgttctttcttttggttcatctattttcggttttgccgcaggatggacgacctatgcagctgactacactgtttatatgccaaagagtacaaacaagtacaagatttttttctccctagttgctggtctagcgttccctctatttttcaccatgattcttggtgctgcttctgctatggctgctcttaatgacccaacctggaaggcatattatgacaaaaatgctatgggtggtgtcatatatgctatcctggttcctaactctctgaacggatttggtcaattctgctgcgttttgttagctctgtccactattgcaaataatgttccaaatatgtatactgttgctttatccgctcaagctttgtgggcacctttggctaaaataccaagagtagtctggacaatggcaggtaatgctgccactttaggtatttccatccctgctacctattactttgatggctttatggagaatttcatggattctatcggttactacttagctatttacattgccatctcatgttcagaacatttcttctacagacgttccttcagtgcttacaatattgatgattgggataattgggagcatctacctatcggtatcgcaggtactgctgccttaattgttggtgccttcggtgttgcattgggtatgtgtcaaacctattgggttggtgaaattggccgcttaattggtaaatatggtggtgacattgggttcgagttgggtgcaagttgggccttcatcatctacaacatcttaagacctttagaattaaaatacttcggtcgttag

SEQ ID NO: 17 - protein, 514 aa, Candida albicans
MSSDPEKNLGMPEKTSVNSYDSMDPSSSSSGADAEIETTKLNFIDRWAHKLNAETKGIELVTDEEKTDTSFWNLATMWLSANLVIATFSLGALGITVFGLAFGQAVLVIIFFSILGGFPVAFFSCFGSALGLRQMLLSKFLIGDLTTRLFAAINVVACVGWGAVNTMSSAQLLHIVNNGTLPPWAGCLIIVVCTVLVTFFGYHVIHAYEKWAWIPNLIIFIIIIVRFAMTNKFTSKSFEGGETTAGNVLSFGGTVFGFATGWTTYSSDYVVYHPRNTNSWKIFFSIFFGLLTPLMFTLILGAACATGIAGDPEWTRLYKEDSVGGLVYAILVHDSLHGFGQFCCVVLALSTVANNVPNMYSMALSAQTVWAGFRKIPRVAWTIAGNGATLAICIPAYYKFEAVMENFMNLISYYLSIYESIMFASHFIWNSGRFDGYDYERWNDKEAYPVGYAGVFGFACGVAGVVLGMNQTWYSGVIGRRIGEFGGDIGFELAIGFAFIGFNVARYFEKKYIR

SEQ ID NO: 18 - DNA, 1545 bp, Candida albicans
atgtctagtgatcctgaaaagaatttaggaatgccagaaaagacttcggtcaatctgtatgattccatggacccatcttcctcatcatcgggtgcagatgcagaaattgaaacaacaaaattaaatttcattgatagatgggcacataaattaaatgctgaaactaaaggtattgaattagtcactgatgaagaaaaaactgatacttcattttggaatttagccaccatgtggttaagtgcaaatttagtcattgcaactttttcattaggtgctcttggtataaccgtatttggattagcttttggtcaagcagtattagtgattattttcttttcaatactaggtgggttccccgtggcatttttttcatgttttggatcagctttaggattaagacaaatgttattatcgaaatttcttattggtgatcttactacaagactttttgctgctattaatgttgttgcttgtgttggttggggtgcagttaataccatgtcttcagctcaattattgcatattgttaataatgggactttaccaccttgggctggttgtcttattattgttgtttgtaccgttttggtgacattttttggatatcatgttattcatgcttatgaaaaatgggcctggattccaaatttgattattttcattattataattgttagatttgccatgacaaataaattcactagtaaatcatttgaaggtggtgaaactactgctggtaatgttttaagttttggtggtacagttttcgggttcgccacgggttggactacttatctgagtgattatgttgtttatcatcctcgtaatactaattcttggaagatttttttcagtattttctttggattattaactccattaatgtttactttaatattgggtgctgcttgtgccactggtattgctggtgatccagaatggactagattatataaggaagattctgttggtgggttagtttatgccattttggttcatgattctttacatggatttggtcaattttgttgtgttgttttggctttatctactgtggctaataatgttcctaatatgtattctatggctttatcagctcaaactgtttgggctggtttcagaaaaatcccaagagttgcttggactattgctggtaatggtgccactttagccatttgtatccccgcatattataaatttgaagccgttatggaaaattttatgaatttaatttcctattatttatcaatttatgaaagtataatgtttgcatctcatttcatttggaatagtggtagatttgatggatatgattatgaaagatggaatgataaagaagcttaccctgttggttatgctggagttttcggatttgcttgtggtgttgctggggttgttttaggtatgaatcaaacttggtatagtggtgttattggtagaagaattggtgaatttggtggtgatattggatttgaactagctattggatttgcctttattgggtttaatgtcgctagatattttgaaaagaaatatattcgatag

SEQ ID NO: 19 - protein, 530 aa, Saccharomyces cerevisiae
MPEKLAMSMVDIKDAGSELRDLESGALDTKSSAADVYYEGVELHRTNEFIDNKPSFFNRIAAALNAETKGIEPVTDDEKNDDSILNAATIWFSANMVIVAYSVGALGPLVFGLNFGQSVLVIIFFNILGLIPVALFSLFGVELGLRQMILSRYLAGNITARFFSLVNVIACVGWCVLNISVSAQLLNMVNEGSGHNCPIWAGCLIIAGGTVLVTFFGYSVVHAYEKWSWVPNFAAFLVIIAQLSRSGKFKGGEWVGGATTAGGVLSFGSSVFGSAAGWATYAADYTVYMPKTTSKYKIFFSVVAGLAFPLFFTMILGVACGMAALNDPTWKSYYDKNAMGGVIYAILVPNSLNGFGQFCCVLLALSTIANNVPNMYTVALSAQALWAPLAKIPRVVWTMAGNAATLGISIPATYYFDGFMENFMDSIGYYLAIYIAIACSEHFIYRRSFSAYNIDDWDNWEHLPIGIAGTAALIAGAFGVALGMCQTYWVGEISRLIGEYGGDIGFELGGSWAFIIYNIVRPLELKYFGR

SEQ ID NO: 20 - DNA, 1593 bp, Saccharomyces cerevisiae
atgccagaaaagttagccatgtcaatggtcgatataaaagatgcgggtagtgaactaagggacctcgaatctggagctctggacacaaaatcttccgccgctgatgtatactatgagggtgtagaattgcatagaaccaacgaattcattgataataagccgtcctttttcaataggattgcagctgctttaaatgctgagacgaaaggtattgaaccagttacagacgatgaaaaaaatgatgactcgatactcaatgccgccactatatggttttcagctaatatggtgattgtagcctattccgtaggtgccttgggtcctctagtatttggcctaaatttcggccaaagtgttttagttatcatttttttcaatattttgggtttgatccctgttgcattattctcactttttggggtagaactgggcctaagacagatgattctatcgagatatttggctggtaacatcacagcaagatttttctctcttgttaatgtcattgcttgtgtcggttggtgtgttttaaatatttctgtttctgctcaacttttgaatatggtgaatgaaggctctgggcacaactgtcctatttgggcaggttgtttgattattgctggtggtaccgtgcttgtgactttttttggttacagtgtcgttcatgcatacgaaaaatggtcgtgggtacccaattttgctgcctttttggtcattattgcccaactatcgagatcaggaaaatttaaaggtggtgaatgggtagggggtgcaactactgcaggtggtgttctttcttttggttcatctgtttttgggtcagctgcgggttgggcgacttatgcagcagattacactgtttatatgccaaagaccacaagtaaatacaaaatttttttttccgtagtagccggtctagcgttccctctatttttcaccatgattcttggtgttgcttgcggtatggcggcccttaatgacccaacctggaagtcatattatgataaaaacgccatgggtggtgtcatatatgctatcctggtccctaactctctaaacggattcggtcaattctgctgtgttttgttggctctgtccactattgcaaataatgttccaaatatgtatactgttgctttatccgctcaagctttgtgggcacctttggctaaaataccaagagtagtctggacaatggcaggtaatgctgccactttaggtatttccatccctgctacctattactttgacggctttatggagaattttatggattccattggttattatttggctatttatattgccattgcatgttcagagcattttatttataggcgttccttcagtgcttacaatattgatgattgggataattgggagcatctacctatcggtatcgcaggtactgctgccttaattgctggtgcctttggagtagcgttgggtatgtgccaaacttattgggttggtgagatcagtcgtttgatcggagagtacggcggtgacattgggttcgagttaggcggaagttgggcgttcatcatctataacattgtaagacctttagaactcaaatatttcggtcgatga

SEQ ID NO: 21 - protein, 519 aa, Komagataella phaffii
MSAYKTDIEKADAGSSDKNSTYQVKTEDFQVDESVAEVSKGKFAWYENFTLMMKAETRGIEIVPEEEKTRTSLWEAASMWFSANLVIGTFALGAISQTVFALDFWSSVLCIIFFNMLGVFPVAFFSVFGVKFGLRQMILTRFLSGDLAMRLFAAINCICCVGWGAVNIMAAAQLLHIVNNKTLPPWAACLIFVVLTILVTFFGYDVIHAYEKWSWIPNMFVFIVIIARMSISGNFTFGTMAGGPTVAGNVLSFGGAIFGYASGWATFAADYTVYMRTDTPPLKIFAWVYFGLFTPLCFTMMLGTACATGIFSDESWATLYYDNGVGGLVYAVLVENSLHGFGQFCCVLLALSTVANNIPNMYSIGLSAQAVTSKARRVPRIVWTLCGNSVTLAISIPAYYHFERFMSNFMNIIGYNLAIYDTVCLSEHFIWKRGFSSKYDVMLEQWHDKTAAPPGYAGLIGFGCGAAGVVLGMNQVWYSGAIGRLIGDFGGDVGFELGAGFSFLGFNTARYFELKYCGR

SEQ ID NO: 22 - DNA, 1560 bp, Komagataella phaffii
atgtctgcttacaaaactgatatcgaaaaggccgacgctgggtcgtctgacaagaactcgacataccaggtcaagacagaggacttccaagtagatgagtccgtggctgaagtttccaagggaaagttcgcctggtatgagaatttcactctcatgatgaaggctgagacaagaggtattgaaattgtccccgaagaagagaagactagaacttccctgtgggaggccgcctccatgtggttctccgccaacttggttatcggtacgtttgcccttggtgctatttctcagactgtgtttgctttggatttttggtcttcagtgctatgcattatcttctttaacatgctgggcgtgttccccgttgcttttttctccgtctttggagtcaagtttggtcttagacagatgattttgaccagatttttgtctggagatctggctatgagactgttcgctgcaattaactgtatctgttgtgttggatggggtgccgtcaatatcatggctgctgcccaattgttgcacattgttaacaacaaaactttgcctccttgggctgcatgtctgattttcgttgtgttgaccattcttgtgacattcttcggttacgatgtcattcatgcctatgagaaatggtcctggattcccaacatgtttgtgttcatcgttatcattgccagaatgtccatctctggaaactttactttcggtaccatggccggaggtccaactgtggcaggaaacgtcctcagtttcggaggtgcaatttttggatacgcttctggttgggctactttcgcagccgactacaccgtttacatgagaacagatacccctcccttaaagatttttgcctgggtttattttggactcttcactccattgtgtttcaccatgatgttgggaactgcatgtgccactggtattttctcagacgaatcatgggctactctctactacgacaacggtgtaggtggcttggtttacgctgtattggtcgagaactccttgcacgggttcggtcaattctgctgtgttttgctggccttgtctactgttgctaacaacattccaaacatgtactccattggtctgtctgctcaggctgtcacctccaaagctagacgtgttcccagaatcgtttggactctttgcggaaacagtgtcactttagccatcagtatccctgcctattaccacttcgaacgtttcatgagtaatttcatgaacattatcggttacaacttggctatctacgacacggtgtgtctttcggagcattttatttggaaaaggggattctcgtccaagtacgacgttatgcttgaacaatggcacgacaagactgctgctcctccaggatatgctggactcattggatttggctgtggtgccgctggtgtcgtcctgggaatgaaccaggtctggtactctggggccataggtaggttgattggagatttcggaggagacgtaggattcgagttgggtgcagggtttagtttcttgggctttaacactgcccgttactttgagctgaaatactgcggacgttaa

SEQ ID NO: 23 - protein, 526 aa, Candida albicans
MTENYDLEQQETQAPENQVNVEKILLDKSNVDEHPITSTIEQPSTSNSSLKKPTNWVDKIGLRINAEIRGIERVPESERHDNSLLSPFLVFLSPNMVISGLSIGSLGPVAYNLDFRTSIIIITIFCIIGSIPVGFFSAFGMRFGIRQQILSRYFTGNIMGRIFALFNVISCIGWNAVNVIPCAQLLNSVGPLPPWAGCLILVGCTCIFAVFGYKTVHLYEKYSWIPNFIVFMIIIAKFSQTHAFNWGEKKSGPTEAGNVLNFISAIFGFTVGWIPSSADYTVYMPANTNPWKVAFAMTTGLSLPAMFTAILGAAIGTSVNLKGSRFEQAYNKNSTGGLIYEILCGDNNNQGYRFIIVVFALGAIANGIPGSYSLSLAIQCIWSQCARVPRIAWCILGNLVALAFSISAYYKFQDTMSNFLSIIAYNVSIYLSISLTEHFIYRRGFSGYDVTDFNNYKTMPVGIAGVVAFCFGICSTVLSMNQTWYQGVIARKIGDNGGDISFEMNIMFAFIGYNLVRPFELKYFGR

SEQ ID NO: 24 - DNA, 1581 bp, Candida albicans
atgacagagaattacgatttagaacaacaagaaacccaggcaccggaaaaccaagtcaatgtggaaaaaattttgcttgataagagtaatgttgatgagcatcccattacatccaccattgaacaaccatcaacgagtaattcctcattaaaaaaaccaacaaattgggttgataaaattggacttagaataaatgctgaaatcagaggtattgaaagagttcccgaatctgaacgtcatgacaattcattattatctcctttcttagtgtttttatcaccaaatatggtcattagtggattatcaattggatcattaggacccgtggcatacaatttagatttcagaactagcattataataataaccatattttgtattattggaagcattccagtggggtttttcagtgcctttggaatgaggtttggcattcgtcaacaaattttatcaagatacttcactggtaatattatggggagaatatttgctttgtttaatgtcatttcgtgtattggttggaatgccgttaatgttattccttgtgctcaattattaaactctgtaggtccattacctccttgggctggttgtttgatcttggttggttgtacatgtatctttgctgtgtttggttataaaaccgttcatttgtatgaaaaatactcttggatacctaatttcattgtgtttatgatcattattgctaaattctctcaaactcatgcatttaattggggtgaaaaaaaatctggtccaactgaagctggtaatgtgttgaatttcatatctgctatatttggttttactgtgggatggattcctctgctggcggattataccgtatatatgcctgcaaatactaatccttggaaagttgcctttgccatgaccaccgggttatcattaccagcgatgtttacagcaattttgggagcagcaataggaactagtgtaaatttaaagggatcaagatttgaacaggcatacaataaaaattccaccggaggattgatttatgagatattatgtggtgataataataatcaaggctatcgatttatcattgttgttttcgctctcggtgctatagcaaatggtattcctggatcttattctttgtctcttgccattcaatgtatatggagtcaatgtgctcgagtcccaagaattgcttggtgtattttgggtaatttggtagctttagcattttccatcctggcatattataaatttcaagacaccatgtcgaattttttgtcaattattgcttataatgtatccatttatttgagtatctcgttgacagaacattttatttatcgaagggggttttctgggtatgatgttactgatttcaacaattataaaaccatgcccgttggaatagccggtgttgtggcattctgttttgggatttgttccactgtgctttcaatgaaccaaacatggtatcaaggtgtcattgcaagaaagattggtgataatggtggtgatatatcctttgaaatgaatatcatgtttgcctttattggttacaacttggttagaccatttgaattgaaatactttggaagatga

SEQ ID NO: 25 - protein, 526 aa, Candida albicans
MTENYDLEQQETQAPENQVNVEKILLDKSNVDEDPITSTIEQSSTSNSSLKKPTNWVDKIGLRINAEIRGIERVPESERHDNSLLSPFLVFLSPNMVISGLSIGSLGPVAYNLDFRTSIIIITIFCIIGSIPVGFFSAFGMRFGIRQQILSRYFTGNIMGRIFALFNVISCIGWNAVNVIPCAQLLNSVGPLPPWAGCLILVGCTCIFAVFGYKTVHLYEKYSWIPNFIVFMIIIAKFSQTHAFNWGEKKSGPTEAGNVLNFISAIFGFTVGWIPSSADYTVYMPANTNPWKVAFAMTTGLSLPAMFTAILGAAIGTSVNLKGSRFEQAYNKNSTGGLIYEILCGDNNNQGYRFIIVVFALGAIANGIPGSYSLSLAIQCIWSQCARVPRIAWCILGNLVALAFSISAYYKFQDTMSNFLSIIAYNVSIYLSISLTEHFIYRRGFSGYDVTDFNNYKTMPVGIAGVVAFCFGICSTVLSMNQTWYQGVIARKIGDNGGDISFEMNIMFAFIGYNLVRPFELKYFGR

SEQ ID NO: 26 - DNA, 1581 bp, Candida albicans
atgacagagaattacgatttagaacaacaagaaacccaggcaccggaaaaccaagtcaatgtggaaaaaattttgcttgataagagtaatgttgatgaagatcccattacatccaccatcgaacaatcatcaacgagtaattcctcattgaaaaaaccaacaaattgggttgataaaattggacttagaataaatgctgaaatcagaggtattgaaagagttcccgaatctgaacgtcatgacaattcattattatctcctttcttagtgtttttatcaccaaatatggtcattagtggattatcaattggatcattaggacccgtggcatacaatttagatttcagaactagcattataataataaccatattttgtattattggaagcattccagtggggtttttcagtgcctttggaatgaggtttggcattcgtcaacaaattttatcaagatacttcactggtaatattatggggagaatatttgctttgtttaatgtcatttcgtgtattggttggaatgccgttaatgttattccttgtgctcaattattaaactctgtaggtccattacctccttgggctggttgtttgatcttggttggttgtacatgtatctttgctgtgtttggttataaaaccgttcatttgtatgaaaaatactcttggatacctaatttcattgtgtttatgatcattattgctaaattctctcaaactcatgcatttaattggggtgaaaaaaaatctggtccaactgaagctggtaatgtgttgaatttcatatctgctatatttggttttactgtgggatggattcctctgctggcggattataccgtatatatgcctgcaaatactaatccttggaaagtcgcctttgccatgaccaccgggttatcattaccagcgatgtttacagcaattttgggagcagcaataggaactagtgtaaatttaaagggatcaagatttgaacaggcatacaataaaaattccaccggaggattgatttatgagatattatgtggtgataataataatcaaggctatcgatttatcattgttgttttcgctctcggtgctatagcaaatggtattcctggatcttattctttgtctcttgccattcaatgtatatggagtcaatgtgctcgagtcccaagaattgcttggtgtattttgggtaatttggtagctttagcattttccatcctggcatattataaatttcaagacaccatgtcgaattttttgtcaattattgcttataatgtatccatttatttgagtatctcgttgacagaacattttatttatcgaagggggttttctgggtatgatgttactgatttcaacaattataaaaccatgcctgttggaatagctggcgttgtggcattctgttttgggatttgttccactgtgctttcaatgaaccaaacatggtatcaaggtgtcattgcaagaaagattggtgataatggtggtgatatatcctttgaaatgaatatcatgtttgcctttattggctataacttggttagaccatttgaattgaaatactttggaagatga

SEQ ID NO: 27 - protein, 526 aa, Candida albicans
MKENYDLEQQETQAPENQVNVEKILLDKSNVDEDPITSTIEQSSTSNSSLKKPTNWVDKIGLRINAEIRGIERVPESERHDNSLLSPFLVFLSPNMVISGLSIGSLGPVAYNLDFRTSIIIITIFCIIGSIPVGFFSAFGMRFGIRQQILSRYFTGNIMGRIFALFNVISCIGWNAVNVIPCAQLLNSVGPLPPWAGCLILVGCTCIFAVFGYKTVHLYEKYSWIPNFIVFMIIIAKFSQTHAFNWGEKKSGPTEAGNVLNFISAIFGFTVGWIPSSADYTVYMPANTNPWKVAFAMTTGLSLPAMFTAILGAAIGTSVNLKGSRFEQAYNKNSTGGLIYEILCGDNNNQGYRFIIVVFALGAIANGIPGSYSLSLAIQCIWSQCARVPRIAWCILGNLVALAFSISAYYKFQDTMSNFLSIIAYNVSIYLSISLTEHFIYRRGFSGYDVTDFNNYKTMPVGIAGVVAFCFGICSTVLSMNQTWYQGVIARKIGDNGGDISFEMNIMFAFIGYNLVRPFELKYFGR

SEQ ID NO: 28 - DNA, 1581 bp, Candida albicans
atgaaagagaattacgatttagaacaacaagaaacccaggcaccggaaaaccaagtcaatgtggaaaaaattttgcttgataagagtaatgttgatgaagatcccattacatccaccatcgaacaatcatcaacgagtaattcctcattgaaaaaaccaacaaattgggttgataaaattggacttagaataaatgctgaaatcagaggtattgaaagagttcccgaatctgaacgtcatgacaattcattattatctcctttcttagtgtttttatcaccaaatatggtcattagtggattatcaattggatcattaggacccgtggcatacaatttagatttcagaactagcattataataataaccatattttgtattattggaagcattccagtggggtttttcagtgcctttggaatgaggtttggcattcgtcaacaaattttatcaagatacttcactggtaatattatggggagaatatttgctttgtttaatgtcatttcgtgtattggttggaacgccgttaatgttattccttgtgctcaattattaaactctgtaggtccattacctccttgggctggttgtttgatcttggttggttgtacatgtatctttgctgtgtttggttataaaaccgttcatttgtatgaaaaatactcttggatacctaatttcattgtgtttatgatcattattgctaaattctctcaaactcatgcatttaattggggtgaaaaaaaatctggtccaactgaagctggtaatgtgttgaatttcatatctgctatatttggttttactgtgggatggattcctctgctggcggattataccgtatatatgcctgcaaatactaatccttggaaagttgcctttgccatgaccaccgggttatcactaccagcgatgtttacagcaattttgggagcagcaataggaactagtgtaaatttaaagggatcaagatttgaacaggcatacaataaaaattccaccggaggattgatttatgagatattatgtggtgataataataatcaaggctatcgatttatcattgttgttttcgctctcggtgctatagcaaatggtattcctggatcttattctttgtctcttgccattcaatgtatatggagtcaatgtgctcgagtcccaagaattgcttggtgtattttgggtaatttggtagctttagcattttccatcctggcatattataaatttcaagacaccatgtcgaattttttgtcaattattgcttataatgtatccatttatttgagtatctcgttgacagaacattttatttatcgaagggggttttctgggtatgatgttactgatttcaacaattataaaaccatgcccgttggaatagccggtgttgtggcattctgttttgggatttgttccactgtgctttcaatgaaccaaacatggtatcaaggtgtcattgcaagaaagattggtgataatggtggtgatatatcctttgaaatgaatatcatgtttgcctttattggttacaacttggttagaccatttgaattgaaatactttggaagatga

SEQ ID NO: 29 - protein, 523 aa, Cryptococcus neoformans var. grubii
MSDIEKGFEPVDPKVSDSYEGLPSVDAGVYSEQHNVGESTSRWAKFDELNRKLEHTMGIESRGIERVSESDRTDTRLHGNLFIWASANTVLPTLGVGILGPLLFGLGLGDSMLSILFFNAATACIPAFMSTFGPKLGLRQMTSARYSWGFWGAKIVALLNCIACVGWSIVNTISGAQTLVAVSEYKISAAVGVVIIALVTLFIGLFGYRFVHQYERYSWLPTFITFLVMLGVSAKHLANVPWGVGQAEAAGVLSFGGTIWGFAIGWSSLSSDFNVYMPAEAKSWKVFAWTYTGLIFPLVLVEWLGTAIGCAALVVTDWRDAYHEHELGGLVGAVFIPSMHNGGKFFMTLLVLSVVANNTVNVYSMGLSVSVIANWLAAIPRLVWPCVITAIYIPIAIVGASSFATSLENFLNVLGYWLSIYATVVIEEHFIFRKGRYENYEAASTWNRSDRLPVGFAAIAAGCCGAAGAVLGMAQAWFTGPIGKKVGGTADPSGGDIGWLLAFAFTGVTYPAFRIIEKKWLRR

SEQ ID NO: 30 - DNA, 3144 bp, Cryptococcus neoformans var. grubii
agcccagattcgctcgcatgactgacgcccgaagcctgcttatatgacttaagaccgaagtgccttttattgcagagaatcctttttcaggaacattattcagggtattccattcgtttatcagtacgtaccgcaccatgtgcgccactccatggcctccacttttcgctccacctccgtatacattattgacacttgaaaatttttcctgccgcccgcaaaagaggccaaatttacaacgacactggtgcaacaacagtaataaataaacgatagggggcgttcggcagcgtcggatagtcagaaggaaggggagtgatcgttcggcggatcaccgtttgaattaccagcaactgtttccattgtcaggcactagtttgaatcgttcatttaataattccaatcgctgccgaacgcacctaaaatcctttctaatgccggaaatatacggactttcgtgaacgggagggcaaaacaagaaaaaaaaaagtagcgcaacgacctaaggacctgcgtcaaggtggagatacggtttttgtaactatcttggcttttgatccgatcgtgtcgggactccagttctgatcttattacttatctctatcttcccgcctttatctcgacgtacatttgagagacgttatcggcttcactctagggggcgcagtgatacaatagtacgatctacaggatatcagcttggacacagaaggacagcgcctggctgatataaaaaaggtgacttcaggattctcccgtgtctttccatgcttacagtcgattttttggctgttccatagcttaaatacggcatttcatatatttcgccacttcagacctcaacatgtcagatattgagaagggatttgagcctgttgatcctaaggtctcagactcatacgagggtcttccttccgttgatgcgggagtttactctgaacagcacaacgtcggagaatcgaccagtcgctgggcgaaatttgacgagctgaataggaagctggaacatacgatgggtattgagtcggtgagtatacgggaacaaggccagcgatgcatggtccatggtctgactccatgataagagaggtatcgaacgcgtttctgagtctgatcgtacagatacgcgacttgtgagttacttctttgcgattatttctgaaatccttcttaatcatcaattttcccggtttagcatggaaatcttttcatatgggcaagtgccaacacggtgctgcctacactaggtaaggtcgttcaggttttgatgctccataaggctgattaggattgggcaggcgtcggtatccttggaccactcctttttggccttggtctgggagattcgatgttgtcaatccttttcttcaacgctgctaccgcctgcatcccggccttcatgtctactttcggacccaaacttggccttcgtcaaatgacttccgcgaggtactcctggggtttttggggggcgaagatagtagccttactcaactgcatcgcgtgtgttggctggtccatcgtaaacaccatatctggcgctcaaacccttgtggcggtctccgaatacaagatctccgctgctgtgggcgtcgtaattattgccctcgtcactcttttcattggcctctttggctatcgatttgtccaccaatatgagagatactcttggttacctactttcatcacattccttgtcatgcttggtgtgtctgcgaagcatctggcaaatgttccttggggtgttggtcaggcagaagccgccggagtgttatccttcggtggtaccatatggggttttgcgataggttggtcttccctctcaagcgatttcaatgtctatatgcctgccgaagccaagagttggaaggttttcgcctggacgtatacaggattgatcttcccacttgtgctggtcgaatggctcggtactgctataggctgcgctgctttggtcgtcaccgactggagagacgcctatcacgagcatgaacttggaggtcttgttggtgccgtattcagtaagtgacatgcctaagacgataatgcttcgctgactgcatagtagtcccttctatgcacaacggagggaagtttttcatgactcttctagtgctctctgttgtcgccaataagtatgtattatctttttttatacattctttccacttaagctgaccgtacaatgaaagtaccgtgaatgtgtactctatggggttgagtgtatctgtgattgccaactggttggctgctattcctcgacttgtgtggccctgcgtcatcactgccatttatatcccaatagctatcgtgggcgcaagctcatttgctacctctctcgaaaacttcctcaacgtcctcgggtactggctttctatctatgctactgtggtaattgaagaacatttcatcttccgcaaagggcggtacgaaaattacgaagcggccagcacttggaaccgatctgacagattgcctgtgggatttgctgctatcgctgccgggtgctgtggagccgctggtgcagttctaggcatggctcaggcgtggttcaccggtcccagtaagttcttgcatatcgttaggaatttggctaacaccgtgtatcccacagttggtaagaaggtcggcggcacggctgacccgtccggtggcgatattggttggctcttggcatttgtgagtcacaagctatctggacgaactgatatccctcgatgcttatatgttcttataggccttcaccggtgttacatatcctgcattccgaataatcgaaaagaaatggctccgccgataaggtctgccagctatagatttgaatttccatacttttgttttagattatttcaatcatttagatcggcgttacttatcctgcattccgaacactaaaatagaaatggcttcctgccgattatagactttagttttggtagttttgttttggatttgttttagcatcattttagattagttgttgaagggtgagatgcctgcctctaaaggcctaacaacttcacgcatattcagggaccggggagtatagggtggggatgtcgaagcataaatctggtatgcattgacataagctcctcacttatgatccgctcaaacacggtgaccaatattattgtaatttgc

SEQ ID NO: 31 - protein, 243 aa, Penicillium chrysogenum
MSEATPSPAQGVGPIYRPDGEKPTATVSKDIFYENVHVLPQSPQLIALLTMIRDRRTSRADFIFYSNRIIRLLVEEGLNHLPVVEQSVTTPVGRVYLGVRFEGKICGVSIMRAGEAMEQGLRDCCRSVRIGKILIQRDEETCKPKLFYEKLPGDIANRWVLLLDPMFATGGSATLAVEVLKAKGVPEDRILFLNLIASPSGVADFAERFPKLRVVTAFIDQGLDDKKYIIPGLGDFGDRYYTL

SEQ ID NO: 32 - DNA, 1108 bp, Penicillium chrysogenum
aattccttagctttaaatgcctgcgttacttcctacacttcaactcaaccccactacctttggaacggtttacagttcgctgcagatatccttcatttcttgtttcttcacctcttagtcgtactttgcatccgtggaagaaggagggggaaagctaccatagccaagacgaacgagatgtcagaagccactccctcccccgcacaaggcgttggccccatctacagacccgatggcgaaaagcccacagcgacagtatcaaaggacatattttacgagaatgtccacgttttacctcagtcccctcagctgatcgctcttttgacgtaagatgaaggccgagtcaaatcatacaggttaacactagtcagtatcatatgctgacaccattcgtagcatgatcagagatagaaggactagccgtgccgatttcatcttctactcgaacaggatcatacgcctcctcgtagaggaggggctcaatcacctgccagtggttgaacaatcggtcacgacacctgtaggtcgcgtctatctcggagtgcggttcgaagggaaaatatgtggtgtttccatcatgagagcaggagaggcaatggagcagggtttgagagattgttgtcgttctgtgcgaattggtaaaatccttatacagagggacgaggaaacctgcaagccaaaactgttctatgaaaagctccctggcgacatcgcaaatcgatgggttcttcttcttgatcctatgtttgcaacaggcaagttcgtcgaaccaacgcactaaacgaactaacatcaactagggggctcggcaacactcgctgttgaggtcttgaaagcaaagggggtccctgaggaccgcattctgtttcttaaccttatcgcaagcccttcaggtgttgcagatttcgcagaacgctttcccaaactcagggtagtgactgctttcatagatcaaggcctagatgacaaaaagtgggtatctcacgttattaaaaaggaaaagataaaatgattacatgtacattgctgaccaaggccattctagatacataataccagggcttggtgacttcggcgaccgctattacacgctgtagccataccaatg

SEQ ID NO: 33 - protein, 246 aa, Penicillium chrysogenum
MASKPESGPSPAQGVGPLYRPDGEKPTATVSKEVSYDNVHVLPQTPQLIALLTMIRDKRTSRADFIFYSNRIIRLLVEEGLNHLPVVEQSITTPVGRSYLGVKFEGKICGVSIMRAGEAMEQGLRDCCRSVRIGKILIQRDEESCMPKLFYDKLPSDIADRWVLLLDPMFATGGSATLAVETLIERGVPEHRILFLNLIASPSGVAEFAERFPKLRVVTSFIDQGLDEKKYIIPGLGDFGDRYYSM

SEQ ID NO: 34 - DNA, 1319 bp, Penicillium chrysogenum
gtgaacttccggcgcttccatattctaacatctctttttctctctttctctagtcctacgacccacacacaggcatctttgggcgcagcaaaatcatggctagcaaaccagagagcggtccctctcctgcgcagggcgttggtcctctgtatcggcctgatggcgagaagcccactgcgacagtttcaaaggaagtttcctacgacaatgtccacgtgctacctcaaacaccccagttgattgctctcttgacgtgaggacccattctctgcatggaaggtcaccctagactcatattttgtaccacaaataggatgatcagagacaagagaaccagccgtgctgatttcatcttctattccaaccgcatcattcggttgctggtggaggaaggcctgaaccacctccccgttgttgagcagtcaatcactacccccgttggacggtcctaccttggtgtcaaatttgaaggaaagatctgcggtgtgtctatcatgcgcgcaggcgaggccatggaacaaggattgcgcgactgctgccgatctgttcgcattggaaaaattctcatccagcgggacgaggagagctgtatgcccaaactgttttacgataagcttcctagtgacattgctgatcgatgggtccttctcctcgaccccatgtttgcaactggtgagttgaacttgttctacaatgcgtccgacaaccaagctgattttttttttccaggtggttctgcaactctggcagtggagacactgatcgagagaggtgttcctgaacacaggatcctattcctcaaccttattgccagcccgtctggtgttgctgagtttgcggagagattccctaaactccgggtcgtgacctctttcattgatcaaggattggatgagaagaagtgagaatattgcactctttggatgttgtgattgccatgctgactggttgataggtatattatccccggcctgggagatttcggcgacagatattactctatgtaaaagcgctgtttcttaccacgttgaaaggagcatcagtggttttaaaggtcacctggaaaatctgagtaattcagtattccattatccatccattgcttgatctgttccagatgccataaggcacttccacttcccatacaagtcacctcttcagagttggcttgatggccggtagtggggtaaaggtctagatgggtgagacactcccaaggttcttggggtttaggatatatgataagcatgcgcaacgagaatatcaaataccatgtacatacaagaatctcacaactcaattgtttttgatatcgtgcc

SEQ ID NO: 35 - protein, 241 aa, Aspergillus oryzae
MADERLEAIPSPAQGVGPIYRPDGEKPTATVSKDIPYENVHVLPQTPHMIRDKRTGRADFIFYSNRIIRLLVEEGLNHLPVVEQAVTTPVGRTYLGVKFEGKICGVSIMRAGEAMEQGLRDCCRSVRIGKILIQRDEETCKPKLFYEKLPADISSRWVLLLDPMFATGGSATLAVEILKAKGVPEDRILFLNLIASPSGVADFAERFPKLRVVTAFIDQGLDEKKYIIPGLGDFGDRYYTL

SEQ ID NO: 36 - DNA, 926 bp, Aspergillus oryzae
atggctgatgaacgattggaagcgatcccttctcctgcccaaggagttggcccaatttacagacccgatggcgagaagccaacagcgacagtgtcgaaagacataccttatgaaaatgtccatgtcttgcctcaaacaccccagttgatcgctcttctaacgcaagtcatacgtctctaaagaattttcaagtcaacatcgggtgcccctactgacaactccatagtatgatcagagataagagaactggacgtgccgatttcatattctattccaacagaatcatccgtttattggtggaagagggtttaaaccaccttccagtggttgagcaagcggtgacaactcctgtaggccgtacctatctcggtgtcaagttcgaaggtaaaatatgcggcgtatccatcatgagagcaggcgaggcgatggagcagggcttgagagattgctgtcgatcagttcgcattgggaaaatacttatacagagagatgaggaaacatgcaagccgaagcttttctacgagaagctccctgctgatatctccagtcgatgggttttactccttgatccaatgttcgcaactggtgggttatataaccttcttacactatagagtgatattgattgctatcgatccagggggttcggcgacgctcgctgtcgaaattctgaaggcgaagggtgtaccagaggaccgtattctattcctcaaccttatcgcaagcccttcaggagttgcagactttgcggaacgcttccctaagctcagggtcgtaactgcttttattgaccaaggcttggatgagaaaaagttgatctacttctatttctccctcttcttgattagcccgctaactggttgctatcacacaggtacattatccctggacttggtgattttggtgatcgatactatacgctgtga

SEQ ID NO: 37 - protein, 246 aa, Trichodema reesei
MAEKPTPAAAPTQCVGPTFRSVADKPTATVSHEVPFENVCVLPQTPQLIALLSMIRNKDTERADFIFYSNRIIRLLVEEGLNHLPVIEKTITTPVGRTYNGLGFQGKICGVSIMRAGEAMEQGLRDCCRSVRIGKILIQRDEDTAQPKLFYDKLPEDIAKRWVLLLDPMFATGGSATMAVEVLKSRGVPEDRILFLNLIASPEGITTFATKFPRLKVVTAFIDEGLDEKNYIVPGLGDFGDRFYTI

SEQ ID NO: 38 - DNA, 1177 bp, Trichodema reesei
agattatatgtacctgtctgggtagctttcaagcaacttgcagctatctcgacctttttttcttctcttctttctcaaagtacaatcacgctcaattccatggccgagaaaccgactcctgctgctgcgcccacgcagtgcgtcggcccgacgtttagatcggtcgccgacaagccgactgcgacggtgtctcatgaagtacccttcgagaacgtctgcgtcctgccgcagactccccagttgattgctcttctctcgtaggtctaaagacggccccttcaaccggcctccagcgttttggcctctgtgctaagcgtcttctgtatgcagcatgataaggaacaaggatacggagcgcgcagacttcatcttttattccaacagaatcatccgactgcttgtggaggagggcctcaaccaccttcccgtcattgaaaagaccatcacgacgcccgtcggccggacttacaacggcctggggttccagggcaagatatgcggcgtgtccattatgcgagcgggagaggcaatggagcagggtctacgagactgctgtcggtccgtaagaattggaaagatcttgattcaacgagacgaggacacggcgcagcccaaattattctacgacaagcttcctgaggatattgcaaagcgatgggttctcctgctggacccgatgtttgcaacaggtagctattcttcccactcatcgtcctcctccttctcctccgtctgcccttgtggtgttcgccacaaccaaatattacccgtgcctgatgtccccccatgactgacaatcgcgaaaaaaaaaaaggtggttccgctacaatggccgtcgaggtcctcaagagtcgaggagtccctgaagaccgcatcctcttcctcaacttgattgccagtccagagggaataacaacgtttgccaccaagtttcctcgcctcaaagttgtgacggcgttcattgacgaggtaagtgctgatctggatcgccattgtccaacggcctgtactaatggaattccgtacgcagggccttgatgagaagaagtattatgaccccgtgagctccacaaagcgttggctgacccctgttgcagttacattgttcctggcctgggagactttggcgaccgattctatacaatttgaagacggcctttttttcccgatgagatcc

SEQ ID NO: 39 - protein, 242 aa, Acremonium chrysogenum
MAPGNAPTQTVGPVFKSLDEKPTATVSSEVGYDNVRVLPQTPQLIALLSMIRNKDTERADFIFYSNRIIRLLVEEGLNHLPVIEHDVVTPVGRTYNGLMFQGKICGVSIMRAGEAMEQGLRDCCRSVRIGKILIQRDDETAQPKLFYDKLPEDIAQRWVLLLDPMFATGGSAIMAVEVLKSRGVPEDHILFLNLIASPEGVKNFATKFPRLKVVTAFIDEGLDEKNYIVPGLGDFGDRFYTI

SEQ ID NO: 40 - DNA, 973 bp, Acremonium chrysogenum
atggctccaggcaatgccccaacgcagacagtcggccccgttttcaagtcgctggatgagaagcctactgccactgtttcatccgaggttggatacgacaatgtccgcgtgctcccccagacgccgcagctgattgcattgttgtcgtacgttgccaccactttccagtccttagaatgtgtactcagaatgtttagcatgatccggaataaggatacggagagggcggatttcatcttctactcgaaccggattattcgtctcctcgtagaagaaggcctgaaccacctgcccgtcatcgaacatgacgttgtcaccccagtcggccgcacatacaacggcctgatgttccaggggaagatctgtggagtctctatcatgagggcaggtgaagccatggagcagggtctccgcgactgctgccggtccgtaaggattggcaagatcttgatccagcgggacgacgagacggcgcagccaaagcttttctacgataagcttcccgaggacattgcgcagcgatgggttctcctcctggaccctatgttcgccaccggtgagagccccctcccgctccccgctccccaacaatcggttcgttgaagtcgtggtaactgacgggaagaccaaaaacaggaggctcagccatcatggccgtggaggtcctcaaatcgaggggcgtccctgaggaccatatcctgtttttgaacctgattgccagccctgaaggagtcaagaacttcgccaccaaattcccacgacttaaggtggtcactgctttcattgacgaggtgagtccaccccctcctatcccacatattcccttgtaacaatgtgctgacatggacatgcagggcctggacgagaagaagtattggtcccaccacccaactcgtaggaatcgactaacatcgtgaccagttatatcgtacccggtctgggagacttcggagacaggttctacaccatctga

SEQ ID NO: 41 - protein, 350 aa, Fusarium oxysporum
MADKTQDTHTTPVGPSYRTHQQKPSATVSVDVKLDNVHVLSQTPQLIALLSKIRSKETERADFIFYSNRIIRLLVEEGLNHLPVIEHTVTTPIGRNYNGLMFQGKICGVSIMRAGEAMEQGLRDCCRSVRIGKILIQRDEETAQPKLFYDKLPEDIADRWVLLLDPMFATGGSATMAVQVLKARGVPEEHILFLNLIASPEGVKNFSAKFPRLRVVTAFIDEGLDEKKYGCSTRMTHDVLTNQQLYRSWPGRLWRQILHHLRKESAQRLYIPSRRRIEKGRRVMGVTSLGIDTRRIKETINSHPLRSARRRLHRPPFGYLVQLLLEMCQCSQAPRCPPWRRHLQVVRDCR

SEQ ID NO: 42 - DNA, 1982 bp, Fusarium oxysporum
tgctgttttgttaaatgacttctaatgcgccatttccattgttctcatcccatctccgttctctataccactcgatatccattctcatctccactgtttattatcatgaatccacgcccacgcccatgccactgccatgcagtgcggtctcctccgattttgtgagtatcgtagggtaagggtccaggcgccgccggccgatttgcatccccgttttaagcatatgcgagaccaatacgattgagcatagcaagcgggcaatgcaatgcaatgcagggctaccgacttgtcttatcctcgagagctctagttgatgccgagctcttctctccgcctttcaattcattcaatcctatactgaacctttcttattttgttcttcaacctctttctaggttatacctacgactgtactccgctcgcccacttggaaatatctgaactttacttcaaccccgccatcaccgctgacttcttacaccacctcgaacactaacaacagctaacatggcggacaagactcaagatactcacacgacacccgtcggtccctcataccgcactcaccagcagaagcccagtgcaaccgtctctgtcgacgtcaagctagacaatgtccatgttctgtcgcagacccctcagctcattgccctactgtcgtatgtccacttccctaattgggcgtcccaagcctcggagctgagaattgtccaggaaaattcgtagcaaggagaccgagagagcagatttcatcttttactccaaccgaatcattcgattgctagttgaggagggtctcaaccacttgcctgtcattgagcacactgtcactacacccattggccgaaattacaatggtctcatgttccagggcaagatctgcggagtgtccatcatgagagctggtgaggctatggaacagggtctccgtgactgctgccgctctgttcgtattggaaagatcttgatccagcgtgatgaggagacggcccagcccaagctgttctacgataagctgcccgaggatattgctgaccgatgggtgcttcttctggatcccatgtttgctactggtacgacgatatcccatttcacatgtcattcatgtcgcaatccgtctaacgcagatatacaggtggctctgctaccatggcggttcaagttctcaaggccaggggcgttcccgaagaacacattctcttcctcaacttgattgcaagccccgagggtgtcaagaacttctctgccaagttcccccgcttgagggtcgtgacggctttcatcgatgaggtttgtcacaccgcctgcacacactgtcgcaacgtattggctaaccaggcatttctagggtctcgacgagaagaagtatggatgctcaacccgaatgacacatgatgtgctgaccaaccaacagttatatcgttcctggcctgggagactttggagacagattttacaccatctgaggaaggaatcagcgcaaaggttatatatcccgagtcggaggcgcatagaaaagggtcgtcgagtcatgggtgtaacaagtcttggtattgatacaaggcgcataaaagaaacgattaatagtcatcctctccgtagtgctcgtcgtcgtcttcatcgaccgccgttcggttacctcgtccagctcctgctcgagatgtgccagtgtagccaggctccgcgttgtcctccttggcgacggcatctacaagttgttagagattgtcgatagagtcgtagaagggatattgtagactgacctgctgcgtcaacccattcaccgtaaacgtcaaccgccgcagacagatctgtcgcaaagttagcacccgatgcgccggaggcaaaggagagtccgtggaagccgtacagttgaccgcgcattggaacttttgtccgcagacacggcagtcgagttgtccgacaccagccttcttgtcgagtttgactgaaaccgagttttcgtggttacagaagaggca

SEQ ID NO: 43 - protein, 231 aa, Ustilago maydis
MASSALQQNGAAAALQGPTLPPNASRLPQTAQLDALLTIIRDASTPRSDFIFYSDRIIRLLVEEGLNHLPTLPQTVMTPTGFEYSGVSFQGRICGVSILRAGEAMEAGLRECCRSVRIGKILIQRDEETAKPKLFYAKLPEDIAERWVLLLDPMLATGGSAIKAIEVLIENGVKAERILFLNLIASPEGLNNMYSKFPQVKVISAWVDERLDEKSYIIPGLGDYGDRYYSG

SEQ ID NO: 44 - DNA, 696 bp, Ustilago maydis
atggcatcgtcggcactacagcagaacggcgctgccgctgcgctacaggggccgacgttgcctccaaatgcatctcgacttccacagacggcgcagctggatgcgctgctgaccatcatacgcgacgcctcgacacctcgctccgacttcatcttttattcggaccgcatcatccgacttttggtcgaagaaggtctcaaccacctgcctacgctaccgcagacagtcatgacgccgaccggattcgaatactcgggcgtctcgttccaaggtcgaatttgcggagtctcgattcttcgcgctggcgaggcaatggaagcgggcctacgcgagtgctgccgcagcgtacgaatcggcaagattctgattcaacgagacgaggagacggccaagcccaagctgttctatgccaagctgccggaagacattgcggaacgatgggtattgctactggatcccatgcttgccactggtggaagcgccatcaaggctatcgaagttctgatcgagaacggcgtaaaggcggagcgcatcctctttctgaatctcattgcaagccccgagggtctgaacaacatgtactcaaaattcccacaggtcaaagtgatctcggcttgggtggatgagaggttggacgaaaagtcttacatcatccctggtctgggtgactacggcgaccgatactacagcggatga

SEQ ID NO: 45 - protein, 216 aa, Komagataella phaffii
MSATNYPNVFMLRQTNQLRGLYTIIRDKNTKRGDFVFYSDRIMRLLVEEGLNQLPVKPTTVKTSQGHEVEGFSFEGKICGVSIIRAGESMEQGLRDCCRSVRIGKILIQRDEETAQPKLFYSKLPDDISERYVFLLDPMLATGGSAMMAVDVLLKKGVKQERILFLNLLAAPEGIEAFYKKYPNVKIITGVIDQGLDENKFVVPGLGDFGDRYYCI

SEQ ID NO: 46 - DNA, 651 bp, Komagataella phaffii
atgtcagcaacaaactatccaaatgtatttatgctacggcagaccaatcagctgcggggattgtacaccattattcgtgataaaaatacaaagcgtggagatttcgtcttttatagtgacagaatcatgaggttactggtggaagaaggtttgaaccaattgccagtgaaaccaacaacagttaagacttcacagggtcatgaggtcgagggtttctcctttgagggcaaaatttgtggtgtttcgattataagagctggtgaatcaatggaacaagggttaagggattgctgtagatcggtgagaattggtaagatattgattcaaagagatgaagagacagctcaacctaagctgttttattccaaacttcctgacgatattagtgaacgttatgttttcttactagacccaatgctggctactggaggttctgccatgatggctgtggatgttcttttgaaaaaaggtgtcaagcaagaaagaatcttgtttttgaacttgctggctgcccctgagggcattgaggccttttataagaagtatccaaacgtcaagatcataactggagtaattgaccaaggtctggatgagaacaagtttgtcgtccctggtttgggtgactttggtgataggtattattgtatttag

SEQ ID NO: 47 - protein, 230 aa, Cryptococcus neoformans var. grubii
MSNITTCLPASGSNIHKAELPGNAFVLPPTSQLQSLLTIIRDETTQRGDFVFTSDRIIRLLVEEGLNHLPVLPKKVVTPVGREFEGVAFQGRICGVSIMRAGEAMEAGLRDCCRSVRIGKILIQRDEETALPKLFYAKLPDDIAQRYILLLDPMLATGGSCIKAIEVLLDHGVQEEKILFLNLIASPEGINKVCTRFPKLTIITAWVDEGLDDHSYIVPGLGDFGDRYFL

SEQ ID NO: 48 - DNA, 1320 bp, Cryptococcus neoformans var. grubii
gattctccaaaaggattttcggattctgtcagagcagcttatccaaccgaagcttctccccccttcatccgcctctatcctcgctttccgctagcctcgctgcccttctccagttttgctgtttgcaatatgtccaacatcacgacttgtctccctgcatctggaagcaatatccacaaggccgagctccccggtaacgcctttgtcctcccccccacctctcaacttcagtcgctcctcactattattcgcgatgagactacccaaaggtaagctccgctccgtactatcatactcttttcgatttggaaatcataataatggtatcataggggtgactttgtcttgtgagtgacgcgtgtatcttcaatagcaattattagttgggatttacttacccttcctgggatagtacttcagataggattatcagattgcttgtagaggaaggtgagttttcgaatgacatctttcaaaaagcttgtccattccagagagttgctttacaatatcaaagacggggagcgacgatagaaattcaagcactaatctggtttggcaggtctcaaccaccttcccgtgctccctaaaaaagttgtcactcctgttggccgtgaatttgaaggtgtggctttccaagggaggatttgcggagtgtcgatcatgcgaggtaagtttcaaaaccaatgcggcggtagcctggagatctgatctcatgcacatgtgccgagccaatcactgagtttaagtgctgatatcgacctgttatagctggagaggtaagcgtccatgatatctcccccaatgatcactgttagagggctaaaatagtttatgttaggcaatggaagcgggtcttcgtgattgctgtcgttccggtgagtgtttaatggtttccgcactatgtttacaatggtgcattacatcatactaatgcacagtattgcagtgcgaattgggaaggttagtcagcaaaggtctttcgcaaccatttgacattttggtgctgaccgcatgggtacacacccacctttcagattcttatacaaagggttcgttgctcttcatcttttttttttttttaccctttcaatgatgtttaccacaatattcgttgcaggatgaggagacagctctacccaagcttttcgtacgtgactcattgtttcataagcgcttggaaaatgagctaacagacatgtgcagtatgctaagttgcccgatgacattgctcaacggtatatccttcttctagaccctatgcttggtaagataatctacatatcatactgtgaaagttcgcagctaagcatggcgatc

SEQ ID NO: 49 - protein, 218 aa, Candida albicans
MSVAKAVSKNVILLPQTNQLIGLYSIIRDQRTKRGDFVFYSDRIIRLLVEEGLNQLPVEEAIIKCHGGYEYKGAKFLGKICGVSIVRAGESMEMGLRDCCRSVRIGKILIQRDEETALPKLFYEKLPEDISERYVFLLDPMLATGGSAMMAVEVLLARGVKMDRILFLNLLAAPEGIKAFQDKYPDVKIITGGIDEKLDENKYIVPGLGDFGDRYYCI

SEQ ID NO: 50 - DNA, 657 bp, Candida albicans
atgtctgttgccaaagctgtgagcaaaaacgttattttattaccgcaaaccaaccaattaattggtttatactcaatcattcgtgatcaacgtactaaacgtggagattttgtattttattcagatagaatcattcgtttattagttgaagaaggtttgaaccaattaccagttgaagaagcaattataaaatgccatggtggatatgaatacaagggagccaaatttttaggtaaaatttgtggtgtatctattgttcgagctggggaatcaatggaaatgggattaagggattgttgtcgttctgtaagaattgggaaaatcttgattcaaagagatgaagaaactgcattaccaaaattgttttatgaaaaattacctgaagatatcagtgaacgttatgtatttttattagatccaatgttggccacaggaggatcagcaatgatggctgttgaagttttattggcaagaggagtgaaaatggacagaattttatttttgaatttattagcagcaccagaaggtattaaagcattccaggataaatacccagatgtcaaaataatcactggtggaattgacgaaaaattagatgaaaataaatacattgttccaggtctaggtgatttcggtgatagatattactgtatttaa

SEQ ID NO: 51 - protein, 216 aa, Saccharomyces cerevisiae
MSSEPFKNVYLLPQTNQLLGLYTIIRNKNTTRPDFIFYSDRIIRLLVEEGLNHLPVQKQIVETDTNENFEGVSFMGKICGVSIVRAGESMEQGLRDCCRSVRIGKILIQRDEETALPKLFYEKLPEDISERYVFLLDPMLATGGSAIMATEVLIKRGVKPERIYFLNLICSKEGIEKYHAAFPEVRIVTGALDRGLDENKYLVPGLGDFGDRYYCV

SEQ ID NO: 52 - DNA, 651 bp, Saccharomyces cerevisiae
atgtcttcggaaccatttaagaacgtctacttgctacctcaaacaaaccaattgctgggtttgtacaccatcatcagaaataagaatacaactagacctgatttcattttctactccgatagaatcatcagattgttggttgaagaaggtttgaaccatctacctgtgcaaaagcaaattgtggaaactgacaccaacgaaaacttcgaaggtgtctcattcatgggtaaaatctgtggtgtttccattgtcagagctggtgaatcgatggagcaaggattaagagactgttgtaggtctgtgcgtatcggtaaaattttaattcaaagggacgaggagactgctttaccaaagttattctacgaaaaattaccagaggatatatctgaaaggtatgtcttcctattagacccaatgctggccaccggtggtagtgctatcatggctacagaagtcttgattaagagaggtgttaagccagagagaatttacttcttaaacctaatctgtagtaaggaagggattgaaaaataccatgccgccttcccagaggtcagaattgttactggtgccctcgacagaggtctagatgaaaacaagtatctagttccagggttgggtgactttggtgacagatactactgtgtttaa

SEQ ID NO: 53 - protein, 214 aa, Rhizopus delemar
MTDSYTLPEQVKLLTQNSQLKGLMTIIRDKDTPRADFIFYADRIIRLLVEEDKSITTPTGNDYKGIAFEGRICGVSIMRAGEAMEQGLRECCRSVRIGKILIQRDEETHEPKLYYSKLPKDIASRYVFLLDPMLATGGSAMQAVQVLLDNNVKEEHIIFLNLIGSPEGIDSFIAKYPKVKIVIGELDAGLNKDKYIVPGCGDFGCSDCHLPLFW

SEQ ID NO: 54 - DNA, 1081 bp, Rhizopus delemar
atgactgacagctacacgctaccagaacaagttaaacttcttacgcaaaactcccaattgaagggattgatgacgattattcgtgataaagatacgcctcgtgctgactttatcttctacgctgatcgtatcattcgtctccttgttgaagaaggttcttactctcttttctcttctttacagggctaacatcaataatttaccctatctaggattaaatcatttacctgttatagacaaatccatcacaacaccaacgggtaatgactacaaaggtatcgcctttgaaggtcgcatctgtggtgtctctattatgcgagctggtgaagctatggagcaaggcttgcgagaatgttgcaggtttgctaaaaataaaaccacattcacatctgtttctcaatttcatttttttctttttatagaagtgtcagaattggcaagattttgattcaacgtgatgaagaaacccacgaacccaaagtaatgaacaaatctgctctataaaaaagtactcactatactttttttttctttttttttttttttttttttttttttttttttactttagctttactattctaaacttcctaaggatatcgcttctcgatatgtgttcttattagatccaatgctcggtataaaaatattcatgttttgtaacatcaccataagctcaccgtattctttttttggatagctactggtgggtctgcaatgcaggctgttcaagttttattagataacaatgtgaaagaagaacatattattttcttaaacttgattggctcccctgaaggaatcgattcctttattgccaaatacccaaaggtcaaaattgttattggcgaattagacgctggtctcaacaaggataaatatattgttcccggttgtggcgattttggttgcaggtaaatataagttattacatttttcttttatctaaattttgagtacagatactttggtacagattaataaaaatacttgttattttccatttttgtgtatattgcataatttgcgcaacgttgtactttcctttcttagcgactgtcatcttcctcttttttggtaa

SEQ ID NO: 55 - protein, 609 aa, Aspergillus oryzae
MVDEKKPVDNDTMPTMEEATPQHHGAVPNAQRDIEEDITYTKDVHSEKRELPESDSLNSCGQKKDEKDEEAGENVPSRSWRRYRPWLKHVVYAVIWLLFTGWWIAGLILHRYDLGWLIPFLIYLAITLRLIFLYVPISIVTRPVYWVWNQTASRFVSLIPEKLRIPSGALLTIAVIIVGSFASPESADNTRANRAVSLFGLVVFLFALWLTSRNRKKIIWHTVIVGMLVQFIVALFVLRTKAGYDIFNFISTLARELLEFSKQGVDFLVETGWANKHSSWFLVSVVPAIIFFVSIVQLLYYTGVLQWAIGKFAVFFFWAMRISGAEAVVAAASPFIGQGESAMLIKPFVPYLTMAEIHQIMCSGFATIAGSVLVSYIGMGLNPQALVSSCVMSIPASLAASKLRWPEEEETLTAGRIVVPEDDSHKAANALHAFSNGAWMGIKIAGMIATTLLCIISLVGLVNGLLTWWGHYLNIYEPDLTIELIVGYICYPIAFLLGVSRDGDLLKVAKLIGTKLVMNEFIAYDYLQNKEEFQSLSPRSRLIATYALCGFANIGSLGNQIGVLAQLAPSRAGDVSRVAVSAMLTGAISTLTSAAIAGLLITNEKQYIS

SEQ ID NO: 56 - DNA, 1974 bp, Aspergillus oryzae
atggtggatgagaagaaaccagtggacaacgacactatgcctacgatggaggaagctacgccacagcatcatggcgcagtccccaatgcgcaacgggatattgaggaagatatcacttacacgaaagatgtgcactccgagaagagagaactaccagagtcggactctctcaatagttgtggccagaagaaggacgagaaagacgaggaggcaggtgaaaacgtcccttctcggtcgtggcgccgctatcgcccgtggttgaagcatgttgtctacgccgtcatctggcttttgttcaccgggtgagttgtgccttcttcgagctgtcggggacacctgccagtgaggtagctgtgatcttttaaaagctcattatgtcacttatagctggtggattgccggtctcatcctccaccggtatgatctgggctggttgatcccattcttgatctacctcgcgattactttgcgcctcatctttctctatgttcctatttcgattgtgacgagaccggtctactgggtatggaaccagaccgcttcacgattcgtcagcctgattccagaaaaactgagaatccccagcggcgcgctcctgacgattgcagtgattatcgtcggctcattcgcctcaccggagtctgcagacaatacccgcgcaaaccgggccgtcagtctctttggactagttgttttcctctttgctttgtggcttacctcaagaaatcggaagaagattatttggcataccgtcatcgtgggaatgcttgtgcaatttatcgttgcactcttcgtcctaagaaccaaggctgggtatgatatctttaacttcatttccacgttggccagagaactcttggaattctccaagcaaggtgtggacttcttggtggaaactggatgggccaataaacacagcagctggttcctcgttagtgttgtcccggctatcatctttttcgtgtctatcgtgcagttgctctactacactggcgttctccagtgggcaattggcaagtttgccgtattctttttctgggctatgcgtatctcgggagctgaggctgtagtagcagctgcatctccctttatcggacaaggcgagtcagcgatgctcatcaagcctttcgttccctatcttacgatggcggaaatccaccaaatcatgtgctccggattcgctactattgcaggatcagtcctggtgtcctacattggaatgggtttgaacccccaagcgctggtgtcctcatgtgtgatgagtattccggcatccctggcagcatctaagctgcgttggcccgaagaagaggaaaccctgacagctggccgtattgtcgtccccgaagacgatagccacaaagcagctaacgccctccacgcattctccaacggagcttggatgggtattaagattgcaggcatgattgccactacgcttttgtgtattatctccctcgtaggtctcgttaacggcctgctgacctggtggggtcactacctgaacatctacgaaccggacttgaccattgaactcattgttggctacatctgctacccgattgccttccttcttggtgtctcccgcgacggtgatctcctgaaggtcgctaaactcatcggaaccaagcttgttatggtaagtcccattggctttgactcaagagaacaacagacatgctaacagactccaaacagaatgaattcatcgcgtacgactacctgcagaacaaagaagagttccaaagcctgtctccccgctcccgactgatcgccacttatgccctgtgtggtttcgccaacatcggttctctcggaaatcaaatcggtgtgctggcccagttagcccccagccgagctggtgatgtttctcgtgttgccgtcagtgccatgctcacgggtgccatcagtactctgacaagtgcggccattgcgggtctcctaatcacaaatgaaaagcaatacatctcataa

SEQ ID NO: 57 - protein, 626 aa, Penicillium chrysogenum
MDKTQEADPTGSHVVHHADPVLDPENQHHHPHQHHTAYATEGRQDEVVYSKDAEFEKGIVPEQTPMDNGSKNSNDEEAGENLPAKRTWYRRVQKQQRHIVHAVVWLVFTGWWIAGLILHRHDIGWLIPFLLYLAITLRILFFYVPISIVSKPMHWVWDHTALPFVRLIPEKLRTASAAALCIGVILIGSFVSEESMDNTRANRAVSLFGMVVFIFGLWLTSRNRKKIVWHTVIVGMLAQFIVALFVLRTTAGYDIFNFISTLARELLGFASEGVIFLTSEDFYAYNTAFVPSSFFIVNVVSAIIFFVSFVQLLYYYNVLQWFIGKFAVFFFWSMRVSGAEAVVAAASPFIGQGESAMLIRPFIAHLTTAEVHQIMCSGFATIAGSVLIAYISIGVSAQALVSACVMSIPASLAVSKIRWPEEEETLTAGRVVIPDDEEHRAANALHAFTGGTWLGLKIAAMMSSILLCIIALVGLINGLLTWWGRYLSINDPPLTLELIVGYICYPIAFLLGVSRDGDLLKVGQLIGLKLVTNEFVAYDRLQHWDDYKDLSDRSRLIVTYALCGFANIGSLGNQIGVLSQIAPGRASDVSRVAFSAMVTGAISTFTSAAVAGLLITNEKQYFGSSS

SEQ ID NO: 58 - DNA, 2169 bp, Penicillium chrysogenum
atggataagacccaggaagctgatcctacgggatcgcatgttgtgcatcatgcagatccggtgctggatccagagaatcaacatcaccatccccatcaacaccatacagcctatgccacagagggacgtcaagatgaagtggtctactctaaagatgccgaattcgagaaaggaattgtgcctgaacaaacaccgatggacaacggcagcaaaaactccaacgacgaagaagccggagagaacttaccagccaagcgcacctggtatcgccgcgtgcaaaagcaacagaggcatatagtccatgctgtggtctggcttgtgtttaccgggtgagtgaactactttgattctactgggattttacagtggtactgtacaccatcgactcaactcggggcttacacatactcttcacctagctggtggattgctggcctgatactacaccgacatgatataggatggctcatcccattcctgctctatcttgccatcacgctgcggatactattcttctatgttcccattagtatcgtgtcaaagcccatgcactgggtttgggatcatactgcgctcccgttcgtgcgcctgattcccgagaagctgagaacagccagtgccgccgcgctatgtatcggtgttattcttattggctcgttcgtgtccgaagaatcaatggataacacacgtgccaaccgtgcagtcagtctgttcggaatggtagtcttcatcttcgggctatggctcacatctcgaaaccgcaagaagatcgtctggcacactgtcattgtcggcatgctggcgcaattcatcgttgctctgtttgtgctgcgcactactgcaggatacgacatcttcaacttcatctcaacgctagccagagaactgcttggttttgcttccgaaggagtcatcttcctgaccagcgaggatttctacgcctataatacagcctttgtcccatcgagtttcttcatcgtcaacgtcgtctccgccatcatcttcttcgtctccttcgtccaactattgtactactacaatgtcctgcagtggttcattggtaaattcgccgtcttcttcttctggagcatgcgcgtttccggcgccgaggccgttgtcgctgccgcatccccattcatcggccagggcgaatcagccatgctgatccgcccctttatcgcccacctcaccacggccgaggtgcaccaaatcatgtgctccggcttcgccaccatcgccggaagtgtcctgatcgcctatatcagcatcggtgtcagcgcacaggcactggtcagcgcctgcgtcatgagtatccccgcttcactggcggtctcgaaaatccgttggcccgaagaggaagaaaccctcactgcaggtcgtgtcgtcatccccgatgacgaggaacaccgcgccgccaatgccctccacgcattcaccggcggcacctggctcggtctgaaaattgcggccatgatgtcgtcgatccttctgtgcatcatcgccctcgtcggtctaatcaacggcctcctcacttggtggggccgatacctgagcatcaacgacccccctctgactctcgagttgatcgtcggatacatctgctacccgattgccttcctgctcggtgtctcccgtgacggtgacctcctcaaggttggccagctgattggtcttaagctggttactgtaagttccccatcttcataaatggtcatttccatctcctaaccagccacagaacgaatttgttgcctatgaccgtcttcaacactgggacgactacaaggacctctccgaccgctctcgtctcattgttacgtacgcgctctgtggattcgctaatatcggctccctgggtaaccagatcggtgttctgtcacagatcgcgcccggtcgagctagcgatgtgtcgcgtgttgcattcagcgctatggttactggtgccattagtactttcaccagtgcggcggttgccggcttgttgattaccaatgagaaacagtactttggttcctccagttaggtacgacctgtatcttaatctgggtggttttttgatttcatacggttacttgagcatttattgatacccattcatgggtttgtccaatcttgatagcgtcgactttggaattatcggcagaatcgagcataattttcacatattct

SEQ ID NO: 59 - protein, 583 aa, Aspergillus niger
MPPACRISKEIADIHAGDPEAPRVDNLGARDVKKTSREEHQGEESDIGEADISRTPQQLYRHCMSRYTKHVAYAGILILFTGWWVAGLVLHRDDLGWLIPFIVYLAITLRIIFLYLPISVLTRPVFWIWKQTISRLVSYVPPRFRIPGAASMTITVIVIGCFVSADTPDNTRADRAVSLFGLIVFLFILWLFSRDRKKIVWHTVIVGMLVQFIVALFVLRTQAGYDIFNFISTLAQKLLGFAEQGVDFLIETGWAEKHKGWFIVSVIPAIMFFVSLVQLLYHAGILQWFVRKFATFFFWSMRVSGAEVVVAAASPFIGQGESAMLIRPFVPHLTMAEIHQIMTSGFATIAGSGLIAYIGMGVNPQALVSSCVMSIPASLAASKIRWPENEETLTAGHVTVPEDEEHQAANALHAFANGAWMGIKIAGMITANLLCIISLVGLANGLLTWWGRYLNINDPPLTIQLIMGYICYPVSFLLGVSRNDDLLKVAQLIGMKLVMNEFIAYSALQTDPQYQSLSPRSRLIATYALCGFANIGSLGNQIGVLAQLAPERAGDVSRVAVSAMITGAISTLMSAAIAGMIAI

SEQ ID NO: 60 - DNA, 1875 bp, Aspergillus niger
atgccgccggcttgcagaatatcgaaagagattgccgatattcatgcaggtgatccagaagcccctcgtgtcgataatctcggtgctcgagatgtgaagaaaacgtccagagaagaacatcagggcgaagaaagtgatatcggagaagccgacataagccgcaccccacagcaactgtatcgccattgcatgtccagatacaccaagcatgtggcatatgctggaatattgatacttttcacagggtgagtctttagtactagctctttcatccctcaaagattcgcaagcttaccaacttattatccccagttggtgggtcgcggggctggtccttcaccgcgatgacctgggatggctaatcccatttatcgtctatctggccatcactctgcgcattatctttctttatcttcccatttctgtgctcaccagaccggtcttctggatctggaaacagaccatctcgcgtcttgtaagctatgtgcctccaaggtttcgtataccaggagcagcgtcaatgacaattaccgtgatcgtgatcgggtgcttcgtctctgcggatacaccagacaacacgcgagccgacagagcagtcagcctattcggcctgatcgtcttcttgtttatcctatggctcttctctcgggatcgcaagaagattgtctggcataccgtcatcgttggcatgttggttcaatttatcgtggcgctattcgtcctgcggacacaggcgggatacgacatctttaacttcatatcaaccctagctcagaagctactgggatttgcagagcaaggcgtcgatttcctcatcgagacaggctgggctgagaaacacaaaggctggttcattgtcagtgtcatccctgcgattatgttcttcgtatctcttgtgcagctcctatatcacgctggtatcttacaatggttcgttcgcaaattcgcgacgttctttttctggtcaatgcgtgtgtccggggcagaagtggttgttgcagcggcctctccgttcatcgggcagggggaatcagccatgcttataaggccctttgtaccgcacctgaccatggctgaaattcatcaaatcatgacctctggatttgccaccatcgccggctccggtctcatcgcatacatcggcatgggtgtcaatcctcaagcccttgtatcttcctgcgtcatgagcatccccgcctccctggcagcatccaagatacgctggcccgaaaacgaggaaaccctcacagcaggccatgtcaccgtaccggaagacgaagagcatcaagccgcaaacgcccttcacgcgtttgcaaatggcgcctggatgggtattaagatcgccggcatgatcaccgccaacctactctgtatcatctccctggtcggcctagccaacggccttctcacctggtggggacgttacttgaatattaacgatcctcctctgaccatccaactcatcatgggatatatctgctaccccgtttcctttctccttggggtttcccgtaacgacgacctcctgaaagtagcgcagctaatcgggatgaagctcgtcatggtaagctccttctgcaatacccagagaccactcctaataaaatgtataaccgttagaacgaattcatagcctactcagctcttcagaccgacccgcaataccagtccttatctcctcgatcaagactaatagccacgtacgcgctctgcggatttgcaaacatcggctccctgggaaaccagatcggggtcctggcacaattggctcccgagcgggcaggagatgtgtctcgtgtggctgttagtgcaatgataacaggggccattagcacgcttatgagcgcagcaattgccggaatgattgctatctag

SEQ ID NO: 61 - protein, 640 aa, Trichodema reesei
MADSAPQQNAVDGIHHNPDPALEPAHQHQHHHKHHSSRVDAAGHDDAVYTKGVNLDPDLVPVQDHTHDEKHHIPTPEYSDHEKIGQTDIVRTGIDSDEADSQRRWRFGIAYRHYRAFVHIFIFLLFTGWWIASLILHRNDKNWVVPFLLWLAITIRLFFFHVPIRYVSNAIKWVWQRTALVIYARIPPKARTPAGAAVALATVLVGSFASEESADNTRENRAVSLFGMLVILFGFWITSNNRKHVNWRTVIGGMLGQYIIGLFVLRTGVGYDIFKFIADRAGDLLGFAHDGVAFLTTADVAALPWFFFGVIPAIIFFISLVQVLYYIGFIQWFIKKVATFVFWALNASGAEAVVAAATPFIGQGESAMLVRPFVPHMTNAELHQVLTCGFATISGSVLVGYIGLGLNAEALVSSCIMSIPASLAISKLRYPETEETLTAGRVVIPDDDEHKAENALHAFANGAWLGIKIGGTIIASLLCILGAVGLINGLLTWWGHYININHPTLTLQTILSYVFYPVAFLLGVPRNGDLLRVAKLIAEKVITNEYNAFNAMATDPYYEDMSPRSQLIATYALCGFGNIGSLGIQIGILSQLAPSRGGDVSRLALSALISGVFSTLTSASVAGLVVTTQLSHFTRPPASG

SEQ ID NO: 62 - DNA, 2511 bp, Trichodema reesei
aggttcagcgtccctgctaacctgctcttgcttcttccaacccctttgcttccgccctctcagtctggttggctgaccttggctcatttggttcaactccagatcagcttcgtgttgcttcaacagaccctgatagccttgctcaacccaaggctacagcacttcggctttgcgaagcgtctgacagacggacaagccaacccgggggagaagaagagaaggaaaggtcgagtcgacaacgacttgacaagtctctcgagacaaacacaggagacgacaaccagagtctagactgacaaaaatcgccatacgcaatggctgactctgccccccagcagaatgccgtcgacggcattcaccacaacccggatccggccctggagcccgcgcaccagcaccagcaccaccacaagcaccactcgtcccgtgtcgacgccgctggccacgacgatgccgtctacaccaagggcgtcaacctggacccggacctggtgccggtgcaggaccacacccacgacgagaagcaccacatccccacgcccgagtacagcgaccacgaaaagattggccagacggacattgtgcgcaccggcatcgactcggacgaggccgactcgcagcgccgctggcgctttggcattgcctaccgccactaccgcgcctttgtccacatcttcatcttcctgctcttcaccggctggtggattgcgtcgctgatcctgcaccgcaacgacaagaactgggtcgtgcccttcctgctgtggctggccatcaccatccgcctcttcttcttccatgtgcccatccgctacgtctctaacgccatcaagtgggtgtggcagcgcaccgccctcgtcatctatgctcgcatccctcccaaggcgcgcacgcctgccggtgccgccgtggcccttgcaactgtcctggtcggctcctttgcctctgaggagagcgccgacaacacgcgcgagaaccgagccgtcagtctgttcggcatgcttgtcattctctttggcttctggatcaccagcaacaaccgcaagcatgtcaactggcgcaccgtcattggcggcatgctgggccagtacatcattggtctctttgtcctgcgcaccggtgttggttacgacatctttaagttcattgcggaccgcgccggcgacttgctcggctttgcccacgatggtgttgccttcctgactaccgctgatgtggccgcgctcccctggttcttctttggcgtcatccccgccatcatcttcttcatctctctcgtgcaggtcctgtactacattggcttcatccagtggttcatcaagaaggtggccacttttgtcttctgggcgctcaacgcctccggtgccgaggctgtcgtcgctgccgccacgcccttcatcggccagggcgagtctgccatgctggtccgccccttcgtccctcacatgaccaatgccgagcttcaccaggtcctgacctgcggcttcgccaccatctcgggttccgtcctggtcggctacattggtctcggtctcaatgccgaggctctggtgtcgtcttgcatcatgtccatccccgcctcgttggccatctccaagcttcgctaccccgagacggaggagacgctcaccgccggccgcgttgtcatccccgacgacgatgagcacaaggctgagaacgccctgcacgccttcgccaatggcgcctggctcggcatcaagattggcggcaccatcatcgcctcgctgctctgcattctcggtgccgtcggtctcatcaacggcctccttacctggtggggccactacatcaacatcaaccaccctacgctcactctccagaccatcctgtcttacgtcttctacccggttgcctttttgctcggtgttccccgcaacggcgatctcctccgggttgccaagctcattgccgagaaggtcatcaccgtaagtgtctcctcgatccgtagtaatcacggttgcgtctccttgcttgtgatgtgataataactctggtgtctcttcagaacgagtacaatgccttcaacgccatggccaccgacccctactacgaggacatgtctccgcgctcccagctcattgctacctatgccctttgcggtttcggcaacattggctctctgggtatccagattggtatcctcagccagctggccccttcgcgtggcggtgacgtttctcgactggccctctctgcgctcatttctggcgttttctcgacgctgacctcggcctctgtcgctggtctggtcgtcactacccagctctcccacttcacccgacctcctgcgtctggttagagtattcttttttttctctcttgtgcgagcagtgttgatgatttgttccatgttcgggacgacatcgtgtgagttttgaatgagcaattagcagaggatgaagaactcgaggaagtggaaaagaagataccccgccacgatctgagctcggatgtgagcggtcaacttaaggagcatctttaatcgccgtgatg

SEQ ID NO: 63 - protein, 643 aa, Acremonium chrysogenum
MTDRTDNPAPTAPDTDPVLLPENQHHHPHRLHAASAEAPDEIVYATSGRQSTDSHVPKQYHPPTHHHCHGAHDTVEKGVATPPDYSDHEKAERGGVISNREASQETTGPAWKTGRFGWVHRHRRLLFTLFTFGLFTGWWIASLILHRNDKNWVIPFLFWLAIMLRLVFLYVPIRYVTKPIKWTWHNTAVRIYDLIPEKFRTLAGGILTVSVILVGSFVTEESADNTRENRAVSLFGMAVIITCFWATSRHRRHVNWRTVFGGMLGQYIIGLFVLRTKVGFDIFKFIADRASDLLGFAKDGVAFLTNPDTAALPMFFFNVIPAIIFFISLVQVLYYVGFLQWFIMKFAKFVFWALEVSGAEAVVAAATPFIGQGESAMLVRPFVPHMTRAEIHQIMTCGFATISGSVLIGYIDLGLNPQALVSSCIMSIPASLAISKLRYPETEETLTAGRVVIPDDDEHKAENALHAFAEGAYLGVKIAGTIIASILCILAAVGLINGLLTWWGSYLDINDPQLTLQTIFGYAFYPVAFLLGVPRDASLLKVSRLIAEKVITNEYVAFTMLAEGPEYADISPRTKLISTYAICGFGNIGSLGIQIGILGQLAPSRGGDVSSLAFSALISGVMATLTSASVAGLVISSQTPIIV

SEQ ID NO: 64 - DNA, 2003 bp, Acremonium chrysogenum
atgaccgaccgcacagacaacccggcgcccacggcgcctgacacggatcccgtgctgctgcccgaaaaccaacaccaccaccctcacagactgcatgccgccagcgccgaggcccccgacgaaatcgtctacgcgacttcggggagacagtccaccgacagtcatgttcccaagcagtaccacccgcccactcaccaccactgccatggcgcccacgataccgtcgagaagggtgtcgctacgccgcccgactacagcgaccacgagaaggccgagcgtggtggtgtcatctccaatagagaggcctcgcaggagacgacgggcccggcatggaagactggccgctttggctgggtacaccggcaccgcaggctgctctttaccctcttcacctttggtctcttcaccggctggtggatcgcctcgctgattctgcatcgcaatgataagaactgggtcattccctttctcttctggctcgccatcatgctgcgtctggtcttcctctatgttcccatccgctatgtcaccaagcccatcaaatggacctggcataacacggctgtcaggatctacgacctgattccagaaaagttccgaaccctggcgggggggattctcaccgtcagcgtcatcctcgtcggttctttcgtcaccgaggagagcgccgacaacacccgcgagaaccgcgctgtcagcttgttcggcatggccgtcatcatcacctgcttctgggcgacaagccgtcaccggaggcacgttaactggcgaacggttttcggtggcatgctgggccagtacattattggtctatttgtcctgcgcacgaaggtcggattcgacattttcaagttcatcgccgaccgcgccagcgacctcttgggcttcgccaaggatggcgttgcattcttgaccaaccctgacacggcggccctgcccatgttctttttcaacgtgattccggccatcatcttcttcatctctctggtccaggtgctctactacgttggattcctccagtggttcatcatgaagtttgccaaatttgtcttctgggccctcgaggtttctggtgccgaggctgtcgtcgctgccgccactccattcatcggccagggcgagtcggccatgctggtgcggcccttcgtgccacacatgaccagagcggagattcaccagatcatgacctgcggtttcgccaccatctcgggttccgtcctcatcggctacatcgatctcggcctcaacccccaggcgctggtgtcttcgtgcatcatgtccatccccgcgtccctcgccatctcgaagctgcgctaccccgagacagaggagaccctgacggcaggtcgcgtggtcatccccgacgacgacgagcacaaggctgagaatgccttgcacgcgtttgccgagggtgcctaccttggtgtcaagattgccggaaccattatcgcctcgatcctctgcattctcgccgccgtcggcctcatcaacggtcttctcacctggtggggcagctacctggacatcaacgacccgcagttgacgctgcagaccatcttcggctacgccttctatcctgtcgccttcctcctcggtgttcctcgcgacgcgagtctcctcaaggtcagcaggttgatagccgagaaagtcatcaccgtaagtactaccataatcaagatacatgaaacccttgtaatcgcctgatcgataacacgcaatgtctgcagaacgagtacgttgccttcacgatgcttgccgaggggcccgagtacgccgacatctcgccgcggacaaagctcatttccacgtacgccatctgcggtttcggaaacattggatctctcggtatccagataggtattctaggccagctggctccttcccgtgggggtgacgtctccagccttgccttctccgccctgatctcgggcgtcatggccaccctgacctcggccagcgtggccggtctggtcattagcagccagaccccgatcattgtgtag

SEQ ID NO: 65 - protein, 648 aa, Fusarium oxysporum
MADSNIHHNPDPALEPSHQHHHQHHHHSPRVDTPGHDDPVYTTGTTDAPSVVPPQRHSHEKEKTGAHTPPDYSDHEKHEAGVVDESARANSHSDVPAVTGWRRRLGPVYRYRRPIIHLFIFCLFTGWWIASLVLHRNDKNWVVPFLLWLAITLRLIFFHVPSRHVSNVIKKVWLNTAVRIYDLIPAHLRTLAGATVTIAAILIGAFVSEEVADNTRENRAVSLFGMAVFLFILWATSKDRKRINWRTVIGGMLTQYVIGLFVLRTTVGYDIFRFIADRAADLLGFAKAGVAFLTSDDVANTGNFFFGVIPAIIFFISLVQVLYYIGFVQWFIVKFATFVFWGLGVSGAEAVVAAATPFIGQGESAMLVRPFVPHMTKAELHQIMTCGFATISGSVLVGYIGLGLNREALVSSCIMSIPASLAISKMRYPETEETLTAGRVVIPDDDEHKAENALHAFANGAWLGIKIAGTIVCSLLCIIALVAFINGLLTWWGRYLNIDGKHPLTLQLILGYLLFPVSFLLGVSRTNGSNDTGDILPVAKLIAEKIITNEYKAFSLLTNPSPVDNEFYGLSPRSQLIATYALCGFGNIGSLGIQIGILSQLAPSRGGDVAKLAVSALISGVLATLTSASVAGLVVTNQLSSFGQNASS

SEQ ID NO: 66 - DNA, 3793 bp, Fusarium oxysporum
aaacattggctcaagtcgttagtctggggtttaattgaaccaacaggccaatccatcaatagataatgaacctacaaggcacacccctccaatcgcgccaactgtatctcaaggtacactccctgtcccggagctggaggcaaaaaaaaaaaaagctgggttaaaaaagtcaagcaatccccacgtttgaagagaaaaaaattgtcgacccacgaagatcgtctcttctgtcagttcagttcagttcagttcagttcagttgctccatcgtctgactccattctcagcgtctccagttttgagggctaaacttttctgaacaaaagcccttgcttcgccatcgcactcccaagggtccttcctttgttcttctcttgtgcttcactgctcaagctttgaagctccgaggtctcacctctacctcttaattatctttataccctttttttttataccccaattagtgttgagttgagagagagcgcggctacaatggcagattcaaacatccaccataatcccgacccagctctcgagccctcccaccaacaccaccaccagcatcatcaccactctccccgcgtcgacacccccggccacgatgatcccgtctacacaaccggcaccacagatgcgcccagtgtagttcctcctcagcgccactcccacgagaaggaaaagactggcgcgcatactcctcctgactacagcgaccacgagaagcacgaagcaggcgtcgtcgacgaatccgcccgcgcaaactcccacagcgacgtccccgctgtcaccggctggcgcagacgtctcggccctgtatatcgctaccgccgccccataatccacctcttcatcttctgcctcttcacaggctggtggattgcgtctctcgtcctgcaccgcaatgacaagaactgggttgttcccttcctcttgtggctcgccattactctccgtctgatcttcttccacgttcccagccgccatgtctccaacgtcatcaagaaggtctggctcaacaccgccgtcaggatctacgacctcatccccgcccacctgcgcactctcgctggcgctaccgtcaccatagctgctattctcatcggcgcttttgtttcggaggaggtcgctgacaacacccgtgagaaccgcgccgttagtcttttcggaatggctgtctttctcttcatcctctgggctactagcaaggaccgcaagcgcatcaactggcgcactgtcattggcggcatgctcacgcagtacgttatcggtctcttcgtcctgcgaacaaccgttggatacgacatcttccgcttcatcgccgaccgcgccgctgatcttcttggcttcgccaaggccggtgttgcattcctgaccagcgacgacgtcgccaacactggcaacttcttcttcggcgtcatccccgccatcatcttcttcatctccctcgttcaggtcctctactacatcggattcgtccagtggttcatcgtcaagttcgctaccttcgtcttctggggtctcggagtctcgggcgccgaagccgtcgtcgcagcagctacccccttcatcggccagggagaatctgccatgcttgtccgccccttcgttcctcacatgaccaaggccgagcttcaccagatcatgacctgtggtttcgctacaatctccggatctgtccttgtcggctatattggtctcggtctcaaccgtgaggctctcgtgtcgtcctgcatcatgtccatccccgcttctctggctatttccaagatgcgatatcccgagactgaagagactctcacagctggtcgtgtagtcattcccgacgatgatgagcacaaggctgagaacgctctccacgctttcgccaatggtgcttggctcggaatcaagatcgctggtaccatcgtctgctctcttctctgcattattgctctggtcgcattcatcaacggtctcttgacctggtggggacgatacctcaacatcgacggtaaacaccctcttactctccagctcattcttggatatcttctcttccccgtgtctttcctcctcggtgtatctcgcaccaacggatccaacgacactggcgatattctccccgtcgccaagctcatcgccgagaagatcatcaccgtacgtttcccccccttctcactctcctatcagatactgacacattctagaacgagtacaaggccttctccctcctcacaaacccttcccccgtggacaacgaattctacggcctctccccccgctcccagctcatcgccacctacgccctctgcggcttcggcaacatcggctcactcggtatccagatcggtatcctcagccagctcgccccctctcgcggcggtgatgttgccaagcttgctgtctcggctctcatctctggtgttctcgccactctcacctctgccagtgtcgctggtctcgtcgtcaccaatcagctttccagtttcggccagaacgcttcctcataattggacctgatgatgatgaaaatgtaagagaatgaggaaagacgtgtgtaaaaaacgtttgaagggtcgatgattatgtcgatatgctatactatactatactatactatactatttacaaatgctatactgaagaccacgagctggtactaattcacgtcactgctctactgtgccttcgctcttcatttgctataatcacttccgctacgccatacttcgtctcacgtttccattgtcatatattcacctacaatgtcacttacaagtggaaacttcgacgatggcactgaaccaccctcgtccgaggcacaggccatcccatgacacatcgtaccacaaaacaccaagcggggtcatcaagtaaagaattttcgtagatctatcatggcatgagaataatcagatacggctaccgaagtcaagtgcattggcccatcatcgtctgatcgacagataagcagaccgaactgccggagatgtactcccagtgggttggtactttataagcgacataggggtgtctatagacggcatggctatagttgatgacgtgcatgaagactaacaagagactgagggtgtggtataagtattcgtgttcatgatctatgtaaattaggtaggtatcaactatttattaaccgtaatcatcatatccacatatatcctcatcctcatcctcatccttcatcccacaacccttcaactgttctgcttcaccaactttttaaactccttcgacgtcctaccactcgatccatcacccctactcgactccccgtccttatctgaccccctctccttttctttctccttctcctcctccttcttcttcttctccatatgcttcatagcatcctgcgtcagctcctgtctatccaacagctcccacacgtccttctttcgcgtcttccgatcaccaatgctgatgagcagaccggctgtgatgttgcaggttatgctcacacagaggatcacggtggtcagagcgaacggacgcgtgtggtagtcgatgccccaggtacatgcgccgagggatatttgaagacaggagtggcagtcgaggagaattacgatggcgatgagatacctttgggggaaagcgaagtgggtgaatgtctcgttgggcttgtagaaactgtgtgacttggccattttcttctgatggtgctgcaggctcttgaactgagcaaacgtcagagcgggcatgtctgactcagggctgtttgacttggcgccttctaaatcaacagtctcgggactttcaggttgcggctgaggttggttgtcgatcgtgccga

SEQ ID NO: 67 - protein, 608 aa, Candida albicans
MVSPSTDKAPSIVELTPETYQQDVSSDSIDLESGTKKLQNESALQSHEYTTNIDETSSSNTASKLTYIQKLKIRFPYYRLAIDIFIGCFFTAWWLSIVIQPKHRHQWLIPTVIWGMIMVRLITWHIKILPWLLNKVKIVWDFFTGYVYKVLSKKYQRLITGAVITVGVILLGTFVPSETEYSKRKDRAISFFGCIVAIFLLFVTSKAPSKINWNAVIGGMLMQFIIALFVLRTKCGYDVFNFISTLARELLGFAKDGVAFLTNKDVSQLGMFFFTVLPSVAFFVAFIHIWYYFGVIQWAIRKFAYFFFWTLRVSGAEAITAAASPFIGIGESAILIKDLMPYLTKAELHQIMTSGFSTISGAVLVGYIGLGLNPQALVSSCVMSIPASLAVSKLRYPELENPISSGTVMIPKVEDSEAAREKSKDEPQNVLQAFSNGATLGLRIAGTMMIQCMCIIGLVALCNGILTWFGNYWNIDHLTLELMLSYIFYPIGFLLGTPRNEILLVSKLIAYKFIQNEYVAYNLLTNEAPYNEMSKRGTLIATYACCGFANLGSLGITLGVLNTLTNNSRAKDISSSIISALFCGAIATMLSAAIAGMVMHDLNTFHIN

SEQ ID NO: 68 - DNA, 1827 bp, Candida albicans
atggtttctccgtccacagataaagcaccatccattgtagagttgactccagaaacatatcaacaagatgtttcctcagattcaatcgatttggaatcaggtacaaaaaagctccaaaatgaatcagctttgcaatcacacgagtatactaccaatatcgatgaaacttcatcaagcaacactgcatccaagttgacttatatccaaaaattgaaaataagatttccttattacagattagcaattgacatttttatcggttgctttttcactgcatggtggttatctatagtcattcaacctaaacatagacatcaatggttgattccaacggttatttggggtatgattatggtgagattgatcacttggcacataaaaattttaccatggttattaaacaaagtcaaaattgtttgggatttctttactggttatgtgtataaagttttatcaaaaaaatatcaaagattaatcactggtgctgtgattactgttggtgttattttattaggtacatttgttccttcagaaactgaatattccaaaaggaaagatagagctatctcctttttcggttgtattgttgccatattcttattgtttgtcacttcaaaagctccttcgaaaattaattggaatgcggttattggcggtatgttgatgcaatttattattgcattatttgttttgagaactaagtgtgggtacgatgtatttaatttcatttccactttggcaagagaattattgggtttcgccaaagatggggtggcatttttaactaataaagatgtctctcaattaggaatgttctttttcaccgtgttaccttcagtggcttttttcgtggcgttcattcatatttggtattatttcggtgttattcaatgggccattagaaaatttgcttactttttcttttggacattaagagtttctggtgctgaagccattacagctgctgcctctccgtttatcggtattggtgaaagtgccattttaattaaagatttgatgccatatttgactaaagcagaattacatcaaatcatgacttcagggtttagtaccattagtggtgctgttcttgttggttatattggtcttggtcttaatccacaagctttggttagtagttgtgtcatgtcaattcctgcatctcttgcagtatcaaaattaagataccctgaacttgaaaacccaatctcaagtggtacagtaatgattccaaaagttgaagactctgaagcagcaagggaaaaatcaaaagatgaacctcaaaatgtcttgcaagcattttcaaatggggccactttaggattgagaattgccgggacaatgatgattcagtgtatgtgtattattggacttgttgccttatgcaatggtattttaacatggtttggtaactattggaacattgatcatttgactttggaattgatgctttcctacattttttacccaattggattcttgttgggtactccgcgtaatgaaattttgcttgttagtaaattgattgcttataaattcattcaaaatgaatatgttgcttataatttgttaacaaatgaagctccttataatgaaatgtctaaaagaggaacattaattgccacctatgcttgttgtgggtttgccaatttaggttctttgggtattactttgggtgttttgaatacattgacaaacaattctagagccaaagatatttcttcaagtattatatctgctttgttctgtggtgccattgccactatgttatctgctgccattgctggtatggttatgcatgatttaaacactttccacattaactag

SEQ ID NO: 69 - protein, 608 aa, Candida albicans
MVSPSTDKAPSIVELTPETYQQDVSSDSIDLESGTKKLQNESALQSHEYTTNIDETSSSNTASKLTYIQKLKIRFPYYRLAIDIFIGCFFTAWWLSIVIQPKHRHQWLIPTVIWGMIMVRLITWHIKILPWLLNKVKIVWDFFTGYVYKVLSKKYQRLITGAVITVGVILLGTFVPSETEYSKRKDRAISFFGCIVAIFLLFVTSKAPSKINWNAVIGGMLMQFIIALFVLRTKCGYDVFNFISTLARELLGFAKDGVAFLTNKDVSQLGMFFFTVLPSVAFFVAFIHIWYYFGVIQWAIRKFAYFFFWTLRVSGAEAITAAASPFIGIGESAILIKDLMPYLTKAELHQIMTSGFSTISGAVLVGYIGLGLNPQALVSSCVMSIPASLAVSKLRYPELENPISSGTVMIPKVEDPEEAREKSKDEPQNVLQAFSNGATLGLRIAGTMMIQCMCIIGLVALCNGILTWFGNYWNIDHLTLELMLSYIFYPIGFLLGTPRNEILLVSKLIAYKFIQNEYVAYNLLTNEAPYNEMSKRGTLIATYACCGFANLGSLGITLGVLNTLTNNSRAKDISSSIISALFCGAIATMLSAAIAGMVMHDLNTFHIN

SEQ ID NO: 70 - DNA, 1827 bp, Candida albicans
atggtttctccgtccacagataaagcaccatccattgtagagttgactccagaaacatatcaacaagatgtttcctcagattcaatcgatttggaatcaggtacaaaaaagctccaaaatgaatcagctttgcaatcacacgagtatactaccaatatcgatgaaacttcatcaagcaacactgcatccaagttgacttatatccaaaaattgaaaataagatttccttattacagattagcaattgacatttttatcggttgctttttcactgcatggtggttatctatagtcattcaacctaaacatagacatcaatggttgattccaacggttatttggggtatgattatggtgagattgatcacttggcacataaaaattttaccatggttattaaacaaagtcaaaattgtttgggatttctttactggttatgtgtataaagttttatcaaaaaaatatcaaagattaatcactggtgctgtgattactgttggtgttattttattaggtacatttgttccttcagaaactgaatattccaaaaggaaagatagagccatctcctttttcggttgtattgttgccatattcttattgtttgtcacttcaaaagctccttcgaaaattaattggaatgcggttattggcggtatgttgatgcaatttattattgcattatttgttttgagaactaagtgtgggtacgatgtatttaatttcatttccactttggcaagagaattattgggtttcgccaaagatggggtggcatttttaactaataaagatgtctctcaattaggaatgttctttttcaccgtgttaccttcagtggcttttttcgtggcgttcattcatatttggtattatttcggtgttattcaatgggccattagaaaatttgcttactttttcttttggacattaagagtttctggtgctgaagccattacagctgctgcctctccgtttatcggtattggtgaaagtgccattttaattaaagatttgatgccatatttgactaaagcagaattacatcaaatcatgacttcagggtttagtaccattagtggtgctgttcttgttggttatattggtcttggtcttaatccacaagctttggttagtagttgtgtcatgtcaattcctgcatctcttgcagtatcaaaattaagataccctgaacttgaaaacccaatctcaagtggtacagtaatgattccaaaagttgaagaccctgaagaagcaagggaaaaatcaaaagatgaacctcaaaatgtcttgcaagcattttcaaatggggccactttagggttgagaattgccgggacaatgatgattcagtgtatgtgtattattggacttgttgccttatgcaatggtattttaacatggtttggtaactattggaacattgatcatttgactttggaattgatgctttcctacattttttacccaattggattcttgttgggtactccgcgtaatgaaattttgcttgttagtaaattgattgcttataaattcattcaaaatgaatatgttgcttataatttgttaacaaatgaagctccttataatgaaatgtctaaaagaggaacattaattgccacctatgcttgttgtgggtttgccaatttaggttctttgggtattactttgggtgttttgaatacattgacaaacaattctagagccaaagatatttcttcaagtattatatctgctttgttctgtggtgccattgccactatgttatctgctgccattgctggtatggttatgcatgatttaaacactttccacattaactag

SEQ ID NO: 71 - protein, 608 aa, Candida albicans
MVSPSTDKAPSIVELTPETYQQDVSSDSIDLESGTKKLQNESALQSHEYTTNIDETSSSNTASKLTYIQKLKIRFPYYRLAIDIFIGCFFTAWWLSIVIQPKHRHQWLIPTVIWGMIMVRLITWHIKILPWLLNKVKIVWDFFTGYVYKVLSKKYQRLITGAVITVGVILLGTFVPSETEYSKRKDRAISFFGCIVAIFLLFVTSKAPSKINWNAVIGGMLMQFIIALFVLRTKCGYDVFNFISTLARELLGFAKDGVAFLTNKDVSQLGMFFFTVLPSVAFFVAFIHIWYYFGVIQWAIRKFAYFFFWTLRVSGAEAITAAASPFIGIGESAILIKDLMPYLTKAELHQIMTSGFSTISGAVLVGYIGLGLNPQALVSSCVMSIPASLAVSKLRYPELENPISSGTVMIPKVEDPEEAREKSKDEPQNVLQAFSNGATLGLRIAGTMMIQCMCIIGLVALCNGILTWFGNYWNIDHLTLELMLSYIFYPIGFLLGTPRNEILLVNKLIAYKFIQNEYVAYNLLTNEAPYNEMSKRGTLIATYACCGFANLGSLGITLGVLNTLTNNSRAKDISSSIISALFCGAIATMLSAAIAGMVMHDLNTFHIN

SEQ ID NO: 72 - DNA, 1827 bp, Candida albicans
atggtttctccgtccacagataaagcaccatccattgtagagttgactccagaaacatatcaacaagatgtttcctcagattcaatcgatttggaatcaggtacaaaaaagctccaaaatgaatcagctttgcaatcacacgagtatactaccaatatcgatgaaacttcatcaagcaacactgcatccaagttgacttatatccaaaaattgaaaataagatttccttattacagattagcaattgacatttttatcggttgctttttcactgcatggtggttatctatagtcattcaacctaaacatagacatcaatggttgattccaacggttatttggggtatgattatggtgagattgatcacttggcacataaaaattttaccatggttattaaacaaagtcaaaattgtttgggatttctttactggttatgtgtataaagttttatcaaaaaaatatcaaagattaatcactggtgctgtgattactgttggtgttattttattaggtacatttgttccttcagaaactgaatattccaaaaggaaagatagagccatctcctttttcggttgtattgttgccatattcttattgtttgtcacttcaaaagctccttcgaaaattaattggaatgcggttattggcggtatgttgatgcaatttattattgcattatttgttttgagaactaagtgtgggtacgatgtatttaatttcatttccactttggcaagagaattattgggtttcgccaaagatggggtggcatttttaactaataaagatgtctctcaattaggaatgttctttttcaccgtgttaccttcagtggctttttttgtggcgttcattcatatttggtattatttcggtgttattcaatgggccattagaaaatttgcttactttttcttttggacattaagagtttctggtgctgaagccattacagctgctgcctctccgtttatcggtattggtgaaagtgccattttaattaaagatttgatgccatatttgactaaagcagaattacatcaaatcatgacttcagggtttagtaccattagtggtgctgttcttgttggttatattggtcttggtcttaatccacaagctttggttagtagttgtgtcatgtcaattcctgcatctcttgcagtatcaaaattaagataccctgaacttgaaaacccaatctcaagtggtacagtaatgattccaaaagttgaagaccctgaagaagcaagggaaaaatcaaaagatgaacctcaaaatgtcttgcaagcattttcaaatggggccactttagggttgagaattgccgggacaatgatgattcagtgtatgtgtattattggacttgttgccttatgcaatggtattttaacatggtttggtaactattggaacattgatcatttgactttggaattgatgctttcctacattttttacccaattggattcttgttgggtactccgcgtaatgaaattttgcttgttaataaattgattgcttataaattcattcaaaatgaatatgttgcttataatttgttaacaaatgaagctccttataatgaaatgtctaaaagaggaacattaattgccacctatgcttgttgtgggtttgccaatttaggttctttgggtattactttgggtgttttgaatacattgacaaacaattctagagccaaagatatttcttcaagtattatatctgctttgttctgtggtgccattgccactatgttatctgctgccattgctggtatggttatgcatgatttaaacactttccacattaactag

SEQ ID NO: 73 - protein, 588 aa, Rhizopus delemar
MSHENELPISRTETANSKNNEVSMAARSHADLNPESVKGMSTAIDMPTVSNEKVDDICVDDDDEVDKKPGYIQKFYRRYIMFFHLAYFLIFTGFLAAAYALQVPKGYNQENLILGLIYAFVVLKIIFNYIPTTIITKPWMYCVRAVGKPIMKIPKTYRTIAYGFLVLCVIVATVFGLPEKPESTRLQRLVALFGMVVFLAILYITSRNRRAINWNTVFSGMLLQFILALFVFRCTVGHDIFQWASTFAQGYLEKASNGTSFVFGETVANSGIFAVSVFPTIIFFAATVQVLYYINALQWLLKKCAVFFMSILQVSGAESIVAVASPFLGQGENALLIKPFLPYLTCSEMHQVMCSGFATISGSVLYGYIAMGVSGEALLTSCIMSIPCSLAVSKMRMPEVDEPLTANTISVPPNDDRPSNILHAAGIGATTGINIVLLMIANLISLLALLYAVNAGLTWIGNFITIENLTLQLITGYIFVPVAWLIGIENKDVVTVGQIMATKIWANEFVAYQALITTYKGILSERSILITTYALCGFANLGSVGMQIGILSTLAPKRSGEIAQLAVSAMLCGAACTFISAAIAGMLS

SEQ ID NO: 74 - DNA, 1835 bp, Rhizopus delemar
atgtctcacgaaaacgaattgcctatctctcgaacagagacggccaatagcaagaacaatgaagtttctatggctgccagatcacatgctgacctgaatcctgaatctgtcaaaggaatgtctaccgccattgacatgcccactgtgtctaacgaaaaggtagatgatatatgcgttgatgatgatgatgaagtagacaagaaacctggatatatccaaaaattttaccgaagatacatcatgttcttccagtaaggcttattccggttacaaaaaaaaaattttttttagggaaattaaattttttttccctttatagtcttgcttactttctcatcttcaccgggttccttgctgctgcttatgctcttcaagtacctaaaggatacaatcaagagaatcttattttgggcttaatctatgcgtttgtcgtccttaaaataattttcaattatatacctacaaccatcattactaaaccatggatgtattgtgttcgtgcagtgggtaaacctatcatgaagattcccaagacctatcgtactattgcctacggttttttggtactttgtgtcattgttgctactgtctttggcttacccgaaaaacctgaatcgactcgtcttcagcgtttggttgccttgtttggtatggttgtctttttggctattctgtatataacatctcgtaatcgcagagcgattaattggaacaccgtcttttctggtatgcttcttcaattcattctcgccttgtttgttttccgctgtactgttggtcatgatattttccagtgggcttccacttttgctcaaggttacctcgaaaaagcctccaatggtacttcatttgtatttggtgaaacagtcgcaaactccggtatttttgcagtctccgttttccctacgatcattttctttgctgcaactgtccaagtgctttactacatcaatgcccttcaatggcttttgaaaaaatgtgctgtttttttcatgtcaattttgcaagtttctggtgcagaatccattgttgctgttgcttcaccttttttgggacaaggtgaaaacgctttactcattaagcctttcttgccttacttgacatgctctgaaatgcaccaagtcatgtgctctggttttgccaccatttctggttccgttttgtatggctatattgccatgggtgtatctggcgaagctcttttgacatcttgtatcatgtctattccttgttcattagcagtttccaagatgcgtatgcctgaagttgatgagccattaactgctaatacaatcagtgtaccacctaatgatgatcgtccatccaacatccttcacgctgctggtattggtgcaacaactggtattaacatcgtcttgcttatgattgccaacttaatctccctccttgctcttttgtatgctgtcaatgctggtttaacctggattggtaattttatcactattgaaaatttaactcttcaattgattactggttacatctttgtgcccgttgcatggctcattggtattgaaaataaggatgtagtaaccgttggacaaatcatggccaccaagatatgggccaatgaatttgttgcatatcaggctctcattacaacttacaagggtatcttgtctgaacgttctattctcatcaccacttatgctctctgtggttttgccaacttgggtagtgtgggtatgcaaattggtattttgagtactttggcccctaaacgtagtggtgaaatcgctcaattggcagtttctgctatgctttgtggtgctgcttgtacttttatttctgccgctattgccggtatgcttagttaa

SEQ ID NO: 75 - protein, 539 aa, Rhizopus delemar
MNTVEQAEYDDVSYQQPSRLGLLYATLKKHAFLIFWILFTGFFIASYAIQIPKGYSQELLILGLIYLYTTLYLFFCFVPNTIVTKPWNYVLNSISDVLCRRFSRRVLTIAWAVIVIVVIVATVFSFPEKDESPRIRRLIALFGFVVLIFGTWITSAHPKAVQWNTISTAMFIQFILALFVFRSSVGSDIFTWLATFAEAFLGYSYFGSDFVFGDTAANSGVFAITVFPAIIFFASVVQMLYYLGTIQFVLKKLSVVCATLLDISGAESIVTIASPFIGSSENALLIEPLIKHLTKSEIHMIMTCGFATISGSTLYGYIAMGVSAKALLTSCIMSIPCSIGISKLRYPETEESIVKNMKTVPTYADSATTTNIIHAAGKGAKVGIEIVFLIMANLIALLSLLNAFNGFLTWAGHFLTIQNLTLQMVTGYVFVPIAWLIGVDDKDLVSVGTLMATKIWANEFAAYQDMTAHYKGLLSARSELVATYALCGFANFGSVGTQVGVLSTLAPNRSGDVAKLAISALICGTLSTWLSASIAGMLV

SEQ ID NO: 76 - DNA, 1837 bp, Rhizopus delemar
atgaatacggtcgaacaagccgaatacgatgatgtgtcctatcaacagccatcaagattgggtctgctttatgcgacattaaaaaaacacgctttgtaaagtatctgatgacccacatacaatgtcttgttaacctattcttagtctcatcttttggatcttattcactggttttttcattgcttcctatgctattcaaatacccaaaggctacagtcaggaacttttaatcttgggtctcatctacctttacaccacactttatttattcttttgctttgtacctaatactattgtcactaaaccctggaactatgtcttgaattccatatccgatgttctctgtcgcaggttcagtcgacgggtgcttacgatcgcttgggcagtcattgtgatcgtggtgatcgtggccaccgtcttttctttccctgaaaaggacgaatcacctcgtatcagaagattgattgctctctttgggtttgttgtattgatctttggcacttggattacatcagctgtaagaagactaaaaaaaattttttttttttcatgtttcacttacctattttcaacagcatcctaaagcagtccagtggaacacgatcagtacggccatgttcatccaattcattctggccctctttgtctttcgatcctctgtcggcagcgacatctttacatggttagctacttttgctgaagcctttttgggatactcttactttggttctgatttcgtgtttggtgacacagctgccaattcgggtgtttttgctatcactgtctttccggccattatctttttcgcttcagttgtacagatgctatattacctcggcacaattcaattcgttctcaagaaattatctgtcgtttgtgcgactcttttggacatctctggtgctgaatccattgttacgattgcttctgtaaatgaaaaaaaaaaaaaaaaatttattgttgtcaccttatttattttttgcctgtagccctttattggttcatccgaaaatgctttattgattgaaccactgatcaagcatttaaccaaatcagagatccatatgatcatgacctgtggatttgctaccatttccggttcgacgctctatggttatattgcgatgggtgtctctgccaaggcgctcttgacgtcttgtatcatgtccattccttgttcgatcggtatttctaaattaagataccctgaaacagaagaatccattgtcaagaacatgaaaaccgtgcctacgtatgctgattctgctacaacaaccaacatcattcacgctgctggtaagggtgctaaagttggtatcgaaatcgtctttttgatcatggccaatttgatcgctctcttgtccctcttgaatgcattcaacggattcttgacgtgggctggtcatttcctgaccattcagaacctcacacttcaaatggtgaccggttacgtctttgtccccgtaagttgccttcttttcttttttatatttactcaccacactttccccagatcgcttggttaataggcgtcgatgacaaggacctcgtgtcggtcgggacactgatggccaccaagatttgggccaacgaattcgctgcttatcaagacatgaccgcacactacaaaggcctgctgtcggctcgatctgaactggtggctacctatgccctttgtggatttgccaattttggttcggtcggtactcaggtcggtgtcttgagcacgttggcgccgaatcgttcgggtgatgtggccaaattggccatctctgctttaatttgtggtacactcagtacttggttatctgcttcgattgcaggcatgcttgtttaa

SEQ ID NO: 77 - protein, 452 aa, Aspergillus niger
MQSISPVYSSATDRRYSPPWADLSIIGIAGSSGSGKTSVAMEIVKSLNLPWVVILVMDSFYKSLSPEDHARAHRNQYDFDCPESLDFDVLVQTLRDLKQGKKADIPIYSFAEHQRQPETSTLYSPRVLILEGILALHDPRIMELLDVKIFVEADMDVCLGRRIMRDVRERGRDVEGIVKQWFTYVKPSYKQYVEPQRAVSDIIIPRGIENRTAIEMVVQHIQRKLDEKSEKHNAELNRLGLIASEEQLSSNVLMMPQTPQFVGMNTILQDPATEQVDFVFYFDRLAALLIEKALDMTNYVSQAVDTPQSTSYDGLNQAGVVSAVAILRGGSCLETALKRTIPDCITGRVLIQTNEKNEEPELHYLKLPPNIENHENVMLLDPQMSSGGAALMAVRVMIDHGVQEDKIIFVTCAAGKIGLKRLTTVFPGIKVIVGRIEEEREPRWIEKRYFGC

SEQ ID NO: 78 - DNA, 1808 bp, Aspergillus niger
atgcagtcaatctcccctgtatactcgtccgcgacagacagacgctactctccaccatgggcggatttgagtatcatcggtatcgcaggcagctccggctccggcaagacttcggtggccatggagattgtgaagtctctgaatcttccctgggttgtgattctcgtaatggtatatgcgaccgctttcataattgtttagcctacattagatccttccacttcgtgttctctaacggcggtgtggtataggactccttctacaagagcttgtccccggaggatcatgccagagcccaccgcaatcaatatgactttgactgtccggagtctctggactttgatgtcctggtccaaaccttaagggatttgaagcaagggtatggtatatgagcctctttctgtaattcctggtggggcacgtgccatatatggaagctgacaggatcccttccaaccatctagaaagaaagcagatatccccatttactcgtttgcagaacaccagcgtcagccagaaacgagtaccctgtattccccgcgcgtattgatcctggagggtattctggccttgcatgacccgaggatcatggagcttctggatgttaaggtaagtttcgtccgccttcctaatctggaccatcgctcactcttcttgatagatctttgttgaagcagacatggatgtctgccttgggcgcagaagtcagtttagcctactcttcaaccatttgccactgctctaacggagaggaagctagtcatgcgcgatgttcgtgagagaggaagagacgtcgaaggaatcgtcaagcagtggttcacttatgtgaagccatcatacaagcaatacgtcgaaccccaacgtgctgtgtctggtatgtggatgttctccaaaacttgaaccgggtctgacaagcttcagacattattattccccgtggtattgaaaaccgaacggccattggtaagagtgtctgctccttctttcggaagatctcacactaatttctgctggagaaatggttgtgcaacatattcagcgcaagcttgacgagaaatcagagaagcataatgcagagctcaaccggcttggtctcatcgcctcggaagagcagctatcttctaacgtcttgatgatgccccaaacgccgcaatttgttggcatgaacactatcctccaggacccggcaacggaacaagttgactttgtgttctacttcgaccgacttgctgctttactgattgaaaagtgagttatgttgctatcctttgcgaacatcgcgtgatactgtggaacaatagcttatgcgtaacttactgcacagggccttggatatgaccaattacgtatcacaagcagtggatacgcctcaatcgactagctatgatggcctgaaccaagctggtgtagtgtctgccgtcgcgattctgcgtggaggctcttgcctcgagaccgctctcaagcggaccatccctgactgcatcactggtcgtgtgctgatccagaccaatgaaaagaacgaagaacccgaacttcactacctgaagctccctcccaatattgagaaccacgaaaacgtcatgctcctcgacccccagatgtccagtggaggagctgctctaatggcggttcgtgttatgattgatcacggtgttcaagaggataagattattttcgtgacctgcgctgcaggaaagatcggattgaagcgactgacgacagtgttccctggaatcaaggttatagttggacggattgaggaggagcgcgaacctcggtggattgagaagagatactttggctgttga

SEQ ID NO: 79 - protein, 452 aa, Penicillium chrysogenum
MESVSPLYSLSTEVHYSPPWQDLSIIGIAGSSGSGKSSVAMEIVKSLNLPWVVILVMDSFYKTLTAEQHKKAHANEYDFDCPESIDFDILVDTLRDLKKGKRANIPVYSFAEHQRQPNTTTLYSPRVIILEGILALHDPRIVEMLDVKIFVEADMDVCLGRRILRDVRERGRDIEGIIKQWFEFVKPSYTRYVEPQRPISDIIIPRGIENTTAIDMVVKHIQRKLQEKSDNHTEELRKLGLVAAEVELPLNVHVLPSTPQFVGMNTILQNPETEQEDFIFYFDRLVSILIEKALDMTLYVSANVETPQGNTYLGLHPKGTVSAVAILRGGSCMETALKRSIPDCLTGRVLIQTNESNEEPELHYLKLPSQIEEHATVLLIDSQMSSGGAALMAVRVLIDHGVEQQRIVFVTCAAGERGLKRLTAVYPRINVVVGRIEEEWEPRWIEKRYFGC

SEQ ID NO: 80 - DNA, 1923 bp, Penicillium chrysogenum
atggaatcagtttcacctttatattctttgtctaccgaagtgcactattctccaccatggcaggacttgagcattatcggcattgctggcagctctggctccggtaagagctctgtggcaatggagatcgtcaagtctttgaacttgccgtgggtggtgatccttgtaatggtacagctcgtggaatccaatgcatcttaatttgcgaccctcttgttctaatatattccatacaggactcattctacaagaccttaactgctgagcaacacaagaaggcccacgcaaatgagtatgatttcgactgcccggaatcgattgacttcgatattctggtggacaccctccgtgatctgaagaaagggtaaatttactgttcgtcctagtcttctatttgacctgactggttatcacacagaaagagagcgaacatcccagtttactcatttgctgagcaccagcggcagcccaatacgaccactctctattctcctcgtgtgatcattctagagggtattttggccctccatgatcctagaatcgtcgagatgttggatgtgaaggtattatggctccttcgctatcctcatgcttatgagtgctaattcaatggcttagatcttcgttgaagcagatatggatgtgtgcttaggtcgcaggagtgggttgccccgatcgtgataccttaattccactgacgatgtatatagtcttgcgtgatgtccgtgagcgagggcgtgacattgaaggaatcatcaaacaatggtttgaatttgtgaagccttcttacactcgatacgtggagcctcaacgccctatctcaggtcggtaatcacatgagtttgcaaagcgtcaaaagactttttgttaactttaaatctcagacatcatcatcccacgtggcattgagaacacgaccgctatcggtatgcaatccctttgagctgctaatcttgagagctacccactaattggtgtagacatggtggtcaaacatatccaacgcaagctgcaggagaaatccgacaaccacaccgaagagcttcgcaaactgggtttggttgcagcagaagtagagctgcctctgaatgtgcatgtcttgccctcgacaccgcagtttgttggcatgaatactattctgcagaacccagaaactgagcaagaggatttcattttctactttgatcgactagtttcgatcttgattgaaaagtactgacctccatctcaaaatgcgcctttagttactctgctaacgtcaccatcgcagggccttggatatgacattgtatgtatccgcaaatgtggaaacaccacaaggcaacacatacctcggtttacatcccaagggtaccgtatcagccgtggctatcttacgtggtggctcttgcatggagaccgcgcttaagcgatccattcccgactgtcttactggtcgcgtgctcatccaaacgaacgagagcaatgaggagccagagttgcattacttgaagctcccgtcgcagatcgaggagcatgcaactgtcctcctaatcgactctcaaatgtccagtggaggtgctgcacttatggctgttcgtgtcttgattgatcatggggtggaacaacaaagaatcgttttcgtaacttgcgcagcaggagaacggggcctcaagaggcttactgctgtttatcccaggatcaatgtggttgttggacgcatcgaagaagagtgggaaccgcgctggatagaaaagcgatacttcgggtgctgaaattttcattccattgttggccccttccacagtatatatgttgatctgctcactatgacttatttctgagaccaagcattggattactgggattgacacttagcaagagcatacttatccatacgtttcatattcaatgaaatctaagcaaataccattacaatttttc

SEQ ID NO: 81 - protein, 448 aa, Acremonium chrysogenum
MSQLESHITVQKRAYYSPPWADVSIIAVAGSSGSGKSTISQAIVKKLNLPWVDSFYKTLTPEQSKLAFANEYDFDSPEAIDFDALVDRLRDLKAGKRADIPVYSFAKHQRMSETTSIYSPHVLILEGIFALYDPRVLELLDMGIYCEADADTCLSRRIVRDVRERGRDIEGCIKQWFGFVKPNFEKYVEPQRKKADLIVPRGIENRVALAEMMVHFIERKLFEKSRHHREALSRLGAACKEEPLSNRVVVLDDTPQLKFMNTILQDIDTNAEDFIFYFDRLASLIVEQALNNVQFKSLTVETPEGYKYQGLVPKGEVSAVIVLRGGSAFETALRKTIPDCRTGRLLIQSDYTTGEPELHYLRLPEDINKHESVLLLDTQMASGGAALMAVQVLVDHGVSLDRIVLATYSAGRVGLHRLTSVFPEITVVVCNILQDQEPRWVEKRYFRC

SEQ ID NO: 82 - DNA, 1855 bp, Acremonium chrysogenum
atgagtcaactcgagagccatatcacagtccagaagcgagcgtactactctccaccatgggccgacgtcagcatcattgccgtggctggcagctcggggtcgggcaagtcgacaatctcacaggcgatcgtgaagaagctcaacctgccttgggttgtcattctgtcaatggcatgctcgccccgtgtccatttctctaaggggcactgatgacattcaagctaacccctatgtcaggactcgttctacaagacactcacccccgaacaatcgaaactggcttttgccaatgagtatgattttgactcgcccgaggtatcttattccggattctcaaggagctgagactttttttttcgttgatcacatttgcgtaggctatcgactttgatgccctggtcgatcggttgcgggatctcaaggctgggtatgtttgagcgttaccgtaccctggagtcacccttctgcctacttactgacccggccctaaggaaacgtgcggacatcccggtgtattcctttgccaagcatcaacgcatgagtgagacgacatcgatctactcgccccacgttctcattttggagggcatctttgcactttacgacccacgggtcctcgagcttctggatatgggtgtacgtgaggcacatggcggcacctccttcaaatacgtacgcaatggtgatcctgacctgttgctgtagatatattgcgaggcggatgccgacacctgtctgtccagacgaagtaagtcctccggtgagcggcgcgggaaacaggcactgatagtctgccagttgtgcgggatgtgcgagagcgaggacgagatatcgagggttgtatcaaacagtggtttggctttgtgaaacccaactttgagaaggtaagtcctttgggaatgtatcaccgggacacgggtacctgacaagtctccagtacgtcgagcctcagcgtaagaaggctgatctcatcgtgccacgaggaatcgagaatcgtgtggcgcttggtaggcggacccctcctgtccggcatacagtactagtatcccatcagcccgctgacaagcagcagagatgatggtgcactttatcgagcgcaaattgtttgagaaatcaagacaccaccgcgaagccctctccagactaggggcagcctgcaaggaagagccgctgtcgaaccgcgtcgtcgtcctagacgatacgccccagctcaaattcatgaacaccattttgcaggacattgacacaaacgccgaagacttcattttttactttgaccggctggccagcctcattgtcgagcagtacgttccccatgctcccctgatcaggtaccgtgctgcctgggacgatcgagtaacgtgattgcagggccctcaacaatgtgcagttcaagagcctgacggttgagacaccggaaggctacaagtaccagggactagtgccaaagggagaggtcagcgccgtcattgtcctccgaggcggatcagccttcgagactgctttgcgaaagacgatcccggattgtagaacaggccgactactcattcagtccgactacacgaccggcgagccagaactccactatctcaggctcccggaggacataaacaagcatgagagcgtcctgcttctcgacactcagatggccagtggaggagcagcgctcatggcggtccaggtcctcgtagaccacggtgtctcgttggacaggatcgtgctggcgacctactcggccgggagggtgggtctgcatcggttgactagtgtcttccctgagatcactgttgtcgtttgcaacatcttgcaggatcaggagcctcgatgggttgagaagaggtatttccggtgttag

SEQ ID NO: 83 - protein, 447 aa, Trichodema reesei
MSQLESHVTVQKRAYYSPPWADVSIIAIAGSSGSGKSTLSQTIVKKLNLPWVDSFYKTLTPAQSKLAFANEYDFDSPDAIDFDALISSLRDLKAGKRAEIPVYSFAHHARLERTTSIYSPHVLVLEGIFALYDPRVRELCDMGIYCEADADTCLSRRIVRDVRERGRDVEGCIKQWFAFVKPNFEKYVEPQRKVADIIVPRGIENRVALDMVTQFIEKKLFEKSTHHREALARLEIKGKEEPLSDRVVVMSEGPQIKFMNTILQDIDTSAEDFIFYFDRLAALIIEQALNNVHFEATTIETPPGYKYNGLRPKGEVSAVIVLRGGSAFEPALRKTIPDCRTGRLLIQSSYSTGEPELHYLRLPEDIHEHESVLLLDTQMASGGAALMAVQVLVDHGVALERIVLATYSAGKVGLHRLMTVFPEITAVVCNLLPHAEQRWVEKRYFRC

SEQ ID NO: 84 - DNA, 1965 bp, Trichodema reesei
atgtcgcagctcgagagccatgtcacggttcagaagcgagcttactactctccgccatgggcagacgtgagcatcattgccatcgctggcagctcgggctccggcaagtcgacgctgtctcagactatcgtaaagaagctcaacctgccctgggttgtcattctttccatggcatgaagtcccctaggagaagcagcgggctttggatcgtcgtggctaacgaccagatcaggactcgttctataagacattgacccctgcccagtccaagttggctttcgccaacgagtacgacttcgactcacccgacgccattgatttcgatgccctcattagctctctacgggatttgaaagctgggtaacgatgacctgccgccaatgaagcttgtttcggacacgctcagttaaccctgcctttccagaaagcgagcagagattcccgtatactccttcgcccatcatgcccgactggagaggaccacatccatttattccccgcacgtgctcgttttggaaggcatctttgccctctacgacccgcgggtccgtgagctgtgtgatatgggagtatgcctgcctcgcccgatgttgatacaacatgggatctgacttgctctctagatctactgtgaagctgatgcagacacttgtttgtcgaggagaagtaggccatctcccctgcccgtagagatgttgacgaagatgcaactgctcactatctgcaccagttgttcgagatgtccgggagcgagggcgagacgtcgagggctgcatcaagcaatggttcgcctttgtgaagcccaatttcgaaaaggtgaggcgccccaatcaagctgcaactgtctgataggaaacatttcacttaccaagctcccttcgcgtggattagtacgtcgagccacagcgcaaagtggcagacatcatcgttccgagaggcattgagaatcgagtagcgcttggtgagtttcagacgcagtggattgactcggggtgagaaatatcttggctgacatggcggttcgaagacatggtgactcaatttattgagaagaagctgtttgaaaagtcgactcatcatcgcgaggcattggccagactcgagatcaagggcaaagaagagccgctttccgaccgtgttgtagtcatgagtgaggggcctcagatcaaattcatgaacaccattcttcaggatatcgacacttcagccgaggacttcatcttctactttgatcgcctcgccgccttgatcattgaacagtaggtcgaaccatggcgtgtgtgacatcaaggccgatgcatgctgacccgactacagggccctaaacaacgtccactttgaagcaacaacgattgagacaccaccggggtacaagtacaacggcttgcgccccaagggagaggtcagcgccgtgattgtgctccgaggaggatcggcgttcgagccggctctcagaaagaccattccggactgcaggacgggacggcttctgattcagtcaagctactcaaccggggagccggagctgcattacctgcgcctaccggaagatattcacgagcatgagagcgtgcttttgttagacacccaaatggccagcggaggagcagctcttatggctgttcaagtcctcgtcgaccacggcgtcgcgttggaacgcatcgtcttggccacttattccgccggaaaagttggcttgcacaggttgatgacggttttcccagaaatcacggccgtggtgtgcaatctcctgccccacgcggagcagcgatgggtggaaaagagatatttccgttgttgagcagcgagcggttttgcaattctaatgacaccaccaccccgttttttcccacatgacaggcgacgaggtgaaccgatctcctcacggccaggtggaatcgcaagtgcttgttactcgagacggccacggctcgaacgatgttgacacttatgacagtgagatga

SEQ ID NO: 85 - protein, 461 aa, Fusarium oxysporum
MSASAILEVNGGLESHVTVQKRAYYSPPWADVSIIGVAGSSGSGKSTLSQAIVKKLNLPWVVILSMDSFYKTLTPEQSKLAFANEYDFDSPDAIDFDVLVDKLRDLKAGKRAEIPVYSFAKHSRLDRTTSIYSPHVLVLEGIFALYDPRVLELLDMGIYCEADADTCLSRRLVRDVRERGRDIEGIIKQWFGFVKPNFEKFVEPQRKVADLIVPRGIENRVALEMMVQFVEKKLFEKSRHHREALSRLEAASKDSPLSERVVVLDDTRQLKFMNTILQDIDTDPEDFIFYFDRLASLIIEQALNNAHFEAKNIVTPQGYEYKGLVSTGEVCAVIVLRGGSAFEPALRKTIPDCRTGRLLIQSDYSTGEPELHYLRLPDDIADQESVLLLDTQMATGGAALMAVQVLVDHGVKQDRIVLATYSAGKVGLHRLTSVFPEITVVVCNMLDYQQERWVEKRYFRC

SEQ ID NO: 86 - DNA, 2709 bp, Fusarium oxysporum
ttcggccttcgccgtctttcgatagacacagatacggcatgaggagaaccctgacgaggtcaggtcgatcgtcaaaacatgagcagggccagggctgagctgatttatcttcttacacttttgctttctaactcaaccaccaaagtgtatctatatatctactatcactctcactctcaccttttcctctgatctatagctacaactgctagcaatagctcatctcatcactgaagctctcactgctgtcccgcttcttcttggccctctgtggatcacacacgcgacaggaccgtgctcactgctccaccaacaacgggcaagccaaaccctcgagtcgaaaccttgaaggcaccatactcgtacagctcgtggacattgcagggctagttttcggagaggaaagtttgagttctccagctaccaaatattctcgcctttcgttcatccgatcctcgtcctcttcgccccaaatcttaggtgacggtgacctcatcgagaactagcccaccgcaatgtccgcctctgcaatccttgaagtcaacggcgggctcgagagtcatgtcacggttcagaagcgagcttactattcacctccttgggccgatgtcagcattatcggcgtcgcgggcagctcaggttcaggcaaatcgaccttgtcccaggctatcgtcaagaagttgaacctaccctgggtcgttattttgtcaatggcatgaatccctgaaactatccttcgaacataagctcatatataacaggactctttttataagaccttgacacccgagcagtctaaactggccttcgccaacgaatatgacttcgactcgcctgacgtatgcttcctgtgacatagtatgaactgccagaataacaactgatgctattcttataggccatcgatttcgatgttctggtcgataagctacgggatcttaaggctgggtatgtgtagttgacgatggcttttggatttacacctcactgacgtctcaccatactcacagaaaacgggctgaaattcctgtttattcattcgccaaacattctcgattggaccgtacaacatccatctactctcctcatgtacttgtcctagagggtatttttgcgctttacgaccctcgagtgcttgaactgcttgatatgggtgtatgttaaaaccgatgccctattgaagcgtatgatttggacctaacactgaacttagatctattgtgaggcagatgcagatacttgcctgtcaaggagacgtaagtcttgacgttacgaagacctatggaagggtttctgaccaggttggtgtagttgtccgtgatgtacgagagcgtgggcgggatatcgagggtatcatcaagcagtggtttggatttgtcaagccaaactttgagaaggtaagttccccgtatcagaaacagaggatttcttatgaaggctaattgacaacttcaagttcgtcgagccccaacggaaggttgctgatttgatcgtaccgcgaggaatcgagaacagagttgcccttggtgagtagtcacaagaccttgaaaacctgactaaaacgctcatgttcatagaaatgatggttcagtttgttgagaagaagctgttcgagaaatcaagacatcaccgagaggcactgtcccgtctggaagcagcaagtaaggattcaccgctttctgaacgggtcgtggtcttggatgatacacgacagctcaaattcatgaacaccatccttcaggatattgacacagatcctgaggatttcatcttctactttgacaggcttgcgagtctgattattgaacagtaagagactgagaggctgttagtgtgaaagactgtttgttcgctaacacttcatagggccctcaacaatgcccatttcgaggccaagaatattgtaacaccccaaggatacgaatacaagggtcttgtatcaacgggtgaggtctgcgccgttattgtactccgaggaggatcagcgtttgagccggcgcttcgaaagactattcccgattgccgaacgggccgtcttcttatccaatccgactattccacgggagagccggaacttcattacttgcgactacctgacgatattgcagaccaagaaagcgtcctacttctggacacccagatggctactggaggtgcagcgttgatggcggtgcaagtcctggtcgatcacggcgtgaaacaggatcgtattgttctagcaacatactcggccggcaaggttggacttcacagattgacatccgttttcccagagattacagtcgtggtttgcaacatgctcgactaccaacaagaacgatgggtcgagaagaggtatttccgctgctgatggaccactcttatataaagtatacgtttgtcatgatagatgatggcggaagcttggaaacaacgcagtgatagtggtagtcaaatcaagattgacgataaggattatcggaatgcccggtaattatatccgtaaaggaagcaaatatgcgaacagcgctgcgtttggttgctgcgtcagctcttcacaatcttgagatccaccacaattggcatctcatcagcatctgtaacgtaactgatccacgactaggatccacgaaacgggacgtccgaaaccaacaggatacttggaaaaaagaggagccactgtgtgatatttgacgcaaaccagccaaagtcatggcatggcggccgacg

SEQ ID NO: 87 - protein, 237 aa, Aspergillus oryzae
MVVKHIQRKLDEKSEKHRAELDQLRKIASQLQLSPNVMVMPSTSQFVGMNTILQDPKTEQVDFVFYFDRLASLLIEKALDCTSYVPAGVETPQKTTYQGLNPEGIISAVAILRGGSCLETALKRTIPDCITGRVLIQTNAQNEVPELHYLKLPENIQKHTTVMLLDPQMSTGGAALMAVRVLIDHGVEEHKIVFVTCAAGKIGLKRLSTVYPKVRVIVGRIEEEQEPRWMERRYFGC

SEQ ID NO: 88 - DNA, 838 bp, Aspergillus oryzae
atggttgtgaaacacattcagcggaaactcgatgagaagtctgagaagcatagagcggagctagaccaattacgcaaaattgcgtcgcaattgcagctgtcgcctaatgtcatggtaatgccttcgacatcacagtttgttggcatgaacacaatccttcaagacccaaaaacggagcaagtcgacttcgtcttttactttgaccgactcgcttccttgctcatagagaagtgagtcaactgcgcatgcaagacagaattataccccaatcacctttaagcattgcttccccttgattctttccaattagcatggcaaaaacgagacggatgactgacattatcatcgaaacagggccttggactgtacctcgtatgttccagctggggtagaaacacctcagaagactacctatcagggtttgaatccggaaggtataatatctgctgttgcaatcctgcgaggaggctcttgccttgagactgccctaaagcgcactattcctgactgcattactggtcgcgtgctcattcaaaccaatgcacagaatgaagtaccggagttgcactatctaaaattgcctgaaaatatccagaagcatactacagttatgctacttgacccccagatgtccaccggaggagctgctctgatggccgttcgagtgttgattgaccacggcgtggaagagcacaagattgtctttgtgacatgtgccgcgggaaagatagggctaaagcgtctctcgacagtgtacccgaaagttagggtcattgtaggacggatcgaagaggagcaagagcctagatggatggagagaagatactttggctgctga

SEQ ID NO: 89 - protein, 545 aa, Candida albicans
MPLHPKSRRRSSRISPLPDEDSLSFINSSVENLDQSPFESIDDLVEDVNKYDLKSPSDEQQQQQQQESQTQNLKHKPSFTSTPKASYIPPWTEPYIIGIAGNSGSGKTSISQKIIQDINQPWTVLLSFDNFYQPLTSEQSKLAFANNYDFDCPDSLDFDLLVETIGNLKKGGKTTIPVYSFTSHNRTSKTNTIYGANVIIVEGLYALHDQRLLDMMDLKIYVDTDLDICLARRLTRDILYRGRDLGGAMQQWEKFVKPNAVKFINPTVQNADLVIPRGLDNSIAINLMIKHIKNQLALKSRNHLQRLKKLGVNIKFDIDKFNIKLLQNTNQVKGINSILFDTSTSRNDFIFYFNRMCGLLIELAQEFMTNYTNVDIDTGKGIYHGKKLLQNQYNAVNIIRSGDCFMASIKKSFPVISIGKLLIQSDSTTGEPQLHFERLPHKLSDKIMLFDSQIISGAGAIMAIQVLLDHHVKEQDIILITYLSTEIGIRRIVNVFPKVKIVVGKLSSMEDSNSNNKVWYNNEGFLDSHWHFRNRFIDSLYFGTE

SEQ ID NO: 90 - DNA, 1638 bp, Candida albicans
atgccactacatccgaaatcaagaagaagatcaagtagaatatcgccacttcctgacgaggattcattgtcatttataaactcatcagtagaaaaccttgatcaatcaccatttgaaagtatcgatgatttagttgaagatgtcaacaaatatgacttgaaaagtcccagtgatgagcaacaacaacaacagcaacaagagctgcaaactcaaaatttgaaacacaaaccatcattcacttctacaccgaaagcatcatatatcccaccatggaccgaaccatatatcattggtattgctggtaattcagggtcagggaaaacctcaatctcccaaaaaatcattcaggatatcaatcagccatggacggtattattatcatttgataatttctatcaaccattgaccctggaacaaagtaaacttgcatttgccaataattatgattttgattgtcctgattcattagattttgatttattagtagagaccattggtaatttgaaaaaaggaggtaaaactactatcccagtttattcattcacttcacataatcgtacttcaaaaactaataccatttatggcgccaatgttattattgtggaaggtttatatgctttacatgatcaacgattattggatatgatggatttgaaaatatatgtcgatacagatttggatatttgtttagcaagaagattaactcgagatatattgtatcgtggtcgagatttaggtggagctatgcaacaatgggagaaatttgttaagccaaatgcggttaaattcattaatccaacggtacaaaatgctgatttggtgattcctcgaggattagataattcaattgccataaatttaatgattaaacatatcaagaatcaattagcattaaaatcaagaaatcatttacaacgattgaagaaattaggggtcaatataaaatttgatattgataaattcaatattaaattattacaaaacactaatcaagtcaagggaatcaattcgattttatttgatacgtcaacttcacgtaatgatttcatattttattttaatcgtatgtgtggattattaattgaattagcacaagagtttatgaccaattataccaatgtcgatatcgatactggtaaagggatttatcatggtaaaaaattattacaaaatcaatataatgcagtcaatataattcgtagtggagattgcttcatggcatcaattaaaaaatcattcccagtaatttctattggtaaattattaattcaaagtgattcaactacgggggaacctcaattacattttgaaagattgcctcataaattatctgataaaattatgttatttgattcacaaattattagtggagccggagccattatggcaattcaagtattattagatcatcatgttaaagaacaagatattattttaattacgtatttatctacagaaattggtataagaagaattgttaatgtgttccctaaagtgaaaattgttgttgggaaattatcaagtatggaagattcaaattcgaataacaaagtttggtataataatgaaggatttttggatagtcattggcatttcagaaatagatttatagatagtttatatttcggcacagaataa

SEQ ID NO: 91 - protein, 545 aa, Candida albicans
MPLHPKSRRRSSRISPLPDEDSLSFINSSVENLDQSPFESIDDLVEDVNKYDLKSPSDEQQQQQQQESQTQNLKHKPSFTSTPKASYIPPWTEPYIIGIAGNSGSGKTSISQKIIQDINQPWTVLLSFDNFYQPLTSEQSKLAFANNYDFDCPDSLDFDLLVETIGNLKKGGKTTIPVYSFTSHNRTSKTNTIYGANVIIVEGLYALHDQQLLDMMDLKIYVDTDLDICLARRLTRDILYRGRDLGGAMQQWEKFVKPNAVKFINPTVQNADLVIPRGLDNSIAINLMIKHIKNQLALKSRNHLQRLKKLGVNIKFDIDKFNIKLLQNTNQVKGINSILFDTSTSRNDFIFYFNRMCGLLIELAQEFMTNYTNVDIDTGKGIYHGKKLLQNQYNAVNIIRSGDCFMASIKKSFPEISIGKLLIQSDSTTGEPQLHFERLPHKLSDKIMLFDSQIISGAGAIMAIQVLLDHHVKEQDIILITYLSTEIGIRRIVNVFPKVKIVVGKLSSMEDSNSNNKVWYNNEGFLDSHWHFRNRFIDSLYFGTE

SEQ ID NO: 92 - DNA, 1638 bp, Candida albicans
atgccactacatccgaaatcaagaagaagatcaagtagaatatccccacttcctgacgaggattcattgtcatttataaactcatcagtagaaaaccttgatcaatcaccatttgaaagtatcgatgatttagttgaagatgtcaacaaatatgacttgaaaagtcccagtgatgagcaacaacaacaacagcaacaagagctgcaaactcaaaatttgaaacacaaaccatcattcacttctacaccgaaagcatcatatatcccaccatggaccgaaccatatatcattggtattgctggtaattcagggtcagggaaaacctcaatctcccaaaaaatcattcaggatatcaatcagccatggacggtattattatcatttgataatttctatcaaccattgaccctggaacaaagtaaacttgcatttgccaataattatgattttgattgtcctgattcattagattttgatttattagtagagaccattggtaatttgaaaaaaggaggtaaaactactatcccagtttattcattcacttcacataatcgtacttcaaaaactaataccatttatggcgccaatgttattattgtggaaggtttatatgctttacatgatcaacaattattggatatgatggatttgaaaatatatgtcgatacagatttggatatttgtttagcaagaagattaactcgagatatattgtatcgtggtcgagatttaggtggagctatgcaacaatgggagaaatttgttaagccaaatgcggttaaattcattaatccaacggtacaaaatgctgatttggtgattcctcgaggattagataattcaattgccataaatttaatgattaaacatatcaagaatcaattagcattaaaatcaagaaatcatttacaacgattgaagaaattaggggtcaatataaaatttgatattgataaattcaatattaaattattacaaaacactaatcaagtcaagggaatcaattcgattttatttgatacgtcaacttcacgtaatgatttcatattttattttaatcgtatgtgtggattattaattgaattagcacaagagtttatgaccaattataccaatgtcgatatcgatactggtaaagggatttatcatggtaaaaaattattacaaaatcaatataatgcagtcaatataattcgtagtggagattgcttcatggcatcaattaaaaaatcattcccagaaatttctattggtaaattattaattcaaagtgattcaactacgggggaacctcaattacattttgaaagattgcctcataaattatctgataaaattatgttatttgattcacaaattattagtggagccggagccattatggcaattcaagtattattagatcatcatgttaaagaacaagatattattttaattacgtatttatctacagaaattggtataagaagaattgttaatgtgttccctaaagtgaaaattgttgttgggaaattatcaagtatggaagattcaaattcgaataacaaagtttggtataataatgaaggatttttggatagtcattggcatttcagaaatagatttatagatagtttatatttcggcacagaataa

SEQ ID NO: 93 - protein, 545 aa, Candida albicans
MPLHPKSRRRSSRISPLPDEDSLSFINSSVENLDQSPFESIDDLVEDVNKYDLKSPSDEQQQQQQQESQTQNLKHKPSFTSTPKASYIPPWTEPYIIGIAGNSGSGKTSISQKIIQDINQPWTVLLSFDNFYQPLTSEQSKLAFANNYDFDCPDSLDFDLLVETIGNLKKGGKTTIPVYSFTSHNRTSKTNTIYGANVIIVEGLYALHDQQLLDMMDLKIYVDTDLDICLARRLTRDILYRGRDLGGAMQQWEKFVKPNAVKFINPTVQNADLVIPRGLDNSIAINLMIKHIKNQLALKSRNHLQRLKKLGVNIKFDIDKFNIKLLQNTNQVKGINSILFDTSTSRNDFIFYFNRMCGLLIELAQEFMTNYTNVDIDTGKGIYHGKKLLQNQYNAVNIIRSGDCFMASIKKSFPVISIGKLLIQSDSTTGEPQLHFERLPHKLSDKIMLFDSQIISGAGAIMAIQVLLDHHVKEQDIILITYLSTEIGIRRIVNVFPKVKIVVGKLSSMEDSNSNNKVWYNNEGFLDSHWHFRNRFIDSLYFGTE

SEQ ID NO: 94 - DNA, 1638 bp, Candida albicans
atgccactacatccgaaatcaagaagaagatcaagtagaatatccccacttcctgacgaggattcattgtcatttataaactcatcagtagaaaaccttgatcaatcaccatttgaaagtatcgatgatttagttgaagatgtcaacaaatatgacttgaaaagtcccagtgatgagcaacaacaacaacagcaacaagagctgcaaactcaaaatttgaaacacaaaccatcattcacttctacaccgaaagcatcatatatcccaccatggaccgaaccatatatcattggtattgctggtaattcagggtcagggaaaacctcaatctcccaaaaaatcattcaggatatcaatcagccatggacggtattattatcatttgataatttctatcaaccattgaccctggaacaaagtaaacttgcatttgccaataattatgattttgattgtcctgattcattagattttgatttattagtagagaccattggtaatttgaaaaaaggaggtaaaactactatcccagtttattcattcacttcacataatcgtacttcaaaaactaataccatttatggcgccaatgttattattgtggaaggtttatatgctttacatgatcaacaattattggatatgatggatttgaaaatatatgtcgatacagatttggatatttgtttagcaagaagattaactcgagatatattgtatcgtggtcgagatttaggtggagctatgcaacaatgggagaaatttgttaagccaaatgcggttaaattcattaatccaacggtacaaaatgctgatttggtgattcctcgaggattagataattcaattgccataaatttaatgattaaacatatcaagaatcaattagcattaaaatcaagaaatcatttacaacgattgaagaaattaggggtcaatataaaatttgatattgataaattcaatattaaattattacaaaacactaatcaagtcaagggaatcaattcgattttatttgatacgtcaacttcacgtaatgatttcatattttattttaatcgtatgtgtggattattaattgaattagcacaagagtttatgaccaattataccaatgtcgatatcgatactggtaaagggatttatcatggtaaaaaattattacaaaatcaatataatgcagtcaatataattcgtagtggagattgcttcatggcatcaattaaaaaatcattcccagtaatttctattggtaaattattaattcaaagtgattcaactacgggggaacctcaattacattttgaaagattgcctcataaattatctgataaaattatgttatttgattcacaaattattagtggagccggagccattatggcaattcaagtattattagatcatcatgttaaagaacaagatattattttaattacgtatttatctacagaaattggtataagaagaattgttaatgtgttccctaaagtgaaaattgttgttgggaaattatcaagtatggaagattcaaattcgaataacaaagtttggtataataatgaaggatttttggatagtcattggcatttcagaaatagatttatagatagtttatatttcggcacagaataa

SEQ ID NO: 95 - protein, 484 aa, Komagataella phaffii
MKQSRLRHTDTILLNSNSFCTSNDFNEPASGTHPQYIPPWTEPYIIGVAGTSGSGKTSVAKHIVKAINQPWTVVLSLDNFYKVLTPEQHILAEHAQYDLDSPTALDFDLMLRCIGDLKTGKPTQLPVYDFCTHSRTEKTTTIYGASVIVVEGLLALHHGQLLDLMDTKVFVDTDLDICMARRVKRDLIERGRDLEGILDQWDRHVKPNTIRYVIPSSKNADLILPRSTDNKIALDMIIRHINNQLEQKSLVHLKRLQELGQISNDETLMNRIARLPLTNQLKCISTILFDRETSRTEFIFYFDRVANMLIHLALEQVEFGPSQDEVLTPQYHCLTDAIRPLQSVVVVTMVRTGDVFMNSIRKTIPDVRVGKLLIQSDLITGEPQLHTKSLPPCEQTTKLLLFDAHIISGAAAIMGIQVLLDHGIEEGNIVIVSYLAEEAGLRRILNAFQKVTIIVGLSSGRMTSLLKEPMFRTRFIDDYYFGST

SEQ ID NO: 96 - DNA, 1455 bp, Komagataella phaffii
atgaaacaatcaagactgagacacactgataccatactcctcaattccaattctttttgtacttcaaacgacttcaacgagccagcatctggaactcatcctcaatatattccgccgtggacagagccctatattataggtgttgccggaacttcggggtctggtaagaccagtgttgctaagcatattgtgaaggcgatcaatcaaccctggacagttgtattgtctctggataacttctacaaggtattgaccccggagcaacatattttggcggagcatgcgcagtatgatcttgattcgccaaccgctttggattttgatttaatgcttcgttgtattggagaccttaaaactggaaaaccaacgcaactgccggtgtatgacttttgcacccattcccgtacagaaaagactacaacgatttatggagcatctgtcattgtggttgaaggtttgttggcactccatcatggacaattactggatttaatggatacaaaagtttttgtggatactgatctagatatatgcatggctcgaagggttaaaagagacctgattgaaaggggtagagatttggaaggcatccttgaccagtgggatcgacatgtgaagcctaacacaattcggtatgtgatccccagctccaagaatgcggatttgatcctacctcgcagcactgataataaaattgcacttgatatgattattcgccatatcaataaccagttggaacaaaagtcattggttcacctgaaaagacttcaagagctggggcagatatctaacgatgagactctcatgaaccggatagcacgtttgccgctaacaaatcagttaaaatgtataagtaccattctttttgacagagaaacttctcgtacagagttcatcttttactttgatcgggttgctaacatgctgatccatctggcattggaacaggtagagttcggaccctcgcaagatgaggtattgaccccgcaataccattgcctaactgatgcgatacgaccgttacaatcggttgtcgttgtgactatggtacggacaggtgatgtatttatgaattcaatcagaaaaactattccagatgtaagagttggtaagttgctaattcaatcagacctaattacaggcgaacctcaattgcatacaaagtcgctgcctccatgtgaacaaactaccaagctactattattcgatgcgcacattatatcgggggccgcagcaattatgggcattcaagtacttctggaccatggtattgaagaaggtaatatcgtgattgtaagttatcttgcagaagaagctggcctacgtcgcatactgaacgctttccaaaaggttactattatcgtaggcttatcctctgggaggatgacctcattattgaaagagccaatgtttcgtacacggttcatcgacgattactacttcggcagtacgtag

SEQ ID NO: 97 - protein, 501 aa, Saccharomyces cerevisiae
MSHRIAPSKERSSSFISILDDETRDTLKANAVMDGEVDVKKTKGKSSRYIPPWTTPYIIGIGGASGSGKTSVAAKIVSSINVPWTVLISLDNFYNPLGPEDRARAFKNEYDFDEPNAINLDLAYKCILNLKEGKRTNIPVYSFVHHNRVPDKNIVIYGASVVVIEGIYALYDRRLLDLMDLKIYVDADLDVCLARRLSRDIVSRGRDLDGCIQQWEKFVKPNAVKFVKPTMKNADAIIPSMSDNATAVNLIINHIKSKLELKSNEHLRELIKLGSSPSQDVLNRNIIHELPPTNQVLSLHTMLLNKNLNCADFVFYFDRLATILLSWALDDIPVAHTNIITPGEHTMENVIACQFDQVTAVNIIRSGDCFMKSLRKTIPNITIGKLLIQSDSQTGEPQLHCEFLPPNIEKFGKVFLMEGQIISGAAMIMAIQVLLDHGIDLEKIRVVVYLATEVGIRRILNAFDNKVNIFAGMIISREKLQNHQYKWALTRFLDSKYFGCD

SEQ ID NO: 98 - DNA, 1506 bp, Saccharomyces cerevisiae
atgtcccatcgtatagcaccttccaaagaacgatcttcatcatttatttcaattttagacgatgaaacaagagacacattgaaagctaatgcagtcatggatggtgaagtagatgtcaaaaaaacaaaaggaaaaagctctcggtatatcccaccatggacaactccatatataataggtataggtggtgcttcaggttcaggcaagacaagcgttgctgctaagattgtgtcgtcaattaatgttccctggacagtattaatatctttggataacttttacaatccattaggcccagaggacagagccagagcctttaaaaatgaatacgatttcgacgagccaaatgccatcaacttagatttggcatataagtgcattttgaacttaaaggagggcaaaaggacaaatatcccagtttatagcttcgtccaccacaatagagttcctgataaaaatatagtcatatacggggccagtgtggtagttatcgaagggatctacgccctttacgatcgccgattgctggatttgatggacttgaaaatttatgttgacgctgatttggatgtctgcttagcaagaagattgtcgagagatatagtttccagagggagagatttggatggttgtattcaacaatgggagaaatttgtgaaaccaaatgcggtaaagtttgtgaaaccaacaatgaagaatgcagatgctatcattccatcgatgagtgataatgctacagcggtaaatttaatcattaaccacatcaagtcaaaactggaactaaaatcaaatgaacacttaagagagctaatcaaattgggctcttctccttcacaagatgtgcttaatcgtaacataattcatgaattgccgcccaccaaccaagttctttcgctgcatactatgcttctaaataaaaatctaaattgcgcggactttgttttctactttgacaggttagcaacaattttgttatcatgggcacttgatgacattcctgtagcacatacgaacataattacacctggtgagcataccatggaaaacgttattgcctgtcaattcgatcaagttacagctgttaatattattcgatctggcgattgttttatgaagtctttgagaaagacgatccccaatatcacaattggtaaattgttgattcagtccgattcacaaactggggaacctcaactgcattgcgaatttttacccccaaatatagaaaagtttggcaaggttttcttaatggaaggtcaaatcataagtggtgcggccatgatcatggccatccaggtgcttttagatcatggtattgatttggaaaagattagggtggtggtttatttggccactgaagttggtatccgacgtatattaaatgcatttgataacaaagtcaacatttttgctggtatgatcatctccagagaaaagttacaaaatcatcaatacaaatgggcattgaccagatttcttgattcaaagtattttggttgtgattga

SEQ ID NO: 99 - protein, 578 aa, Cryptococcus neoformans var. grubii
MEQTAGHRTPRTQQHFVYDPTQPQSKNQVLISHGRAPWYGPDGRNVEAYVVGIAGGSASGKTSVARAILSALNYIPTVLILSQDSFYNAHSPEEVELAFKNDLDLDHPDAIDMTLFAQCIKDLKQGKATEIPVYSFHHHQRMSEKKYIYGASVIIVEGIMALQSAELRELYDLKVFVNCDSDLMLARRIKRDVKERGRDVEGILDQYLRFVKSSYDTFVQPSSRYADIIVPGSSNQLAIELLVSHIKRQLESRSLRFRRVLADIGENRGSSTPSVEKFDKQIVLLEQRNQLRGIMTILRDRTTCREEFIFHIDRLSTIIVEKALTLVPCEPKVVKTPNKNIYKGISQTNNLVGVSILRSGLPFSQGLRRVIRDVPIGGILIQSDPKTGEPLLLKSDLPHCLRSRETNGDVRCLLLDSQMGTGAAAMMAIRVLLDHGISQDRIIFLTYLISRSASYSVLRAFPNIQIVTAAIDPGLDEVKIPYMPGSLIMGEAAGEGDFAVRLVDQLGHEEDKKGDRVKDLLKTDEEMAADGFKMNILKGTEELKFSRKHKRTHSPTGEKRAWVISPGMGHVGDRYYLV

SEQ ID NO: 100 - DNA, 2596 bp, Cryptococcus neoformans var. grubii
tcttgctttatttcgctgcagcgagcagtaactgttcagaagtgcttcagactcgtcgaggggccacacggaacagtcagcatggaacagacagcaggacatcgcactcccaggacacaacaacactttgtctacgaccccacgcagccgcagagcaaaaaccaagtcctgatatcccacggacgggcaccctggtacggccctgatggcaggaatgtcgaggcatatgtggtgggtattgccggaggtagtgcgtccggaaaggtacgtccgcagtcttgaaatgatatgcgtctttctaagcaattttggcagacttctgtagcgcgggctatcctttcggccctcaactacatccccacggttcttatcttgtcccaggattcgttttacaatgctcattccccagaagaggtcgaactggctttcaaaaatgacttggacctcggtgcgcgaaatcagttgcgagaaagatttatgaagacactcaattatgtactttatcagaccatcccgacgccattgacatgacgctgtttgctcaggtgcgacaaatgatcaataattattataaaggaaaggctgattttcccttggtagtgcatcaaggacctgaagcaaggcaaagctacagaggtgcgtccaactcagccgtccctacagtatgttgtcttacaggacgtgtcagatcccggtctactctttccatcatcatcgtttgtatttcctgatctctagttggctatcaactgatgaacactgtagaacgtatgtcggagaaaaagtatatctacggtgcaagcgtcatcattgtgtgagttacccttttttgtcgagaaacagttctaatataatttcagtgaaggtatcatggctcttcaatcagcagagttgagggaattgtacgacctcaaggtctttgtggtatggtatatctggtactaccattggggttacttttaattcaggcgttgtagaattgcgattcagatctcatgctagcaagacgtataaagcgagatgtcaaggaacgagggagggatgtggaaggcatcctcgaccaggcaagtgactcttcgacctcttccatcatcagatcaaactcaatccctattcctagtatttgaggtttgttaaaagcagctacgatacttttgtccagccttcatcgcgctatgcggatattgtaaatctttcccatcttttctacctaattttgtcctcatcaaccttgtcagattgttccggggtcctcaaatcaacttgccatcgaacttcttgtctcgcatatcaagcgccaactcgagtctcgttctcttcggttccgtagagtgttagccgatatcggtgaaaaccgcggcagcagcactccttcagtagaaaagtttgacaagcagattgttttgcttgagcaaagaaaccagctgcgcgtacgtgttgtgataaattccaacccgcaaaaccatttctgactgtctatttccagggtatcatgacaatacttcgagatcgtaccacgtgccgagaggaattcatcttccatatcgatcgtttatcaaccattatcgtggagaaggcccttacacttgttccgtgtgagcctaaggttgtgaaaacaccgaataaaaatatttacaaaggtatttcccagaccaacgtaagtagttcctctttgacagattcctgtgtgctcatcgcccgtagaatcttgtgggggtatccattcttcgctctggtcttccgttctctcaaggtcttcgtcgggtcatccgtgatgtgcctatcggtggtatactcatccagtccgatcccaagactggtgagccattgttattgaaaagtgatctccctcattgcttgaggtcacgggagacaaatggagacgtcaggtgtttgctgttggattcgcagatgggtacgggagcggctgctagtgagttctatcctccgtccagtataaacatgaagttaatatacgaggggatattagtgatggccatccgcgtactccttgaccatggcatttctcaggatcgtatcatattcctcacatacctcatttctcgctcagcctcatattctgtcctgcgcgcattcccgaatattcaaattgttaccgcagctattgaccctggcctggacgaggtgaagattccctacatgccaggaagtcttattatgggcgaggcggctggtgagggtgatttcgcggtaaggttagtcgatcagttgggacatgaagaggacaagaaaggggatagggtgaaagatctgttgaagacggatgaagagatggctgcggatggttttaagatgaacattttgaagggcacagaggaattgaaattttctagaaagcataaaaggacacattcaccaacaggagagaaaagggcatgggtcatttctccaggtgagtatgctattgtcatcggtattaaaggccacgctcatcagcagtaaaggcatgggccatgttggagatcggtattacctggtttgatccaactttacccacaatcactcgtcgctgttatattatctgtcttcatcatatgtgtcccatgtatatgtatggtgttggctgtatcagtcc

SEQ ID NO: 101 - protein, 330 aa, Rhizopus delemar
MIGLGTSGSASGKTSVAERVLKNLSVPWVVILSMDSFYNILTPEQSKLAHQSRFDFDHPSAFDYDLLFDTLKKLKEGKSVTVPIYNFSTHAREEKTTTIYGANVIIFEGIFALYDKRIRDMMDVKVFVDTDADIQLARRLQRDTLYRGRDVEGILDIIPRGLENVIAIDLMTKHIQTQLNENVINFRFGLLDTPVNEEIPSNVHVLPGTNQIKGIHTILRDCKTERDEFVFYADRLAVLLMEYAINLLPSVPLTVTTPINEIYQGDATIGKLLIQTDPNTGDPELHYCKLPKDVCDYNIVLMDAMVGTGAAALMAIRVLLDHEVPEHKSD

SEQ ID NO: 102 - DNA, 1820 bp, Rhizopus delemar
atgataggccttggtaccagtaagttttccactacccatcatatagacaaaaaaaaaaaactagatctaaaatcagatatttagtcctgatggatcaattgctgatccttacttgattggcatcgcaggtttctattttaaacagcagtatttattattatgtactcatattacttaaaaggcggtagtgcaagtggaaagacaagtgttgcagagtaagaataaagacaagagaaaagaaaaaaaaaatgaataaatgattttttctgtgtagacgtgtcttgaaaaatcttagtgtaccttgggttgtaattttatctatggattcattttacaacatcttaacacccgaacaaagcaaattggctcaccaaaggtaaaatatataaaaaatacgagaagcgttatttatgtgttataattagtcgctttgattttgatcatccttctgcttttgattatgacttattatttgatactttaaaaaaattaaaagaagggtaaaaattcattatttagtaatagaagtgggaaattgatcgtatgattagaaaatcagtaactgtaccaatctataacttttctactcacgcaagagaagaaaagacaacgaccatttatggagccaatgtgattatatttgaaggtatttttgctttgtatgataaaaggattagagacatgatggatgtgaaggtacacactttttgcataatgtgtcgtacagaaattcatctttaacctaggtatttgtggacaccgatgcagatattcaattagcaagaagattacaaagagatactctttatagaggacgtgatgttgaaggcattttagatgtaaggttacttgtatatgtttatttgtatacttacatcacatagcaatatacgagatatgttaaaccttcttatgataactatgttagacctaccatgaaatttgctgatatggtatcttcatacactaacattatacaaatacatatatagattataactaatgtgttttataatagattattccaagaggtctagaaaatgtgattgccattgatttaatgacgaagcatattcagacgcaattaaatgaaaacgtcattaattttcgctttggtttgttagatacaccagtaaatgaagagataccaagcaacgtccatgtgctacctggaacaaaccagataaagggtattcataccattttaagagattgtaaaacagaaagagatgaatttgtattttatgctgatcgactggctgtgttattaatggaatagtaaaataatattaattaaacaatttatatttaaaactaacaaaacaaaattttgtatagtgcaattaatttgcttccttctgtgcccttaacagtaactacacctattaatgaaatctatcaagggttaaaatacagtcaaaaggtttctactactatccttgtcatattattcaatgctaatcttgcaagtagatctgcggtgtttctattttacgcgctggtggaaccatggaagctggtttaaaacgtgtatttagtgatgcgacaattggtaaattgttgattcagacagatccaaatactggggacccggaacttcactactgcaaactacccaaagatgtatgcgactacaatattgtcttgatggatgctatggtaggtacaggagctgctgcactcatggccatccgtgtcttgctggaccatgaagtaccagaggtaaataaatgccattcagtcttttcgtttattaatatgtacccaattataggatcgaatcatctttgtatcttttcttgcagcacaaatcggactga

SEQ ID NO: 103 - DNA, 20 bp, Artificial sequence
  Oligo fcyB-1
cgctatcccagcaatagagc

SEQ ID NO: 104 - DNA, 40 bp, Artificial sequence
  Oligo fcyB-2
tagttctgttaccgagccggactgagtcaatccccaccac

SEQ ID NO: 105 - DNA, 40 bp, Artificial Sequence
  Oligo fcyB-3
gctctgaacgatatgctccctgcggtttttgggttttatc

SEQ ID NO: 106 - DNA, 20 bp, Artificial sequence
  Oligo fcyB-4
cacactgggtctgaagacga

SEQ ID NO: 107 - DNA, 20 bp, Artificial sequence
  Oligo fcyB-N1
cagagaattgccaagctggt

SEQ ID NO: 108 - DNA, 20 bp, Artificial sequence
  Oligo fcyB-N2
gcggtatgaaacaacggtct

SEQ ID NO: 109 - DNA, 41 bp, Artificial sequence
  Oligo P1 (reporter cassette)
ccggctcggtaacagaactactgatgcgagcaacagtatgc

SEQ ID NO: 110 - DNA, 42 bp, Artificial sequence
  Oligo P2 (reporter cassette)
gggagcatatcgttcagagctgagggttgagtacgagattgg

SEQ ID NO: 111 - DNA, 20 bp, Artificial sequence
  Oligo uprt-1
ggaaggacaggtacgccata

SEQ ID NO: 112 - DNA, 40 bp, Artificial sequence
  Oligo uprt-2
tagttctgttaccgagccggcggagcactctgaaaattgg

SEQ ID NO: 113 - DNA, 40 bp, Artificial sequence
  Oligo uprt-3
gctctgaacgatatgctccctcccatcgtgtagcgacata

SEQ ID NO: 114 - DNA, 20 bp, Artificial sequence
  Oligo uprt-4
tactaccttcgccctctgga

SEQ ID NO: 115 - DNA, 20 bp, Artificial sequence
  Oligo uprt-N1
tttgagcgattaaggtgcaa

SEQ ID NO: 116 - DNA, 20 bp, Artificial sequence
  Oligo uprt-N2
gccccactacttgtttccag

SEQ ID NO: 117 - DNA, 20 bp, Artificial sequence
  Oligo cntA-1
actggggctttttctggact

SEQ ID NO: 118 - DNA, 40 bp, Artificial sequence
  Oligo cntA-2
tagttctgttaccgagccggttaagaacgcgacgaccttt

SEQ ID NO: 119 - DNA, 40 bp, Artificial sequence
  Oligo cntA-3
gctctgaacgatatgctccctgcctgcaaatcacaagaac

SEQ ID NO: 120 - DNA, 20 bp, Artificial sequence
  Oligo cntA-4
atacatcgtccacggagagc

SEQ ID NO: 121 - DNA, 20 bp, Artificial sequence
  Oligo cntA-N1
tttaacgcgacgacagaatg

SEQ ID NO: 122 - DNA, 20 bp, Artificial sequence
  Oligo cntA-N2
caaggtgggtggatttgtct

SEQ ID NO: 123 - DNA, 20 bp, Artificial sequence
  Oligo uk-1
ataggtggtagggcaggagg

SEQ ID NO: 124 - DNA, 40 bp, Artificial sequence
  Oligo uk-2
tagttctgttaccgagccggattagaatgcggcgcaacag

SEQ ID NO: 125 - DNA, 40 bp, Artificial sequence
  Oligo uk-3
gctctgaacgatatgctcccggtctatagtgtcaggcggc

SEQ ID NO: 126 - DNA, 20 bp, Artificial sequence
  Oligo uk-4
gccaaactcactcgggtaca

SEQ ID NO: 127 - DNA, 20 bp, Artificial sequence
  Oligo uk-N1
gccagaatgaatcgcagtgc

SEQ ID NO: 128 - DNA, 20 bp, Artificial sequence
  Oligo uk-N2
tgcgattcgtgacttctccc

SEQ ID NO: 129 - protein, 419 aa, Escherichia coli
MSQDNNFSQGPVPQSARKGVLALTFVMLGLTFFSASMWTGGTLGTGLSYHDFFLAVLIGNLLLGIYTSFLGYIGAKTGLTTHLLARFSFGVKGSWLPSLLLGGTQVGWFGVGVAMFAIPVGKATGLDINLLIAVSGLLMTVTVFFGISALTVLSVIAVPAIACLGGYSVWLAVNGMGGLDALKAVVPAQPLDFNVALALVVGSFISAGTLTADFVRFGRNAKLAVLVAMVAFFLGNSLMFIFGAAGAAALGMADISDVMIAQGLLLPAIVVLGLNIWTTNDNALYASGLGFANITGMSSKTLSVINGIIGTVCALWLYNNFVGWLTFLSAAIPPVGGVIIADYLMNRRRYEHFATTRMMSVNWVAILAVALGIAAGHWLPGIVPVNAVLGGALSYLILNPILNRKTTAAMTHVEANSVE

SEQ ID NO: 130 - DNA, 1260 bp, Escherichia coli
gtgtcgcaagataacaactttagccaggggccagtcccgcagtcggcgcggaaaggggtattggcattgacgttcgtcatgctgggattaaccttcttttccgccagtatgtggaccggcggcactctcggaaccggtcttagctatcatgatttcttcctcgcagttctcatcggtaatcttctcctcggtatttacacttcatttctcggttacattggcgcaaaaaccggcctgaccactcatcttcttgctcgcttctcgtttggtgttaaaggctcatggctgccttcactgctactgggcggaactcaggttggctggtttggcgtcggtgtggcgatgtttgccattccggtgggtaaggcaaccgggctggatattaatttgctgattgccgtttccggtttactgatgaccgtcaccgtcttttttggcatttcggcgctgacggttctttcggtgattgcggttccggctatcgcctgcctgggcggttattccgtgtggctggctgttaacggcatgggcggcctggacgcattaaaagcggtcgttcccgcacaaccgttagatttcaatgtcgcgctggcgctggttgtggggtcatttatcagtgcgggtacgctcaccgctgactttgtccggtttggtcgcaatgccaaactggcggtgctggtggcgatggtggcctttttcctcggcaactcgttgatgtttattttcggtgcagcgggcgctgcggcactgggcatggcggatatctctgatgtgatgattgctcagggcctgctgctgcctgcgattgtggtgctggggctgaatatctggaccaccaacgataacgcactctatgcgtcgggtttaggtttcgccaacattaccgggatgtcgagcaaaaccctttcggtaatcaacggtattatcggtacggtctgcgcattatggctgtataacaattttgtcggctggttgaccttcctttcggcagctattcctccagtgggtggcgtgatcatcgccgactatctgatgaaccgtcgccgctatgagcactttgcgaccacgcgtatgatgagtgtcaattgggtggcgattctggcggtcgccttggggattgctgcaggccactggttaccgggaattgttccggtcaacgcggtattaggtggcgcgctgagctatctgatccttaacccgattttgaatcgtaaaacgacagcagcaatgacgcatgtggaggctaacagtgtcgaataa

SEQ ID NO: 131 - protein, 208 aa, Escherichia coli
MKIVEVKHPLVKHKLGLMREQDISTKRFRELASEVGSLLTYEATADLETEKVTIEGWNGPVEIDQIKGKKITVVPILRAGLGMMDGVLENVPSARISVVGMYRNEETLEPVPYFQKLVSNIDERMALIVDPMLATGGSVIATIDLLKKAGCSSIKVLVLVAAPEGIAALEKAHPDVELYTASIDQGLNEHGYIIPGLGDAGDKIFGTK

SEQ ID NO: 132 - DNA, 627 bp, Escherichia coli
atgaagatcgtggaagtcaaacacccactcgtcaaacacaagctgggactgatgcgtgagcaagatatcagcaccaagcgctttcgcgaactcgcttccgaagtgggtagcctgctgacttacgaagcgaccgccgacctcgaaacggaaaaagtaactatcgaaggctggaacggcccggtagaaatcgaccagatcaaaggtaagaaaattaccgttgtgccaattctgcgtgcgggtcttggtatgatggacggtgtgctggaaaacgttccgagcgcgcgcatcagcgttgtcggtatgtaccgtaatgaagaaacgctggagccggtaccgtacttccagaaactggtttctaacatcgatgagcgtatggcgctgatcgttgacccaatgctggcaaccggtggttccgttatcgcgaccatcgacctgctgaaaaaagcgggctgcagcagcatcaaagttctggtgctggtagctgcgccagaaggtatcgctgcgctggaaaaagcgcacccggacgtcgaactgtataccgcatcgattgatcagggactgaacgagcacggatacattattccgggcctcggcgatgccggtgacaaaatctttggtacgaaataa

SEQ ID NO: 133 - protein, 486 aa, A. thaliana
MPEDSSSLDYAMEKASGPHFSGLRFDGLLSSSPPNSSVVSSLRSAVSSSSPSSSDPEAPKQPFIIGVSGGTASGKTTVCDMIIQQLHDHRVVLVNQDSFYRGLTSEELQRVQEYNFDHPDAFDTEQLLHCAETLKSGQPYQVPIYDFKTHQRRSDTFRQVNASDVIILEGILVFHDSRVRNLMNMKIFVDTDADVRLARRIRRDTVERGRDVNSVLEQYAKFVKPAFDDFVLPSKKYADVIIPRGGDNHVAVDLITQHIHTKLGQHDLCKIYPNVYVIQSTFQIRGMHTLIREKDISKHDFVFYSDRLIRLVVEHGLGHLPFTEKQVVTPTGAVYTGVDFCKKLCGVSIIRSGESMENALRACCKGIKIGKILIHRDGDNGKQLIYEKLPHDISERHVLLLDPVLATGNSANQAIELLIQKGVPEAHIIFLNLISAPEGIHCVCKRFPALKIVTSEIDQCLNQEFRVIPGLGEFGDRYFGTDEEDQ

SEQ ID NO: 134 - DNA, 3854 bp, A. thaliana
gccaaatattaaaataaaatctaatataataagatttgttcctatcccaaatcctaaatgtaagtacaacacattatttataataaacaagacagaggtctgatatttgcgttgaacagaatcgttgtcacttgttcagtcttcactacggagctgttttttttcgattcgccggaaaaatcataaaatccaaatctacaaccacctttttatcgcaatccaatgccggaagattcttcttctttagactacgcgatggagaaggcatcagggcctcacttctccggtcttcgcttcgacggccttctctcttcttctccacccaattcctccgtcgtctcttcactccgatcagctgtttcttcttcttctccgtcttcttccgatcccgaagcgcctaagcagcccttcattatcggtgatttctcaaaccccatttcgctctagcgttgtttaggttttatcaaatttgggaaattgggttctgtctctcttgatttcgagagtttcttctgcattaacttcgttggaatctagggttttgatgatagaacctctgagtatggtagcattttttgtgctcctgagatttaacagaactaatttggtttcaattgtattaaagtatcgtttttttttttttttgacgttttgtttgatttgaccttttgacaggggtttctggtggtacggcttctggtaagaccacggtctgtgacatgataatccagcaacttcatgaccatcgtgtcgttttagttaatcaggtttgattctattgcttcaatcttgttctcaatgtcccactttattctgtatgagattcacatcttataatgtttgatatcttttgttgattaggattccttttatcgtggtttgacatctgaggagttgcagcgtgtgcaagagtataactttgatcatcctggtaaactaacttaatcgtttttcttgaattttcttctcactgcttgcttgtattttggttttaagtacactgcggttcagctgacagcagcatttggtattcttttgcagatgcgtttgacactgagcagcttttgcattgtgctgagactctcaagagcgggcaaccctatcaagttccaatctatgactttaagactcatcaacgtagatctgatactttccgccaggtgcttactcttatgtccaagaccattattgtggttcttgtgctcatgtgatttgaatctcttatttatgtgttcctattttaacttaatgggcatattagcatatattcattccctttaaacttaatatctgttgggagcctctgtcaggtcaatgcttctgatgtaataatattggaagggattctagttttccatgactcacgagttcggaatctgatgaatatgaagatctttgttgatacaggtatcaaccgctatgcctttttgtttccccaaaacatgcatatgtcttgcgctactcatcctttttcactctttggataaccttattagttttgtatacctggaaatgatattttgtaatcaaagttgtctatctccttggaatctttttcacttctctctagtcctcaacataagctattaaatggtttcagatgctgatgtaaggcttgctcgcagaattaggcgtgacacagttgagaggggtagggatgtcaattctgtgcttgaacaggtcagtcctcttttcgatttatgctgtaggatttgtaagcataggttcatctaacctagttaggtagctttattttgtgtgtgtatatgagacatgagtaaatacatggaaattctgctctagggaacatctcattgtgtctcttctctggtttcttcttccttttctgcttcctactgttgtttttttactagataactctatatgttcttattccatcctttgagatgctcctctgtaaaacatctcttttatcttctgagcttcccatgtgcctaatttgttttctgaggatgttatgttcgtttacagtatgcaaagtttgtgaagccggcatttgatgacttcgtgctcccttcaaagaaatatgctgacgtgatcattcctcgaggaggtgataatcacgttgcagtcgatttgattacgcaacatatccacacaaaacttgggcagcatgatctctgcaaaatctacccaaatgtttatgttatccagtcaacatttcaggtattttctcactcttgctctctttgatacttgatttagatggtaagtgttatcgatggtctcactataggatcatgattgcagataagaggcatgcatacacttattcgggaaaaggacatatcaaagcatgactttgtgttttattcagatagactcattcgtctggtatatctctcccttatgtcttctctttcaacgtcactcacttttcaggctttcttattttaactaatatcagttcaatttcaggtcgtggagcatggtcttggtcatttgccattcactgagaaacaagtagttactccaacaggtatgagaagagaatgcatgagctgtaacaatactgtatacacttttttatataacttggtcatcagatgtccgtctccttcacgtcaatctagttagagttagcttattcaactcatttaaacttatcttttccacaccaggagctgtatataccggtgttgatttctgcaagaaactttgtggggtctcaattattagaaggtgagcctttgaaaggtattgtatttggttaagtttgatatgataagatgcttattcttccaactgttttacatcatgcaaggcataaatttacttgtttgcgattaaattatctctcctacgtggtttctgttttgtatgtttttcctagtggtgaaagcatggaaaatgcattacgcgcttgctgcaaaggaattaaaatagggaagattctcatccaccgtgatggcgataatggaaaacaggtcttcctattaatattttgctctgtttttaaaagtatcaagtataatctctttttataaacacgaattttgaagtttctcttgtcgtgataatcagcttatttatgagaagcttcctcacgacatatctgaacgccatgtcctgcttctagatcctgtcttagccacaggtactctgtctctctacgctgtatatttcgattttggtcctttttgatggatttttggttctcgaaattcatatctcgtgatattgcttcttcttgaaggtaactcggctaatcaagccattgaactactcatacagaaaggtgttcctgaagctcacattatattcctcaaccttatatcggtgagtgtaaaaagctatatatcgatctttcgctcttttgtgaatgtagaagctaaagatctcattatgtatctgcttctctatctctttgcttctctctttccctcgtcatgaccttgctttctgacaggcgccggagggaatccactgtgtctgcaaacgttttccagcattgaaaattgtgacgtctgaaatagaccaatgtctgaaccaagaattcagagttataccgggcttaggcgagtttggcgatcgttacttcggcaccgacgaggaagaccagtagccaccactcaacactgtgactggtttcaaaggaaaagcctaaatttatgactagagcgacagtagaggcacttgcatgtctttgtagtttgtgctaaagaatctttatcttattgtttatgaagctcctcttgcttacttgaattatttttgaaaaactacgttaattttctcattaaaaaaatgatcgggacaatgctagttttctaaaccggtggaaaaagacataaccaatccttttatacatgattttgacaatcgtacgaaaa

SEQ ID NO: 135 - protein, 148 aa, Aspergillus fumigatus
METDAGFIAALEEAKKGAAEGGVPIGAALVSKDGKILGRGHNMRVQKGSATLHAEMSALENSGRLPASAYEGATMYTTLSPCDMCTGACILYKVKRVVIGENKNFMGGEEYLLNRGKEVVVLDNEECKQLMEKFIKEKPELWNEDIAV

SEQ ID NO: 136 - DNA, 652 bp, Aspergillus fumigatus
atggaaacagacgctggcttcatcgccgccttggaggaggcaaagaaaggcgccgctgaaggtggagtccctatcggtgcagctctggtgtccaaggatggcaagattctcggccgtggacacaatatgcgcgttcagaaaggaagcgccaccttgcatgttagtcccaattgatacagtttacttctgttttggatcgctgtcgatcattcctatacgcgctggcattgcacatggcgcaagcgaagctcacattcgactcatctgtaggctgagatgtctgctctcgagaattccggccgtcttcccgcgtccgcctacgagggtgcgactatgtacaccacgctgtctccatgcgacatgtgcacgggtgcctgcatactctacaaggttaagcgggttgtcatcggggagaacaagaacttcatgggcggcgaggagtatcttctaaatcggggtaaagaagttgtagtgctggataatgaagagtgcaagcaactgatggagaagtttatcaaggagaagccggagctttggtacgtctttcagtctcaactctggttttcttgcggcatcatcccgctgctactctcttgcagttgaagatattgatactaattgccattgtaggaatgaggacattgcagtctaa

SEQ ID NO: 137 - protein, 148 aa, Aspergillus niger
METDPGFIAAVEEAKQGAAEGGVPIGACLVSKDGKILGRGHNMRVQKGSPVLHAEMSALENSGRLPASAYEGATMYTTLSPCDMCTGACILYKVKRVVVGENKSFMGGEDYLKSRGKEVVVLDNAECKQLMEKFMKEKPELWNEDISV

SEQ ID NO: 138 - DNA, 761 bp, Aspergillus niger
ctatatttcatatctcaatacagcatacaacaagcacataccatcatggagaccgatcccggattcatcgctgctgtggaagaagccaagcaaggcgctgctgagggtggtgtgcccattggagcttgtttggtctccaaggatggcaagattctaggccgcggccacaatatgcgcgtccagaagggtagtcccgtgttgcatgttcgttgatcccatcccttgccttctgagggtcgtctggggttctaattctaatctctaccgtcataggctgagatgtccgcgctcgagaactccggtcgtctgcccgcttcggcctacgaaggcgctactatgtacacgaccctgtcgccatgcgacatgtgcaccggtgcctgcatcctctacaaggttaagcgcgttgttgtgggcgagaacaagagcttcatgggtggcgaggactatcttaagagccgtgggaaggaggttgtggttttggataatgcagagtgtaagcagctgatggagaagttcatgaaggagaagccggagctttggtaggtttcccatgcatctcactggactggtctagtcttttgttggaatgtacgctgactgtacgatgtctttgcaggaatgaggacatttccgtctgagcttttgaattcgtgaaggtgtcaactatattgctggctaggctctcatgtacataataaagaattgaaagctagttctggtcgcattgagcacccaatttagaccgtcagacggtggatctc

SEQ ID NO: 139 - protein, 148 aa, Penicillium chrysogenum
MEQDPGFIAAVEEAKQGLSEGGVPIGAALVSKDGKILGRGHNMRVQKGSAVLHAEMSALENSGRLPASAYEGATMYTTLSPCDMCTGACILYKVKRVVIGENKNFMGGEELLLNKGKEVVVLDNAECKEFMTKFMKEKPELWNEDIAV

SEQ ID NO: 140 - DNA, 873 bp, Penicillium chrysogenum
atggaacaggatcccggattcattgccgctgtggaagaggcgaagcagggcctctcagagggaggtgtgccaatcggtgctgccctggtctcgaaggatggaaagatcctgggccgcggtcacaacatgcgcgtccagaagggaagcgctgtcttgcatgtcagtacagcgtgatatactccaattgataacaattgtcgatctatatctatatccactattatagcgttcatgaactaccgctaatacaaacaaggccgagatgtccgccctcgaaaactctggccgtcttcctgcctctgcttacgagggtgctactatgtacactaccctctcaccctgtgatatgtgtaccggtgcttgtatcctctacaaggtgaagcgggttgtcattggtgagaacaagaacttcatgggtggtgaggagcttctgctcaacaagggcaaggaggttgttgtcttggataacgccgagtgcaaggaatttatgaccaagttcatgaaggagaagcctgagctatggtatgtcatgtttggtgcttggtctatcgcaatacgtaaaacggctctcggtctccttgcagttgcatcaagaatttttggccgtttcatgatcatcaatactaactgtagtgtctgctatacaggaacgaggatatcgctgtctaaatttcataaggcctgcacatatcatagccttaaataccccgagaatagaaaatattcacgcgcaaaagttgcgtttctagatccaatgtagtcttcagatctgagtcggggaagaagccgactgattttaagtttcaggaaacagacacttatacggtcaagcggtggatttcttcaaagaagaacttgaggttctcggggtcg

SEQ ID NO: 141 - protein, 142 aa, Aspergillus oryzae
MESDPGFVAALEEAKQGYAEGGVPIGAALVSKDGKILGRGHNMRVQKGSATLHAEMSALENSGRLPASAYEGATMYTTLSPCDMCTGACILYKVKRVVIGENKSFMGGEEYLKNRGKELVVLNNEECKQLMEKFMKEKPELW

SEQ ID NO: 142 - DNA, 504 bp, Aspergillus oryzae
atggagtcagaccctgggtttgttgctgccctcgaggaggcaaagcaaggttacgccgagggtggagttcccatcggcgcggcgttggtctccaaagatggcaaaatccttggccgtggacataatatgcgcgttcagaaggggagtgcaacattacatgttagtcaatcatacaccagttctccaattcctaggtgattggacggatgtccgtggcttactttaacctaacaggccgagatgtctgctctggagaactcaggccgccttcctgcttcggcctatgaaggtgctaccatgtatacgaccttgtctccctgtgatatgtgcacgggtgcctgcatcctctataaggtgaagcgcgtggtcattggagagaacaagagcttcatgggtggggaagaatatctcaagaaccgtggcaaggaactggttgtcttgaacaacgaggagtgcaagcagttgatggagaaatttatgaaggagaagccggagctctggtaa

SEQ ID NO: 143 - protein, 151 aa, Komagataella phaffii
KMTFSDEDGIKLALKEAQKGYEDGGIPIGAALVSEDGTVLGVGHNLRIQKGSSVFHAEMSALENAGRLPGKTYKNCTMYTTLSPCHMCSGACLMYGIKRVVLGENETFQGAEELLRSKGVEVVNAKNDECKELISKFIKERPADWSEDIGE

SEQ ID NO: 144 - DNA, 453 bp, Komagataella phaffii
atgacattcagcgatgaagacggtattaaacttgccttgaaggaggctcaaaaaggttatgaagatggaggcattcctattggcgcagctctagtgtccgaagatggtactgttttaggcgttgggcataatttgagaattcaaaaaggttcgtcagtgtttcatgccgagatgtcagctcttgaaaatgccggaagattgccaggaaagacatacaagaactgcactatgtacactaccctgagcccttgccatatgtgtagtggggcttgtttgatgtacgggattaagagagttgtccttggagaaaatgaaactttccaaggggctgaagaattacttagatctaagggtgttgaagttgtcaatgccaaaaacgatgagtgcaaggagttaatttccaagttcatcaaggagaggccggcagattggtctgaggatattggtgagtaa

SEQ ID NO: 145 - protein, 150 aa, Candida albicans
MTFDDKKGLQIALDQAKKSYSEGGIPIGSCIISSDGTVLGQGHNERIQKHSAILHGEMSALENAGRLPGKTYKDCTIYTTLSPCSMCTGAILLYGFKRVVMGENVNFLGNEKLLIENGVEVVNLNDQECIDLMAKFIKEKPQDWNEDIGE

SEQ ID NO: 146 - DNA, 523 bp, Candida albicans
atgacgtttgacgacaaaaaaggtttacaaatcgctcttgatcaagccaagaaaagtatgtgtatatcatgcccactctgaactttcaattaaaatccagctttactaacacacttgctgtccaggttactcagaaggtgggatacctattggttcatgtattatttcatccgatggcacagtattaggccaaggtcacaacgaaagaatccaaaaacattcagctattttacatggagaaatgtcagcattagaaaacgcaggaagattaccaggaaaaacttataaagattgtaccatctatactactttatcaccatgtagtatgtgtactggggccattttattatatggattcaaaagagttgtgatgggagaaaatgtcaatttcttgggtaatgaaaagttattgattgaaaatggtgtcgaagttgtgaatttaaatgatcaagaatgtattgatttgatggccaaattcattaaagagaaaccacaagattggaatgaagatattggagaataa

SEQ ID NO: 147 - protein, 158 aa, Saccharomyces cerevisiae
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 148 - DNA, 477 bp, Saccharomyces cerevisiae
atggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtag

SEQ ID NO: 149 - protein, 165 aa, Cryptococcus neoformans
MSPVEGSPAKPEDYPHFMSVAHEQALKSLSEGGIPIGAALVHLPTSRIISRGHNNRVQLSSNVRHGEMDCLENLGRVPEGLLRDCAMFTTLSPCIMCSATCILYKIRTVVLAENENFLGGEQLLRDNGANVINLDCDEIKNMMKDWINSPGGKVWNEDIGEVTRS

SEQ ID NO: 150 - DNA, 2243 bp, Cryptococcus neoformans
agtcaaccgtcaaccgtcacgtacacacacaccttgtacagaaaaccaacggcgcacctttcttggcccaatagcaacatgtcccccgtagaaggatccccagccaaaccagaggactaccctcactttatgagcgtgtacgttcttattcccatttaagccccaggcggttcaatcacacacacacacaaacgaacgacaaccggtgggagagtgtagtgagagatatgtatagatgtactggttcaagagctgattaaagagctggtgaaaaaactcgattccatttgtagtgcccatgaacaagctttaaagagtctttcagagggcggtatccccattgggtaagccctgttttttttgtttacttatctacaacaagttaatcccgatcaactcgtcgtcatcaaacccctgccccccccccccccccccccgccgcacacgcacagcgccgctttggtccaccttcctacctcccgcatcatctcccgtggacacaacaaccgtgtccaactctcgtccaacgtccgacatggagagatggactgtctcgaaaacttgggacgggtgccagaggggttgttaagagattgtgcaatgtttaccaccctctcgccttgcatcatgtaaagtttacttctctctcactctctaaagtcgggctgatattttgatatttctaattataaaaaggtgctcggcgacttgtatcctgtacaaaatccgtaccgtcgtgctcgccgaaaacgaaaactttttgggcggggaacaactcctccgtgataacggcgccaacgtgatcaacctcgattgtgacgagatcaaaaacatgatgaaagactggatcaacagtcctggcgggaaagtgtggaacgaggatatcggcgaggtcacacgatcgtaaattgtgtgtgttacaacagacgagttatccatgaattttttttttatctactgctacaaaaggtatgaaaaaccgcttttttcaaaaaaaaaaaatatgccaatgttgtgtttagaattcggtctcacgttgcttcttggccgccgccaattctgtttttctctttttgaaaatcgaatggctatctttcccactactcgatccccctgccctcttattctgccgcacttctcttgacaactccttgctggaaagacgaaatccctgattagtttacattgcccctcagaaagaaaaaaagaagaaaaaaaaactcacttgacactcttgttctgttccctataactgatattctgccctccactcaaatgctccaaaggtgccatgaacctctgcaccgtcatcccactcgtactcggctgagcgcccgcaatcctcttgagatgcttacccccattcaacctcatataatggtccgccgtctccgctgcggcatcgccctggttcaatactgactcgagcaatgacagctgttcgtccttggaagggaaaacgaggttgggcatgctatgaagaccgtgaagttcggtgatcactttgaacgcgtagccttggtcaatcaagaacccttgccgctttgaggaatagaacatctcctgggtgtctttggaaacgagcgaataaaaaaatgcgttgaagccctcgtcattccttcgctttgccctcagaattctacccaatcgctgagcttcttgtcgtcgagaaccaaagtgggaagatatctgaatcaagcaagtagcttcaggcaagtcgatagaggtatcaccgaccttggagaggaagatggtgttcagttgggggtcgtgttggaatcgcgaaagaatccgtaatcgttcgccttcaggcgtcccgccgtggatgaaagatttgcccagctttttcgcatacgcctaattcgtcccgtcagtacaaattaaaattaaaattcctacaagacccaaactcacctcgagtgcaaacacattgtcggaaaatacgatcaccttgtcgcctcggctctcatgataattgatcaagaactgacatgcttggatcttgttcgggttcatggcgtgcaagaggatgcgtttgcgagaaggattccgtaaatattcgcgataaaattctggagtcatggggcaccaaacttcggcacactggtcttgttagtaatgtatggaaaaaaggaaaaaaagttggacaatgaaaacatacctggacggtggcaatatgaccatttttagcgagatccatccaattggcttcgtacaactttggaccgatcaagtatcccaaa

SEQ ID NO: 151 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 152 - DNA, 1284 bp, Escherichia coli
gtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttga

SEQ ID NO: 153 - DNA, 41 bp, Artificial Sequence; identical to SEQ ID NO: 109
  Oligo P1

SEQ ID NO: 154 - DNA, 42 bp, Artificial Sequence; identical to SEQ ID NO: 110
  Oligo P2

SEQ ID NO: 155 - DNA, 40 bp, Artificial Sequence
  Oligo hph-FW
ccggctcggtaacagaactaacggcgtaaccaaaagtcac

SEQ ID NO: 156 - DNA, 40 bp, Artificial Sequence
  Oligo hph-RV
gggagcatatcgttcagagctcttgacgaccgttgatctg

SEQ ID NO: 157 - DNA, 45 bp, Artificial Sequence
  Oligo FoGFP-FW
gttgtaggggctgtattaggtctcggctgttgttagtgttcgagg

SEQ ID NO: 158 - DNA, 43 bp, Artificial Sequence
  Oligo FoGFP- RV
gagtcgtttacccagaatgcacagggaaggaatcagcgcaaag

SEQ ID NO: 159 - DNA, 40 bp, Artificial Sequence
  Oligo 5′ fcyB-FW
tgtggcggccgcgtttaaaccgctatcccagcaatagagc

SEQ ID NO: 160 - DNA, 40 bp, Artificial Sequence
  Oligo 5′ fcyB-RV
ttacgccaagcttgcatgccactgagtcaatccccaccac

SEQ ID NO: 161 - DNA, 40 bp, Artificial Sequence
  Oligo 3′ fcyB-FW
agtgaattcgagctcggtactgcggtttttgggttttatc

SEQ ID NO: 162 - DNA, 40 bp, Artificial Sequence
  Oligo 3′ fcyB RV
agcggtttaaacgcggccgccacactgggtctgaagacga

SEQ ID NO: 163 - DNA, 24 bp, Artificial Sequence
  Oligo BB-pfcyB-FW
tgtgaaattgttatccgctcacaa

SEQ ID NO: 164 - DNA, 24 bp, Artificial Sequence
  Oligo BB-pfcyB RV
aaacagctatgaccatgattacgc

SEQ ID NO: 165 - DNA, 40 bp, Artificial Sequence
  Oligo PcFrag1-FW
aatcatggtcatagctgtttaaaggggagagagcgaaaag

SEQ ID NO: 166 - DNA, 20 bp, Artificial Sequence
  Oligo PcFrag1-RV
gcatggggacaatctcactt

SEQ ID NO: 167 - DNA, 22 bp, Artificial Sequence
  Oligo PcFrag2-FW
aagtgagattgtccccatgcag

SEQ ID NO: 168 - DNA, 40 bp, Artificial Sequence
  Oligo PcFrag2-RV
gagcggataacaatttcacacgcgtgatatcctgtcttca

SEQ ID NO: 169 - DNA, 20 bp, Artificial Sequence
  Oligo Pc-fcyA-1
tgaccttgatggcatctgaa

SEQ ID NO: 170 - DNA, 40 bp, Artificial Sequence
  Oligo Pc-fcyA-2
tagttctgttaccgagccggtcagtgcgggctacagagta

SEQ ID NO: 171 - DNA, 40 bp, Artificial Sequence
  Oligo Pc-fcyA-3
gctctgaacgatatgctcccggcctgcacatatcatagcc

SEQ ID NO: 172 - DNA, 20 bp, Artificial Sequence
  Oligo Pc-fcyA-4
agccgtaaaattcgcatcac

SEQ ID NO: 173 - DNA, 20 bp, Artificial Sequence
  Oligo Pc-fcyA-N1
gtcgaggtgctcaatgtgaa

SEQ ID NO: 174 - DNA, 20 bp, Artificial Sequence
  Oligo Pc-fcyA N2
ttgttttgacttccccttcg

SEQ ID NO: 175 - DNA, 20 bp, Artificial Sequence
  Oligo Pc-uprt-1
ggacagtttggacaatgcag

SEQ ID NO: 176 - DNA, 40 bp, Artificial Sequence
  Oligo Pc-uprt-2
tagttctgttaccgagccggtttgaagggcaagagtccag

SEQ ID NO: 177 - DNA, 40 bp, Artificial Sequence
  Oligo Pc-uprt-3
gctctgaacgatatgctcccaccacgttgaaaggagcatc

SEQ ID NO: 178 - DNA, 20 bp, Artificial Sequence
  Oligo Pc-uprt-4
agaccgtggaagttggtcag

SEQ ID NO: 179 - DNA, 20 bp, Artificial Sequence
  Oligo Pc-uprt-N1
ttttgcaagggtcgagaaag

SEQ ID NO: 180 - DNA, 20 bp, Artificial Sequence
  Oligo Pc-uprt N2
cagttcttgccctggatctc

SEQ ID NO: 181 - DNA, 18 bp, Artificial Sequence
  Oligo Fo-uprt-1
catacgtcaccaccttgc

SEQ ID NO: 182 - DNA, 40 bp, Artificial Sequence
  Oligo Fo-uprt-2
tagttctgttaccgagccgggctgttgttagtgttcgagg

SEQ ID NO: 183 - DNA, 38 bp, Artificial Sequence
  Oligo Fo-uprt-3
gctctgaacgatatgctcccgaaggaatcagcgcaaag

SEQ ID NO: 184 - DNA, 20 bp, Artificial Sequence
  Oligo Fo-uprt-4
cacgtatagaatcacggagg

SEQ ID NO: 185 - DNA, 17 bp, Artificial Sequence
  Oligo Fo-uprt-N1
gacgccatagtgtgctc

SEQ ID NO: 186 - DNA, 18 bp, Artificial Sequence
  Oligo Fo-uprt-N2
gcttgatgcatgcactag